SEQUENCE LISTING (67 sequences)

SEQ ID NO: 1 - DNA, 20 bp, Artificial Sequence
  P5 primer
aatgatacggcgaccaccga

SEQ ID NO: 2 - DNA, 24 bp, Artificial Sequence
  P7 primer
caagcagaagacggcatacgagat

SEQ ID NO: 3 - protein, 723 aa, Thermomonas hydrothermalis
  Dipeptidyl peptidase from Thermomonas hydrothermalis (with the signal peptide)
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 4 - protein, 700 aa, Thermomonas hydrothermalis
  Dipeptidyl peptidase from Thermomonas hydrothermalis (without the signal peptide)
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 5 - protein, 710 aa, Caldithrix abyssi
  Dipeptidyl peptidase from Caldithrix abyssi (with the signal peptide)
MKIRLFGVLLLFTFSLFAEEGMYPITEIHKLNLKKLGIELSADQIFSENEVSLSDAIVQIGGCTGSFISPEGLILTNHHCAFRAIQNISSTENDYLTNGFVAHTLQEERPAKGYTVRITERVEDVSQRVLNAVKHIEDPIEREKAIEKITKQIVKEQEQKHPGKRAAVSEMFPGKTYYLFIYTYLKDVRLVYAPPRSIGEFGGEFDNWEWPRHTGDFTLMRAYVAPDGSPSDYSEENVPYRPKSYLKVAAKGVEEGDRVFILGYPGRTYRHRTSAFLAFEYEFRMPFVVDWYQWQIDLLTTLGKDDADRSLKFSSWIKGLANTEKNYRGKLQGIRRIGLLEQKKNEEEKIQVFIAENNLKKYQHVLTEIKQIYHTYRQSAVREMLLSYFGRSPVLPAVARTLVLAAEERQKEDLERERAFMDRNFKRTQTYTLLRLKNFDSQADQLILQELLKKAAALPEDQRISALRSIFKLDDAAETRQVISEAYRKTRLSDPEFVKTCFAKTPDELKALNDPLINWMLALKEDYETLKNIRKERNGKLRRLRALWLEAKQAYLKTDFIPDANGTYRMTFGFIEGYAPADAVYKAPITTGRGILEKHTGKSPFDTPEKLLALLKAKQFGPFVSKTVGTLPVGILYSCDTTGGNSGSPVLNARGQLVGLNFDRAFEATINDYAWNHQYSRSIGVDIRYILFLLKYFSGAEHLLEEMGVQ

SEQ ID NO: 6 - protein, 692 aa, Caldithrix abyssi
  Dipeptidyl peptidase from Caldithrix abyssi (without the signal peptide)
EEGMYPITEIHKLNLKKLGIELSADQIFSENEVSLSDAIVQIGGCTGSFISPEGLILTNHHCAFRAIQNISSTENDYLTNGFVAHTLQEERPAKGYTVRITERVEDVSQRVLNAVKHIEDPIEREKAIEKITKQIVKEQEQKHPGKRAAVSEMFPGKTYYLFIYTYLKDVRLVYAPPRSIGEFGGEFDNWEWPRHTGDFTLMRAYVAPDGSPSDYSEENVPYRPKSYLKVAAKGVEEGDRVFILGYPGRTYRHRTSAFLAFEYEFRMPFVVDWYQWQIDLLTTLGKDDADRSLKFSSWIKGLANTEKNYRGKLQGIRRIGLLEQKKNEEEKIQVFIAENNLKKYQHVLTEIKQIYHTYRQSAVREMLLSYFGRSPVLPAVARTLVLAAEERQKEDLERERAFMDRNFKRTQTYTLLRLKNFDSQADQLILQELLKKAAALPEDQRISALRSIFKLDDAAETRQVISEAYRKTRLSDPEFVKTCFAKTPDELKALNDPLINWMLALKEDYETLKNIRKERNGKLRRLRALWLEAKQAYLKTDFIPDANGTYRMTFGFIEGYAPADAVYKAPITTGRGILEKHTGKSPFDTPEKLLALLKAKQFGPFVSKTVGTLPVGILYSCDTTGGNSGSPVLNARGQLVGLNFDRAFEATINDYAWNHQYSRSIGVDIRYILFLLKYFSGAEHLLEEMGVQ

SEQ ID NO: 7 - protein, 178 aa, Rattus norvegicus
  Neutrophil gelatinase-associated lipocalin, Rattus norvegicus
QDSTQNLIPAPPLISVPLQPGFWTERFQGRWFVVGLAGNAVQKERQSRFTMYSTIYELQEDNSYNVTSILVRGQGCRYWIRTFVPSSRPGQFTLGNIHSYPQIQSYDVQVADTDYDQFAMVFFQKTSENKQYFKVTLYGRTKGLSDELKERFVSFAKSLGLKDNNIVFSVPTDQCIDN

SEQ ID NO: 8 - protein, 164 aa, Homo sapiens
  Prostaglandin-H2 D-isomerase, Homo sapiens
QVSVQPNFQQDKFLGRWFSAGLASNSSWLREKKAALSMAKSVVAPATDGGLNLTSTFLRKNQCETRTMLLQPAGSLGSYSYRSPHFGSTYSVSVVETDYDQYALLYSQGSKGPGEDFRMATLYSRTQTPRAELKEKFTAFSKAQGFTEDTIVFLPQTDKCMTEQ

SEQ ID NO: 9 - protein, 175 aa, Sus scrofa
  Salivary lipocalin (SAL), Sus scrofa
HKEAGQDVVTSNFDASKIAGEWYSILLASDAKENIEENGSMRVFVEHIRVLDNSSLAFKFQRKVNGECTDFYAVSDKVGDGVYTVAYYGENKFRLLEVNYSDYVILHLVDVNGDKTFQLMEFYGRKPDVEPKLKDKFVEISQQYGIIKENIIDLTKIDRCFQLRGSGGVQESSAE

SEQ ID NO: 10 - protein, 156 aa, Coturnix japonica
  Extracellular fatty acid-binding protein
TVPDRSEIAGKWYVVALASNTEFFLREKDKMKMAMARISFLGEDELKVSYAVPKPNGCRKWETTFKKTSDDGEVYYSEEAKKKVEVLDTDYKSYAVIYATRVKDGRTLHMMRLYSRSPEVSPAATAIFRKLAGERNYTDEMVAMLPRQEECTVDEV

SEQ ID NO: 11 - protein, 154 aa, Homo sapiens
  Lipocalin-1 (Tear lipocalin)
ASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYSEGELHGKPVRGVKLVGRDPKNNLEALEDFEKAAGARGLSTESILIPRQSETCSPGSD

SEQ ID NO: 12 - protein, 179 aa, Rhodnius prolixus
  Nitrophorin-2
DCSTNISPKQGLDKAKYFSGKWYVTHFLDKDPQVTDQYCSSFTPRESDGTVKEALYHYNANKKTSFYNIGEGKLESSGLQYTAKYKTVDKKKAVLKEADEKNSYTLTVLEADDSSALVHICLREGSKDLGDLYTVLTHQKDAEPSAKVKSAVTQAGLQLSQFVGTKDLGCQYDDQFTSL

SEQ ID NO: 13 - protein, 183 aa, Homo sapiens
  Alpha-1-acid glycoprotein 2
QIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLRIPRSDVMYTDWKKDKCEPLEKQHEKERKQEEGES

SEQ ID NO: 14 - protein, 174 aa, Homo sapiens
  Protein AMBP
GPVPTPPDNIQVQENFNISRIYGKWYNLAIGSTSPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQ

SEQ ID NO: 15 - protein, 173 aa, Homo sapiens
  Complement component C8 gamma chain
ASPISTIQPKANFDAQQFAGTWLLVAVGSAARFLQEQGHRAEATTLHVAPQGTAMAVSTFRKLDGICWQVRQLWGDTGVLGRFLLQARGARGAVHVVVAETDYQSFAVLYLERAGQLSVKLYARSLPVSDSVLSGFEQRVQEAHLTEDQIFYFPKYGFCEAADQFHVLDEVRR

SEQ ID NO: 16 - protein, 124 aa, Bos taurus
  Odorant-binding protein
AQEEEAEQNLSELSGPWRTVYIGSTNPEKIQENGPFRTYFRELVFDDEKGTVDFYFSVKRDGKWKNVHVKATKQDDGTYVADYEGQNVFKIVSLSRTHLVAHNINVDKHGQTTELTELFVKLNV

SEQ ID NO: 17 - protein, 173 aa, Gallus gallus
  Retinol-binding protein 4
DCRVSSFKVKENFDKNRYSGTWYAMAKKDPEGLFLQDNVVAQFTVDENGQMSATAKGRVRLFNNWDVCADMIGSFTDTEDPAKFKMKYWGVASFLQKGNDDHWVVDTDYDTYALHYSCRELNEDGTCADSYSFVFSRDPKGLPPEAQKIVRQRQIDLCLDRKYRVIVHNGFCS

SEQ ID NO: 18 - protein, 156 aa, Bos taurus
  Allergen Bos d 2
QETPAEIDPSKIPGEWRIIYAAADNKDKIVEGGPLRNYYRRIECINDCESLSITFYLKDQGTCLLLTEVAKRQEGYVYVLEFYGTNTLEVIHVSENMLVTYVENYDGERITKMTEGLAKGTSFTPEELEKYQQLNSERGVPNENIENLIKTDNCPP

SEQ ID NO: 19 - protein, 190 aa, Mus musculus
  Neutrophil gelatinase-associated lipocalin
QDSTQNLIPAPSLLTVPLQPDFRSDQFRGRWYVVGLAGNAVQKKTEGSFTMYSTIYELQENNSYNVTSILVRDQDQGCRYWIRTFVPSSRAGQFTLGNMHRYPQVQSYNVQVATTDYNQFAMVFFRKTSENKQYFKITLYGRTKELSPELKERFTRFAKSLGLKDDNIIFSVPTDQCIDNSAWSHPQFEK

SEQ ID NO: 20 - protein, 184 aa, Homo sapiens
  Human alpha(1)-acid glycoprotein (hAGP)
MQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLRIPRSDVMYTDWKKDKCEPLEKQHEKERKQEEGES

SEQ ID NO: 21 - protein, 164 aa, Artificial Sequence
  Prostaglandin-H2 D-isomerase mutant
QVSVQPNFQQDKFLGRWFYAGHASNSSWYREKVAALSMAKLVVAPATDGGLNATETFLRKNQCETITSLLQPAGSLGSYSSRSPHGGSTWSTSVVETDYDQYALLYLQGSKGPGEDFRGAILASRTQTPRAELKEKFTAFSKAQGFTEDTIVFLPQTDKCMTEQ

SEQ ID NO: 22 - protein, 164 aa, Artificial Sequence
  Prostaglandin-H2 D-isomerase mutant
QVSVQPNFQQDKFLGRWFTAGLASNSSWLREKFAALSHAKTVVAPATDGGLNLTVTVLRKNQCETSTLLLQPAGSLGSYSRRSPHVGSTGSVSVVETDYDQYALIYDQGSKGPGEDWRIAELVSRTQTPRAELKEKFTAFSKAQGFTEDTIVFLPQTDKCMTEQ

SEQ ID NO: 23 - protein, 164 aa, Artificial Sequence
  Prostaglandin-H2 D-isomerase mutant
QVSVQPNFQQDKFLGRWFLAGAASNSSWLREKNAALSYAKLVVAPATDGGLNSTVTRLRKNQCETETRLLQPAGSLGSYSWRSPHLGSTLSLSVVETDYDQYALVYAQGSKGPGEDVRGAELLSRTQTPRAELKEKFTAFSKAQGFTEDTIVFLPQTDKCMTEQ

SEQ ID NO: 24 - protein, 164 aa, Artificial Sequence
  Prostaglandin-H2 D-isomerase mutant
QVSVQPNFQQDKFLGRWFYAGLASNSSWLREKCAALSSAKAVVAPATDGGLNVTETWLRKNQCETGTELLQPAGSLGSYSFRSPHRGSTLSLSVVETDYDQYALLYRQGSKGPGEDERRALLMSRTQTPRAELKEKFTAFSKAQGFTEDTIVFLPQTDKCMTEQ

SEQ ID NO: 25 - protein, 164 aa, Artificial Sequence
  Prostaglandin-H2 D-isomerase mutant
QVSVQPNFQQDKFLGRWFIAGLASNSSWVREKSAALSQAKVVVAPATDGGLNVTVTVLRKNQCETYTVLLQPAGSLGSYSSRSPHFGSTVSLSVVETDYDQYALWYAQGSKGPGEDMRHAELWSRTQTPRAELKEKFTAFSKAQGFTEDTIVFLPQTDKCMTEQ

SEQ ID NO: 26 - protein, 164 aa, Artificial Sequence
  Prostaglandin-H2 D-isomerase mutant
QVSVQPNFQQDKFLGRWFAAGYASNSSWRRERSAALSMAKVVVAPATDGGLNWTETILRKNQCETATNLLQPAGSLGSYSIRSPHAGSTSSMSVVETDYDQYALLYAQGSKGPGEDFRLAILLSRTQTPRAELKEKFTAFSKAQGFTEDTIVFLPQTDKCMTEQ

SEQ ID NO: 27 - protein, 164 aa, Artificial Sequence
  Prostaglandin-H2 D-isomerase mutant
QVSVQPNFQQDKFLGRWFIAGQASNSSWQREKCAALSRAKVVVAPATDGGLNLTMTLLRKNQCETRTTLLQPAGSLGSYSYRSPHLGSTVSWSVVETDYDQYALMYVQGSKGPGEDQRFAILWSRTQTPRAELKEKFTAFSKAQGFTEDTIVFLPQTDKCMTEQ

SEQ ID NO: 28 - protein, 164 aa, Artificial Sequence
  Prostaglandin-H2 D-isomerase mutant
QVSVQPNFQQDKFLGRWFLAGMASNSSWMREKFAALSVAKFVVAPATDGGLNTTTTLLRKNQCETFTRLLQPAGSLGSYSSRSPHLGSTVSGSVVETDYDQYALLYEQGSKGPGEDGRKAWLGSRTQTPRAELKEKFTAFSKAQGFTEDTIVFLPQTDKCMTEQ

SEQ ID NO: 29 - protein, 164 aa, Artificial Sequence
  Prostaglandin-H2 D-isomerase mutant
QVSVQPNFQQDKFLGRWFVAGVASNSSWMREKVAALSGAKVVVAPATDGGLNYTETWLRKNQCETWTGLLQPAGSLGSYSQRSPHGGSTNSLSVVETDYDQYALLYVQGSKGPGEDPRVAVLWSRTQTPRAELKEKFTAFSKAQGFTEDTIVFLPQTDKCMTEQ

SEQ ID NO: 30 - protein, 178 aa, Artificial Sequence
  Neutrophil gelatinase-associated lipocalin mutant
QDSTQNLIPAPPLISVPLQPGFWTERFQGRWFNVGIAGNAVQKERQSRFTMLSLISELQEDNSYNWTLISVRGQGCRYRILTWVPSSRPGQFTLGNIHSYPQIQSLDVQVADTDYDQFAMVFAQGTSENKQYFEVILMGRTKGLSDELKERFVSFAKSLGLKDNNIVFSVPTDQCIDN

SEQ ID NO: 31 - protein, 178 aa, Artificial Sequence
  Neutrophil gelatinase-associated lipocalin mutant
QDSTQNLIPAPPLISVPLQPGFWTERFQGRWFLVGFAGNAVQKERQSRLTMQSLILELQEDNSYNITAINVRGQGCRYIISTLVPSSRPGQFTLGNIHSYPQIQSLDVQVADTDYDQFAMVFLQGTSENKQYFEVALAGRTKGLSDELKERFVSFAKSLGLKDNNIVFSVPTDQCIDN

SEQ ID NO: 32 - protein, 178 aa, Artificial Sequence
  Neutrophil gelatinase-associated lipocalin mutant
QDSTQNLIPAPPLISVPLQPGFWTERFQGRWFSVGMAGNAVQKERQSRFTMGSCIMELQEDNSYNLTFIGVRGQGCRYRILTWVPSSRPGQFTLGNIHSYPQIQSADVQVADTDYDQFAMVFWQTTSENKQYFAVELWGRTKGLSDELKERFVSFAKSLGLKDNNIVFSVPTDQCIDN

SEQ ID NO: 33 - protein, 154 aa, Artificial Sequence
  Lipocalin-1 (Tear lipocalin) mutant, M=M44
ASDEEIQDVSGTWYLKASTVDRERLATWLESNTPMTLTTLEGGNLEAKGTRLISGRCAETKAVLEKTDEPGKYTTDDTKAVAYIIRSHVKDHYIFYEEGELHGKPWRSVSLVGRDPKNNLEALEDFEKAAGARGLSTESILIPRQSETCSPGSD

SEQ ID NO: 34 - protein, 154 aa, Artificial Sequence
  Lipocalin-1 (Tear lipocalin) mutant, M=M52
ASDEEIQDVSGTWYLKASTVDREFLFFIDESVTPMTLTTLEGGNLEAKATYLISGRCAEWKAVLEKTDEPGKYTLDTPKLVAYIIRSHVKDHYIFYGEGELHGKPLRFVRLVGRDPKNNLEALEDFEKAAGARGLSTESILIPRQSETCSPGSD

SEQ ID NO: 35 - protein, 154 aa, Artificial Sequence
  Lipocalin-1 (Tear lipocalin) mutant, M=M52
ASDEEIQDVSGTWYLKAATVDRERTSLLAESQTPMTLTTLEGGNLEAKLTRLISGRCTEWKAVLEKTDEPGKYTHDFAKAVAYIIRSHVKDHYIFYGEGELHGKPWRSVRLVGRDPKNNLEALEDFEKAAGARGLSTESILIPRQSETCSPGSD

SEQ ID NO: 36 - protein, 178 aa, Artificial Sequence
  Neutrophil gelatinase-associated lipocalin mutant, M=M44
QDSTQNLIPAPPLISVPLQPGFWTERFQGRWFIVGAAGNAVQKERQSRVTMGSVIWELQEDNSYNLTFISVRGQGCRYWIETWVPSSRPGQFTLGNIHSYPQIQSADVQVADTDYDQFAMVFVQYTSENKQYFMVRLLGRTKGLSDELKERFVSFAKSLGLKDNNIVFSVPTDQCIDN

SEQ ID NO: 37 - protein, 178 aa, Artificial Sequence
  Neutrophil gelatinase-associated lipocalin mutant, M=M44
QDSTQNLIPAPPLISVPLQPGFWTERFQGRWFLVGLAGNAVQKERQSRRTMSSLILELQEDNSYNETWINVRGQGCRYRIGTVVPSSRPGQFTLGNIHSYPQIQSLDVQVADTDYDQFAMVFTQFTSENKQYFYVRLMGRTKGLSDELKERFVSFAKSLGLKDNNIVFSVPTDQCIDN

SEQ ID NO: 38 - protein, 164 aa, Artificial Sequence
  Prostaglandin-H2 D-isomerase mutant, M=M44
QVSVQPNFQQDKFLGRWFPAGFASNSSWDREKPAALSLAKMVVAPATDGGLNWTETYLRKNQCETTTHLLQPAGSLGSYSYRSPHFGSTRSLSVVETDYDQYALFYSQGSKGPGEDHRGAWLLSRTQTPRAELKEKFTAFSKAQGFTEDTIVFLPQTDKCMTEQ

SEQ ID NO: 39 - protein, 164 aa, Artificial Sequence
  Prostaglandin-H2 D-isomerase mutant, M=M44
QVSVQPNFQQDKFLGRWFLAGLASNSSWAREKIAALSDAKFVVAPATDGGLNGTVTVLRKNQCETATGLLQPAGSLGSYSWRSPHDGSTLSLSVVETDYDQYALVYEQGSKGPGEDLRIAALWSRTQTPRAELKEKFTAFSKAQGFTEDTIVFLPQTDKCMTEQ

SEQ ID NO: 40 - protein, 164 aa, Artificial Sequence
  Prostaglandin-H2 D-isomerase mutant, M=M44
QVSVQPNFQQDKFLGRWFYAGLASNSSWIREKYAALSLAKSVVAPATDGGLNWTETSLRKNQCETGTVLLQPAGSLGSYSLRSPHHGSTISVSVVETDYDQYALVYRQGSKGPGEDSRRAILMSRTQTPRAELTEKFTAFSKAQGFTEDTIVFLPQTDKCMTEQ

SEQ ID NO: 41 - protein, 164 aa, Artificial Sequence
  Prostaglandin-H2 D-isomerase mutant, M=M44
QVSVQPNFQQDKFLGRWFYAGMASNSSWLREKSAALSAAKFVVAPATDGGLNFTETFLRKNQCETTTILLQPAGSLGSYSVRSPHVGSTVSISVVETDYDQYALFYVQGSKGPGEDNRMALLGSRTQTPRAELKEKFTAFSKAQGFTEDTIVFLPQTDKCMTEQ

SEQ ID NO: 42 - protein, 164 aa, Artificial Sequence
  Prostaglandin-H2 D-isomerase mutant, M=M44
QVSVQPNFQQDKFLGRWFVAGLASNSSWDREKVAALSGAKMVVAPATDGGLNVTRTMLRKNQCETYTGLLQPAGSLGSYSWRSPHPGSTGSESVVETDYDQYALVYGQGSKGPGEDLRAAALWSRTQTPRAELKEKFTAFSKAQGFTEDTIVFLPQTDKCMTEQ

SEQ ID NO: 43 - protein, 156 aa, Artificial Sequence
  Extracellular fatty acid-binding protein mutant, M=M44
TVPDRSEIAGKWYVVALASNTEFVLREKDWLKLAMARISFLGEDELKVSMTTPGPNGCTKWERTLKKTSDDGEVYSSPRAKEKVEVLDTDYKSYAVIYATAVKDGRTLHEMWLYSRSPEVSPAATAIFRKLAGERNYTDEMVAMLPRQEECTVDEV

SEQ ID NO: 44 - protein, 156 aa, Artificial Sequence
  Extracellular fatty acid-binding protein mutant, M=M44
TVPDRSEIAGKWYVVALASNTEFFLREKDRIKTAMARISFLGEDELKVSLARPAPNGCHKAEWTFKKTSDDGEVYTSFLAKFKVEVLDTDYKSYAVIYATPVKDGRTRHEMLLYSRSPEVSPAATAIFRKLAGERNYTDEMVAMLPRQEECTVDEV

SEQ ID NO: 45 - protein, 156 aa, Artificial Sequence
  Extracellular fatty acid-binding protein mutant, M=M44
TVPDRSEIAGKWYVVALASNTEFLLREKDSLKSAMARISFLGEDELKVSYARPLPNGCLKLEATMKKTSDDGEVYLSRSAKLKVEVLDTDYKSYAVIYATPVKDGRTRHYMALYSRSPEVSPAATAIFRKLAGERNYTDEMVAMLPRQEECTVDEV

SEQ ID NO: 46 - protein, 156 aa, Artificial Sequence
  Extracellular fatty acid-binding protein mutant, M=M44
TVPDRSEIAGKWYVVALASNTEFHLREKDNLKVAMARISFLGEDELKVSEAVPTPNGCMKHESTWKKTSDDGEVYRSRKAKQKVEVLDTDYKSYAVIYATRVKDGRTHHWMLLYSRSPEVSPAATAIFRKLAGERNYTDEMVAMLPRQEECTVDEV

SEQ ID NO: 47 - protein, 156 aa, Artificial Sequence
  Extracellular fatty acid-binding protein mutant, M=M44
TVPDRSEIAGKWYVVALASNTEFFLREKDNFKMAMARISFLGEDELKVSEAVPTPNGCFKAEFTGKKTSDDGEVYWSQTAKYKVEVLDTDYKSYAVIYATNVKDGRTVHAMNLYSRSPEVSPAATAIFRKLAGERNYTDEMVAMLPRQEECTVDEV

SEQ ID NO: 48 - protein, 190 aa, Artificial Sequence
  Neutrophil gelatinase-associated lipocalin mutant, M= M2014
QDSTQNLIPAPSLLTVPLQPDFRSDQFRGRWYVVGLAGNAVQKKTEGSFTMLSLIYELQENNSYNVTAIFVRDQDQGCRYVIDTLVPSSRAGQFTPGSMGRFPQVQSYNVQVATTDYNQFAMVFVRVTSENKQYFEITLLGRTKELSPELKERFTRFAKSLGLKDDNIIFSVPTDQCIDNSAWSHPQFEK

SEQ ID NO: 49 - protein, 164 aa, Artificial Sequence
  Prostaglandin-H2 D-isomerase mutant, M=M12
QVSVQPNFQQDKFLGRWFVAGIASNSSWSREKIAALSFAKGVVAPATDGGLNYTETVLRKNQCETQTVLLQPAGSLGSYSARSPHQGSTRSFSVVETDYDQYALLYAQGSKGPGEDDRVAGLWSRTQTPRAELKEKFTAFSKAQGFTEDTIVFLPQTDKCMTEQ

SEQ ID NO: 50 - protein, 174 aa, Artificial Sequence
  Protein AMBP mutant, M=M12
GPVPTPPDNIQVQENFNISRIYGKWYPLAMGSTSPYLKKSMDIMTPSYLVLGEGATEAEISWTETFWRKGVCEEGSGALEKTDTDGKFLYHKSKWNITEERYVVHTNYDEYAIVLVKKFSRHHGPTIEAVLMGRAPQLRETLLQDFRVVAQGVGIPEDSIFTMADRGECVPGEQ

SEQ ID NO: 51 - protein, 183 aa, Artificial Sequence
  Human alpha(1)-acid glycoprotein mutant, M=M90
QIPLCANLVPVPITNATLDRITGKWFHIASAIRNEEYNKSVQRQQSTHFYITPNKTEDTIFTRVYQTRQNQCFYNSSYLNVQRENGTVSLYSGGREVVGHLLFLRDTKTLMWGTYLDDEKNWGLEFWADKPETTKEQLGEFYEALDCLRIPRSDVMYTDWKKDKCEPLEKQHEKERKQEEGES

SEQ ID NO: 52 - protein, 183 aa, Artificial Sequence
  Human alpha(1)-acid glycoprotein mutant, M=M90
QIPLCANLVPVPITNATLDRITGKWFIIASASRNEEYNKSVQQAQSTAFYITPNKTEDTIFLRRYQTRQNQCFYNSSYLNVQRENGTVSYYRGGRERVSHLLFLRDTKTLMIGSYLDDEKNWGLVFGADKPETTKEQLGEFYEALDCLRIPRSDVMYTDWKKDKCEPLEKQHEKERKQEEGES

SEQ ID NO: 53 - protein, 183 aa, Artificial Sequence
  Human alpha(1)-acid glycoprotein mutant, M=M90
QIPLCANLVPVPITNATLDRITGKWFLIASASRNEEYNKSVQGSQNTAFYFTPNKTEDTIFLRRYQTRQNQCFYNSSYLNVQRENGTVSSYFGGRENVIHLLFLRDTKTLMLGRYLDDEKNWGLEFYADKPETTKEQLGEFYEALDCLRIPRSDVMYTDWKKDKCEPLEKQHEKERKQEEGES

SEQ ID NO: 54 - protein, 183 aa, Artificial Sequence
  Human alpha(1)-acid glycoprotein mutant, M=M90
QIPLCANLVPVPITNATLDRITGKWFVIASATRNEEYNKSVQRQQGTVFYYTPNKTEDTIFVRRYQTRQNQCFYNSSYLNVQRENGTVSFYEGGRELVFHLLFLRDTKTLMWGLYLDDEKNWGLEFFADKPETTKEQLGEFYEALDCLRIPRSDVMYTDWKKDKCEPLEKQHEKERKQEEGES

SEQ ID NO: 55 - protein, 183 aa, Artificial Sequence
  Human alpha(1)-acid glycoprotein mutant, M=M90
QIPLCANLVPVPITNATLDRITGKWFRIASAFRNEEYKKSVQEVQTTSFYFTPNKTEDTIFARFYQTRQNQCFYNSSYLNVQRENGTVSLYSGGREAVLHLLFLRDTKTLMLGVYLDDEKNWGLSFYADKPETTKEQLGEFYEALDCLRIPRSDVMYTDWKKDKCEPLEKQHEKERKQEEGES

SEQ ID NO: 56 - protein, 183 aa, Artificial Sequence
  Human alpha(1)-acid glycoprotein mutant, M=M90
QIPLCANLVPVPITNATLDRITGKWFLIASAIRNEEYNKSVQAVQSTAFYWTPNKTEDTIFARYYQTRQNQCFYNSSYLNVQRENGTVSWYAGGREFVSHLLFLRYTKTLMWGGYLDDEKNWGLEFYADKPETTKEQLGEFYEALDCLRIPRSDVMYTDWKKDKCEPLEKQHEKERKQEEGES

SEQ ID NO: 57 - protein, 175 aa, Artificial Sequence
  Salivary lipocalin mutant, M=M12
HKEAGQDVVTSNFDASKIAGEWYSILLASDAKENIEENGVGRMFVEHIRVLDNSSLAMKMQRKVNGECTDYYAVWDKVGDGVYTNALGGEAKLRLLEVNYSDYVIMHGVDVNGDKTFQYMMFSGRKPDVEPKLKDKFVEISQQYGIIKENIIDLTKIDRCFQLRGSGGVQESSAE

SEQ ID NO: 58 - protein, 164 aa, Artificial Sequence
  Prostaglandin-H2 D-isomerase mutant, M=M12
QVSVQPNFQQDKFFGRWFVAGIASNSSWSREKIAALSFVKGVVAPATDGGLNYTETVLRKNQCETQTVLLQPAGSLGSYSARSLHQGSTRSFSVVETDYDQYALLYAQGSKGPGEDDRVAGLWSRTQTPRAELKEKFTAFSKAQGFTGDTIVFLPQTDKCMTEQ

SEQ ID NO: 59 - protein, 164 aa, Artificial Sequence
  Prostaglandin-H2 D-isomerase mutant, M=M12
QVSVQPNFQQDKFLGRWFVAGLASNSSWHREKSAALSTAKTVVAPATDGGLNFTWTPLRKNQCETVTALLQPAGSLGSYSSRSPHLGSTGSLSVVETDYDQYALMYFQGSKGPGEDLREAALFSRTQTPRAELKEKFTAFSKAQGFTEDTIVFLPQTDKCMTEQ

SEQ ID NO: 60 - protein, 190 aa, Artificial Sequence
  Neutrophil gelatinase-associated lipocalin mutant, M=M12
QDSTQNLIPAPSLLTVPLQPDFRSDQFRGRWYTVGLAGNAVQKKTEGSFTMSSSIYELQENNSYNVTTITVRDQDQGCGYRIETAVPSSRAGQFTPGIMMGNPQVQSTNVQVATTDYNQFAMVFYRLTSENKQYFNITLLGRTKELSPELKERFTRFAKSLGLKDDNIIFSVPTDQCIDNSAWSHPQFEK

SEQ ID NO: 61 - protein, 156 aa, Artificial Sequence
  Extracellular fatty acid-binding protein mutant, M=M17
TVPDRSEIAGKWYVVALASNTEFYLREKDWVKSAMARISFLGEDELKVSSAVPSPNGCYKVERTWKKTSDDGEVYNSRAAKLKVEVLDTDYKSYAVIYATRVKDGRTDHQMILYSRSPEVSPAATAIFRKLAGERNYTDEMVAMLPRQEECTVDEV

SEQ ID NO: 62 - protein, 156 aa, Artificial Sequence
  Extracellular fatty acid-binding protein mutant, M=M17
TVPDRSEIAGKWYVVALASNTEFYLRGKDRVKSAMVRISFQGEDELKVSSAVPSPNGCYKVERTWKKTSDDGEVYNSRAAKLKVEVLDTDYKSYAVIYATRVKDRRTDHQMILYSRSPDVSPAATAIFRKLAGERNYTDEMVAMLPRQEECTVDEV

SEQ ID NO: 63 - protein, 259 aa, Homo sapiens
  Carbonic anhydrase 2
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 64 - protein, 259 aa, Artificial Sequence
  Carbonic anhydrase 2 mutant, M=M64
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVRQPDGLAVLGIFLKVGSAKPGLQKVVDVLDSIKTKGKTADFTNFDPRGLLPESLDYWTYPGSRTTPPLLESVTWIVLKEPISVSSEQVLKFRKLNFNGEGEPEELMVDNWRPAQPLKNRQIKASFK

SEQ ID NO: 65 - protein, 259 aa, Artificial Sequence
  Carbonic anhydrase 2 mutant, M=M64
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGTAKPGLQKVVDVLDSIKTRGKSADFTNFDPRGLLPESLDYWTYPGSRTTPPLLESVTWIVLKEPISVSSEQVLKFRKLNFNGEGEPEELMVDNWRPAQPLKNRQIKASFK

SEQ ID NO: 66 - protein, 259 aa, Artificial Sequence
  Carbonic anhydrase 2 mutant, M=M64
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQHPDGLAVLGIFLKVGSAKPGLQKVVDVLDSIKTKGKSADFTNFDPRGLLPESLDYWTYPGSRTTPPLLESVTWIVLKEPISVSSEQVLKFRKLNFNGEGEPEELMVDNWRPAQPLKNRQIKASFK

SEQ ID NO: 67 - protein, 7 aa, Artificial Sequence
  Test peptide
IHAGYAW

(19) United States
(12) Patent Application Publication
(10) Pub. No.: US 2023/0220589 A1
(43) Pub. Date: Jul. 13, 2023

(54) POLYPEPTIDE TERMINAL BINDERS AND USES THEREOF

(71) Applicant: Encodia, Inc., San Diego, CA (US)

(72) Inventors: Kevin L. GUNDERSON, San Diego, CA (US); Soumya GANGULY, San Diego, CA (US); Robert C. JAMES, San Diego, CA (US); Kenneth KUHN, San Diego, CA (US); Zachary MILES, San Diego, CA (US); Lei SHI, San Diego, CA (US); Stephen VERESPY, III, San Diego, CA (US); Aaron WISE, San Diego, CA (US); Zongxiang ZHOU, San Diego, CA (US)

(73) Assignee: Encodia, Inc., San Diego, CA (US)

(21) Appl. No.: 17/539,033

(22) PCT Filed: Sep. 29, 2021
(86) PCT No.: PCT/US21/52739

§ 371 Date: Nov. 30, 2021

Related U.S. Application Data
(60) Provisional application No. 63/085,977, filed on Sep. 30, 2020.

(51) Int. Cl. C40B 40/10

(52) U.S. Cl. CPC C40B 40/10

(57) ABSTRACT

The present disclosure relates to a binder that specifically binds to an N-terminally modified polypeptide through interaction with a modified N-terminal amino acid. Also provided herein is a method and related kits for treating a polypeptide using or comprising the binder and/or modified cleavase. In some embodiments, also provided herein is a method and related kits for transferring information using a plurality of enzymes, including for performing a ligation, extension, and cleavage reaction with nucleic acid molecules associated with the polypeptide for analysis.

### CROSS-REFERENCE TO RELATED APPLICATIONS

The present application claims priority to U.S. provisional patent application No. 63/085,977, filed on Sep. 30, 2020, the disclosure and content of which is incorporated herein by reference in its entirety for all purposes.

### STATEMENT REGARDING FEDERALLY SPONSORED RESEARCH

This invention was made with Government support awarded by National Institute of General Medical Sciences of the National Institutes of Health under Grant Numbers R43GM130185 and R44GM123836. The United States Government has certain rights in this invention pursuant to this grant.

### SEQUENCE LISTING ON ASCII TEXT

This patent or application file contains a Sequence Listing submitted in computer readable ASCII text format (file name: 4614-2002840 SeqList ST25.txt, date recorded: September 29, 2021, size: 119,054 bytes). The content of the Sequence Listing file is incorporated herein by reference in its entirety.

### TECHNICAL FIELD

This disclosure generally relates to biotechnology, and in particular to analysis of peptides, polypeptides, and protein employing or using N-terminal modifying reagents, N-terminal binding agents, and N-terminal cleavases for analyzing, sequencing or cyclic sequencing of polypeptides or peptides.

### BACKGROUND

Protein expression, post-translational modifications (PTMs), and interactions all play key roles in cell biology. Understanding the proteome is essential to elucidate the molecular mechanisms and cellular processes involved in signal transduction, cell differentiation, and disease. Improved tools for proteomics are likely to greatly speed the development of novel therapeutics, vaccines, and diagnostics. As such, there is an acute need for scalable proteomics technologies that can transform healthcare by providing access to the “personal proteome”.

High-throughput technologies such as Next Generation Sequencing (NGS) have transformed research. Despite advances in Mass Spectroscopy (MS), corresponding innovation in proteomics is needed to have a similar broad ranging impact on biomedical research. Yet this protein information is direly needed for a better understanding of proteome dynamics in health and disease and to help enable precision medicine. As such, there is great interest in developing “next-generation” tools to miniaturize and highly-parallelize collection of this proteomic information.

Highly-parallel macromolecular characterization and recognition of proteins is challenging for several reasons. The use of affinity-based assays is often difficult due to several key challenges. One significant challenge is multiplexing the readout of a collection of affinity agents to a collection of cognate macromolecules; another challenge is minimizing cross-reactivity between the affinity agents and off-target macromolecules; a third challenge is developing an efficient high-throughput read out platform. An example of this problem occurs in proteomics in which one goal is to identify and quantitate most or all the proteins in a sample. Additionally, it is desirable to characterize various post-translational modifications (PTMs) on the proteins at a single molecule level. Currently this is a formidable task to accomplish in a high-throughput way.

Molecular recognition and characterization of a protein or peptide macromolecule is typically performed using an immunoassay. There are many different immunoassay formats including ELISA, multiplex ELISA (e.g., spotted antibody arrays, liquid particle ELISA arrays), digital ELISA, reverse phase protein arrays (RPPA), and many others. These different immunoassay platforms all face similar challenges including the development of high affinity and highly-specific (or selective) antibodies (binding agents), limited ability to multiplex at both the sample and analyte level, limited sensitivity and dynamic range, and cross-reactivity and background signals. Binding agent agnostic approaches such as direct protein characterization via peptide sequencing (Edman degradation or Mass Spectroscopy) provide useful alternative approaches. However, neither of these approaches is very parallel or high-throughput.

In the last 10-15 years, peptide analysis using MALDI, electrospray mass spectroscopy (MS), and LC-MS/MS has largely replaced Edman degradation. Despite the recent advances in MS instrumentation, MS still suffers from several drawbacks including high instrument cost, requirement for a sophisticated user, poor quantification ability, and limited ability to make measurements spanning the dynamic range of the proteome. For example, since proteins ionize at different levels of efficiencies, absolute quantitation and even relative quantitation between sample is challenging. The implementation of mass tags has helped improve relative quantitation, but requires labeling of the proteome. Dynamic range is an additional complication in which concentrations of proteins within a sample can vary over a very large range (over 10 orders for plasma). MS typically only analyzes the more abundant species, making characterization of low abundance proteins challenging.

Recently, methods have been disclosed that utilize use of binding agents for high-throughput polypeptide sequencing, for example, U.S. Pat. No. 9,435,810 B2, WO2010065531A1, US 20190145982 A1, US 20200217853 A1, US 20200348308 A1, US 20200400677 A1. These methods utilize N-terminal amino acid (NTAA) recognition by binding agents as a critical step in a polypeptide sequencing assay. A number of methods to evolve specific NTAA binders from different scaffolds for recognizing a particular terminal amino acid have also been proposed, including directed evolution approaches to derive amino acyl tRNA synthetases, N-recognins such as ClpS and ClpS2, and aminopeptidases, which are disclosed, for example, in US 20190145982 A1, US 20210079398 A1 and U.S. Pat. No. 9,435,810 B2. However, identifying binding agents that afford amino acid specificity with sufficiently strong affinity has proven challenging. Binding affinity and/or specificity towards a terminal amino acid residue (P1) can vary depending on neighboring amino acids of the polypeptide to be analyzed, e.g. the penultimate terminal amino acid residue (P2) and the antepenultimate amino acid residue (P3). In some cases, crosslinking reagents and methods exist for applications involving binding agents recognizing polypeptides. It may be preferred that binding agents and detection assays are performed in a manner that allows specificity and stability in a controllable manner that allows processing of a plurality of binding agents and polypeptides at the same time. Additionally, speed and reversibility may also be a desired feature for these binding reactions. However, current reagents and methods are somewhat limited in these aspects.

Accordingly, there remains a need in the art for improved techniques relating to polypeptide sequencing and/or analysis, as well as to products, methods and kits for accomplishing the same. There is a need for proteomics technology that is highly-parallelized, accurate, sensitive, and high-throughput. The present disclosure describes the development of polypeptide or peptide sequencing reagents and methods that fulfills these and other needs.

These and other aspects of the invention will be apparent upon reference to the following detailed description. To this end, various references are set forth herein which describe in more detail certain background information, procedures, compounds and/or compositions, and are each hereby incorporated by reference in their entireties.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

The present disclosure relates to a binder that specifically binds to an N-terminally modified polypeptide via interaction with modified N-terminal amino acid (NTAA) residue of the polypeptide. Also provided herein is a method and related kits for treating a polypeptide using or comprising the binder and/or the modified or engineered cleavase. In some embodiments, also provided herein is a method and related kits for transferring information using a plurality of enzymes, including for performing a ligation, extension, and cleavage reaction with nucleic acid molecules associated with the polypeptide for analysis.

In one embodiment, provided herein is a binder that specifically binds to an N-terminally modified target polypeptide, wherein: said N-terminally modified target polypeptide is derived from a target polypeptide and said N-terminally modified target polypeptide has a formula: M-P1-P2-polypeptide, said M being an N-terminal modification, said P1 being the N-terminal amino acid residue of said target polypeptide, and P2 being a penultimate terminal amino acid residue of said target polypeptide; and said binder specifically binds to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

In another embodiment, provided herein is an engineered binder that specifically binds to an N-terminally modified target polypeptide modified by an N-terminal modifier agent, wherein: (i) the N-terminally modified target polypeptide has a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; (ii) the engineered binder specifically binds to the N-terminally modified target polypeptide through interaction between the engineered binder and the M-P1 of the N-terminally modified target polypeptide; and (iii) the engineered binder comprises an amino acid sequence having at least about 80% sequence identity to the amino acid sequence selected from the group consisting of SEQ ID NO: 7—SEQ ID NO: 20.

In yet another embodiment, provided herein is an isolated nucleic acid molecule comprising a polynucleotide having a sequence encoding the engineered binder described in the previous paragraph.

In yet another embodiment, provided herein is a set of engineered binders, comprising at least two engineered binders, wherein: (i) each engineered binder from the set of engineered binders is configured to specifically bind to an N-terminally modified target polypeptide modified with an N-terminal modifier agent and having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; (ii) each engineered binder from the set of engineered binders is configured to specifically bind to the N-terminally modified target polypeptide through interaction between the engineered binder and the modified NTAA residue of the N-terminally modified target polypeptide, wherein engineered binders from the set of engineered binders are configured to specifically bind to different modified NTAA residues of target polypeptides modified with the same or different N-terminal modifier agents; and (iii) at least one engineered binder from the set of engineered binders comprises an amino acid sequence having at least about 80% sequence identity to the amino acid sequence selected from the group consisting of SEQ ID NO: 7—SEQ ID NO: 20.

In yet another embodiment, provided herein is a method of treating a target polypeptide, the method comprises: contacting a target polypeptide with an N-terminal modifier agent to form an N-terminally modified polypeptide having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; and contacting an engineered binder with the N-terminally modified target polypeptide to allow the engineered binder to specifically bind to the N-terminally modified target polypeptide through interaction between the engineered binder and the modified NTAA residue of the N-terminally modified target polypeptide, wherein the engineered binder comprises an amino acid sequence having at least about 80% sequence identity to the amino acid sequence selected from the group consisting of SEQ ID NO: 7—SEQ ID NO: 20.

In yet another embodiment, provided herein is a modified or an engineered cleavase comprising a mutation, e.g., one or more amino acid modification(s), deletion(s), addition(s) or substitution(s), in an unmodified cleavase, wherein: said modified or engineered cleavase is derived from a dipeptidyl peptidase of Thermomonas hydrothermahs or Caldithrix abyssii and removes or is configured to remove a single N-terminally modified amino acid from a target polypeptide.

In yet another embodiment, provided herein is a method of treating a target polypeptide, which method comprises: a) contacting a target polypeptide with an N-terminal modifier agent to form an N-terminally modified target polypeptide having a formula: M-P1-P2-polypeptide, said M being an N-terminal modification, said P1 being the N-terminal amino acid residue of said target polypeptide, and P2 being a penultimate terminal amino acid residue of said target polypeptide; and b) contacting a binder with said N-terminally modified target polypeptide to allow said binder to specifically bind to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

In yet another embodiment, provided herein is a kit of treating a target polypeptide, which kit comprises: a) an N-terminal modifier agent that is configured to contact a target polypeptide to form an N-terminally modified target polypeptide having a formula: M-P1-P2-polypeptide, said M being an N-terminal modification, said P1 being the N-terminal amino acid residue of said target polypeptide, and P2 being a penultimate terminal amino acid residue of said target polypeptide; and/or b) a binder that is configured to specifically bind to said N-terminally modified target polypeptide through interaction between said binder and said M and P1 of said N-terminally modified target polypeptide, and optionally wherein the binding specificity between said binder and said N-terminally modified target polypeptide is predominantly or substantially determined by said interaction between said binder and said P1 of said N-terminally modified target polypeptide.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1A. Exemplary ProteoCode peptide sequencing assay with native NTAA binders. (1) Peptide molecules are each labeled with a DNA recording tag and attached to beads at a low molecular density, a sparsity that permits only intramolecular information transfer to occur. (2) Next, an NTAA binding agent labelled with a DNA coding tag binds to the native NTAA residue. After binding and washing, the coding tag information is transferred enzymatically to the recording tag (via extension or ligation). (3) Next, the peptide N-terminal amino acid (NTAA) is labeled with an N-terminal modification and removed by using mild Edman-like elimination chemistry or by a Cleavase enzyme. After n cycles, a DNA library element representing the n amino acids of the peptide sequence is formed and can be sequenced by NGS. A representative structure of an NGS library element after 7 cycles is shown.

FIG. 1B. Exemplary ProteoCode peptide sequencing assay with modified NTAA binders. Peptide molecules are each labeled with a DNA recording tag and attached to beads at a low molecular density, a sparsity that permits only intramolecular information transfer to occur. (1) At the beginning of a sequencing cycle, the peptide N-terminal amino acid (NTAA) is functionalized with an N-terminal modification (NTM). (2) Next, a NTM-NTAA binding agent labelled with a DNA coding tag binds to the labeled NTAA residue. After binding and washing, the coding tag information is transferred enzymatically to the recording tag (via extension or ligation). (3) Removal of the NTM-labeled N-terminal residue is accomplished by using mild Edman-like elimination chemistry or by a Cleavase enzyme. After n cycles, a DNA library element representing the n amino acids of the peptide sequence is formed and can be sequenced by NGS. A representative structure of an NGS library element after 7 cycles is shown.

FIG. 2A-B. Design of exemplary N-terminal modifications (NTMs) to enable NTM-NTAA (P1) binding with minimal P2 bias. FIG. 2A. Structure of a bipartite NTM comprised of an amino acid portion (NTMaa) and an N-terminal blocking group (NTM_{blk}) connected with an amide bond. The NTM can also be a small chemical entity (NTM_{B}) with a similar shape configuration as NTM_{A}. As a third option, NTMc does not comprise amino acid-like moiety. FIG. 2B. The NTM is activated using standard methods (activated ester) and couples to the N-terminal amine on the P1 residue of the polypeptide. An engineered anticalin binds to the NTM-NTAA of the peptide by interacting with the P1 residue and the NTM. In the case of the bipartite NTMA, the anticalin interacts with the N moiety and NTM_{blk} moiety of the NTM. Alternatively, anticalin substrate-binding pocket structure can be adopted to accommodate NTM_{B} or NTMc. The size and shape of the NTM is designed to fill the anticalin pocket such that only the P1 residue of the polypeptide makes substantial contact with the binding pocket, but not the P2 residue.

FIG. 3 depicts a model of an exemplary anticalin scaffold bound with N-terminal modified amino acid. The NTM shown in spheres occupies part of a surface accessible substrate-binding pocket of the anticalin scaffold. The P1 sidechain is surrounded by amino acid residues of the scaffold that can be mutated to provide specificity towards the NTM-P1 located at the N-terminus of target peptides.

FIG. 4. Exemplary Lipocalin sequence clusters are shown (from #22 to # 66) selected for phage display library generation and binder engineering. Sequences of selected scaffolds are present in Sequence Listing (shown scaffold# and corresponding SEQ ID NO): #22—SEQ ID NO: 12; #24—SEQ ID NO: 13; #31—SEQ ID NO: 14; #32—SEQ ID NO: 7; #41—SEQ ID NO: 15; #43—SEQ ID NO: 16; #44—SEQ ID NO: 8; #49—SEQ ID NO: 9; #53—SEQ ID NO: 17; #59—SEQ ID NO: 10; #61—SEQ ID NO: 11; # 63—SEQ ID NO: 18; #66—SEQ ID NO: 19.

FIG. 5. Exemplary LC-MS analysis of modifying 17 different NTAA peptide with M17 reagent. Mass intensity units are plotted for unmodified (bottom panel) and M17-modified peptides (top panel) in the control case (no treatment) and when the peptides are functionalized with active M17 reagent. The y-axis shows extracted mass counts for each individual peptide fragment. The x-axis plots the NTAA residue of the corresponding XA-peptide (X=A, D, E, F, G, H, I, L, M, N, P Q, S, T, V, W, Y). All NTAA peptides were modified efficiently by the M17 reagent.

FIG. 6A-F illustrates exemplary Luminex-based binding affinity profile of anticalin clones chosen from a phage display screen against M17-P1 peptides. Six exemplary engineered anticalin binders are shown to have mostly mono-specificity for P1 residues. The anticalin clones were isolated from phage library panning as described in Example 2. Candidate clones were also isolated with specificity to different P1 residues of the peptides, including F, G, H, I, L, P, W and multi-specific Pls, including T/S, A/T/S, T/V/I/A, F/L.

FIG. 7 illustrates exemplary analysis of P2 dependence via ProteoCode™ encoding assay using the M15-L-G binder clone on various M17-G-P2 peptides.

FIG. 8 illustrates specificity profiling of M90-NTAA anticalin-based binders obtained using the Proteocode assay as described in Example 6. Sequences of engineered binders specific for M90-modified H, M, W, A and L were as follows: SEQ ID NO: 55, SEQ ID NO: 56, SEQ ID NO: 52, SEQ ID NO: 54, SEQ ID NO: 53.

FIG. 9 illustrates heatmap data showing encoding efficiency (calculated as fraction of recording tags encoded) for the engineered M12-F binder (SEQ ID NO: 50) in the multiplex encoding assay on immobilized set of 288 peptides (17X17 combination of different P1 and P2 residues). Arrow on the left shows primary binder's specificity. The more intense black indicates higher encoding efficiency.

FIG. 10 illustrates heatmap data showing encoding efficiency (calculated as fraction of recording tags encoded) for the engineered M12-L binder (SEQ ID NO: 49) in the multiplex encoding assay on immobilized set of 288 peptides (17X17 combination of different P1 and P2 residues). Arrow on the left shows primary binder's specificity. The more intense black indicates higher encoding efficiency.

FIG. 11 illustrates heatmap data showing encoding efficiency (calculated as fraction of recording tags encoded) for the engineered M44-L binder (SEQ ID NO: 31) in the multiplex encoding assay on immobilized set of 288 peptides (17X17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 12 illustrates heatmap data showing encoding efficiency (calculated as fraction of recording tags encoded) for the engineered M44-L binder (SEQ ID NO: 21) in the multiplex encoding assay on immobilized set of 288 peptides (17X17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 13 illustrates heatmap data showing encoding efficiency (calculated as fraction of recording tags encoded) for the engineered M44-A binder (SEQ ID NO: 42) in the multiplex encoding assay on immobilized set of 288 peptides (17X17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 14A illustrates heatmap data showing encoding efficiency (calculated as fraction of recording tags encoded) for the engineered M17-Y binder (SEQ ID NO: 61) in the multiplex encoding assay on immobilized set of 288 peptides (17X17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded). FIG. 14B illustrates similar heatmap data the upgraded binder after additional maturation round (introducing six additional mutations, SEQ ID NO: 62).

FIG. 15 illustrates heatmap data showing encoding efficiency (calculated as fraction of recording tags encoded) for the engineered M64-D binder (SEQ ID NO: 66) in the multiplex encoding assay on immobilized set of 288 peptides (17X17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 16 shows results of encoding reactions for the set of four M12-NTAA anticalin-based binders having different specificities obtained using the Proteocode assay as described in Example 5. Sequences of engineered binders specific for M12-modified F/Y; I/L/V; W and D/N residues were as follows: SEQ ID NO: 57, SEQ ID NO: 58, SEQ ID NO: 59, SEQ ID NO: 60. On the x-axis, specific NTAA residues (modified with M12 for the assay) are shown that generated the encoding signal above the background, indicating specific binding.

FIG. 17A-B. FIG. 17A illustrates exemplary cleavage of M17-modified NTAAs of a model polypeptide (M17-P1-AR) with the disclosed Cleavase enzymes. A compilation of seven different modified Cleavase clones was used to generate the spectrum of cleavage profiles across the M17-modified NTAAs as shown. Data were generated by HPLC analysis (UV absorbance) of cleaved versus intact peptides after cleavase assay. FIG. 17B shows the same cleavage events using SDS-PAGE analysis.

### DETAILED DESCRIPTION

The present disclosure relates to a binder that specifically binds to an N-terminally modified polypeptide and modified or an engineered cleavase that removes or is configured to remove a single N-terminally modified amino acid from a polypeptide. Also provided herein is a method and related kits for treating a polypeptide using or comprising the binder and/or modified cleavase. In some embodiments, also provided herein is a method and related kits for transferring information using a plurality of enzymes, including for performing a ligation, extension, and cleavage reaction with nucleic acid molecules associated with the polypeptide for analysis. Also provided are kits containing components and/or reagents for performing the provided methods for macromolecule, e.g., polypeptide and/or polynucleotide, sequencing and/or analysis. In some embodiments, the kits also include instructions for using the kit to perform any of the methods provided herein.

Highly-parallel characterization and recognition of macromolecules such as proteins remains a challenge. In proteomics, one goal is to identify and quantitate numerous proteins in a sample, which is a formidable task to accomplish in a high-throughput way. Assays such as immunoassays and mass spectrometry based methods have been used but are limited at both the sample and analyte level, limited sensitivity and dynamic range, and cross-reactivity and background signals. Multiplexing the readout of a collection of affinity agents to a collection of cognate macromolecules, for example using affinity agents with detectable labels, remains challenging. There remains a need for improved techniques relating to macromolecule analysis, with applications to protein sequencing and/or analysis, as well as to products, methods and kits for accomplishing the same. There is a need for proteomics technology for performing macromolecule analysis that is efficient, highly-parallelized, accurate, sensitive, and high-throughput. The present disclosure fulfills these and other related needs.

In some embodiments, the present disclosure provides, in part, methods for analyzing a macromolecule (polypeptide) which includes information transfer, with direct applications to protein and peptide characterization, quantitation, and/or sequencing. In some examples, the information transferred comprises identifying information regarding a binding agent that is configured to bind to the macromolecule. In some embodiments, a plurality of macromolecules obtained from a sample is analyzed. In some embodiments, the sample is obtained from a subject. In some embodiments, the macromolecule sequencing or analysis method includes using a plurality of binding agents associated with coding tags to detect a plurality of macromolecules to be analyzed.

In some embodiments, the information transfer methods provided herein utilize a plurality of enzymes to perform ligation, extension, and cleavage reactions with nucleic acid molecules. In some embodiments, the provided methods includes an oligonucleotides that comprise hairpin structure and a restriction enzyme site (or portion thereof). In some embodiments, the methods include the use of a reaction system wherein mixed enzymes are provided to the reaction. For example, the activities of the polymerase, the nucleic acid joining reagent and the double strand nucleic acid cleaving reagent, are provided with suitable conditions, transferring information from a coding tag to the recording tag to generate an extended recording tag. In the provided methods, the recording tag used comprises at least a partially double stranded DNA structure. Some advantages using the described methods include high information transfer (encoding) success, simple design for a step-wise reaction, option to perform in a single step/as a single pot reaction, reducing the need for spacers or reducing spacer length, and/or minimizing DNA-DNA interactions in the system.

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### DEFINITIONS

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

As used herein, the term “polypeptide” is used interchangeably with “peptide”, encompassing both peptides and proteins, and refers to a molecule comprising a chain of two or more amino acids joined by peptide bonds. In some embodiments, a polypeptide comprises 2 to 200 amino acid residues. In some embodiments, a polypeptide does not comprise a secondary, tertiary, or higher structure. In some embodiments, the polypeptide is a protein and comprises a secondary or tertiary structure. In some embodiments, a protein comprises 50 or more amino acids. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid-containing polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent”, or “binder”, refers to a nucleic acid molecule, a peptide, a polypeptide, a protein, carbohydrate, or a small molecule that binds to, associates, unites with, recognizes, or combines with a binding target, e.g., a polypeptide or a component or feature of a polypeptide. A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a polypeptide (e.g., a single amino acid of a polypeptide) or bind to a plurality of linked subunits of a polypeptide (e.g., a di-peptide , tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid (e.g., an amino acid that has been labeled by a the N-terminal modifier agent comprising a compound of Embodiments 47 or 61 as described below) over a non-modified or unlabeled amino acid. For example, a binding agent may preferably bind to an amino acid that has been labeled or modified over an amino acid that is unlabeled or unmodified. A binding agent may bind to a post-translational modification of a peptide molecule. A binding agent may exhibit selective binding to a component or feature of a polypeptide (e.g., a binding agent may selectively bind to one of the 20 possible natural amino acid residues and bind with very low affinity or not at all to the other 19 natural amino acid residues). A binding agent may exhibit less selective binding, where the binding agent is capable of binding or configured to bind to a plurality of components or features of a polypeptide (e.g., a binding agent may bind with similar affinity to two or more different amino acid residues). A binding agent may comprise a coding tag, which may be joined to the binding agent by a linker.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a binding agent with a coding tag, a recording tag with a polypeptide, a polypeptide with a support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

The terminal amino acid at one end of a peptide or polypeptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the nth amino acid (also referred to herein as the “n NTAA”). Using this nomenclature, the next amino acid is the n-1 amino acid, then the n-2 amino acid, and so on down the length of the peptide from the N-terminal end to C-terminal end. In certain embodiments, an NTAA, CTAA, or both may be modified or labeled with a moiety or a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp' refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the macromolecule (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds to a polypeptide, information from a coding tag linked to a binding agent can be transferred to the recording tag associated with the polypeptide while the binding agent is bound to the polypeptide. In other embodiments, after a binding agent binds to a polypeptide, information from a recording tag associated with the polypeptide can be transferred to the coding tag linked to the binding agent while the binding agent is bound to the polypeptide. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each macromolecule, polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5” or “sense”. The term “reverse” when used in context with a “universal priming site” or “universal primer” may also be referred to as “3′” or “antisense”.

As used herein, the term “extended recording tag” refers to a recording tag to which information of at least one binding agent's coding tag (or its complementary sequence) has been transferred following binding of the binding agent to a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35% , 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), yPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, the term “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Detectable labels include any labels that can be utilized and are compatible with the provided polypeptide analysis assay format and include, but not limited to, a bioluminescent label, a biotin/avidin label, a chemiluminescent label, a chromophore, a coenzyme, a dye, an electro-active group, an electrochemiluminescent label, an enzymatic label (e.g. alkaline phosphatase, luciferase or horseradish peroxidase), a fluorescent label, a latex particle, a magnetic particle, a metal, a metal chelate, a phosphorescent dye, a protein label, a radioactive element or moiety, and a stable radical.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times) — this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” the polypeptide means to identify, detect, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n-1, n-2, n-3, and so forth). This is accomplished by elimination of the n NTAA, thereby converting the n-1 amino acid of the peptide to an N-terminal amino acid (referred to herein as the “n-1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The term “unmodified” (also “wild-type” or “native”) as used herein is used in connection with biological materials such as nucleic acid molecules and proteins (e.g., binders), refers to those which are found in nature and not modified by human intervention.

The term “modified” or “engineered” (or “variant”, or “mutant”) as used in reference to nucleic acid molecules and protein molecules, e.g., an engineered binder, implies that such molecules are created by human intervention and/or they are non-naturally occurring. The variant, mutant or engineered anticalin-derived binder is a polypeptide having an altered amino acid sequence, relative to an unmodified or wild-type protein, such as starting anticalin scaffold, or a portion thereof. An engineered anticalin-derived binder is a polypeptide which differs from a wild-type anticalin scaffold sequence, or a portion thereof, by one or more amino acid substitutions, deletions, additions, or combinations thereof. Sequence of an engineered anticalin-derived binder can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences (e.g., mutations) compared to the sequence of starting anticalin scaffold. An engineered anticalin-derived binder generally exhibits at least 70%, 80%, 85%, 86%, 87%, 88%, 89%, 90%, 91%, 92%, 93%, 94%, 95%, 96%, 97%, 98%, 99% or more sequence identity to a corresponding wild-type starting anticalin scaffold. Non-naturally occurring amino acids as well as naturally occurring amino acids are included within the scope of permissible substitutions or additions. An engineered anticalin-derived binder is not limited to any engineered anticalin-derived binders made or generated by a particular method of making and includes, for example, an engineered anticalin-derived binder made or generated by genetic selection, protein engineering, directed evolution, de novo recombinant DNA techniques, or combinations thereof. The term “variant” in the context of variant or engineered anticalin-derived binder is not to be construed as imposing any condition for any particular starting composition or method by which the variant or engineered anticalin-derived binder is created. Thus, variant or engineered anticalin-derived binder denotes a composition and not necessarily a product produced by any given process. A variety of techniques including genetic selection, protein engineering, recombinant methods, chemical synthesis, or combinations thereof, may be employed.

In some embodiments, variants of an engineered binder displaying only non-substantial or negligible differences in structure can be generated by making conservative amino acid substitutions in the engineered binder. By doing this, further engineered binder variants that comprise a sequence having at least 90% (90%, 91%, 92%, 93%, 94%, 95%, 96%, 97%, 98%, and 99%) sequence identity with the initial engineered binder sequences can be generated, retaining at least one functional activity of the engineered binder, e.g. ability to specifically bind to the N-terminally modified target polypeptide. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 7 positions in a sequence of 10 nucleotides in length are identical to the corresponding positions in a second 10-nucleotide sequence, then the two sequences have 70% sequence identity. Methods for the alignment of sequences for comparison are well known in the art, such methods include the BLAST algorithm, which calculates percent sequence identity and performs a statistical analysis of the similarity between the two sequences. The software for performing BLAST analysis is publicly available through the National Center for Biotechnology Information (NCBI) website.

The terms “corresponding to position(s)” or “position(s) ... with reference to position(s)” of or within a polypeptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the polypeptide upon alignment with the disclosed sequence using a standard alignment algorithm, such as the BLAST algorithm (NCBI). One skilled in the art can identify any given amino acid residue in a given polypeptide at a position corresponding to a particular position of a reference sequence, such as set forth in the Sequence Listing, by performing alignment of the polypeptide sequence with the reference sequence (for example, by using BLASTP publicly available through the NCBI website), matching the corresponding position of the reference sequence with the position in polypeptide sequence and thus identifying the amino acid residue within the polypeptide.

The term “peptide bond” as used herein refers to a chemical bond formed between two molecules (such as two amino acids) when the carboxyl group of one molecule reacts with the amino group of the other molecule, releasing a water molecule (H2O).

The term “modified amino acid residue” as used herein refers to an amino acid residue within a polypeptide that comprises a modification that distinguish it from the corresponding original, or unmodified, amino acid residue. In some embodiments, the modification can be a naturally occurring post-translational modification of the amino acid residue. In other embodiments, the modification is a non-naturally occurring modification of the amino acid residue; such modified amino acid residue is not naturally present in polypeptides of living organisms (represents an unnatural amino acid residue). Such modified amino acid residue can be made by modifying a natural amino acid residue within the polypeptide by a modifying reagent, or can be chemically synthesized and incorporated into the polypeptide during polypeptide synthesis.

The term “unnatural amino acid” or “unnatural amino acid moiety” as used herein refers to a compound selected from the group consisting of: 3-(3′-pyridyl)-L-alanine, L-cyclohexylglycine, α-aminoisobutyric acid, 3-(4′-pyridyl)-L-alanine, L-azetidine-2-carboxylic acid, isonipecotic acid, L-phenylglycine, β-(2-thienyl)-L-alanine, 3-(4-thiazolyl)-L-alanine, 1-aminocyclopentane-1-carboxylic acid, (2-trifluoromethyl)-L-Phenylalanine, L-cyclopropylalanine, 3-(2′-pyridyl)-L-alanine, beta-cyano-L-alanine, α-methyl-L Fluorophenylalanine, α-methyl-D-4-fluorophenylalanine, 3-amino-2,2-difluoro-propionic acid, O-sulfo-L-tyrosine sodium salt, L-2-furylalanine, 1-aminocyclopropane-1-carboxylic acid, 3,5-dinitro-L-tyrosine, pentafluoro-L-phenylalanine, 3,5-difluoro-L-phenylalanine, 3-fluoro-L-phenylalanine, N-cyclopentylglycine, 1-(amino)cyclohexanecarboxylic acid, N-methylalanine, 4-amino-tetrahydropyran-4-carboxylic acid, 4-amino-1,1-dioxothiane-4-carboxylic acid, 4-amino-1-methyl-4-piperidinecarboxylic acid, 2-amino-N-(2,4-dimethoxybenzyl)acetamido)acetic acid, and a N-alkylated derivative thereof.

The terms “specifically binding” and “specifically recognizing” are used interchangeably herein and generally refer to an engineered binder that binds to a cognate target polypeptide or a portion thereof more readily than it would bind to a random, non-cognate polypeptide. The term “specificity” is used herein to qualify the relative affinity by which an engineered binder binds to a cognate target polypeptide. Specific binding typically means that an engineered binder binds to a cognate target polypeptide at least twice more likely that to a random, non-cognate polypeptide (a 2:1 ratio of specific to non-specific binding). Non-specific binding refers to background binding, and is the amount of signal that is produced in a binding assay between an engineered binder and an N-terminally modified target polypeptide when the modified NTAA residue cognate for the engineered binder is not present at the N-terminus of the target polypeptide. In some embodiments, specific binding refers to binding between an engineered binder and an N-terminally modified target polypeptide with a dissociation constant (Kd) of 200 nM or less.

In some embodiments, binding specificity between an engineered binder and an N-terminally modified target polypeptide is predominantly or substantially determined by interaction between the engineered binder and the modified NTAA residue of the N-terminally modified target polypeptide, which means that there is only minimal or no interaction between the engineered binder and the penultimate terminal amino acid residue (P2) of the target polypeptide, as well as other residues of the target polypeptide. In some embodiments, the engineered binder binds with at least 5 fold higher binding affinity to the modified NTAA residue of the target polypeptide than to any other region of the target polypeptide. In some embodiments, the engineered binder has a substrate binding pocket with certain size and/or geometry matching the size and/or geometry of the modified NTAA residue of the N-terminally modified target polypeptide, to which the engineered binder specifically binds to. In such embodiments, the modified NTAA residue occupies a volume encompassing a substrate binding pocket of the engineered binder that effectively precludes the P2 residue of the target polypeptide from entering into the substrate binding pocket or interacting with affinity-determining residues of the engineered binder. In some embodiments, the engineered binder specifically binds to N-terminally modified target polypeptides, wherein the target polypeptides share the same modified NTAA residue that interacts with the engineered binder, but have different P2 residues. In some embodiments, the engineered binder is capable of specifically binding to each N-terminally modified target polypeptide from a plurality of N-terminally modified target polypeptides, wherein the plurality of N-terminally modified target polypeptides contains at least 3, at least 5, or at least 10 N-terminally modified target polypeptides that were modified with the same N-terminal modifier agent, have the same modified NTAA residue, and have different P2 residues. Thus, in preferred embodiments, the engineered binder possesses binding affinity towards the modified NTAA residue of the N-terminally modified target polypeptide, but has little or no affinity towards P2 or other residues of the target polypeptide.

As used herein, the term “heterocycle”, “heterocyclic”, or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

The term “substituted” means that the specified group or moiety bears one or more substituents in place of a hydrogen atom of the unsubstituted group, including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents and thus includes both substituted and unsubstituted versions of the group. Where the term “substituted” is used to describe a structural system, the substitution is meant to occur at any valency-allowed position on the system.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### I. BINDERS FOR N-TERMINALLY MODIFIED POLYPEPTIDES

In one embodiment, provided herein is a binder that specifically binds to an N-terminally modified target polypeptide, wherein: said N-terminally modified target polypeptide is derived from a target polypeptide and said N-terminally modified target polypeptide has a formula: M-P1-P2-polypeptide, said M being an N-terminal modification, said P1 being the N-terminal amino acid residue of said target polypeptide, and P2 being a penultimate terminal amino acid residue of said target polypeptide; and said binder specifically binds to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

The present binders can specifically bind to any suitable N-terminally modified target polypeptide or N-terminally modified target polypeptide with any suitable length. For example, the length of the target polypeptide and/or the N-terminally modified target polypeptide can be greater than 4 amino acids, greater than 5 amino acids, greater than 6 amino acids, greater than 7 amino acids, greater than 8 amino acids, greater than 9 amino acids, greater than 10 amino acids, greater than 11 amino acids, greater than 12 amino acids, greater than 13 amino acids, greater than 14 amino acids, greater than 15 amino acids, greater than 20 amino acids, greater than 25 amino acids, or greater than 30 amino acids.

The P1 or the N-terminal amino acid residue of a target polypeptide can be any suitable amino acid residue. In some embodiments, the P1 can comprise a naturally-occurring amino acid residue. In some embodiments, the P1 can be selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the P1 can comprise a modification, e.g., a naturally-occurring or a non-natural modification. In some embodiments, the P1 can comprise an amino acid with a post-translational modification.

The P2 or the penultimate terminal amino acid residue of a target polypeptide can be any suitable amino acid residue. In some embodiments, the P2 can comprise a naturally-occurring amino acid residue. In some embodiments, the P2 can be selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the P2 can comprise a modification, e.g., a naturally-occurring or a non-natural modification. In some embodiments, the P2 can comprise an amino acid with a post-translational modification.

The M can comprise any suitable N-terminal modification. For example, the M can comprise a synthetic N-terminal modification. In another example, the M can comprise an amino acid moiety and/or has a size, e.g., length axis or volume, shape, and/or configuration similar to or exceeding a natural amino acid.

In some embodiments, the M can comprise an amino acid moiety (NTMaa). The M can be a bipartite N-terminal modification (NTM) that comprises a natural or unnatural amino acid portion (NTMaa) and an N-terminal blocking group (NTM_{blk}). The amino acid portion (NTMaa) and the N-terminal blocking group (NTM_{blk}) can be connected or linked by any suitable bond or linkage. For example, the amino acid portion (NTMaa) and the N-terminal blocking group (NTM_{blk}) can be connected with an amide bond. In some embodiments, the bipartite N-terminal modification (NTM) is installed under conditions to minimize racemization and subsequent generation of stereoisomers in the final modified peptide (Ramu, Vasanthakumar G., et al., “DEPBT as Coupling Reagent To Avoid Racemization in a Solution-Phase Synthesis of a Kyotorphin Derivative.” 2014, Synthesis 46 (11): 1481-86). Binding is typically stereospecific and is most efficient using a single stereoisomer as a binding target. In some embodiments, L- or D- configurations of NTMaa portion of the N-terminal modifier agent were alkylated to prevent racemization during installation of the N-terminal modification.

In some embodiments, the M does not comprise an amino acid moiety. The M can be a bipartite N-terminal modification (NTM) that comprises a small (or small molecule) chemical entity having a size, e.g., length axis or volume, shape, and/or configuration similar to or exceeding a natural amino acid, and an N-terminal blocking group (NTM_{blk}). The small (or small molecule) chemical entity and the N-terminal blocking group (NTM_{blk}) can be connected or linked by any suitable bond or linkage. For example, the small (or small molecule) chemical entity and the N-terminal blocking group can be connected with an amide bond. The small (or small molecule) chemical entity can have any suitable size, e.g., length axis or volume. For example, the small (or small molecule) chemical entity can have a size, e.g., length axis of about 5-10 Å and volume of about 100-1000 Å³. In some embodiments, the small (or small molecule) chemical entity has a length axis of about 5, 6, 7, 8, 9 or 10 Å, or any range thereof. In some embodiments, the small (or small molecule) chemical entity has a volume of about 100, 200, 300, 400, 500, 600, 700, 800, 900, 1000 Å³ or any range thereof.

The present N-terminally modified target polypeptide can comprise any suitable N-terminal modification (M). In some embodiments, the present N-terminally modified target polypeptide can comprise an N-terminal modification (M) as described in Section V below.

In some embodiments, the interaction between the binder and the M has minimal or no impact or influence on the binding specificity between the binder and the N-terminally modified target polypeptide. In some embodiments, the interaction between the binder and the M at least partially determines the binding strength or binding efficiency between the binder and the N-terminally modified target polypeptide.

In some embodiments, there is minimal or no interaction between the binder and the P2. In some embodiments, there is minimal interaction between the binder and the P2, and the minimal interaction between the binder and the P2 has minimal or no impact or influence on the binding specificity, strength and/or efficiency between the binder and the N-terminally modified target polypeptide; and/or P1-P2 occupies a volume or shape encompassing a cavity or pocket of the binder that effectively precludes the P2 from entering into or interacting with an affinity determining region of the binder. In some embodiments, the volume of the cavity or pocket is greater than the volume occupied by a glycine residue. In some embodiments, the volume of the pocket or cavity is less than about 1,000 Å³.

The present binders can specifically bind to N-terminally modified target polypeptide that contains a particular or specific N-terminal amino acid residue and have the ability to distinguish N-terminally modified target polypeptides that contain different N-terminal amino acid residues. The present binders can also specifically bind to N-terminally modified target polypeptides that contain a group of N-terminal amino acid residues, and have the ability to distinguish such N-terminally modified target polypeptides from other N-terminally modified target polypeptides that contain N-terminal amino acid residue(s) outside the recognized group of the N-terminal amino acid residues. In some embodiments, the present binder specifically binds to multiple N-terminally modified target polypeptides that comprise the same P1 residue. In some embodiments, the present binder specifically binds to multiple N-terminally modified target polypeptides that comprise different P1 residues. For example, the present binder can specifically bind to multiple N-terminally modified target polypeptides that comprise 2, 3, 4, 5, 6, 7, 8, 9 or 10 different P1 residues.

The present binders can comprise any suitable type of composition or molecule. For example, the present binders can comprise or can be a polypeptide, e.g., an antibody or a fragment or derivative thereof. In another example, the present binders can comprise or can be an engineered anticalin.

The engineered anticalin can be derived or evolved from any suitable parent or natural lipocalin. For example, the engineered anticalin can be derived or evolved from an eukaryotic lipocalin, such as a lipocalin from a human, a cow, a pig, an insect, e.g., a butterfly. The engineered anticalin can also be derived or evolved from any suitable types of lipocalin. For example, the engineered anticalin can be derived or evolved from a core or kernel lipocalin, or from an outlier lipocalin.

The engineered anticalin can have any suitable binding region, core or pocket. For example, the engineered anticalin can comprise an anticalin b-barrel core. In some embodiments, upon binding to an N-terminally modified target polypeptide, the M-P1 residues of the N-terminally modified target polypeptide occupy the anticalin b-barrel core. The pocket volume of the lipocalin from which the anticalin is derived can span volumes ranging from 200 Å³-3,000 Å³ encompassing a range of M-P1 sizes. For example, The pocket volume of the lipocalin from which the anticalin is derived can span volumes ranging from 200 Å³-500 Å³, 500 Å³-1000 Å³, 1,000 Å³-2,000 Å³, 2,000 Å³-3,000 Å³, or any subrange thereof, encompassing a range of M-P1 sizes.

The engineered anticalin in the present binders can specifically binds to an N-terminally modified target polypeptide with any suitable P1 residue. For example, the engineered anticalin can specifically bind to an N-terminally modified target polypeptide with a P1 residue that is selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the engineered anticalin specifically binds to an N-terminally modified target polypeptide with a P1 residue that is: Isoleucine (I or Ile) or Leucine (L or Leu); T/S; A/T/S; T/V/I/A; or F/L.

In some embodiments, the engineered anticalin is derived or evolved from a lipocalin that comprises an amino acid sequence set forth in SEQ ID NO:19 and has a mutation selected from the group consisting of V33T, L36R, Y52R, T54L, L70M, R79S, W81E, F85Q, L96E, N₉₈L, H100T, R101W, Y102H, Y108W, F125S, K127P, K136R, Y140L and a combination thereof. In some embodiments, the engineered anticalin comprises an amino acid sequence that has at least 80%, 90%, 95% or more identity to an amino acid sequence set forth in SEQ ID NO:19 or to an amino acid sequence of the engineered anticalin having a mutation of V33T, L36R, Y52R, T54L, L70M, R79S, W81E, F85Q, L96E, N₉₈L, H100T, R101W, Y102H, Y108W, F125S, K127P, K136R, or Y140L.

In some embodiments, the present binders can have a binding signal and/or affinity towards a modified target polypeptide comprising a specific P1 residue that is at least 2-fold or higher as compared to the binder's binding signal and/or affinity towards an otherwise identical modified target polypeptide but comprising a different P1 residue. In some embodiments, the present binders can have a binding signal and/or affinity towards a modified target polypeptide comprising a specific P1 residue that is at least 2-fold, 3-fold, 4-fold, 5-fold, 6-fold, 7-fold, 8-fold, 9-fold, 10-fold, 15-fold, 20-fold, 50-fold, 100-fold, or higher, as compared to the binder's binding signal and/or affinity towards an otherwise identical modified target polypeptide but comprising a different P1 residue.

A nucleic acid encoding the above engineered anticalin is provided herein. A vector, e.g., an expression vector, comprising the nucleic acid encoding the above engineered anticalin is also provided herein. A host cell comprising the above nucleic acid or the vector is further provided herein. The host cell can be any suitable type of cell. For example, the host cell can be a mammalian or human host cell.

### II. MODIFIED OR ENGINEERED CLEAVASES

In another embodiment, provided herein is a modified or an engineered cleavase comprising a mutation, e.g., one or more amino acid modification(s), deletion(s), addition(s) or substitution(s), in an unmodified cleavase, wherein: said modified or engineered cleavase is derived from a dipeptidyl peptidase of Thermomonas hydrothermahs or Caldithrix abyssii and removes or is configured to remove a single N-terminally modified amino acid from a target polypeptide. In some embodiments, the present modified or engineered cleavase is configured to cleave the peptide bond between an N-terminally modified amino acid residue and a penultimate terminal amino acid residue of the target polypeptide.

The present modified or engineered cleavase can comprise any suitable active site. For example, the present modified or engineered cleavase can comprise an active site that interacts with the amide bond between the N-terminally modified amino acid residue and a penultimate terminal amino acid residue of the target polypeptide.

The present modified or engineered cleavase can be derived from any suitable type of dipeptidyl peptidase. For example, the present modified or engineered cleavase can be derived from a protein or enzyme classified as a S46 dipeptidyl peptidase (see e.g., Shakh M.A. Rouf, Yuko Ohara-Nemoto, Tomonori Hoshino, Taku Fujiwara, Toshio Ono, Takayuki K. Nemoto, Discrimination based on Gly and Arg/Ser at position 673 between dipeptidyl-peptidase (DPP) 7 and DPP11, widely distributed DPPs in pathogenic and environmental gram-negative bacteria, Biochimie, Volume 95, Issue 4, 2013, Pages 824-832, ISSN 0300-9084), or a functional homolog or fragment thereof.

The present modified or engineered cleavase can remove or can be configured to remove any suitable single N-terminally modified amino acid from a target polypeptide. For example, the present modified or engineered cleavase can remove or can be configured to remove an N-terminal amino acid that is labeled with a chemical or an enzymatic reagent or moiety.

The present modified or engineered cleavase can remove or can be configured to remove any suitable single N-terminally modified amino acid from a target polypeptide containing any suitable N-terminal modification. For example, the N-terminal modification can comprise a blocked amino acid. In another example, the N-terminal modification can comprise a chemical label or reagent.

In some embodiments, the chemical reagent for the N-terminal modification is selected from the group consisting of: 2-aminobenzamide, 2-(N-methylamino)-benzamide, 2-(N-acetylamine)-benzamide, 2-(N-benzylamine)-benzamide, 4-methylbenzamide, 4-(dimethylamino)benzamide, nicotinamide, 3-aminonicotinamide, 2-pyrazinecarbonyl, 5-amino-2-fluoro-i sonicotinamide, 2-carboxylic acid pyrazinecarbonyl, 3,6-difluoro-2-carboxybenzamide, 4-chloro-2-aminobenzamide, 4-nitro-2-aminobenzamide, 4-methoxy-2-aminobenzamide, 4-carboxylic acid-2-aminobenzamide, 5-(trifluoromethyl-2-aminobenzamide, 4-(trifluoromethyl-2-aminobenzamide, 6-fluoro-2-aminobenzamide, 4-fluoro-2-aminobenzamide, 5-methoxy-2-aminobenzamide, 4-fluorobenzamide, 4-(trifluoromethyl)benzamide, 8-fluoroisoquinolinium, 1-hydroxy-2,3,1-benzodiazaborinine-2(1H)-carbonyl, Succinamide, 3,6-Difluoropyridine-2-carbamide, 2-Fluoronicotinamide, 5-Bromo-2-hydroxynicotinamide, 4-(Trifluoromethyl)pyrimidine-5-carbamide, 2-Oxo-1,2-dihydropyridine-3-carbamide, 5-Methyl-2-aminobenzamide, 6-Fluoropicolinamide, 3-Methyl aminobenzamide, 4-Methyl--2-aminobenzamide, 2-Amino-6-methylbenzamide, 2-Amino fluorobenzamide, 2-Amino-5-fluorobenzoamide, 2-Amino-3-fluorobenzoamide, 2-Amino fluorobenzoamide, 2-Aminonicotinamide, 4-Aminonicotinamide, 3-Aminopicolinamide, or a derivative thereof. In some embodiments, the chemical reagent for the N-terminal modification is an isatoic anhydride, an isonicotinic anhydride, an azaisatoic anhydride, a succinic anhydride, an aryl activated ester, a heteroaryl activated ester, a non-aromatic ring activated ester, or a derivative thereof. In some embodiments, the chemical reagent for the N-terminal modification is selected from the group consisting of wherein the chemical reagent is selected from the group consisting of 4-Nitrophenyl Anthranilate, N-Methyl-isatoic anhydride, N-acetyl-isatoic anhydride, N-benzyl-isatoic anhydride, 4-methylbenzoic acid, 4-(dimethylamino)benzoyl chloride, nicotinic acid-NETS, 3-aminonicotinic acid, 2-pyrazinecarbonyl chloride, 5-amino-2-fluoro-isonicotinic acid, 2,3-pyrazinedicarboxylic anhydride, 3,6-difluorophthalic anhydride, 4-chloroisatoic anhydride, 4-nitroisatoic anhydride, 7-methoxy-1h-benzo[d][1,3]oxazine-2,4-dione, 4-carboxylic acid isatoic anhydride, 6-(Trifluoromethyl)-2,4-dihydro-1h-3,1-benzoxazine-2,4-dione, 7-(Trifluoromethyl)-1h-benzo[d][1,3]oxazine-2,4-dione, 6-fluoroisatoic anhydride, 4-fluoroisatoic anhydride, 5-methoxyisatoic anhydride, 4-fluorobenzoic acid anhydride, 4-(trifluoromethyl)benzoic acid anhydride, 2-ethynyl-6-fluorobenzaldehyde, 1-hydroxy-2,3,1-benzodiazaborinine-2(1H)-carboxylic acid, Isatoic anhydride, Succinic anhydride 3,6-Difluoropyridine-2-carboxylic acid, 2-Fluoronicotinic acid, 5-Bromo-2-hydroxynicotinic acid, 4-(Trifluoromethyl)pyrimidine-5-carboxylic acid, 2-Oxo-1,2-dihydropyridine-3-carboxylic acid, 5-Methylisatoic anhydride, 6-Fluoropicolinic acid, 3-Methylisatoic anhydride, 4-Methyl-isatoic anhydride, 2-Amino-6-methylbenzoic acid, 2-Amino-6-fluorobenzoic acid, 2-Amino-5-fluorobenzoic acid, 2-Amino-3-fluorobenzoic acid, 2-Amino-4-fluorobenzoic acid, 2-Aminonicotinic acid, 4-Aminonicotinic acid, 3-Aminopicolinic acid, or a derivative thereof.

The present modified or engineered cleavase can comprise any suitable amino acid sequence variation(s) as compared with the amino acid sequence of the unmodified cleavase. For example, the present modified or engineered cleavase can comprise an amino acid sequence that exhibits at least 50% identity, at least 60% identity, at least 70% identity, at least 80% identity, or at least 90%, or at least 95%, or more identity with the unmodified cleavase.

The present or engineered modified cleavase can comprise any suitable type of mutation(s). For example, wherein the mutation can comprise an amino acid substitution, deletion, addition, or a combination thereof.

The present modified or engineered cleavase can remove or can be configured to remove a single N-terminally modified amino acid from a target polypeptide with any suitable length. For example, the length of the target polypeptide can be greater than 4 amino acids, greater than 5 amino acids, greater than 6 amino acids, greater than 7 amino acids, greater than 8 amino acids, greater than 9 amino acids, greater than 10 amino acids, greater than 11 amino acids, greater than 12 amino acids, greater than 13 amino acids, greater than 14 amino acids, greater than 15 amino acids, greater than 20 amino acids, greater than 25 amino acids, or greater than 30 amino acids.

The present modified or engineered cleavase can comprise mutation(s) at any suitable site(s). For example, the present modified or engineered cleavase can comprise a modification within its substrate binding site. In another example, the present modified or engineered cleavase can comprise a modification within its catalytic domain. In still another example, the present modified or engineered cleavase can comprise a modification within its chymotrypsin fold. In yet another example, the present modified or engineered cleavase can comprise a modification at an amine binding site(s). In yet another example, the present modified or engineered cleavase can comprise a modification in its S1 and/or S2 sites. In yet another example, the present modified or engineered cleavase can comprise a modification for improving accessibility to the active site of the modified or engineered cleavase.

In some embodiments, the present modified or engineered cleavase is derived from a dipeptidyl peptidase of Thermomonas hydrothermahs comprising an amino acid sequence set forth in SEQ ID NO:3 (WT sequence with the signal peptide) or SEQ ID NO:4 (WT sequence without the signal peptide).

The present modified or engineered cleavase can comprise any suitable amino acid sequence variations as compared with the amino acid sequence of the unmodified cleavase. For example, the present modified or engineered cleavase can comprise an amino acid sequence that exhibits at least 20% identity, at least 30% identity, at least 40% identity, at least 50% identity, at least 60% identity, at least 70% identity, at least 80% identity, at least 90% or more identity or at least 95% or more identity to the amino acid sequence set forth in SEQ ID NO:3 or SEQ ID NO:4, or a specific binding fragment thereof.

In some embodiments, the present modified or engineered cleavase has a mutation, with reference to numbering of SEQ ID NO: 3 or SEQ ID NO: 4, selected from the group consisting of N₂₁₄X, W215X, R219X, N₃₂₉X, N₃₃₃X, A671X, D673X, G674X, N₆₈₂X, M692X, I651X, and a combination thereof, X being one of the 20 naturally occurring amino acids other than the amino acid residue of the unmodified dipeptidyl peptidase at the mutated position. In some embodiments, the present modified or engineered cleavase has one or more amino acid modification(s) of N₂₁₄M, W215G, R219T, N₃₂₉R, D673A, and/or G674V.

In some embodiments, the present modified or engineered cleavase exhibits the substrate specificity of the above modified or engineered cleavase. In present some embodiments, the modified or engineered cleavase comprises an amino acid sequence that comprises a catalytic domain, an amine binding site, or Si and/or S2 sites with at least 20% identity, at least 30% identity, at least 40% identity, at least 50% identity, at least 60% identity, at least 70% identity, at least 80% identity, or at least 90%, 95%, or more identity with the catalytic domain, the amine binding site, or the Si and/or S2 sites of the above modified or engineered cleavase. By definition (or at least in some embodiments), when referring to proteases, the S1 site is defined as the region of the protease that binds to the amino acid just upstream (amino side) of the cleavage position and the S2 site binds to the amino acid two residues upstream (amino side) to the cleavage position. For a native DPP which binds to the N-terminal dipeptide of a peptide, the S2 site binds to the N-terminal amino acid and the Si site binds to the penultimate amino acid. In the modified or engineered Cleavase, derived from a DPP, the S2 site can be used to partake in binding to the N-terminal modification (NTM), and the Si site used to bind to the NTAA residue.

In some embodiments, the present modified or engineered cleavase is derived from a dipeptidyl peptidase of Caldithrix abyssii comprising an amino acid sequence set forth in SEQ ID NO: 5 (WT sequence with the signal peptide) or SEQ ID NO: 6 (WT sequence without the signal peptide).

The present modified or engineered cleavase can comprise any suitable amino acid sequence variations as compared with the amino acid sequence of the unmodified cleavase. For example, the present modified or engineered cleavase can comprise an amino acid sequence that exhibits at least 20% identity, at least 30% identity, at least 40% identity, at least 50% identity, at least 60% identity, at least 70% identity, at least 80% identity, at least 90% or more identity or at least 95% or more identity to the amino acid sequence set forth in SEQ ID NO: 5 or SEQ ID NO: 6, or a specific binding fragment thereof.

In some embodiments, the present modified or engineered cleavase has a mutation, with reference to numbering of SEQ ID NO: 5 or SEQ ID NO: 6, selected from the group consisting of N₂₀₇M, W208X, R212X, N₃₂₂X, D663X, and a combination thereof, X being one of the 20 naturally occurring amino acids other than the amino acid residue of the unmodified dipeptidyl peptidase at the mutated position. In some embodiments, the present modified or engineered cleavase has one or more amino acid modification(s) of N₂₀₇M, W208G, R212V, N₃₂₂I, D663A, or a combination thereof.

In some embodiments, the present modified or engineered cleavase exhibits the substrate specificity of the above modified or engineered cleavase. In present some embodiments, the modified or engineered cleavase comprises an amino acid sequence that comprises a catalytic domain, an amine binding site, or Si and/or S2 sites, with at least 20% identity, at least 30% identity, at least 40% identity, at least 50% identity, at least 60% identity, at least 70% identity, at least 80% identity, or at least 90% or more identity with the catalytic domain, the amine binding site, or the Si and/or S2 sites of the above modified or engineered cleavase.

A nucleic acid encoding the above modified or engineered cleavase is provided herein. A vector, e.g., an expression vector, comprising the nucleic acid encoding the above modified or engineered cleavase is also provided herein. A host cell comprising the above nucleic acid or the vector is further provided herein. The host cell can be any suitable type of cell. For example, the host cell can be a mammalian or human host cell.

### III. METHODS OF TREATING TARGET POLYPEPTIDES

In still another embodiment, provided herein is a method of treating a target polypeptide, which method comprises: a) contacting a target polypeptide with an N-terminal modifier agent to form an N-terminally modified target polypeptide having a formula: M-P1-P2-polypeptide, said M being an N-terminal modification, said P1 being the N-terminal amino acid residue of said target polypeptide, and P2 being a penultimate terminal amino acid residue of said target polypeptide; and b) contacting a binder with said N-terminally modified target polypeptide to allow said binder to specifically bind to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

The present methods can be used to treat any suitable target polypeptide or a target polypeptide with suitable length. For example, the length of the target polypeptide and/or the N-terminally modified target polypeptide can be greater than 4 amino acids, greater than 5 amino acids, greater than 6 amino acids, greater than 7 amino acids, greater than 8 amino acids, greater than 9 amino acids, greater than 10 amino acids, greater than 11 amino acids, greater than 12 amino acids, greater than 13 amino acids, greater than 14 amino acids, greater than 15 amino acids, greater than 20 amino acids, greater than 25 amino acids, or greater than 30 amino acids.

The M can comprise any suitable N-terminal modification. For example, the M can comprise a synthetic N-terminal modification. In another example, the M can comprise an amino acid moiety and/or has a size, e.g., length axis or volume, shape, and/or configuration similar to or exceeding a natural amino acid.

In some embodiments, the N-terminal modifier agent comprises an amino acid moiety (NTMaa). The N-terminal modifier agent can be a bipartite N-terminal modifier agent that comprises a natural or unnatural amino acid portion (NTMaa) and an N-terminal blocking group (NTMblk). The amino acid portion (NTMaa) and the N-terminal blocking group (NTMblk) can be connected or linked by any suitable bond or linkage. For example, the amino acid portion (NTMaa) and the N-terminal blocking group (NTMblk) can be connected with an amide bond. In some embodiments, L- or D- configurations of the NTMaa portion of the N-terminal modifier agent are alkylated to prevent racemization during installation of the N-terminal modification to target polypeptides.

In some embodiments, the N-terminal modifier agent does not comprise an amino acid moiety. The N-terminal modifier agent can be a bipartite N-terminal modifier agent that comprises a small (or small molecule) chemical entity having a size, e.g., length axis or volume, shape, and/or configuration similar to or exceeding a natural amino acid, and an N-terminal blocking group (NTM_{blk}). The small (or small molecule) chemical entity and the N-terminal blocking group (NTM_{blk}) can be connected or linked by any suitable bond or linkage. For example, the small (or small molecule) chemical entity and the N-terminal blocking group can be connected with an amide bond. The small (or small molecule) chemical entity can have any suitable size, e.g., length axis or volume. For example, the small (or small molecule) chemical entity has a size, e.g., length axis of ˜5-10 Å and volume of 100-1000 Å³. In some embodiments, the small (or small molecule) chemical entity has a length axis of about 5, 6, 7, 8, 9 or 10 Å, or any range thereof. In some embodiments, the small (or small molecule) chemical entity has a volume of about 100, 200, 300, 400, 500, 600, 700, 800, 900, 1000 Å³. or any range thereof.

The P1 or the N-terminal amino acid residue of a target polypeptide can be any suitable amino acid residue. In some embodiments, the P1 can comprise a naturally-occurring amino acid residue. In some embodiments, the P1 can be selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the P1 can comprise a modification, e.g., a naturally-occurring or a non-natural modification. In some embodiments, the P1 can comprise an amino acid with a post-translational modification.

The P2 or the penultimate terminal amino acid residue of a target polypeptide can be any suitable amino acid residue. In some embodiments, the P2 can comprise a naturally-occurring amino acid residue. In some embodiments, the P2 can be selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the P2 can comprise a modification, e.g., a naturally-occurring or a non-natural modification. In some embodiments, the P2 can comprise an amino acid with a post-translational modification.

In some embodiments, the interaction between the binder and the M has minimal or no impact or influence on the binding specificity between the binder and the N-terminally modified target polypeptide. In some embodiments, the interaction between the binder and the M at least partially determines the binding strength or binding efficiency between the binder and the N-terminally modified target polypeptide.

In some embodiments, there is minimal or no interaction between the binder and the P2. In some embodiments, there is minimal interaction between the binder and the P2, and the minimal interaction between the binder and the P2 has minimal or no impact or influence on the binding specificity, strength and/or efficiency between the binder and the N-terminally modified target polypeptide; and/or P1-P2 occupies a volume or shape encompassing a cavity or pocket of the binder that effectively precludes the P2 from entering into or interacting with an affinity determining region of the binder.

The present binders used in the present methods can specifically bind to N-terminally modified target polypeptide that contains a particular or specific N-terminal amino acid residue and have the ability to distinguish N-terminally modified target polypeptides that contain different N-terminal amino acid residues. The present binders used in the present methods can also specifically bind to N-terminally modified target polypeptides that contain a group of N-terminal amino acid residues, and have the ability to distinguish such N-terminally modified target polypeptides from other N-terminally modified target polypeptides that contain N-terminal amino acid residue(s) outside the recognized group of the N-terminal amino acid residues. In some embodiments, the present binder used in the present methods specifically binds to multiple N-terminally modified target polypeptides that comprise the same P1 residue. In some embodiments, the present binder used in the present methods specifically binds to multiple N-terminally modified target polypeptides that comprise different P1 residues. For example, the present binder used in the present methods can specifically bind to multiple N-terminally modified target polypeptides that comprise 2, 3, 4, 5, 6, 7, 8, 9 or 10 different P1 residues.

The present binders used in the present methods can comprise any suitable type of composition or molecule. For example, the present binders used in the present methods can comprise or can be a polypeptide, e.g., an antibody or a fragment or derivative thereof. In another example, the present binders used in the present methods can comprise or can be an engineered anticalin. In some embodiments, the present binders used in the present methods can comprise an above descried engineered anticalin (see e.g., the above Section I.)

The present methods can further comprise a step c) cleaving the peptide bond between the P1 and P2 to form a polypeptide wherein the P2 becomes N-terminal amino acid residue of the nascent polypeptide.

The peptide bond between the P1 and P2 can be cleaved using any agent or reaction. For example, the peptide bond between the P1 and P2 can be cleaved using a chemical agent or reaction. In some embodiments, the peptide bond between the P1 and P2 is cleaved using a chemical agent or reaction.

In another example, the peptide bond between the P1 and P2 can be cleaved using a modified cleavase. In some embodiments, the peptide bond between the P1 and P2 is cleaved using an above descried modified or engineered cleavase (see e.g., the above Section I.) In some embodiments, the peptide bond between the P1 and P2 is cleaved using a modified or an engineered cleavase described and/or claimed in U.S. provisional application Ser. Nos. 62/823,927, filed Mar. 26, 2019, 62/824,157, filed Mar. 26, 2019, and 62/931,737, filed Nov. 6, 2019, and PCT application No. PCT/US2020/24521, filed Mar. 24, 2020.

In some embodiments, step c) can be conducted while the binder is bound with the N-terminally modified target polypeptide. In some embodiments, step c) can be conducted after the binder is released and/or removed from the N-terminally modified target polypeptide.

In some embodiments, steps a)- c) can be repeated one or more times to form a polypeptide having newly exposed N-terminal amino acid residue.

In the present methods, any suitable number of binder(s) can be used. In some embodiments, step b) can comprise contacting a single binder with the N-terminally modified target polypeptide to allow the binder to specifically bind to the N-terminally modified target polypeptide. In some embodiments, step b) can comprise contacting a plurality of binders with the N-terminally modified target polypeptide to allow the binders to specifically bind to the N-terminally modified target polypeptide.

In some embodiments, the binder used in the present methods can comprise a coding tag with identifying information regarding the binder. The coding tag can comprise any suitable type of molecule or composition. For example, the coding tag can comprise or can be a DNA molecule, an RNA molecule, a PNA molecule, a BNA molecule, an XNA, molecule, an LNA molecule, a yPNA molecule, or a combination thereof. In another example, the coding tag can comprise a unique molecular identifier (UMI) and/or a universal priming site. The binding agent and the coding tag can be joined or linked directly, or indirectly, e.g., via a linker.

The present methods can further comprise step d) transferring the identifying information of the coding tag to a recording tag attached to the N-terminally modified target polypeptide, thereby generating an extended recording tag on the N-terminally modified target polypeptide.

The recording tag can comprise any suitable type of molecule or composition. For example, the recording tag can comprise or can be a DNA molecule, an RNA molecule, a PNA molecule, a BNA molecule, an XNA, molecule, an LNA molecule, a yPNA molecule, or a combination thereof. In another example, the recording tag can comprise a unique molecular identifier (UMI) and/or a universal priming site.

Transferring the identifying information of the coding tag to the recording tag (or vice versa) can be effected using any agent or reaction. For example, transferring the identifying information of the coding tag to the recording tag (or vice versa) can be effected by primer extension or ligation.

The polypeptide can be directly or indirectly joined to a solid support. Any suitable solid support can be used. For example, the solid support can be a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere. In another example, the solid support can comprise a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.

In the present methods, the various steps can be conducted or performed in any suitable order. For example, step d) can be performed after step b), but before step c).

In some embodiments, the steps of: a) contacting a target polypeptide with an N-terminal modifier agent; b) contacting a binder with the N-terminally modified target polypeptide; d) transferring the identifying information of the coding tag to a recording tag attached to the N-terminally modified target polypeptide; and c) cleaving the peptide bond between the P1 and p2 to form a polypeptide wherein the P2 becomes N-terminal amino acid residue of the nascent polypeptide, can be repeated in sequential order to generate one or more additional extended recording tags.

In some embodiments, the present methods can further comprise releasing the binder from the N-terminally modified target polypeptide and/or removing the released binder after step b) and before step c) or d). In some embodiments, the present methods can further comprise releasing the binder from the N-terminally modified target polypeptide and/or removing the released binder after step d) and before step c).

In some embodiments, the present methods can further comprise analyzing the one or more extended recording tag(s). The one or more extended recording tags can be amplified prior to analysis. The one or more extended recording tags can be analyzed using any suitable agent or reaction. For example, the one or more extended recording tags can be analyzed using a nucleic acid sequencing method. Any suitable nucleic acid sequencing method can be used. In some embodiments, the nucleic acid sequencing method can be sequencing by synthesis, sequencing by ligation, sequencing by hybridization, polony sequencing, ion semiconductor sequencing, or pyrosequencing. In some embodiments, the nucleic acid sequencing method can be single molecule real-time sequencing, nanopore-based sequencing, or direct imaging of DNA using advanced microscopy.

### IV. KITS OF TREATING TARGET POLYPEPTIDES

In yet another embodiment, provided herein is a kit of treating a target polypeptide, which kit comprises: a) an N-terminal modifier agent that is configured to contact a target polypeptide to form an N-terminally modified target polypeptide having a formula: M-P1-P2-polypeptide, said M being an N-terminal modification, said P1 being the N-terminal amino acid residue of said target polypeptide, and P2 being a penultimate terminal amino acid residue of said target polypeptide; and/or b) a binder that is configured to specifically bind to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

The present kits can be used to treat any suitable target polypeptide or a target polypeptide with suitable length. For example, the length of the target polypeptide and/or the N-terminally modified target polypeptide is greater than 4 amino acids, greater than 5 amino acids, greater than 6 amino acids, greater than 7 amino acids, greater than 8 amino acids, greater than 9 amino acids, greater than 10 amino acids, greater than 11 amino acids, greater than 12 amino acids, greater than 13 amino acids, greater than 14 amino acids, greater than 15 amino acids, greater than 20 amino acids, greater than 25 amino acids, or greater than 30 amino acids.

The M can comprise any suitable N-terminal modification. For example, the M can comprise a synthetic N-terminal modification. In another example, the M can comprise an amino acid moiety and/or has a size, e.g., length axis or volume, shape, and/or configuration similar to or exceeding a natural amino acid.

In some embodiments, the M can comprise an amino acid moiety. The M can be a bipartite N-terminal modification (NTM) that comprises a natural or unnatural amino acid portion (NTMaa) and an N-terminal blocking group (NTM_{blk}). The amino acid portion (NTMaa) and the N-terminal blocking group (NTM_{blk}) can be connected or linked by any suitable bond or linkage. For example, the amino acid portion (NTMaa) and the N-terminal blocking group (NTM_{blk}) can be connected with an amide bond.

In some embodiments, the M does not comprise an amino acid moiety. The M can be a bipartite N-terminal modification (NTM) that comprises a small (or small molecule) chemical entity having a size, e.g., length axis or volume, shape, and/or configuration similar to or exceeding a natural amino acid, and an N-terminal blocking group (NTM_{blk}). The small (or small molecule) chemical entity and the N-terminal blocking group (NTM_{blk}) can be connected or linked by any suitable bond or linkage. For example, the small (or small molecule) chemical entity and the N-terminal blocking group can be connected with an amide bond. The small (or small molecule) chemical entity can have any suitable size, e.g., length axis or volume. For example, the small (or small molecule) chemical entity has a size, e.g., length axis of ˜5-10 Å and volume of 100-1,000 Å³. In some embodiments, the small (or small molecule) chemical entity has a length axis of about 5, 6, 7, 8, 9 or 10 A, or any range thereof. In some embodiments, the small (or small molecule) chemical entity has a volume of about 100, 200, 300, 400, 500, 600, 700, 800, 900, 1000 Å³. or any range thereof.

The present N-terminally modified target polypeptide can comprise any suitable N-terminal modification (M). In some embodiments, the present N-terminally modified target polypeptide can comprise an N-terminal modification (M) as described in Section V below. The present kits can comprise any suitable N-terminal modifier agent. In some embodiments, the present kits can comprise any suitable N-terminal modifier agent as described in Section V below.

The P1 or the N-terminal amino acid residue of a target polypeptide can be any suitable amino acid residue. In some embodiments, the P1 can comprise a naturally-occurring amino acid residue. In some embodiments, the P1 can be selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the P1 can comprise a modification, e.g., a naturally-occurring or a non-natural modification. In some embodiments, the P1 can comprise an amino acid with a post-translational modification.

The P2 or the penultimate terminal amino acid residue of a target polypeptide can be any suitable amino acid residue. In some embodiments, the P2 can comprise a naturally-occurring amino acid residue. In some embodiments, the P2 can be selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the P2 can comprise a modification, e.g., a naturally-occurring or a non-natural modification. In some embodiments, the P2 can comprise an amino acid with a post-translational modification.

In some embodiments, the interaction between the binder and the M has minimal or no impact or influence on the binding specificity between the binder and the N-terminally modified target polypeptide. In some embodiments, the interaction between the binder and the M at least partially determines the binding strength or binding efficiency between the binder and the N-terminally modified target polypeptide.

In some embodiments, there is minimal or no interaction between the binder and the P2. In some embodiments, there is minimal interaction between the binder and the P2, and the minimal interaction between the binder and the P2 has minimal or no impact or influence on the binding specificity, strength and/or efficiency between the binder and the N-terminally modified target polypeptide; and/or P1-P2 occupies a volume or shape encompassing a cavity or pocket of the binder that effectively precludes the P2 from entering into or interacting with an affinity determining region of the binder.

The present binders used in the present kits can specifically bind to N-terminally modified target polypeptide that contains a particular or specific N-terminal amino acid residue and have the ability to distinguish N-terminally modified target polypeptides that contain different N-terminal amino acid residues. The present binders used in the present kits can also specifically bind to N-terminally modified target polypeptides that contain a group of N-terminal amino acid residues, and have the ability to distinguish such N-terminally modified target polypeptides from other N-terminally modified target polypeptides that contain N-terminal amino acid residue(s) outside the recognized group of N-terminal amino acid residues. In some embodiments, the present binder used in the present kits specifically binds to multiple N-terminally modified target polypeptides that comprise the same P1 residue. In some embodiments, the present binder used in the present kits specifically binds to multiple N-terminally modified target polypeptides that comprise different P1 residues. For example, the present binder used in the present kits can specifically bind to multiple N-terminally modified target polypeptides that comprise 2, 3, 4, 5, 6, 7, 8, 9 or 10 different P1 residues.

The present binders used in the present kits can comprise any suitable type of composition or molecule. For example, the present binders used in the present kits can comprise or can be a polypeptide, e.g., an antibody or a fragment or derivative thereof. In another example, the present binders used in the present kits can comprise or can be an engineered anticalin. In some embodiments, the present binders used in the present kits can comprise an above descried engineered anticalin (see e.g., the above Section I.)

In some embodiments, the present kits can further comprises: c) an agent that is configured to cleave the peptide bond between the P1 and P2 to form a polypeptide wherein the P2 becomes N-terminal amino acid residue of the nascent polypeptide.

The present kits can further comprise: c) an agent that is configured to cleave the peptide bond between the P1 and P2. The peptide bond between the P1 and P2 can be cleaved using any agent or reaction. For example, the present kits can comprise a chemical agent for cleaving the peptide bond between the P1 and P2. In some embodiments, the peptide bond between the P1 and P2 is cleaved using a chemical agent or reaction. In another example, the present kits can comprise an enzyme for cleaving the peptide bond between the P1 and P2. In some embodiments, the present kits can comprise an above descried modified or engineered cleavase (see e.g., the above Section I.) In some embodiments, the present kits can comprise a modified or an engineered cleavase described and/or claimed in U.S. provisional application Ser. No. 62/823,927, filed Mar. 26, 2019, 62/824,157, filed Mar. 26, 2019, and 62/931,737, filed Nov. 6, 2019, and PCT application No. PCT/US2020/24521, filed Mar. 24, 2020.

The present kits can comprise any suitable number of binder(s). In some embodiments, the present kits can comprise a single binder that is configured to specifically bind to the N-terminally modified target polypeptide. In some embodiments, the present kits can comprise a plurality of binders that are configured to specifically bind to the N-terminally modified target polypeptide.

In some embodiments, the binder used in the present kits can comprise a coding tag with identifying information regarding the binder. The coding tag can comprise any suitable type of molecule or composition. For example, the coding tag can comprise or can be a DNA molecule, an RNA molecule, a PNA molecule, a BNA molecule, an XNA, molecule, an LNA molecule, a yPNA molecule, or a combination thereof. In another example, the coding tag can comprise a unique molecular identifier (UMI) and/or a universal priming site. The binding agent and the coding tag can be joined or linked directly, or indirectly, e.g., via a linker.

In some embodiments, the present kits can further comprise: d) a reagent for transferring the identifying information of the coding tag to a recording tag attached to the N-terminally modified target polypeptide, thereby generating an extended recording tag on the N-terminally modified target polypeptide.

The recording tag can comprise any suitable type of molecule or composition. For example, the recording tag can comprise or can be a DNA molecule, an RNA molecule, a PNA molecule, a BNA molecule, an XNA, molecule, an LNA molecule, a γPNA molecule, or a combination thereof. In another example, the recording tag can comprise a unique molecular identifier (UMI) and/or a universal priming site.

Transferring the identifying information of the coding tag to the recording tag (or vice versa) can be effected using any agent or reaction. For example, the present kits can further comprise a chemical ligation reagent or a biological ligation reagent for transferring the identifying information. In some embodiments, the present kits can further comprise a reagent for primer extension of single-stranded nucleic acid or double-stranded nucleic acid for transferring the identifying information.

In some embodiments, the present kits can further comprise a reagent for releasing the binder from the N-terminally modified target polypeptide and/or for removing the released binder.

In some embodiments, the present kits can further comprise an amplification reagent for amplifying the one or more extended recording tag(s).

In some embodiments, the present kits can further comprise a reagent for analyzing the one or more extended recording tag(s). The present kits can comprise any suitable reagent for analyzing the one or more extended recording tag(s), e.g., a reagent for nucleic acid sequencing analysis. In some embodiments, the present kits can comprise a reagent for conducting sequencing by synthesis, sequencing by ligation, sequencing by hybridization, polony sequencing, ion semiconductor sequencing, pyrosequencing, single molecule real-time sequencing, nanopore-based sequencing, or direct imaging of DNA using advanced microscopy, or any combination thereof.

In some embodiments, the present kits can further comprise a solid support selected from the group consisting of a bead, a porous bead, a magnetic bead, a paramagnetic bead, a porous matrix, an array, a surface, a glass surface, a silicon surface, a plastic surface, a slide, a filter, nylon, a chip, a silicon wafer chip, a flow through chip, a biochip including signal transducing electronics, a well, a microtitre well, a plate, an ELISA plate, a disc, a spinning interferometry disc, a membrane, a nitrocellulose membrane, a nitrocellulose-based polymer surface, a nanoparticle (e.g., comprising a metal such as magnetic nanoparticles (Fe₃O₄), gold nanoparticles, and/or silver nanoparticles), quantum dots, a nanoshell, a nanocage, a microsphere, and any combination thereof. In some embodiments, the solid support can comprise a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.

### V. EXEMPLARY N-TERMINAL MODIFICATIONS AND N-TERMINAL MODIFIER AGENTS

In some embodiments, the use of the present binder and modified or engineered cleavase involves an N-terminally modified target polypeptide comprising an N-terminal modification (M). In some embodiments, the present methods comprise contacting a target polypeptide with an N-terminal modifier agent to form an N-terminally modified target polypeptide comprising an N-terminal modification (M).

In some embodiments, the methods and uses of the present disclosure include a step of attaching a modification comprising an N-terminal modification (NTM) to the NTAA of a target polypeptide. In some embodiments, the step is performed using a chemical reagent or combination of chemical reagents to form an amide bond attaching the N-terminal modification to the NTAA.

The N-terminal modification can be any suitably sized and shaped chemical group that is readily connected to the NTAA and that promotes binding of the N-terminally modified target polypeptide to a binder of the invention, such that the binder specifically binds to the N-terminally modified target polypeptide, and the binding specificity is predominantly determined by the NTAA rather than the second (penultimate) amino acid residue of the target polypeptide.

In some embodiments, the N-terminal modification comprises a free carboxylic acid that is useful for attaching the NTM to a free terminal amine group of the NTAA of the target polypeptide. In such embodiments, use of the N-terminal modifier agent is typically combined with a chemical reagent that promotes formation of an amide bond between the carboxylic acid of the NTM and the free amine of the NTAA of the target polypeptide. Suitable chemical reagents are included in Formulas (3)-(8) described below, particularly when Q in each formula represents —OH or —OM.

Suitable chemical reagents that are known in the art for performing the coupling reaction (amide bond formation) between the NTM and the NTAA include conventional peptide coupling reagents such as carbodiimides (e.g., dicyclohexyl carbodiimide (DCC), diisopropyl carbodiimide (DIPC), 1-ethyl-3-(3-dimethylaminopropyl)carbodiimide (EDC), 1-cyclohexyl-(2-morpholinoethyl)carbodiimide tosylate (CMCT), and the like), aminium/uronium salts (e.g., COMU, HATU, HBTU, TBTU, HCTU, and TSTU), phosphonium coupling reagents including PyBOP, PyAOP, PyOxim, and BOP, and phosphonate coupling reagents such as (3-(diethoxyphosphoryloxy)-1,2,3-benzotriazin-4(3H)-one) (DEPBT), and propylphosphonic anhydride (T3P). Suitable carbodiimide reagents include compounds of Formula (1) described below. Suitable aminium/uronium coupling reagents include compounds of Formula (2) described below.

In some embodiments, coupling conditions are used to minimize racemization of the NTMaa moiety of the N-terminal modifier agent during installation onto target polypeptides (Ramu, Vasanthakumar G., et al., “DEPBT as Coupling Reagent To Avoid Racemization in a Solution-Phase Synthesis of a Kyotorphin Derivative.” 2014, Synthesis 46 (11): 1481-86).

In some embodiments, the N-terminal modifier agent reacts with a free amine of the NTAA of the target polypeptide with a help of a chemical reagent comprising an activated ester of a carboxylic acid that is suitable for reacting directly with the free amine. Such activated esters are known in the field of peptide coupling. Suitable chemical reagents are included in Formulas (3)-(8) described below, particularly when Q in each formula represents—ORQ.

In some embodiments, the chemical reagent comprises compound of Formula (1):

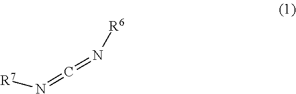

or a salt or conjugate thereof,

wherein

R⁶ and R⁷ are each independently C₁₋₆ alkyl, —CO₂Cl₁₋₄ alkyl, —OR^{k}, aryl, heteroaryl, cycloalkyl or heterocyclyl, wherein the C₁₋₆ alkyl, —CO₂C₁₋₄ alkyl, —OR^{k}, aryl, and cycloalkyl are each unsubstituted or substituted; and

R^{k} is H, C₁₋₆ alkyl, or heterocyclyl, wherein the C₁₋₆ alkyl and heterocyclyl are each unsubstituted or substituted; wherein heterocyclyl can be 5-8 membered ring comprising one or two heteroatoms selected from N, O and S as ring members, where the heteroaryl can be a 5-6 membered single ring or 8-10 membered bicyclic ring, each of which comprises one to three heteroatoms selected from N, O and S as ring members. Cycloalkyls include 3-7 membered carbocyclic rings, optionally substituted. Heterocyclyl can be 5-8 membered ring comprising one or two heteroatoms selected from N, O and S as ring members, and include tetrahydrofuranyl, piperidinyl, piperazinyl, dihydropyranyl, dioxanyl, and the like. Heteroaryl can be a 5-6 membered single ring or 8-10 membered bicyclic ring, each of which comprises one to three heteroatoms selected from N, O and S as ring members. Aryl includes phenyl, which can be substituted or unsubstituted. Heteroaryl includes pyridinyl, pyrimidinyl, or pyrazinyl; oxazolyl, isoxazolyl, thiazolyl, isothiazolyl, furanyl, thienyl, pyrrolidinyl, imidazolyl, pyrazolyl, and triazolyl, as well as a bicyclic group comprising any one of these fused to phenyl. Suitable substituents for the alkyl, cycloalkyl, heterocyclyl, aryl and heteroaryl groups include halo, hydroxy, amino, C₁-C₂ alkylamino, di-(C₁-C₂ alkyl)amino, C₁-C₂ alkyl, C₁-C₂ alkoxy, NO₂, CN, C₁-C₂ haloalkyl, C₁-C₂ haloalkoxy, and halo, and for non-aromatic groups also oxo.

In some embodiments of Formula (1), R⁶ and R⁷ are each independently C₁₋₆ alkyl, 3-7 membered cycloalkyl, —CO₂C₁₋₄ alkyl, or aryl, especially phenyl. In some embodiments, R⁶ and R⁷ are each independently H, C₁₋₆ alkyl, phenyl, or cycloalkyl. In some embodiments, R⁶ and R⁷ are the same. In some embodiments, R⁶ and R⁷ are different.

In some embodiments, one of R⁶ and R⁷ is C₁₋₆ alkyl and the other is selected from the group consisting of C₁₋₆ alkyl, —CO₂C₁₋₄ alkyl, and —OR^{k}, wherein the C₁₋₆ alkyl, —CO₂C₁₋₄ alkyl, and -OR' are each unsubstituted or substituted. In some embodiments, one or both of R⁶ and R⁷ is C₁₋₆ alkyl, optionally substituted with aryl, such as phenyl. In some embodiments, one or both of R⁶ and R⁷ is C₁₋₆ alkyl, optionally substituted with heterocyclyl. In some embodiments, one of R⁶ and R⁷ is —CO₂C₁₋₄ alkyl and the other is selected from the group consisting of C₁₋₆ alkyl, —CO₂C₁₋₄ alkyl, and —OR^{k}, wherein the C₁₋₆ alkyl, —CO₂C₁₋₄ alkyl, and —OR^{k} are each unsubstituted or substituted. In some embodiments, one of R⁶ and R⁷ is optionally substituted aryl and the other is selected from the group consisting of C₁₋₆ alkyl, —CO₂C₁₋₄ alkyl, —OR^{k}, aryl, heteroaryl, cycloalkyl and heterocyclyl, wherein the C₁₋₆ alkyl, —CO₂C₁₋₄ alkyl, —OR^{k}, aryl, and cycloalkyl are each unsubstituted or substituted. In some embodiments, one or both of R⁶ and R⁷ is aryl, optionally substituted with up to three groups selected from C₁₋₆ alkyl, halo, and NO₂.

In some embodiments, the compound of formula (1) is selected from the group consisting of

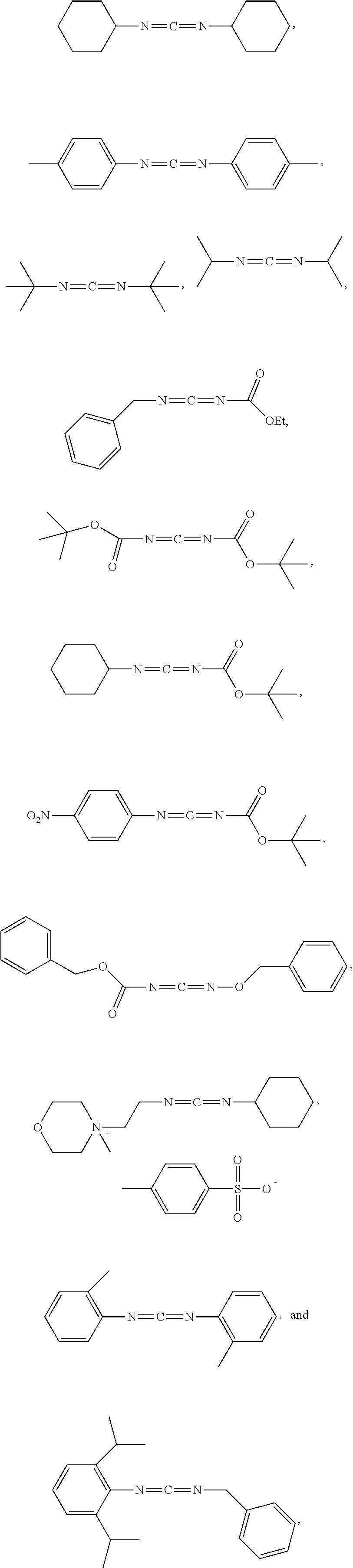

or a salt or conjugate thereof.

In some embodiments, the chemical reagent comprises a compound of Formula (2):

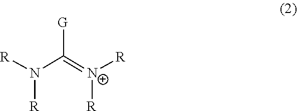

wherein:

each R is independently C₁₋₄ alkyl, optionally substituted with up to three groups selected from halo, C₁₋₂ alkoxy, C₁₋₄ alkylamino, di-(C₁₋₄ alkyl)amino, C₁₋₂ haloalkyl, and C₁₋₂ haloalkoxy;

and two R groups on the same N can optionally cyclize to form a 5-7 membered ring optionally containing an additional heteroatom selected from N, O and S as a ring member, and optionally substituted with one or two groups selected from oxo, C₁₋₂ alkyl, C₁₋₂ alkoxy, C₁₋₂ haloalkyl, oxo, and C₁₋₂ haloalkoxy; and

G is selected from halo, benzotriazolyloxy, halobenzotriazolyloxy, pyridinotriazolyloxy, benzotriazolyl-N-oxide, pyridinotriazolyl-N-oxide, —O-(N-succinimide), 1-cyano-2-ethoxy-2-oxoethylideneaminooxy, and —O-(N-phthalimide).

Compounds of Formula (2) typically also comprise an unreactive anionic counterion, such as halo, tetrafluoroborate, hexafluorophosphate, fluorosulfonate, trifluoromethylsulfonate, and the like.

Examples of suitable compounds of Formula (2) include well-known peptide coupling reagents such as COMU, HATU, HBTU, TBTU, HCTU, TFFH, and TSTU.

Phosphonium coupling reagents including PyBOP, PyAOP, PyOxim, and BOP, can also be used in the methods of the invention, as well as phosphonate coupling reagents such as DEPBT.

In some embodiments, the chemical reagent for use in the methods of modifying the NTAA of a target polypeptide comprises a compound selected from compounds of Formulas (3)-(8), where these formulas represent groups that react with and/or attach to the NTAA, providing the modifying group on the NTAA. When Q is OH or OM in these formulas, the chemical reagent may also comprise a peptide coupling reagent such as an aminium, uronium, or phosphonium peptide coupling reagent, including a compound of formula (1) or formula (2).

In some embodiments, the chemical reagent comnrises a compound of Formula (3):

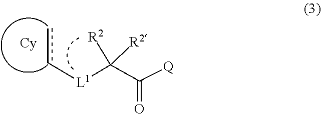

wherein Q is ORQ, OH, or OM, where M is cationic counterion;

wherein Q is ORQ, OH, or OM, where M is a cationic counterion;

each RQ is independently aryl or heteroaryl, each of which is optionally substituted with one or more groups selected from halo, nitro, cyano, sulfonate, carboxylate, alkylsulfonyl, and N of heteroaryl is optionally oxidized; or RQ can be —C(═O)R or —C(═O)—OR;

Cy is a 5 to 7 membered ring or an 8-10 membered bicyclic ring system, and Cy may be absent or present;

when present, ring Cy may be saturated, unsaturated, or aromatic, and the dashed bond may be a single bond, double bond, or aromatic bond;

when Cy is present, it may be a carbocyclic ring, or it may contain one to three heteroatoms selected from N, O, B, and S as ring members; and Cy is optionally substituted with one to six groups (or with one to four groups when Cy is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, —SR⁴, —S(O)ₙR⁴, —NR⁴SO₂R⁴, —SO₂N(R⁴)₂, heteroaryl, C₁-C₂ alkyl, C₁-C₂ haloalkyl, C₁-C₂ haloalkoxy, and —OR⁴;

when ring Cy is absent, the dashed bond may be a single bond or a double bond, and the dashed bond is optionally substituted by one or two groups selected from halo, CN, C₁-C₂ alkyl, C₁-C₂ haloalkyl, C₁-C₂ haloalkoxy, CO₂R⁴, and —OR⁴; each L¹ is independently a bond or C₁-C₂ alkylene, C₁-C₂ haloalkylene, NHC(O), SO₂, or NHSO₂;

R² and R^{2′} can each be H or a side chain of an amino acid, e.g. one of the side chains of the 20 common amino acid side chains, optionally protected amino acid side chains, post-translationally modified amino acid side chains, unnatural amino acid sidechains;

or R² or R^{2′} can be an aryl, heteroaryl, bicyclic aryl, or bicyclic heteroaryl, each of which is optionally substituted with up to three groups independently selected from halo, cyano, azido, amino, C₁-C₂ alkyl, C₁-C₂ haloalkyl, C₁-C₂ alkoxy, and C₁-C₂ haloalkoxy;

[symbol] represents an optional link between R² and L¹, forming a 5-6 membered ring;

n at each occurrence is independently 1 or 2; and

each R and R⁴ is independently selected from H, C₁₋₂ alkyl, and C₁-C₂ haloalkyl. Suitable cationic counterions (M) for compounds of Formula (3)-(8) include metal cations such as Li, Na and K, and ammonium species such as tetra-(C₁₋₆ alkyl)ammonium.

In some embodiments, the chemical reagent comprises a compound of Formula (4):

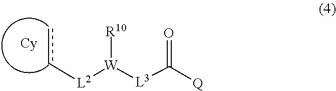

wherein;

wherein Q is OH, OR^{Q} or OM,

each R^{Q} is independently aryl or heteroaryl, each of which is optionally substituted with one or more groups selected from halo, nitro, cyano, sulfonate, carboxylate, alkylsulfonyl, and N of heteroaryl is optionally oxidized; or R^{Q} can be —C(═O)R or —C(═O)—OR;

and M is cationic counterion;

W is a bond or a group selected from alkyl, cycloalkyl, heterocyclyl, aryl, heteroaryl, and bicyclic heteroaryl, each of which is optionally substituted with up to four groups independently selected from halo, OH, cyano, azido, —SR⁴, —S(O)ₙR⁴, —NR⁴SO₂R⁴, —SO₂N(R⁴)₂, —B(OR⁴)₂, oxo (unless W is aromatic), amino, C₁-C₂ alkyl, C₁-C₂ haloalkyl, C₁-C₂ alkoxy, and C₁-C₂ haloalkoxy;

when W is a ring, ring W may be saturated, unsaturated, or aromatic; when W is a heterocyclic or heteroaromatic ring, it may contain one or two heteroatoms selected from N, O and S as ring members;

curved dotted line represents an optional linkage connecting R¹⁰ and L² into a 5-6 membered ring, optionally including an additional N, O or S as a ring member;

R¹⁰ is selected from H, halo, CN, NH₂, NH(CH₃), N(CH₃)₂, NO₂ NHFmoc, NHBoc, C(O)NR₂, NHC(O)R, NHC(O)OR, B(OR)₂, aryl, heteroaryl, C₁-C₂ alkyl, C₁-C₂ haloalkyl, C₁-C₂ haloalkoxy, and —OR⁴; and R¹⁰ is absent when W is a bond;

L² and L³ are independently selected from a bond, CH₂, SO₂R, NHSO₂R, C(═O)R, RNHC(═O), RNCH₃C(═O), C₁-C₂ alkylene, C₁-C₂ haloalkylene, or triazole;

each R is independently selected from C₁₋₆ alkyl, phenyl, and benzyl, each of which is optionally substituted with up to three groups selected from halo, CN, C₁-C₂ haloalkyl, C₁-C₂ haloalkoxy, CO₂R⁴, and —OR⁴;

Cy is a 5 to 7 membered ring or an 8-10 membered bicyclic ring system, and Cy may be absent or present;

when present, ring Cy may be saturated, unsaturated, or aromatic, and the dashed bond may be a single bond, double bond, or aromatic bond;

when Cy is present, it may be a carbocyclic ring, or it may contain one to three heteroatoms selected from N, O, B and S as ring members; and Cy is optionally substituted with one to six groups (or with one to four groups when Cy is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, —SR⁴, —S(O),R⁴, —NR⁴SO₂R⁴, —SO₂N(R⁴)₂, and —OR⁴;

when ring Cy is absent, the dashed bond may be a single bond or a double bond, and the dashed bond is optionally substituted by one or two groups selected from halo, CN, C₁-C₂ alkyl, C₁-C₂ haloalkyl, C₁-C₂ haloalkoxy, CO₂R⁴, and —OR⁴;

each L¹ is independently a bond or C₁-C₂ alkylene, C₁-C₂ haloalkylene, NHC(O), SO₂, or NHSO₂;

n at each occurrence is independently 1 or 2; and

R⁴ is independently selected at each occurrence from H, C₁-C₂ alkyl, and C₁-C₂ haloalkyl.

When W in Formula (4) represents a bond, it directly links L² to L³, and R¹⁰ is absent.

In some embodiments, the chemical reagent comprises a compound of Formula (5):

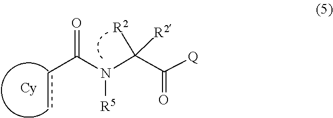

wherein Q is OH, OR^{Q} or OM,

each R^{Q} is independently aryl or heteroaryl, each of which is optionally substituted with one or more groups selected from halo, nitro, cyano, sulfonate, carboxylate, alkylsulfonyl, and N of heteroaryl is optionally oxidized; or RQ can be —C(═O)R or —C(═O)—OR; and M is cationic counterion;

dotted line represents an optional link between R² and nitrogen, forming a 5-6 membered ring: when the optional link is present, R⁵ is absent;

Cy is a 5 to 7 membered ring or an 8-10 membered bicyclic ring system, and Cy may be absent or present;

when present, ring Cy may be saturated, unsaturated, or aromatic, and the dashed bond may be a single bond, double bond, or aromatic bond;

when Cy is present, it may be a carbocyclic ring, or it may contain one to three heteroatoms selected from N, O, B, and S as ring members; and Cy is optionally substituted with one to six groups (or with one to four groups when Cy is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, —SR⁴, —S(O)ₙR⁴, —NR⁴SO₂R⁴, —SO₂N(R⁴)₂, and —OR⁴;

when ring Cy is absent, the dashed bond may be a single bond or a double bond, and the dashed bond is optionally substituted by one or two groups selected from halo, CN, C₁-C₂ alkyl, C₁-C₂ haloalkyl, C₁-C₂ haloalkoxy, CO₂R⁴, and —OR⁴;

R² and R^{2′} can each be the side chain of an amino acid, e.g. one of the side chains of the 20 common amino acid side chains, optionally protected amino acid side chains, post-translationally modified amino acid side chains, unnatural amino acid sidechains; or

R² and R^{2′} can each be H or a group selected from aryl, heteroaryl, bicyclic aryl, bicyclic heteroaryl, and heterocyclyl, each of which is optionally substituted with one to six groups (or with one to four groups when R² or R^{2′} is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, and —OR⁴;

each R and R⁴ is independently selected at each occurrence from H, C₁-C₂ alkyl, and C₁-C₂ haloalkyl;

n at each occurrence is independently 1 or 2; and

R⁵ is independently selected at each occurrence from H, C₁-C₂ alkyl, C₁-C₂ haloalkyl, C₁-C₂ alkoxy, and C₁-C₂ haloalkoxy.

In some embodiments, the chemical reagent comprises a compound of Formula (6);

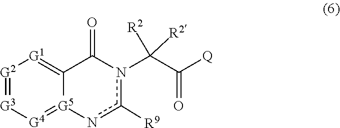

wherein Q is OH, OR^{Q} or OM,

each R^{Q} is independently aryl or heteroaryl, each of which is optionally substituted with one or more groups selected from halo, nitro, cyano, sulfonate, carboxylate, alkylsulfonyl, and N of heteroaryl is optionally oxidized; or R^{Q} can be —C(═O)R or —C(═O)—OR;

M is a cationic counterion;

G¹-G⁵ are each independently selected from CH, CJ, BN, BO, and N, provided not more than 3 of G¹-G⁵ are N;

the dashed bonds can be single bonds or double bonds;

J at each occurrence is independently selected from H, C₁-C₂ alkyl, NO₂, C₁-C₂ haloalkyl, C₁-C₂ haloalkoxy, halo, —OR⁸, —N(R⁸)₂, —NR⁸SO₂R⁸, —SO₂N(R⁸)₂, SO₃R⁸, —B(OR⁸)₂, C(═O)R⁸, CN, CON(R⁸)₂, —COOR^{S}, —C(—O)Ar, and tetrazole, where Ar represents a phenyl or 5-6 membered heteroaryl ring that is optionally substituted with one or two groups selected from halo, CN, R⁸ and OR⁸;

R² and R²′ can each be the side chain of an amino acid, e.g. one of the side chains of the 20 common amino acid side chains, optionally protected amino acid side chains, post-translationally modified amino acid side chains, unnatural amino acid sidechains; or

R² and R^{2′} can each be H or a group selected from aryl, heteroaryl, bicyclic aryl, bicyclic heteroaryl, and heterocyclyl, each of which is optionally substituted with one to six groups (or with one to four groups when R² or R^{2′} is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, and —OR⁴;

each R, R⁴ and R⁸ is independently selected at each occurrence from H, C₁-C₂ alkyl, and C₁-C₂ haloalkyl; and

n at each occurrence is independently 1 or 2; and

R⁹ is H, CH₃, benzyl, substituted benzyl;

In some embodiments, the chemical reagent comprises a compound of Formula (7):

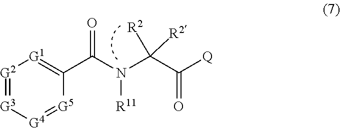

wherein Q is OH, OR^{Q} or OM,

each R^{Q} is independently aryl or heteroaryl, each of which is optionally substituted with one or more groups selected from halo, nitro, cyano, sulfonate, carboxylate, alkylsulfonyl, and N of heteroaryl is optionally oxidized; or R^{Q} can be —C(═O)R or —C(═O)—OR;

in some embodiments, R^{Q} is 4-nitrophenyl, 2,4-dinitrophenyl, 4-fluorophenyl, 2,4-difluorophenyl, 2,3,4,5,6-pentafluorophenyl, 2,3,5,6-tetrafluorophenyl, 4-sulfo-2,3,5,6,tetrafluorophenyl, halogen, imidazole, pyrazole, benzotriazole, and triazole; and M is a cationic counterion;

G¹-G⁵ are each independently selected from CH, CJ, BN, BO, and N, provided not more than 3 of G¹-G⁵ are N;

[symbol] represents an optional link between R² and the nitrogen atom, forming a 5-6 membered ring: when the link is present, R¹¹ is absent;

J at each occurrence is independently selected from H, C₁-C₂ alkyl, NO₂, C₁-C₂ haloalkyl, C₁-C₂ haloalkoxy, halo, —OR⁸, —N(R⁸)₂, —SR⁸, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR⁸, —C(—O)Ar, and tetrazole, where Ar represents a phenyl or 5-6 membered heteroaryl ring that is optionally substituted with one or two groups selected from halo, CN, R⁸ and OR⁸;

R² and R^{2′} can each be the side chain of an amino acid, e.g. one of the side chains of the 20 common amino acid side chains, optionally protected amino acid side chains, post-translationally modified amino acid side chains, unnatural amino acid sidechains;

or R² and R^{2′} can each be H or a group selected from aryl, heteroaryl, bicyclic aryl, bicyclic heteroaryl, and heterocyclyl, each of which is optionally substituted with one to six groups (or with one to four groups when R² or R^{2′} is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, and —OR⁴;

each R, R⁴ and R⁸ is independently selected at each occurrence from H, C₁-C₂ alkyl, and C₁-C₂ haloalkyl;

n at each occurrence is independently 1 or 2; and

R¹¹ is H, CH₃, benzyl, or substituted benzyl.

In some embodiments, the chemical reagent comprises a compound of Formula (8):

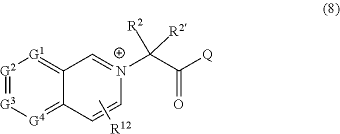

wherein Q is OH, OR^{Q}, or OM,

each R^{Q} is independently aryl or heteroaryl, each of which is optionally substituted with one or more groups selected from halo, nitro, cyano, sulfonate, carboxylate, alkylsulfonyl, and N of heteroaryl is optionally oxidized; or R^{Q} can be —C(═O)R or —C(═O)—OR;

M is a cationic counterion;

G¹-G⁵ are each independently selected from CH, CJ, BN, BO, and N, provided not more than 3 of G¹-G⁵ are N;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR^{S}, —C(—O)Ar, and tetrazole, where Ar represents a phenyl or 5-6 membered heteroaryl ring that is optionally substituted with one or two groups selected from halo, CN, R⁸ and OR⁸;

R² and R^{2′} can each be the side chain of an amino acid, e.g. one of the side chains of the 20 common amino acid side chains, optionally protected amino acid side chains, post-translationally modified amino acid side chains, unnatural amino acid sidechains;

or R² and R^{2′} can each be H or a group selected from aryl, heteroaryl, bicyclic aryl, bicyclic heteroaryl, and heterocyclyl, each of which is optionally substituted with one to six groups (or with one to four groups when R² or R^{2′} is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, and —OR⁴;

each R, R⁴ and R⁸ is independently selected at each occurrence from H, C₁-C₂ alkyl, and C₁-C₂ haloalkyl;

n at each occurrence is independently 1 or 2; and

R¹² represents one or two optional substituents on the pyridinium ring, which are independently selected from C₁-C₂ alkyl, C₁-C₂ alkoxy, C₁-C₂ haloalkyl, C₁-C₂ haloalkoxy, and halo.

Compounds of Formula (8) also comprise an anionic counterion, typically an unreactive anionic counterion, such as halo, tetrafluoroborate, hexafluorophosphate, fluorosulfonate, trifluoromethylsulfonate, and the like.

In some embodiments, the chemical reagent comprises a compound of Formula (9):

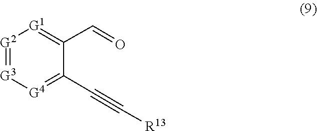

wherein:

G¹-G⁴ are each independently selected from CH, CJ, and N, provided not more than 3 of G¹-G⁴ are N;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, -NR⁸SO₂R⁸, —SO₂N(R⁸)₂, SO₃R⁸, —B(OR⁸)₂, C(═O)R⁸, CN, CON(R⁸)₂, —COOR⁸, —C(—O)Ar, and tetrazole, where Ar represents a phenyl or 5-6 membered heteroaryl ring that is optionally substituted with one or two groups selected from halo, CN, R⁸ and OR⁸;

each R⁸ is independently selected from H, C₁-C₂ alkyl, and C₁-C₂ haloalkyl;

n at each occurrence is independently 1 or 2; and

R¹³ is selected from H, C₁-C₂ alkyl, C₁-C₂ alkoxy, C₁-C₂ haloalkyl, and C₁-C₂ haloalkoxy.

In some embodiments, the chemical reagent (the N-terminal modifier agent) comprises a compound selected from the group consisting of compounds of the following formula:
(A)

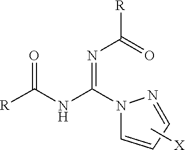

wherein R is CH₃, CF₃, OC(CH₃)3, or OCH₂C₆H₅,

and X is H, CH₃, CF₃, CF₂H, or OCH₃;
(B)

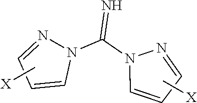

wherein X is H, CH₃, CF₃, CF₂H, OCH₃, or SO₂NH₂;
(C)

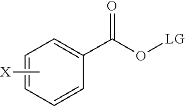

wherein X is H, F, Cl, OCH₃, OCF₃, CN, or SO₂NH₂,

and LG is succinimide, pentafluorophenyl, or tetrafluorophenyl; and
(D)

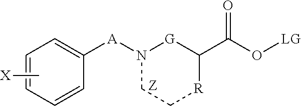

wherein X is H, F, Cl, NH₂, OCH₃, OCF₃, CN, or SO₂NH₂, A=CONH or SO₂, G=0 or 1 CH₂,

R is any amino acid or unnatural amino acid, and

Z ring=0 (not there), 1, 2, or 3 CH₂.

A compound of Formula (9) can be used to generate a modified target polypeptide by contacting the target polypeptide with a 2-ethynyl benzaldehyde derivative of Formula (9) in a polar aprotic solvent such as DMSO, DMF, DMA and the like in 10-500 mM buffer (e.g., PBST, IVIES, acetate, etc., where the PBST buffer is 1X phosphate-buffered saline having 0.1% Tween 20 detergent) at pH 6-9 and a reaction temperature of about 20-80° C., preferably 25-60° C. Typically a concentration of 1-100 mM of the compound of Formula (9) is used.

When a compound of Formula (9) reacts with a target polypeptide, it forms a bicyclic group of Formula (10):

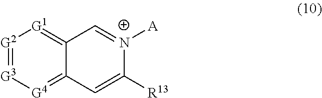

wherein A represents the point of attachment of the group to P1 of a target polypeptide;

G¹-G⁴ are each independently selected from CH, CJ, and N, provided not more than 3 of G¹-G⁴ are N;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR⁸, —C(—O)Ar, and tetrazole, where Ar represents a phenyl or 5-6 membered heteroaryl ring that is optionally substituted with one or two groups selected from halo, CN, R⁹ and OR⁹;

each R⁸ and each R⁹ is independently selected from H, C₁-C₂ alkyl, and C₁-C₂ haloalkyl;

n at each occurrence is independently 1 or 2; and

R¹³ is selected from H, C₁-C₂ alkyl, C₁-C₂ alkoxy, C₁-C₂ haloalkyl, and C₁-C₂ haloalkoxy.

Therefore, in some embodiments the modified target polypeptides of the Formula M-P1-P2-polypeptide, M can be a group of Formula (10). In some embodiments, the reagent of Formula (9) used to attach M to the target polypeptide NTAA is 2-ethynyl-6 fluorobenzaldehyde, and the group M in the modified target polypeptides of the Formula M-P1-P2-polypeptide can be an 8-fluoroisoquinolinium of Formula (10), where G¹ is CF; G²-G⁴ are all CH; and R¹³ is H.

Compounds of Formula (10) also comprise an anionic counterion, typically an unreactive anionic counterion, such as halo, tetrafluoroborate, hexafluorophosphate, fluorosulfonate, trifluoromethylsulfonate, and the like.

Unlike compounds of Formulas (3)-(8) where Q is OH or OM, which require a peptide coupling agent, when the NTM is a compound of Formula (9), no coupling agent is needed, as the ethynyl arylaldehyde reacts directly with the free amine of the NTAA.

R² for compounds of Formulas (3)-(8) can in particular be a side chain of an amino acid selected from Alanine, aspartic acid, asparagine, glutamic acid, glutamine, glycine, (2-, 3-, or 4-pyridyl-)alanine, phenylglycine, 4-fluorophenylglycine, leucine, norleucine, isoleucine, cycloleucine, valine, dimethylglycine, methionine, methionine sulfoxide, phenylalanine, halophenylalanine, haloalkylphenylalanine, cyclopropylalanine, (2-thienyl)alanine, cyclopropylglycine, serine, phosphoserine, threonine, phosphothreonine, cysteine, carbamidomethylcysteine, trifluoromethylcysteine, tyrosine, phosphotyrosine, tryptophan, histidine, acetyllysine, proline, (2- or 3-)azetidine carboxylic acid, piperidine carboxylic acid, methylated lysine, citrulline, nitroarginine, and norvaline. In some embodiments, R^{2′} is H.

In Formula (3), (4), or (5), the ring Cy can be aromatic, heteroaromatic, cycloalkyl, or heterocyclic. Suitable options for Cy include phenyl, pyridinyl, pyrimidinyl, pyrazinyl, oxazolyl, isoxazolyl, thiazolyl, isothiazolyl, furanyl, thienyl, pyrrolidinyl, imidazolyl, pyrazolyl, and triazolyl, as well as a bicyclic group comprising any one of these fused to a phenyl or pyridinyl ring; naphthyl, quinolinyl, isoquinolinyl, benzodiazaborininyl and the like, and all of these are optionally substituted. Suitable substituents for the alkyl, cycloalkyl, heterocyclyl, aryl and heteroaryl groups include halo, C₁-C₂ alkyl, hydroxy, C₁-C₂ alkoxy, amino, C₁-C₂ alkylamino, di-(C₁-C₂ alkyl)amino, NO₂, CN, C₁-C₂ haloalkyl, C₁-C₂ haloalkoxy, and halo, and for non-aromatic groups also including oxo.

In some embodiments of Formula (3), (4), or (5), the ring Cy is phenyl or pyridinyl, and is optionally substituted with one or two groups selected from amino, halo, hydroxy, CF₃, OCF₃ NO₂, SO₂Me, SO₂NH₂, methoxy, methyl, phenyl, and —B(OR)₂.

Where Q is —OR^{Q} in any of Formulas (3)-(8), R^{Q} is typically an electron-deficient aryl or heteroaryl group. Suitable options include benzotriazolyl, halobenzotriazolyl, pyridinotriazolyl, benzotriazolyl-N-oxide, pyridinotriazolyl-N-oxide, —N-succinimide, 1-cyano-2-ethoxy-2-oxoethylideneamino, -N-phthalimide, 4-nitrophenyl, 2,4-dinitrophenyl, 4-fluorophenyl, 2,4-difluorophenyl, 2,3,4,5,6-pentafluorophenyl, 2,3,5,6-tetrafluorophenyl, and 4-sulfo-2,3,5,6,tetrafluorophenyl.

In compounds of Formulas (6)-(9), each of G¹-G⁵ is typically N or CJ, and preferably no more than two of them in any compound is N. In some embodiments, each J is selected from H, amino, halo, hydroxy, CF₃, OCF₃ NO₂, SO₂Me, SO₂NR₂, methoxy, methyl, phenyl, and -B(OR)2 where each R is independently H or C₁₋₂ alkyl.

### VI. TARGET POLYPEPTIDE ASSAYS

In some embodiments, the methods provided include using the macromolecules, especially target polypeptide(s) associated with a recording tag in a macromolecule analysis assay. In some particular embodiments, the macromolecules with associated and/or attached recording tags are subjected to a polypeptide analysis assay. In some cases, the macromolecule analysis assay is performed to assess the macromolecule, or to identify or determine at least a portion of the sequence of the polypeptide macromolecule. In some embodiments, a plurality of macromolecules are analyzed using the described methods.

In some embodiments, the macromolecule analysis assay is performed to identify, quantify, characterize, distinguish, or a combination thereof, all or a portion of the components of the macromolecule. In some embodiments, the macromolecule analysis assay is performed for analysis of proteins, polypeptides, peptides, nucleic acid molecules, carbohydrates, lipids, macrocycles, chimeric macromolecules, or any combinations thereof. In some embodiments, the macromolecule analysis assay is performed to analyze two or more macromolecules. In some cases, the macromolecule analysis assay is performed to analyze two or more interacting, contacting, or neighboring macromolecules. In some examples, the macromolecule analysis assay includes the binding or contacting of a probe to a macromolecule. In some embodiments, the probe is labeled with an oligonucleotide such as a nucleic acid tag. In some embodiments, the probe comprises a small molecule. In some cases, the macromolecule analysis assay includes a small molecule reactive probe. In some embodiments, the probe interacts with, reacts with, or binds to at least a portion of the macromolecule. In some embodiments, the probe binds or interacts with the macromolecule at a reactive site. In some embodiments, the probe binds a binding site of a macromolecule. In some embodiments, the probe binds to an enzyme.

In some embodiments, the macromolecule analysis assay is a next generation protein assay using multiple binding agents and enzymatically or chemically mediated sequential information transfer. In some cases, the analysis assay is performed on immobilized protein molecules simultaneously bound by two or more cognate binding agents (e.g., antibodies). After multiple cognate antibody binding events, a combined primer extension and DNA nicking step is used to transfer information from the coding tags of bound antibodies to the recording tag. In some cases, polyclonal antibodies (or mixed population of monoclonal antibody) to multivalent epitopes on a protein can be used for the assay.

In some embodiments, the macromolecule comprises a polypeptide and the method include performing a polypeptide analysis assay. In some embodiments, the sequence (or a portion of the sequence thereof) and/or the identity of a protein is determined using a polypeptide analysis assay. In some embodiments, the macromolecules may be processed or treated, such as with one or more enzymes and/or reagents. In some examples, the polypeptide analysis assay includes assessing at least a partial sequence or identity of the polypeptide using suitable techniques or procedures. For example, at least a partial sequence of the polypeptide can be assessed by N-terminal amino acid analysis or C-terminal amino acid analysis. In some embodiments, at least a partial sequence of the polypeptide can be assessed using a ProteoCode assay. In some examples, at least a partial sequence of the polypeptide can be assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Publication Nos. WO 2017/192633, WO 2019/089836, WO 2019/089846, and WO 2019/089851.

In some embodiments, the provided methods are for generating a nucleic acid encoded library representation of the binding history of the macromolecule. This nucleic acid encoded library can be amplified, and analyzed using high-throughput next generation digital sequencing methods, enabling millions to billions of molecules to be analyzed per run. The creation of a nucleic acid encoded library of binding information is useful in another way in that it enables enrichment, subtraction, and normalization by DNA-based techniques that make use of hybridization. These DNA-based methods are easily and rapidly scalable and customizable, and more cost-effective than those available for direct manipulation of other types of macromolecule libraries, such as protein libraries. Thus, nucleic acid encoded libraries of binding information can be processed prior to sequencing by one or more techniques to enrich and/or subtract and/or normalize the representation of sequences. This enables information of maximum interest to be extracted much more efficiently, rapidly and cost-effectively from very large libraries whose individual members may initially vary in abundance over many orders of magnitude. Importantly, these nucleic-acid based techniques for manipulating library representation are orthogonal to more conventional methods, and can be used in combination with them.

In an exemplary workflow for analyzing peptides or polypeptides, the method generally includes contacting and specific binding of a binding agent comprising a coding tag to terminal amino acid (e.g., NTAA) of a peptide and transferring the binding agent's coding tag information to the recording tag associated with the peptide, thereby generating a first order extended recording tag. The terminal amino acid bound by the binding agent may be a chemically labeled or modified terminal amino acid. In some embodiments, the terminal amino acid (e.g., NTAA) is eliminated after the information from the coding tag is transferred. The terminal amino acid eliminated may be a chemically labeled or modified terminal amino acid. Removal of the NTAA by contacting with an enzyme or chemical reagents converts the penultimate amino acid of the peptide to a terminal amino acid. The polypeptide analysis may include one or more cycles of binding with additional binding agents to the terminal amino acid, transferring information from the additional binding agents to the extended nucleic acid thereby generating a higher order extended recording tag containing information from two or more coding tags, and eliminating the terminal amino acid in a cyclic manner. Additional binding, transfer, labeling, and removal, can occur as described above up to n amino acids to generate an n^{th} order extended nucleic acid, which collectively represent the peptide. In some embodiments, steps including the NTAA in the described exemplary approach can be performed instead with a C-terminal amino acid (CTAA). In some embodiments, the order of the steps in the process for a degradation-based peptide or polypeptide sequencing assay can be reversed or be performed in various orders. For example, in some embodiments, the terminal amino acid labeling can be conducted before and/or after the polypeptide is bound to the binding agent. In some embodiments, the workflow may include one or more wash steps before and/or after binding of the binding agents, transfer of information, labeling or modifying of the terminal amino acid, and/or removal of the terminal amino acid.

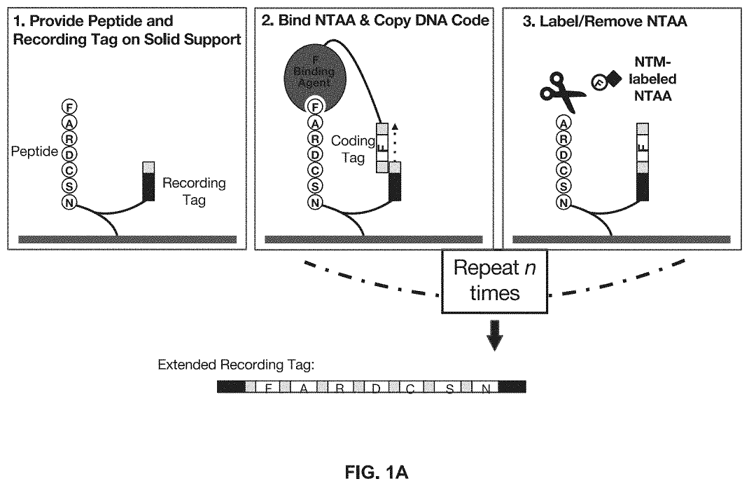

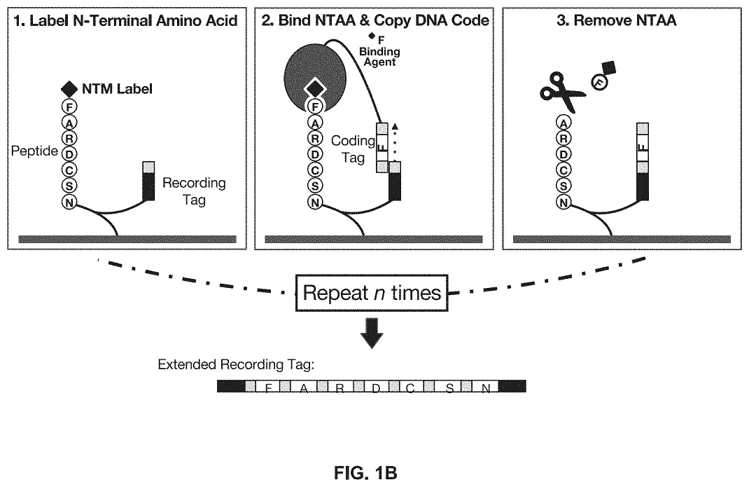

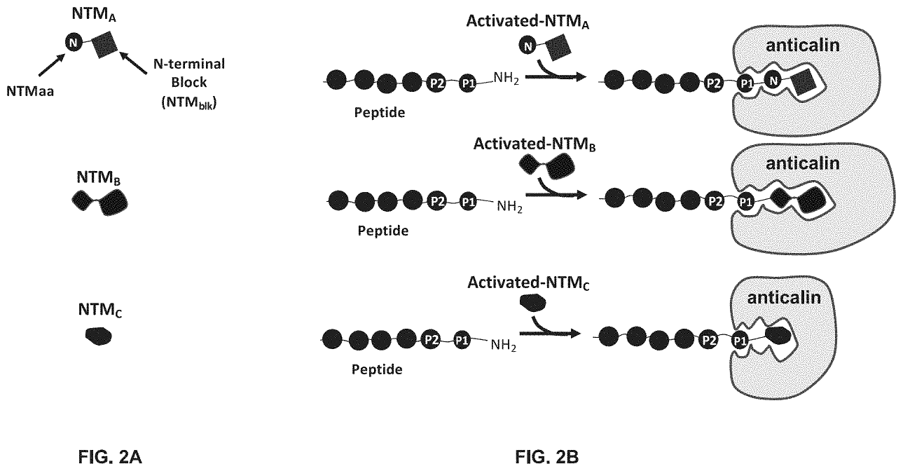

FIG. 1A and 1B depicts a degradation-like approach using a cyclic process including coding tag information transfer to a recording tag attached to the polypeptide, terminal amino acid elimination (e.g., NTAA elimination), and repeating the process in a cyclic manner. In some embodiments, the polypeptide is attached, directly or indirectly, on a solid support. For example, the polypeptide is immobilized on a solid support via a capture agent. Either the protein or capture agent may co-localize or be labeled with a recording tag, and proteins with associated recording tags are directly immobilized on a solid support. Information can be transferred from the coding tag on the bound binding agent to a proximal recording tag using any suitable means including by ligation or primer extension. In one embodiment as depicted, the coding tag includes spacer that is complementary to the spacer in the recording tag and can be used to initiate a primer extension reaction to transfer recording tag information to the coding tag. The final extended recording tag is optionally flanked by universal priming sites to facilitate downstream amplification and/or DNA sequencing. The forward universal priming site (e.g., Illumina's P5-S1 sequence) can be part of the original recording tag design and the reverse universal priming site (e.g., Illumina's P7-S2′ sequence) can be added (e.g., by extension) to the final extended recording tag. This final step may be done independently of a binding agent. In some embodiments, the terminal amino acid bound by the binding agent is a natural or unmodified terminal amino acid as depicted in FIG. 1A. In some embodiments, the cyclic process includes modification of the terminal amino acid (e.g., N-terminal amino acid (NTAA) functionalization) as depicted in FIG. 1A and 1B.

In a workflow which includes binding of a natural or unmodified terminal amino acid as depicted in FIG. 1A, the analysis method includes contacting the polypeptide with a binding agent that is attached to a DNA coding tag. Upon binding of the binding agent to the NTAA of the polypeptide, information of the coding tag is transferred to the recording tag (e.g., via primer extension or ligation) to generate an extended recording tag. The NTAA is labeled and eliminated via chemical or biological (e.g., enzymatic) means to expose a new NTAA.

In a workflow which includes a modified terminal amino acid as depicted in FIG. 1B, the first step includes labeling or modifying the N-terminal amino acid (NTAA) with a functionalization reagent to enable removal of the NTAA in a later step; the functionalizing reagent generates an NTAA residue containing a functionalization moiety (e.g., a modification or label). A second step includes contacting the polypeptide with a binding agent that is attached to a DNA coding tag. In some embodiments, the labeling or modification of the NTAA may be performed prior to or after contacting the polypeptide with a binding agent. Upon binding of the binding agent to the NTAA of the polypeptide, information of the coding tag is transferred to the recording tag (e.g., via primer extension or ligation) to generate an extended recording tag. Lastly, the functionalized NTAA is eliminated via chemical or biological (e.g., enzymatic) means to expose a new NTAA.

As illustrated, the cycle is repeated “n” times to generate a final extended recording tag. In some embodiments, the order in the steps in the process for a degradation-based peptide polypeptide sequencing assay can be reversed or moved around. In some embodiments, the terminal amino acid functionalization can be conducted after the polypeptide is bound to a support. In some aspects, the analysis assay may include one or more additional steps, such as a wash step and/or treatment with other reagents. In some embodiments, the provided methods may be performed such that the C-terminal amino acid is modified, labeled, contacted by a binding agent, and/or eliminated from the polypeptide.

Engineered binders of the present invention can be used in the workflows of FIG. 1A and FIG. 1B. Characteristics of such engineered binders are described below.

Disclosed herein is an engineered binder that specifically binds to an N-terminally modified target polypeptide modified by an N-terminal modifier agent, wherein:
(i) the N-terminally modified target polypeptide has a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;
(ii) the engineered binder specifically binds to the N-terminally modified target polypeptide through interaction between the engineered binder and the M-P1 of the N-terminally modified target polypeptide; and
(iii) the engineered binder comprises an amino acid sequence having at least about 80% sequence identity to the amino acid sequence selected from the group consisting of SEQ ID NO: 7—SEQ ID NO: 20.

Disclosed herein is also an isolated nucleic acid molecule comprising a polynucleotide having a sequence encoding the engineered binder as described in the previous paragraph.

Given that multiple particular amino acid sequences of the scaffolds and engineered binders according to the present invention are disclosed herein (for example, in SEQ ID NO: 7—SEQ ID NO: 62), a skilled person would be able to create corresponding nucleic acid sequences encoding the engineered binders using standard methods known in the art. Since known degeneracy exists for nucleic acid sequences that encode a particular amino acid sequence, multiple variations in corresponding nucleic acid sequences exist for a given binder's sequence. In some embodiments, a nucleic acid molecule that encodes an engineered binder is a part of an expression vector. Such nucleic acid molecules can be optimized for expression in a particular cell type, or in a particular organism, such as bacteria cells, insect cells, yeast cells and so on, by methods known in the art. Engineered binders can be also expressed via in vitro translation.

Sequences of engineered binders can differ significantly from corresponding starting lipocalin scaffolds, and each of the engineered binders with sequences as set forth in SEQ ID NOs: 21-62 of the Sequence Listing contains 15-22 amino acid substitutions from the corresponding starting scaffold. Since most amino acid substitutions were designed to be on the substrate-interaction region of the binders, geometry of substrate-binding pockets of the scaffolds and atomic interactions within them were significantly changed during engineering and maturation process.

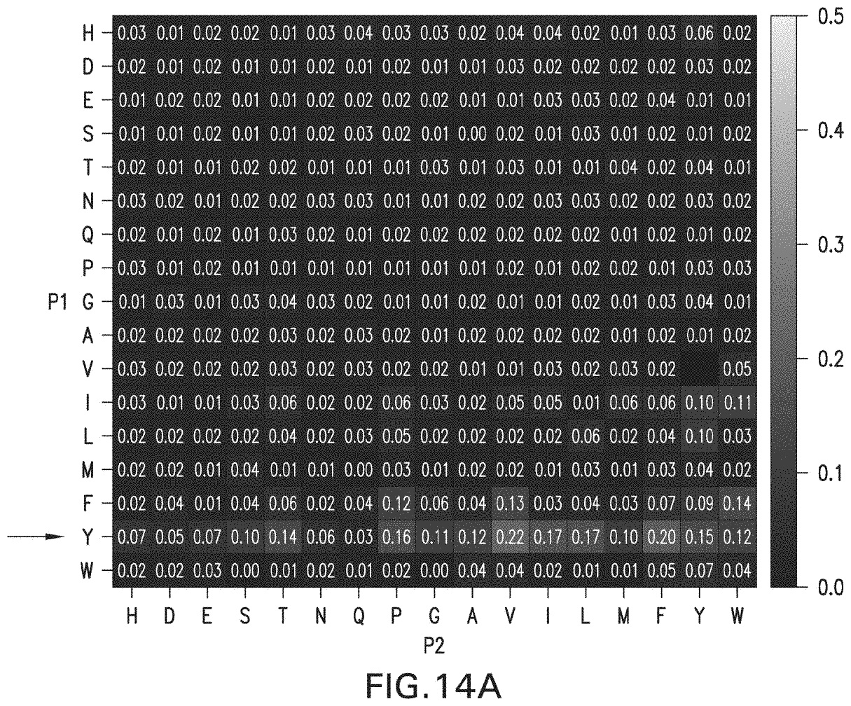

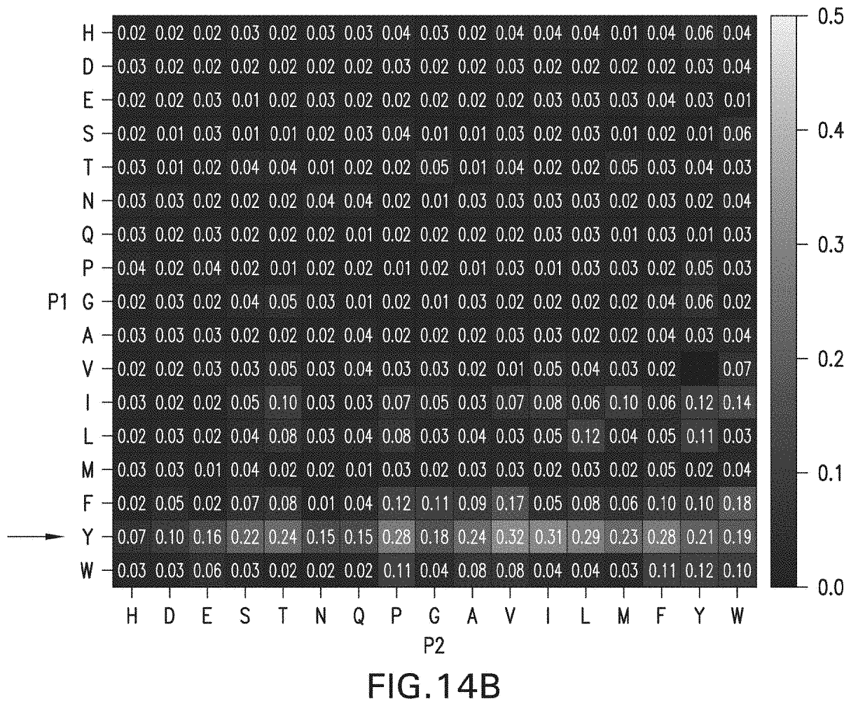

Engineered binders with sequences as set forth in SEQ ID NOs: 21-62 typically have about 89-91% sequence identity with corresponding starting scaffolds. Note that these binders may be further processed through another maturation round for improving their characteristics, such as M-P1 affinity, P1 selectivity and/or P2 tolerance, as shown for example in FIG. 14A-B. In the next maturation round, new amino acid substitutions will likely be introduced, and the updated binder's sequence may be further away from the sequence of the corresponding starting scaffold, such that it may have 80, 81, 82, 83, 84 or 85% sequence identity with the corresponding starting scaffold. Moreover, conservative amino acid substitutions can be made in the binder's sequence that would improve its characteristics unrelated to the M-P1 binding, such as improve binder's stability or increase expression level of the binder in bacterial cells. Such conservative amino acid substitutions are known to skilled in the art, and the updated binder's sequence may have less than 80% sequence identity with the corresponding starting scaffold (for example, may have 70% or 75% sequence identity with the corresponding starting scaffold).

To engineer M-P1 specific binders, different N-terminal modifications (M) can be used, and preferably, M is a chemical entity having a volume from about 100 Å³ to about 1000 Å³.

In some embodiments, the N-terminal modification comprises an amino acid moiety. In other embodiments, the N-terminal modification does not comprise an amino acid moiety.

In some embodiments, the N-terminal modifier agent is selected from the group consisting of compounds of the following formula:
(A)

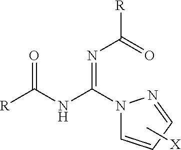

wherein R is CH₃, CF₃, OC(CH₃)₃, or OCH₂C₆H₅,

and X is H, CH₃, CF₃, CF₂H, or OCH₃;
(B)

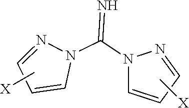

wherein X is H, CH₃, CF₃, CF₂H, OCH₃, or SO₂NH₂;
(C)

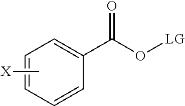

wherein X is H, F, Cl, OCH₃, OCF₃, CN, or SO₂NH₂,

and LG is succinimide, pentafluorophenyl, or tetrafluorophenyl; and
(D)

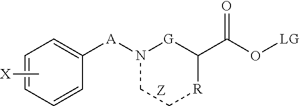

wherein X is H, F, Cl, NH₂, OCH₃, OCF₃, CN, or SO₂NH₂, A=CONH or SO₂, G=0 or 1 CH₂,

R is any amino acid or unnatural amino acid, and

Z ring=0 (not there), 1, 2, or 3 CH₂.

In some preferred embodiments, the N-terminal modifier agent is selected from the group consisting of compounds of the following formula:

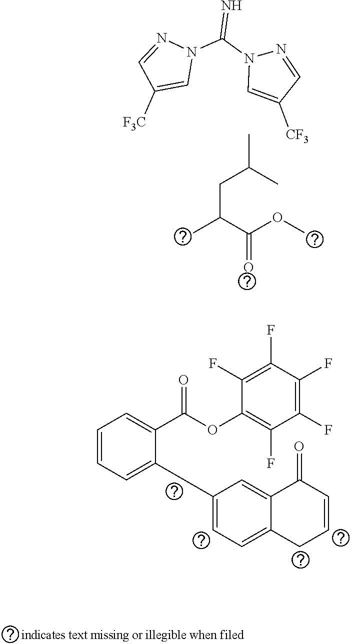

In some embodiments, the N-terminal modifier agent comprises an amino acid moiety (NTMaa). The N-terminal modifier agent can be a bipartite N-terminal modifier agent that comprises a natural or unnatural amino acid portion (NTMaa) and an N-terminal blocking group (NTMblk). The amino acid portion (NTMaa) and the N-terminal blocking group (NTMblk) can be connected or linked by any suitable bond or linkage. For example, the amino acid portion (NTMaa) and the N-terminal blocking group (NTMblk) can be connected with an amide bond. In some embodiments, L- or D- configurations of the NTMaa portion of the N-terminal modifier agent are alkylated to prevent racemization during installation of the N-terminal modification to target polypeptides.

In preferred embodiments, binding specificity between the engineered binder and the N-terminally modified target polypeptide is predominantly or substantially determined by interaction between the engineered binder and the M-P1 of the N-terminally modified target polypeptide.

In some embodiments, the engineered binder is capable of specifically binding to

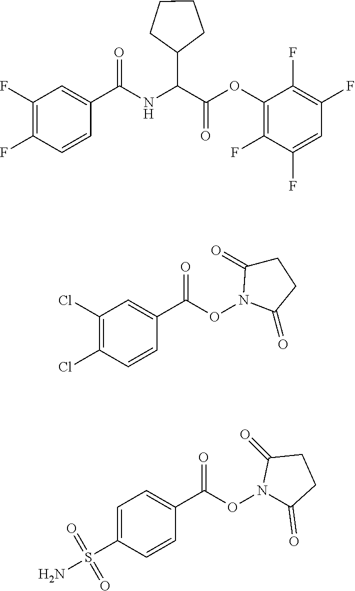 [SMILES: O=C(NC(C(=O)Oc1c(F)c(F)cc(F)c1F)C1CCCC1)c1ccc(F)c(F)c1 ; O=C(ON1C(=O)CCC1=O)c1ccc(Cl)c(Cl)c1 ; NS(=O)(=O)c1ccc(C(=O)ON2C(=O)CCC2=O)cc1]

each N-terminally modified target polypeptide from a plurality of N-terminally modified target polypeptides, wherein the plurality of N-terminally modified target polypeptides comprises at least 10 N-terminally modified target polypeptides that are modified with the same N-terminal modifier agent, have the same modified NTAA residue, and have different P2 residues.

Disclosed herein is also a set of engineered binders, comprising at least two engineered binders, wherein: (i) each engineered binder from the set of engineered binders is configured to specifically bind to an N-terminally modified target polypeptide modified with an N-terminal modifier agent and having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; (ii) each engineered binder from the set of engineered binders is configured to specifically bind to the N-terminally modified target polypeptide through interaction between the engineered binder and the modified NTAA residue of the N-terminally modified target polypeptide, wherein engineered binders from the set of engineered binders are configured to specifically bind to different modified NTAA residues of target polypeptides modified with the same or different N-terminal modifier agents; and (iii) at least one engineered binder from the set of engineered binders comprises an amino acid sequence having at least about 80% sequence identity to the amino acid sequence selected from the group consisting of SEQ ID NO: 7-SEQ ID NO: 20.

In preferred embodiments, the engineered binders from the set of engineered binders are configured to specifically bind to different modified NTAA residues of target polypeptides modified with the same N-terminal modifier agent. In other embodiments, target polypeptides may be modified with different N-terminal modifier agents, such as they can be modified as separate groups and then pooled together for the binding assay.

In some embodiments, engineered binders from the set of engineered binders are configured to specifically bind to at least two, at least 3, at least 4, at least 5, at least 6 or at least 7 different modified NTAA residues of target polypeptides. In some embodiments, engineered binders from the set of engineered binders are configured to specifically bind to at least 10 different modified NTAA residues of target polypeptides.

In some embodiments, at least one or at least two engineered binders from the set of engineered binders comprise an amino acid sequence having at least about 89% sequence identity to the amino acid sequence selected from the group consisting of SEQ ID NO: 7—SEQ ID NO: 20. Examples of such binders are binders comprising an amino acid sequence selected from the group consisting of SEQ ID NO: 21-SEQ ID NO: 62.

In some embodiments, the set of engineered binders may contain lipocalin-based engineered binders (such as derived from lipocalin scaffolds of SEQ ID NO: 7-SEQ ID NO: 20). In other embodiments, the set of engineered binders may contain at least one engineered binder derived from a non-lipocalin-based scaffold. This may be a preferred embodiment, since lipocalin-based engineered binders have better specificity and selectivity towards hydrophobic NTAA residues, whereas additional engineered binders derived from non-lipocalin-based scaffolds can be specific towards charged or polar NTAA residues of target polypeptide. An example of such engineered binder derived from a non-lipocalin-based scaffold is shown in the Example 9 (engineered binders of SEQ ID NO: 64-SEQ ID NO: 66 having specificity towards M64-D located at the N-terminus of target peptides, and derived from the non-lipocalin-based scaffold of SEQ ID NO: 63).

In some embodiments, the set of engineered binders further comprises least one engineered binder comprising an amino acid sequence having at least about 80% sequence identity to the amino acid sequence set forth in SEQ ID NO: 63.

In some embodiments, each engineered binder from the set of engineered binders is configured to specifically bind to each N-terminally modified target polypeptide from a plurality of N-terminally modified target polypeptides, wherein the plurality of N-terminally modified target polypeptides comprises at least 10 N-terminally modified target polypeptides that are modified with the same N-terminal modifier agent, have the same modified NTAA residue, and have different P2 residues.

In some embodiments, each engineered binder from the set of engineered binders comprises a detectable label or a nucleic acid coding tag; such binders can be used in the ProteoCode™ assay, or another assay that utilizes detection of a signal generated after binding of the binder to the NTAA of a target polypeptide. In some embodiments, engineered binders from the set of engineered binders are configured to specifically bind to at least 5, 6, 7, 8, 9, 10 or 15 different modified NTAA residues of target polypeptides, and each engineered binder from the set of engineered binders comprises a nucleic acid coding tag comprising identifying information regarding the corresponding engineered binder. In these embodiments, such set of engineered binders is used in a peptide sequencing assay, such as ProteoCode™ assay.

Further details of the described methods are shown below.

A. Samples

In some aspects, the present disclosure relates to the analysis of macromolecules from a sample. A macromolecule can be a large molecule composed of smaller subunits. In certain embodiments, a macromolecule is a protein, a protein complex, polypeptide, peptide, nucleic acid molecule, carbohydrate, lipid, macrocycle, or a chimeric macromolecule. A macromolecule (e.g., protein, polypeptide, peptide) analyzed according the methods disclosed herein may be obtained from a suitable source or sample. In some embodiments, the macromolecules (e.g., proteins, polypeptides, or peptides) are obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, the sample comprises two or more cells.

In some embodiments, the biological sample may contain whole cells and/or live cells and/or cell debris. In some examples, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids.

In some embodiments, the method includes obtaining and preparing macromolecules (e.g., polypeptides and proteins) from a single cell type or multiple cell types. In some embodiments, the sample comprises a population of cells. In some embodiments, the macromolecules (e.g., proteins, polypeptides, or peptides) are from a cellular or subcellular component, an extracellular vesicle, an organelle, or an organized subcomponent thereof. In some embodiments, the polypeptides are from one or more packaging of molecules (e.g., separate components of a single cell or separate components isolated from a population of cells, such as organelles or vesicles). The macromolecules (e.g., proteins, polypeptides, or peptides) may be from organelles, for example, mitochondria, nuclei, or cellular vesicles. In one embodiment, one or more specific types of single cells or subtypes thereof may be isolated. In some embodiments, the sample may include but are not limited to cellular organelles, (e.g., nucleus, golgi apparatus, ribosomes, mitochondria, endoplasmic reticulum, chloroplast, cell membrane, vesicles, etc.).

In certain embodiments, a macromolecule is a protein, a protein complex, a polypeptide, or peptide. Amino acid sequence information and post-translational modifications of a peptide, polypeptide, or protein are transduced into a nucleic acid encoded library that can be analyzed via next generation sequencing methods. A peptide may comprise L-amino acids, D-amino acids, or both. A peptide, polypeptide, protein, or protein complex may comprise a standard, naturally occurring amino acid, a modified amino acid (e.g., post-translational modification), an amino acid analog, an amino acid mimetic, or any combination thereof. In some embodiments, a peptide, polypeptide, or protein is naturally occurring, synthetically produced, or recombinantly expressed. In any of the aforementioned peptide embodiments, a peptide, polypeptide, protein, or protein complex may further comprise a post-translational modification. Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, (3-amino acids, homo-amino acids, Proline and Pyruvic acid derivatives, 3-substituted Alanine derivatives, Glycine derivatives, ring-substituted Phenylalanine and Tyrosine Derivatives, linear core amino acids, and N-methyl amino acids.

A post-translational modification (PTM) of a peptide, polypeptide, or protein may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation (e.g., N-linked, 0-linked, C-linked, phosphoglycosylation), glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide, polypeptide, or protein. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini of a peptide, polypeptide, or protein. Post-translational modification can regulate a protein's “biology” within a cell, e.g., its activity, structure, stability, or localization. For example, phosphorylation plays an important role in regulation of protein, particularly in cell signaling (Prabakaran et al., 2012, Wiley Interdiscip Rev Syst Biol Med 4: 565-583). In another example, the addition of sugars to proteins, such as glycosylation, has been shown to promote protein folding, improve stability, and modify regulatory function and the attachment of lipids to proteins enables targeting to the cell membrane. A post-translational modification can also include peptide, polypeptide, or protein modifications to include one or more detectable labels.

In certain embodiments, a peptide, polypeptide, or protein can be fragmented. For example, the fragmented peptide can be obtained by fragmenting a protein from a sample, such as a biological sample. The peptide, polypeptide, or protein can be fragmented by any means known in the art, including fragmentation by a protease or endopeptidase. In some embodiments, fragmentation of a peptide, polypeptide, or protein is targeted by use of a specific protease or endopeptidase. A specific protease or endopeptidase binds and cleaves at a specific consensus sequence (e.g., TEV protease). In other embodiments, fragmentation of a peptide, polypeptide, or protein is non-targeted or random by use of a non-specific protease or endopeptidase. A non-specific protease may bind and cleave at a specific amino acid residue rather than a consensus sequence (e.g., proteinase K is a non-specific serine protease). In some embodiments, proteinases and endopeptidases, such as those known in the art, can be used to cleave a protein or polypeptide into smaller peptide fragments include proteinase K, trypsin, chymotrypsin, pepsin, thermolysin, thrombin, Factor Xa, furin, endopeptidase, papain, pepsin, subtilisin, elastase, enterokinase, Genenase™ I, Endoproteinase LysC, Endoproteinase AspN, Endoproteinase GluC, etc. (Granvogl et al., 2007, Anal Bioanal Chem 389: 991-1002). In certain embodiments, a peptide, polypeptide, or protein is fragmented by proteinase K, or optionally, a thermolabile version of proteinase K to enable rapid inactivation. In some cases, Proteinase K is stable in denaturing reagents, such as urea and SDS, and enables digestion of completely denatured proteins. Protein and polypeptide fragmentation into peptides can be performed before or after attachment of a DNA tag or DNA recording tag.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, ·NTCB+Ni (2-nitro-5-thiocyanobenzoic acid), etc.

In certain embodiments, following enzymatic or chemical cleavage, the resulting peptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. A cleavage reaction may be monitored, preferably in real time, by spiking the protein or polypeptide sample with a short test FRET (fluorescence resonance energy transfer) peptide comprising a peptide sequence containing a proteinase or endopeptidase cleavage site. In the intact FRET peptide, a fluorescent group and a quencher group are attached to either end of the peptide sequence containing the cleavage site, and fluorescence resonance energy transfer between the quencher and the fluorophore leads to low fluorescence. Upon cleavage of the test peptide by a protease or endopeptidase, the quencher and fluorophore are separated giving a large increase in fluorescence. A cleavage reaction can be stopped when a certain fluorescence intensity is achieved, allowing a reproducible cleavage endpoint to be achieved.

A sample of macromolecules (e.g., peptides, polypeptides, or proteins) can undergo protein fractionation methods where proteins or peptides are separated by one or more properties such as cellular location, molecular weight, hydrophobicity, isoelectric point, or protein enrichment methods. In some embodiments, a subset of macromolecules (e.g., proteins) within a sample is fractionated such that a subset of the macromolecules is sorted from the rest of the sample. For example, the sample may undergo fractionation methods prior to attachment to a solid support. Alternatively, or additionally, protein enrichment methods may be used to select for a specific protein or peptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. In the case of immunoglobulin molecules, analysis of the sequence and abundance or frequency of hypervariable sequences involved in affinity binding are of particular interest, particularly as they vary in response to disease progression or correlate with healthy, immune, and/or or disease phenotypes. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, including depletion spin columns that remove top 2-20 plasma proteins (Pierce, Agilent), or PROTIA and PROT20 (Sigma-Aldrich).

In certain embodiments, a protein sample dynamic range can be modulated by fractionating the protein sample using standard fractionation methods, including electrophoresis and liquid chromatography (Zhou et al., 2012, Anal Chem 84(2): 720-734), or partitioning the fractions into compartments (e.g., droplets) loaded with limited capacity protein binding beads/resin (e.g. hydroxylated silica particles) (McCormick, 1989, Anal Biochem 181(1): 66-74) and eluting bound protein. Excess protein in each compartmentalized fraction is washed away. Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivatized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

In some embodiments, a partition barcode is used which comprises assignment of a unique barcode to a subsampling of macromolecules from a population of macromolecules within a sample. This partition barcode may be comprised of identical barcodes arising from the partitioning of macromolecules within compartments labeled with the same barcode (e.g. a barcoded bead population in which multiple beads share the same barcode). The use of physical compartments effectively subsamples the original sample to provide assignment of partition barcodes. For instance, a set of beads labeled with 10,000 different compartment barcodes is provided. Furthermore, suppose in a given assay, that a population of 1 million beads are used in the assay. On average, there are 100 beads per compartment barcode (Poisson distribution). Further suppose that the beads capture an aggregate of 10 million macromolecules. On average, there are 10 macromolecules per bead, with 100 compartments per compartment barcode, there are effectively 1000 macromolecules per partition barcode (comprised of 100 compartment barcodes for 100 distinct physical compartments).

In another embodiment, single molecule partitioning and partition barcoding of polypeptides is accomplished by labeling polypeptides (chemically or enzymatically) with an amplifiable DNA UMI tag (e.g., recording tag) at the N or C terminus, or both. DNA tags are attached to the body of the polypeptide (internal amino acids) via non-specific photo-labeling or specific chemical attachment to reactive amino acids such as lysines. Information from the recording tag attached to the terminus of the peptide is transferred to the DNA tags via an enzymatic emulsion PCR (Williams et al., Nat Methods, (2006) 3(7):545-550; Schutze et al., Anal Biochem. (2011) 410(1):155-157) or emulsion in vitro transcription/reverse transcription (IVT/RT) step. In the preferred embodiment, a nanoemulsion is employed such that, on average, there is fewer than a single polypeptide per emulsion droplet with size from 50 nm-1000 nm (Nishikawa et al., J Nucleic Acids. (2012) 2012: 923214; Gupta et al., Soft Matter. (2016) 12(11):2826-41; Sole et al., Langmuir (2006, 22(20):8326-8332). Additionally, all the components of PCR are included in the aqueous emulsion mix including primers, dNTPs, Mg2+, polymerase, and PCR buffer. If IVT/RT is used, then the recording tag is designed with a T7/SP6 RNA polymerase promoter sequence to generate transcripts that hybridize to the DNA tags attached to the body of the polypeptide (Ryckelynck et al., RNA. (2015) 21(3):458-469). A reverse transcriptase (RT) copies the information from the hybridized RNA molecule to the DNA tag. In this way, emulsion PCR or IVT/RT can be used to effectively transfer information from the terminus recording tag to multiple DNA tags attached to the body of the polypeptide.

In some embodiments, a sample of macromolecules (e.g., peptides, polypeptides, or proteins) can be processed into a physical area or volume e.g., into a compartment. In some embodiments, the compartment separates or isolates a subset of macromolecules from a sample of macromolecules. In some examples, the compartment may be an aqueous compartment (e.g., microfluidic droplet), a solid compartment (e.g., picotiter well or microtiter well on a plate, tube, vial, bead), or a separated region on a surface. In some cases, a compartment may comprise one or more beads to which macromolecules may be immobilized. In some embodiments, macromolecules in a compartment is labeled with a compartment tag including a barcode. For example, the macromolecules in one compartment can be labeled with the same barcode or macromolecules in multiple compartments can be labeled with the same barcode. See e.g., Valihrach et al., Int J Mol Sci. 2018 Mar. 11;19(3). pii: E807. Encapsulation of cellular contents via gelation in beads is a useful approach to single cell analysis (Tamminen et al., Front Microbiol (2015) 6: 195; Spencer et al., ISME J (2016) 10(2): 427-436). Barcoding single cell droplets enables all components from a single cell to be labeled with the same identifier (Klein et al., Cell (2015) 161(5): 1187-1201; Zilionis et al., Nat Protoc (2017) 12(1): 44-73; International Patent Publication No. WO 2016/130704). Compartment barcoding can be accomplished in a number of ways including direct incorporation of unique barcodes into each droplet by droplet joining (Bio-Rad Laboratories), by introduction of barcoded beads into droplets (10× Genomics), by barcode bead-templated emulsion generation (see US 20200261879 Al and Hatori, Makiko N., et al., “Particle-Templated Emulsification for Microfluidics-Free Digital Biology.” 2018, Analytical Chemistry 90 (16): 9813-20), or by combinatorial barcoding of components of the droplet post encapsulation and gelation using and split-pool combinatorial barcoding as described by Gunderson et al. (US 20180273933 A1). A similar combinatorial labeling scheme can also be applied to nuclei (Vitak et al., Nat Methods (2017) 14(3):302-308).

The above droplet barcoding approaches have been used for DNA analysis but not for protein analysis. Adapting the above droplet barcoding platforms to work with proteins requires several innovative steps. The first is that barcodes are primarily comprised of DNA sequences, and this DNA sequence information needs to be conferred to the protein analyte. In the case of a DNA analyte, it is relatively straightforward to transfer DNA information onto a DNA analyte. In contrast, transferring DNA information onto proteins is more challenging, particularly when the proteins are denatured and digested into peptides for downstream analysis. This requires that each peptide be labeled with a compartment barcode. The challenge is that once the cell is encapsulated into a droplet, it is difficult to denature the proteins, protease digest the resultant polypeptides, and simultaneously label the peptides with DNA barcodes. Encapsulation of cells in polymer forming droplets and their polymerization (gelation) into porous beads, which can be brought up into an aqueous buffer, provides a vehicle to perform multiple different reaction steps, unlike cells in droplets (Tamminen et al., Front Microbiol (2015) 6: 195; Spencer et al., ISME J (2016) 10(2): 427-436; International Patent Publication No. WO 2016/130704). Preferably, the encapsulated proteins are crosslinked to the gel matrix to prevent their subsequent diffusion from the gel beads. This gel bead format allows the entrapped proteins within the gel to be denatured chemically or enzymatically, labeled with DNA tags, protease digested, and subjected to a number of other interventions. In some embodiments, encapsulation and lysis of a single cell in a gel matrix can be performed.

In some embodiments, the macromolecules (e.g., polypeptides) are joined to a support before performing a polypeptide analysis assay. In some embodiments, a plurality of proteins is attached to a solid support prior to the polypeptide analysis assay. A solid support can be any support surface including, but not limited to, a bead, a microbead, an array, a glass surface, a silicon surface, a plastic surface, a filter, a membrane, a PTFE membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, silica, polyanhydrides, polyglycolic acid, polyvinylchloride, polylactic acid, polyorthoesters, functionalized silane, polypropylfumerate, collagen, glycosaminoglycans, polyamino acids, or any combination thereof. In certain embodiments, a solid support is a bead, for example, a polystyrene bead, a polymer bead, a polyacrylate bead, an agarose bead, a cellulose bead, a dextran bead, an acrylamide bead, a solid core bead, a porous bead, a paramagnetic bead, a glass bead, a silica-based bead, or a controlled pore bead, or any combinations thereof. In some specific embodiments, the solid support is a porous agarose bead.

In some embodiments, the support may comprise any suitable solid material, including porous and non-porous materials, to which a macromolecule, e.g., a polypeptide, can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, or a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

Various reactions may be used to attach the polypeptides to a solid support. The polypeptides may be attached directly or indirectly to the solid support. In some cases, the polypeptide is attached to the solid support via a nucleic acid. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a solid support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NETS ester) reagent or alkyne-benzophenone to generate an alkyne-labeled polypeptide. In some embodiments, an alkyne can also be a strained alkyne, such as cyclooctynes including Dibenzocyclooctyl (DBCO), etc.

In certain embodiments where multiple proteins are immobilized on the same solid support, the proteins can be spaced appropriately to accommodate methods of analysis to be used to assess the proteins. For example, it may be advantageous to space the proteins that optimally to allow a nucleic acid-based method for assessing and sequencing the proteins to be performed. In some embodiments, the method for assessing and sequencing the proteins involve a binding agent which binds to the protein and the binding agent comprises a coding tag with information that is transferred to a nucleic acid attached to the proteins (e.g., recording tag). In some cases, information transfer from a coding tag of a binding agent bound to one protein may reach a neighboring protein.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS) +self-assembled Tween-20 (Hua et al., 2014, Nat. Methods 11:1233-1236), diamond-like carbon (DLC), DLC +PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of macromolecules (e.g., proteins, polypeptide, or peptides) can be titrated on the surface or within the volume of a solid substrate by spiking a competitor or “dummy” reactive molecule when immobilizing the proteins, polypeptides or peptides to the solid substrate.

To control protein spacing on the solid support, the density of functional coupling groups for attaching the protein (e.g., TCO or carboxyl groups (COOH)) may be titrated on the substrate surface. In some embodiments, multiple proteins are spaced apart on the surface or within the volume (e.g., porous supports) of a solid support such that adjacent proteins are spaced apart at a distance of about 50 nm to about 500 nm, or about 50 nm to about 400 nm, or about 50 nm to about 300 nm, or about 50 nm to about 200 nm, or about 50 nm to about 100 nm. In some embodiments, multiple a proteins are spaced apart on the surface of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple a proteins are spaced apart on the surface of a solid support with an average distance of at least 50 nm. In some embodiments, proteins are spaced apart on the surface or within the volume of a solid support such that, empirically, the relative frequency of inter- to intra-molecular events (e.g. transfer of information) is <1:10; <1:100; <1:1,000; or <1:10,000.

In some embodiments, the plurality of proteins is coupled on the solid support spaced apart at an average distance between two adjacent proteins which ranges from about 50 to 100 nm, from about 50 to 250 nm, from about 50 to 500 nm, from about 50 to 750 nm, from about 50 to 1,000 nm, from about 50 to 1,500 nm, from about 50 to 2,000 nm, from about 100 to 250 nm, from about 100 to 500 nm, from about 200 to 500 nm, from about 300 to 500 nm, from about 100 to 1000 nm, from about 500 to 600 nm, from about 500 to 700 nm, from about 500 to 800 nm, from about 500 to 900 nm, from about 500 to 1,000 nm, from about 500 to 2,000 nm, from about 500 to 5,000 nm, from about 1,000 to 5,000 nm, or from about 3,000 to 5,000 nm.

In some embodiments, appropriate spacing of the polypeptides on the solid support is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEGₙ-mTet is added to the activated beads (wherein n is any number, such as 1-100). The ratio between the mPEG₃-NH₂ (not available for coupling) and NH₂-PEG24-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEG₃-NH₂ is about or greater than 1:1000, about or greater than 1:10,000, about or greater than 1:100,000, or about or greater than 1:1,000,000. In some further embodiments, the recording tag attaches to the NH₂-PEGₙ-mTet. In some embodiments, the spacing of the polypeptides on the solid support is achieved by controlling the concentration and/or number of available COOH or other functional groups on the solid support.

B. Recording Tag

In one embodiment, the macromolecule (e.g., protein or polypeptide) is labeled with a DNA recording tag. In some embodiments, the sample is provided with a plurality of recording tags. In some aspects, a plurality of macromolecules in the sample is provided with recording tags. The recording tags may be associated or attached, directly or indirectly to the macromolecules using any suitable means. In some embodiments, a macromolecule may be associated with one or more recording tags. In some aspects, the recording tag may be any suitable sequenceable moiety to which identifying information can be transferred (e.g., information from one or more coding tags).

In some embodiments, at least one recording tag is associated or co-localized directly or indirectly with the macromolecule (e.g., polypeptide). In a particular embodiment, a single recording tag is attached to a polypeptide, such as via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag.

A recording tag may comprise DNA, RNA, PNA, gPNA, GNA, BNA, XNA, TNA, polynucleotide analogs, or a combination thereof. A recording tag may be single stranded, or partially or completely double stranded. A recording tag may have a blunt end or overhanging end. In certain embodiments, all or a substantial amount of the macromolecules (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of macromolecules within a sample are labeled with recording tags. In a particular embodiment, a subset of macromolecules from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to the macromolecules prior to providing the sample on a solid support. In some embodiments, the recording tags are attached to the macromolecules after providing the sample on the solid support.

In some embodiments, the recording tag may comprise other nucleic acid components. In some embodiments, the recording tag may comprise a unique molecular identifier, a compartment tag, a partition barcode, sample barcode, a fraction barcode, a spacer sequence, a universal priming site, or any combination thereof. In some embodiments, the recording tag can further comprise other information including information from a macromolecule analysis assay, such as binder identifier (e.g., from a coding tag), cycle identifier (e.g., from a coding tag), etc. In some embodiments, the recording tag may comprise a blocking group, such as at the 3′-terminus of the recording tag. In some cases, the 3′-terminus of the recording tag is blocked to prevent extension of the recording tag by a polymerase.

In some embodiments, the recording tag can include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, macromolecules from many different samples can be labeled with recording tags with sample-specific barcodes, and then all the samples pooled together prior to immobilization to a solid support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In certain embodiments, a recording tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual macromolecules. In some embodiments, within a library of macromolecules, each macromolecule is associated with a single recording tag, with each recording tag comprising a unique UMI. In other embodiments, multiple copies of a recording tag are associated with a single macromolecule, with each copy of the recording tag comprising the same UMI. In some embodiments, a UMI has a different base sequence than the spacer or encoder sequences within the binding agents' coding tags to facilitate distinguishing these components during sequence analysis. In some embodiments, the UMI may provide function as a location identifier and also provide information in the macromolecule analysis assay. For example, the UMI may be used to identify molecules that are identical by descent, and therefore originated from the same initial molecule. In some aspects, this information can be used to correct for variations in amplification, and to detect and correct sequencing errors.

In some embodiments, the recording tag comprises a spacer polymer. In certain embodiments, a recording tag comprises a spacer at its terminus, e.g., 3′ end. As used herein reference to a spacer sequence in the context of a recording tag includes a spacer sequence that is identical to the spacer sequence associated with its cognate binding agent, or a spacer sequence that is complementary to the spacer sequence associated with its cognate binding agent. The terminal, e.g., 3′, spacer on the recording tag permits transfer of identifying information of a cognate binding agent from its coding tag to the recording tag during the first binding cycle (e.g., via annealing of complementary spacer sequences for primer extension or sticky end ligation). In one embodiment, the spacer sequence is about 1-20 bases in length, about 2-12 bases in length, or 5-10 bases in length. The length of the spacer may depend on factors such as the temperature and reaction conditions of the primer extension reaction for transferring coding tag information to the recording tag.

In some embodiments, the recording tags associated with a library of polypeptides share a common spacer sequence. In other embodiments, the recording tags associated with a library of polypeptides have binding cycle specific spacer sequences that are complementary to the binding cycle specific spacer sequences of their cognate binding agents. In some aspects, the spacer sequence in the recording tag is designed to have minimal complementarity to other regions in the recording tag; likewise, the spacer sequence in the coding tag should have minimal complementarity to other regions in the coding tag. In some cases, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′ — SEQ ID NO: 1) or an Illumina P7 primer (5′-CAAGCAGAAGACGGCATACGAGAT — 3′-SEQ ID NO: 2).

In certain embodiments, a recording tag comprises a compartment tag. In some embodiments, the compartment tag is a component within a recording tag. In some embodiments, the recording tag can also include a barcode which represents a compartment tag in which a compartment, such as a droplet, microwell, physical region on a solid support, etc. is assigned a unique barcode. The association of a compartment with a specific barcode can be achieved in any number of ways such as by encapsulating a single barcoded bead in a compartment, e.g., by direct merging or adding a barcoded droplet to a compartment, by directly printing or injecting a barcode reagents to a compartment, etc. The barcode reagents within a compartment are used to add compartment-specific barcodes to the macromolecule or fragments thereof within the compartment. Applied to protein partitioning into compartments, the barcodes can be used to map analyzed peptides back to their originating protein molecules in the compartment. This can greatly facilitate protein identification. Compartment barcodes can also be used to identify protein complexes. In other embodiments, multiple compartments that represent a subset of a population of compartments may be assigned a unique barcode representing the subset. In some embodiments, the recording tag comprises fraction barcode which contains identifying information for the macromolecules within a fraction.

In some embodiments, the one or more tags or information of the one or more tags are transferred to the recording tag (e.g., via primer extension or ligation) to extend the recording tag. In some embodiments, one or more of the tags (e.g., compartment tag, a partition barcode, sample barcode, a fraction barcode, etc.) further comprise a functional moiety capable of reacting with an internal amino acid, the peptide backbone, or N-terminal amino acid on the plurality of protein complexes, proteins, or polypeptides. In some embodiments, the functional moiety is a click chemistry moiety, an aldehyde, an azide/alkyne, or a malemide/thiol, or an epoxide/nucleophile, an inverse electron demain Diels-Alder (iEDDA) group, or a moiety for a Staudinger reaction. In some specific embodiments, a plurality of compartment tags is formed by printing, spotting, ink-jetting the compartment tags into the compartment, or a combination thereof. In some embodiments, the tag is attached to a polypeptide to link the tag to the macromolecule via a polypeptide-polypeptide linkage. In some embodiments, the tag-attached polypeptide comprises a protein ligase recognition sequence.

In certain embodiments, a peptide or polypeptide macromolecule can be immobilized to a solid support by an affinity capture reagent (and optionally covalently crosslinked), wherein the recording tag is associated with the affinity capture reagent directly, or alternatively, the macromolecule can be directly immobilized to the solid support with a recording tag. In one embodiment, the macromolecule is attached to a bait nucleic acid which hybridizes to a capture nucleic acid and is ligated to a capture nucleic acid which comprises a reactive coupling moiety for attaching to the solid support. In some examples, the bait or capture nucleic acid may serve as a recording tag to which information regarding the polypeptide can be transferred. In some embodiments, the macromolecule is attached to a bait nucleic acid to form a nucleic acid-macromolecule chimera. In some embodiments, the immobilization methods comprise bringing the nucleic acid-macromolecule chimera into proximity with a solid support by hybridizing the bait nucleic acid to a capture nucleic acid attached to the solid support, and covalently coupling the nucleic acid-macromolecule chimera to the solid support. In some cases, the nucleic acid-macromolecule chimera is coupled indirectly to the solid support, such as via a linker. In some embodiments, a plurality of the nucleic acid-macromolecule chimeras is coupled on the solid support and any adjacently coupled nucleic acid-macromolecule chimeras are spaced apart from each other at an average distance of about 50 nm or greater.

In some embodiments, the density or number of macromolecules provided with a recording tag is controlled or titrated. In some examples, the desired spacing, density, and/or amount of recording tags in the sample may be titrated by providing a diluted or controlled number of recording tags. In some examples, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a macromolecule in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some examples, the ratio of functional recording tags is adjusted based on the reaction rate of the functional recording tags compared to the reaction rate of the competitor molecules.

In some examples, the labeling of the macromolecule with a recording tag is performed using standard amine coupling chemistries. For example, the e-amino group (e.g., of lysine residues) and the N-terminal amino group may be susceptible to labeling with amine-reactive coupling agents, depending on the pH of the reaction (Mendoza et al., Mass Spectrom Rev (2009) 28(5): 785-815). In a particular embodiment, the recording tag comprises a reactive moiety (e.g., for conjugation to a solid surface, a multifunctional linker, or a macromolecule), a linker, a universal priming sequence, a barcode (e.g., compartment tag, partition barcode, sample barcode, fraction barcode, or any combination thereof), an optional UMI, and a spacer (Sp) sequence for facilitating information transfer to/from a coding tag. In another embodiment, the protein can be first labeled with a universal DNA tag, and the barcode-Sp sequence (representing a sample, a compartment, a physical location on a slide, etc.) are attached to the protein later through and enzymatic or chemical coupling step. A universal DNA tag comprises a short sequence of nucleotides that are used to label a protein or polypeptide macromolecule and can be used as point of attachment for a barcode (e.g., compartment tag, recording tag, etc.). For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein. In a particular embodiment, the protein is labeled with a universal DNA tag prior to proteinase digestion into peptides. The universal DNA tags on the labeled peptides from the digest can then be converted into an informative and effective recording tag.

The recording tags may comprise a reactive moiety for a cognate reactive moiety present on the target macromolecule, e.g., the target protein, (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a macromolecule. A suitable linker can be attached to various positions of the recording tag, such as the 3′ end, at an internal position, or within the linker attached to the 5′ end of the recording tag.

C. Cyclic Transfer of Coding Tag Information to Recording Tag

In some embodiments, the macromolecule analysis assay (e.g., polypeptide analysis assay) includes extending the recording tag associated with the macromolecule, e.g., the polypeptide by transferring identifying information from one or more coding tags to the recording tag. In the methods described herein, upon binding of a binding agent to a macromolecule, e.g., a protein or peptide, identifying information of its linked coding tag is transferred to the recording tag (e.g., recording tag) associated with the peptide, thereby generating an extended recording tag. In some embodiments, the recording tag further comprises barcodes and/or other nucleic acid components. In particular embodiments, the identifying information from the coding tag of the binding agent is transferred to the recording tag or added to any existing barcodes (or other nucleic acid components) attached thereto. The transfer of the identifying information may be performed using extension or ligation. In some embodiments, a spacer is added to the end of the recording tag, and the spacer comprises a sequence that is capable of hybridizing with a sequence on the coding tag to facilitate the transfer of the identifying information from the coding tag. In some embodiments, the identifying information from the coding tag comprises information regarding the identity of the one or more amino acid(s) on the peptide or polypeptide bound by the binding agent.

In some embodiments, in a cyclic manner, the terminal amino acid (e.g., N-terminal amino acid) of each peptide is labeled (e.g., phenylthiocarbamoyl (PTC), modified-PTC, Cbz, dinitrophenyl (DNP) moiety, sulfonyl nitrophenyl (SNP), acetyl, guanidinyl, amino guanidine, heterocyclic methanimine). In some cases, the labeling of the terminal amino acid (e.g., N-terminal amino acid) can be performed as before or after the binding of a binding agent to the peptide or polypeptide. The N-terminal amino acid (or labeled N-terminal amino acid, e.g., PTC-NTAA, Cbz-NTAA, DNP-NTAA, SNP-NTAA, acetyl-NTAA, guanidinylated-NTAA, amino guanidine-NTAA, heterocyclic methanimine-NTAA) of each immobilized peptide is bound by a cognate NTAA binding agent which is attached to a coding tag, and identifying information from the coding tag associated with the bound NTAA binding agent is transferred to the bait or capture nucleic acid associated with the immobilized peptide analyte, thereby generating an extended nucleic acid containing information from the coding tag.

In some embodiments, the bound binding agents are released from the polypeptide after identifying information from the coding tag of the binding agent is transferred to the recording tag. In some embodiments, the one or more binding agents are removed from the polypeptide after identifying information from the coding tag of the binding agent is transferred to the recording tag. In some aspects, after identifying information from the coding tag of the binding agent is transferred to the recording tag, a wash step is performed.

In some embodiments, the binding agents are associated with a coding tag and other optional nucleic acid components. The coding tag associated with the binding agent is or comprises a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence or a sequence with identifying information, which is optionally flanked by one spacer on one side or optionally flanked by a spacer on each side. A coding tag may also be comprised of an optional

UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended nucleic acid on the recording tag. In certain embodiments, a coding tag may further comprise a binding cycle specific spacer or barcode, a unique molecular identifier, a universal priming site, or any combination thereof.

Coding tag information associated with a specific binding agent may be transferred to a recording tag using a variety of methods. In any of the preceding embodiments, the transfer of identifying information (e.g., from a coding tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

In certain embodiments, information of a coding tag is transferred to a recording tag via primer extension (See e.g., Chan et al. (2015) Curr Opin Chem Biol 26: 55-61). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites. Alternatively, the nascent recording tag can also be coated with RecA (or related homologues such as uvsX) to facilitate invasion of the 3′ terminus into a completely double stranded coding tag (Bell et al., 2012, Nature 491:274-278). This configuration prevents the double stranded coding tag from interacting with internal recording tag elements, yet is susceptible to strand invasion by the RecA coated 3′ tail of the extended recording tag (Bell et al., 2015, Elife 4: e08646). The presence of a single-stranded binding protein can facilitate the strand displacement reaction.

In some embodiments, a DNA polymerase that is used for primer extension possesses strand-displacement activity and has limited or is devoid of 3′-5 exonuclease activity. Several of many examples of such polymerases include Klenow exo- (Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9° N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage T7 gene 2.5 protein, phage Pf3 SSB, replication protein A RPA32 and RPA14 subunits (Wold, Annu. Rev. Biochem. (1997) 66:61-92); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage T7 helicase/primase, phage T4 gene 41 helicase, E. coli Rep helicase, E. coli recBCD helicase, recA, E. coli and eukaryotic topoisomerases (Annu Rev Biochem. (2001) 70:369-413).

Mis-priming or self-priming events, such as when the terminal spacer sequence of the recoding tag primes extension self-extension may be minimized by inclusion of single stranded binding proteins (T4 gene 32, E. coli SSB, etc.), DMSO (1-10%), formamide (1-10%), BSA(10-100 ug/ml), TMAC₁ (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, T7 DNA polymerase exo- (Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In some embodiments, using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. In this manner, the extended recording tag associated with the immobilized peptide (with or without a non-templated adenosine base) can anneal to the coding tag and undergo primer extension.

Alternatively, addition of non-templated base can be reduced by employing a mutant polymerase (mesophilic or thermophilic) in which non-templated terminal transferase activity has been greatly reduced by one or more point mutations, especially in the 0-helix region (see U.S. Pat. No. 7,501,237) (Yang et al., Nucleic Acids Res. (2002) 30(19): 4314-4320). Pfu exo-, which is 3′ exonuclease deficient and has strand-displacing ability, also does not have non-templated terminal transferase activity.

In some embodiments, various conditions for one or more steps of the method may be modified by one skilled in the art. For example, the temperature for contacting of the binding agents to the macromolecules or for hybridization of the spacer sequences on the recording tag and coding tag can be increased or decreased to modify specificity or stringency of the interactions. In some embodiments, to minimize non-specific interaction of the coding tag labeled binding agents in solution with the nucleic acids of immobilized proteins, competitor (also referred to as blocking) oligonucleotides complementary to nucleic acids containing spacer sequences (e.g., on the recording tag) can be added to binding reactions to minimize non-specific interactions. In some embodiments, the blocking oligonucleotides contain a sequence that is complementary to the coding tag or a portion thereof attached to the binding agent. In some embodiments, blocking oligonucleotides are relatively short. In some embodiments, the blocking oligonucleotide is directly or indirectly attached to the coding tag. In some examples, the coding tag comprises a hairpin nucleic acid, and the hairpin includes a sequence that is complementary to a spacer and/or barcode of the coding tag. Excess competitor oligonucleotides are washed from the binding reaction prior to primer extension, which effectively dissociates the annealed competitor oligonucleotides from the nucleic acids on the recording tag, especially when exposed to slightly elevated temperatures (e.g., 30-50° C.). In some embodiments, blocking oligonucleotides may comprise a terminator nucleotide at its 3′ end to prevent primer extension.

In certain embodiments, the annealing of the spacer sequence on the recording tag to the complementary spacer sequence on the coding tag is metastable under the primer extension reaction conditions (i.e., the annealing Tm is similar to the reaction temperature). This allows the spacer sequence of the coding tag to displace any blocking oligonucleotide annealed to the spacer sequence of the recording tag (or extensions thereof).

Self-priming/mis-priming events initiated by self-annealing of the terminal spacer sequence of the extended recording tag with internal regions of the extended recording tag may be minimized by including pseudo-complementary bases in the recording/extended recording tag (Lahoud et al., Nucleic Acids Res. (2008) 36:3409-3419), (Hoshika et al., Angew Chem Int Ed Engl (2010) 49(32): 5554-5557). Pseudo-complementary bases show significantly reduced hybridization affinities for the formation of duplexes with each other due to the presence of chemical modification. However, many pseudo-complementary modified bases can form strong base pairs with natural DNA or RNA sequences. In certain embodiments, the coding tag spacer sequence is comprised of multiple A and T bases, and commercially available pseudo-complementary bases 2-aminoadenine and 2-thiothymine are incorporated in the recording tag using phosphoramidite oligonucleotide synthesis. Additional pseudocomplementary bases can be incorporated into the extended recording tag during primer extension by adding pseudo-complementary nucleotides to the reaction (Gamper et al., Biochemistry. (2006) 45(22):6978-6986).

Coding tag information associated with a specific binding agent may be transferred to a nucleic acid on the recording tag associated with the immobilized peptide via ligation. Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase, 9° N DNA ligase, Electroligase° (See e.g., U.S. Patent Publication No. US20140378315). Alternatively, a ligation may be a chemical ligation reaction. In the illustration, a spacer-less ligation is accomplished by using hybridization of a “recording helper” sequence with an arm on the coding tag. The annealed complement sequences are chemically ligated using standard chemical ligation or “click chemistry” (Gunderson et al., Genome Res (1998) 8(11): 1142-1153; Peng et al., European J Org Chem (2010) (22): 4194-4197; El-Sagheeret al., Proc Natl Acad Sci U S A (2011) 108(28): 11338-11343; El-Sagheer et al., Org Biomol Chem (2011) 9(1): 232-235; Sharma et al., Anal Chem (2012) 84(14): 6104-6109; Roloff et al., Bioorg Med Chem (2013) 21(12): 3458-3464; Litovchick et al., Artif DNA PNA XNA (2014) 5(1): e27896; Roloff et al., Methods Mol Biol (2014) 1050:131-141).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, coding tag information can be transferred using topoisomerase. Topoisomerase can be used be used to ligate a topo-charged 3′ phosphate on the recording tag (or extensions thereof or any nucleic acids attached) to the 5′ end of the coding tag, or complement thereof (Shuman et al., 1994, J. Biol. Chem. 269:32678-32684).

The extended recording tag is any nucleic acid molecule or sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) that comprises identifying information for a polypeptide to which it is associated. In some examples, the extended recording tag may comprise a unique molecular identifier, a compartment tag, a partition barcode, sample barcode, a fraction barcode, a spacer sequence, a universal priming site, or any combinations thereof. In certain embodiments, after a binding agent binds a polypeptide, information from a coding tag linked to a binding agent can be transferred to the nucleic acid associated with the polypeptide while the binding agent is bound to the polypeptide. In some examples, the final extended recording tag containing information from one or more binding agents is optionally flanked by universal priming sites to facilitate downstream amplification and/or DNA sequencing. The forward universal priming site (e.g., Illumina's P5-S 1 sequence) can be part of the original design of the recording tag and the reverse universal priming site (e.g., Illumina's P7-S2′ sequence) can be added as a final step in the extension of the nucleic acid. In some embodiments, the addition of forward and reverse priming sites can be done independently of a binding agent.

An extended nucleic acid associated with the macromolecule, e.g., the peptide, with identifying information from the coding tag may comprise information from a binding agent's coding tag representing each binding cycle performed. However, in some cases, an extended nucleic acid may also experience a “missed” binding cycle, e.g., if a binding agent fails to bind to the polypeptide, because the coding tag was missing, damaged, or defective, because the primer extension reaction failed. Even if a binding event occurs, transfer of information from the coding tag may be incomplete or less than 100% accurate, e.g., because a coding tag was damaged or defective, because errors were introduced in the primer extension reaction). Thus, an extended nucleic acid may represent 100%, or up to 95%, 90%, 85%, 80%, 75%, 70%, 65%, 60%, 65%, 55%, 50%, 45%, 40%, 35%, 30%, or any subrange thereof, of binding events that have occurred on its associated polypeptide. Moreover, the coding tag information present in the extended nucleic acid may have at least 30%, 35%, 40%, 45%, 50%, 55%, 60%, 65%, 70%, 75%, 80%, 85%, 90%, 95%, or 100% identity the corresponding coding tags.

In certain embodiments, an extended recording tag associated with the immobilized peptide may comprise information from multiple coding tags representing multiple, successive binding events. In these embodiments, a single, concatenated extended recording tag associated with the immobilized peptide can be representative of a single polypeptide. As referred to herein, transfer of coding tag information to the recording tag associated with the immobilized peptide also includes transfer to an extended recording tag as would occur in methods involving multiple, successive binding events.

In certain embodiments, the binding event information is transferred from a coding tag to the recording tag associated with the immobilized peptide in a cyclic fashion. Cross-reactive binding events can be informatically filtered out after sequencing by requiring that at least two different coding tags, identifying two or more independent binding events, map to the same class of binding agents (cognate to a particular protein). The coding tag may contain an optional UMI sequence in addition to one or more spacer sequences. Universal priming sequences may also be included in extended nucleic acids on the recording tag associated with the immobilized peptide for amplification and NGS sequencing.

D. Binding Agents

In certain embodiments, the methods for the macromolecule, e.g., the protein or polypeptide), analysis assay provided in the present disclosure comprise one or more binding cycles, where the polypeptides are contacted with a plurality of binding agents, and successive binding of binding agents transfers historical binding information in the form of a nucleic acid based coding tag to at least one nucleic acid (e.g., recording tag) associated with the polypeptides. In this way, a historical record containing information about multiple binding events is generated in a nucleic acid format.

The methods described herein use a binding agent capable of binding to the macromolecule, e.g., the polypeptide. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. In some embodiments, the scaffold used to engineer a binding agent can be from any species, e.g., human, non-human, transgenic. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule).

In certain embodiments, a binding agent may be designed to bind covalently. Covalent binding can be designed to be conditional or favored upon binding to the correct moiety. For example, an NTAA and its cognate NTAA-specific binding agent may each be modified with a reactive group such that once the NTAA-specific binding agent is bound to the cognate NTAA, a coupling reaction is carried out to create a covalent linkage between the two. Non-specific binding of the binding agent to other locations that lack the cognate reactive group would not result in covalent attachment. In some embodiments, the polypeptide comprises a ligand that is capable of forming a covalent bond to a binding agent. In some embodiments, the polypeptide comprises a functionalized NTAA which includes a ligand group that is capable of covalent binding to a binding agent. Covalent binding between a binding agent and its target allows for more stringent washing to be used to remove binding agents that are non-specifically bound, thus increasing the specificity of the assay.

In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding or Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some examples, a binding agent binds to an N-terminal amino acid residue, a C-terminal amino acid residue, or an internal amino acid residue.

In some specific examples, binding agents with different specificities can share the same coding tag. In some embodiments, a binding agent may exhibit flexibility and variability in target binding preference in some or all of the positions of the targets. In some examples, a binding agent may have a preference for one or more specific target terminal amino acids and have a flexible preference for a target at the penultimate position. In some other examples, a binding agent may have a preference for one or more specific target amino acids in the penultimate amino acid position and have a flexible preference for a target at the terminal amino acid position. In some embodiments, a binding agent is selective for a target comprising a terminal amino acid and other components of a macromolecule. In some examples, a binding agent is selective for a target comprising a terminal amino acid and at least a portion of the peptide backbone. In some particular examples, a binding agent is selective for a target comprising a terminal amino acid and an amide peptide backbone. In some cases, the peptide backbone comprises a natural peptide backbone or a post-translational modification. In some embodiments, the binding agent exhibits allosteric binding.

In the practice of the methods disclosed herein, the ability of a binding agent to selectively bind a feature or component of a macromolecule, e.g., a polypeptide, need only be sufficient to allow transfer of its coding tag information to the recording tag associated with the polypeptide. Thus, selectively need only be relative to the other binding agents to which the polypeptide is exposed. It should also be understood that selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a feature or component of a macromolecule is characterized by comparing binding abilities of binding agents. For example, the binding ability of a binding agent to the target can be compared to the binding ability of a binding agent which binds to a different target, for example, comparing a binding agent selective for a class of amino acids to a binding agent selective for a different class of amino acids. In some examples, a binding agent selective for non-polar side chains is compared to a binding agent selective for polar side chains. In some embodiments, a binding agent selective for a feature, component of a peptide, or one or more amino acid exhibits at least 1×, at least 2×, at least 5×, at least 10×, at least 50×, at least 100×, or at least 500× more binding compared to a binding agent selective for a different feature, component of a peptide, or one or more amino acid.

In a particular embodiment, the binding agent has a high affinity and high selectivity for the macromolecule, e.g., the polypeptide, of interest. In particular, a high binding affinity with a low off-rate may be efficacious for information transfer between the coding tag and recording tag. In certain embodiments, a binding agent has a Kd of about <500 nM, <200 nM, <100 nM, <50 nM, <10 nM, <5 nM, <1 nM, <0.5 nM, or <0.1 nM. In a particular embodiment, the binding agent is added to the polypeptide at a concentration >1×, >5×, >10×, >100×, or >1000× its Kd to drive binding to completion. For example, binding kinetics of an antibody to a single protein molecule is described in Chang et al., J Immunol Methods (2012) 378(1-2): 102-115.

In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native (e.g., natural) amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified NTAA, high specificity for a native or unmodified NTAA, or both. In some embodiments, binding agents can be developed through directed evolution of promising affinity scaffolds using phage display.

In certain embodiments, a binding agent may bind to a post-translational modification of an amino acid. In some embodiments, a peptide comprises one or more post-translational modifications, which may be the same of different. The NTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. A list of lectins recognizing various glycosylation states (e.g., core-fucose, sialic acids, N-acetyl-D-lactosamine, mannose, N-acetyl-glucosamine) include: A, AAA, AAL, ABA, ACA, ACG, ACL, AOL, ASA, BanLec, BC₂L-A, BC₂LCN, BPA, BPL, Calsepa, CGL2, CNL, Con, ConA, DBA, Discoidin, DSA, ECA, EEL, F17AG, Gall, Gall-S, Ga12, Ga13, Gal3C-S, Ga17-S, Ga19, GNA, GRFT, GS-I, GS-II, GSL-I, GSL-II, HHL, HIHA, HPA, I, II, Jacalin, LBA, LCA, LEA, LEL, Lentil, Lotus, LSL-N, LTL, MAA, MAH, MAL I, Malectin, MOA, MPA, MPL, NPA, Orysata, PA-IIL, PA-IL, PALa, PHA-E, PHA-L, PHA-P, PHAE, PHAL, PNA, PPL, PSA, PSL1a, PTL, PTL-I, PWM, RCA120, RS-Fuc, SAMB, SBA, SJA, SNA, SNA-I, SNA-II, SSA, STL, TJA-I, TJA-II, TxLCI, UDA, UEA-I, UEA-II, VFA, VVA, WFA, WGA (see, Zhang et al., 2016, MABS 8:524-535).

In some embodiments, a binding agent may bind to a native or unmodified or unlabeled terminal amino acid. In certain embodiments, a binding agent may bind to a modified or labeled terminal amino acid (e.g., an NTAA that has been functionalized or modified). In some embodiments, a binding agent may bind to a chemically or enzymatically modified terminal amino acid. A modified or labeled NTAA can be one that is functionalized with PITC, 1-fluoro-2,4-dinitrobenzene (Sanger's reagent, DNFB), benzyloxycarbonyl chloride or carbobenzoxy chloride (Cbz-C₁), N-(B enzyloxycarbonyloxy)succinimide (Cbz-OSu or Cbz NHS), dansyl chloride (DNS-C₁, or 1-dimethylaminonaphthalene-5-sulfonyl chloride), 4-sulfonyl-2-nitrofluorobenzene (SNFB), an acetylating reagent, a guanidinylation reagent, a thioacylation reagent, a thioacetylation reagent, or a thiobenzylation reagent, or a diheterocyclic methanimine reagent. In some examples, the binding agent binds an amino acid labeled by contacting with a reagent or using a method as described in International Patent Publication No. WO 2019/089846. In some cases, the binding agent binds an amino acid labeled by an amine modifying reagent.

In some embodiments, the binding agent binds to a chemically modified N-terminal amino acid residue or a chemically modified C-terminal amino acid residue. To increase the affinity of a binding agent to small N-terminal amino acids (NTAAs) of peptides, the NTAA may be modified with an “immunogenic” hapten, such as dinitrophenol (DNP). This can be implemented in a cyclic sequencing approach using Sanger's reagent, dinitrofluorobenzene (DNFB), which attaches a DNP group to the amine group of the NTAA. Commercial anti-DNP antibodies have affinities in the low nM range (˜8 nM, LO-DNP-2) (Bilgicer et al., J Am Chem Soc (2009) 131(26): 9361-9367); as such it stands to reason that it should be possible to engineer high-affinity NTAA binding agents to a number of NTAAs modified with DNP (via DNFB) and simultaneously achieve good binding selectivity for a particular NTAA. In another example, an NTAA may be modified with sulfonyl nitrophenol (SNP) using 4-sulfonyl-2-nitrofluorobenzene (SNFB). Similar affinity enhancements may also be achieved with alternative NTAA modifiers, such as an acetyl group or an amidinyl (guanidinyl) group.

In certain embodiments, a binding agent can be an aptamer (e.g., peptide aptamer, DNA aptamer, or RNA aptamer), a peptoid, an antibody or a specific binding fragment thereof, an amino acid binding protein or enzyme, an antibody binding fragment, an antibody mimetic, a peptide, a peptidomimetic, a protein, or a polynucleotide (e.g., DNA, RNA, peptide nucleic acid (PNA), a gPNA, bridged nucleic acid (BNA), xeno nucleic acid (XNA), glycerol nucleic acid (GNA), or threose nucleic acid (TNA), or a variant thereof).

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactive component(s) of an antibody molecule or portion thereof that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab′)₂ fragments, single chain antibody fragments (scFv), miniantibodies, nanobodies, diabodies, crosslinked antibody fragments, Affibody™, nanobodies, single domain antibodies, DVD-Ig molecules, alphabodies, affimers, affitins, cyclotides, molecules, and the like. Immunoreactive products derived using antibody engineering or protein engineering techniques are also expressly within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize a macromolecule, e.g., a peptide or a polypeptide, can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres. (see, Jayasena, 1999, Clin Chem 45:1628-50; Kusser2000, J. Biotechnol. 74: 27-39; Colas, 2000, Curr Opin Chem Biol 4:54-9). Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC, 1-fluoro-2,4-dinitrobenzene (using Sanger's reagent, DNFB), dansyl chloride (using DNS-C₁, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

In some embodiments, a binding agent that selectively binds to a labeled or functionalized NTAA can be utilized. For example, the NTAA may be reacted with phenylisothiocyanate (PITC) to form a phenylthiocarbamoyl-NTAA derivative. In this manner, the binding agent may be fashioned to selectively bind both the phenyl group of the phenylthiocarbamoyl moiety as well as the alpha-carbon R group of the NTAA. Use of PITC in this manner allows for subsequent elimination of the NTAA by Edman degradation as discussed below. In another embodiment, the NTAA may be reacted with Sanger's reagent (DNFB), to generate a DNP-labeled NTAA. Optionally, DNFB is used with an ionic liquid such as 1-ethyl-3-methylimidazolium bis[(trifluoromethyl)sulfonyl]imide ([emim][Tf2N]), in which DNFB is highly soluble. In this manner, the binding agent may be engineered to selectively bind the combination of the DNP and the R group on the NTAA. The addition of the DNP moiety provides a larger “handle” for the interaction of the binding agent with the NTAA, and should lead to a higher affinity interaction.

In yet another embodiment, a binding agent may be a modified aminopeptidase. In some embodiments, the binding agent may be a modified aminopeptidase that has been engineered to recognize the DNP-labeled NTAA providing cyclic control of aminopeptidase degradation of the peptide. Once the DNP-labeled NTAA is eliminated, another cycle of DNFB derivatization is performed in order to bind and eliminate the newly exposed NTAA. In preferred particular embodiment, the aminopeptidase is a monomeric metallo-protease, such an aminopeptidase activated by zinc (Calcagno et al., Appl Microbiol Biotechnol. (2016) 100(16):7091-7102). In another example, a binding agent may selectively bind to an NTAA that is modified with sulfonyl nitrophenol (SNP), e.g., by using 4-sulfonyl-2-nitrofluorobenzene (SNFB). Other reagents that may be used to functionalize the NTAA include trifluoroethyl isothiocyanate, allyl isothiocyanate, and dimethylaminoazobenzene isothiocyanate, or a reagent as described in International Patent Application No. PCT/US2018/58575.

A binding agent may be engineered for high affinity for a modified NTAA, high specificity for a modified NTAA, or both. In some embodiments, binding agents can be developed through directed evolution of promising affinity scaffolds using phage display.

In another example, highly-selective engineered ClpSs have also been described in the literature. Emili et al. describe the directed evolution of an E. coli ClpS protein via phage display, resulting in four different variants with the ability to selectively bind NTAAs for aspartic acid, arginine, tryptophan, and leucine residues (U.S. Patent Publication 9,566,335, incorporated by reference in its entirety). In one embodiment, the binding moiety of the binding agent comprises a member of the evolutionarily conserved ClpS family of adaptor proteins involved in natural N-terminal protein recognition and binding or a variant thereof. See e.g., Schuenemann et al., (2009) EMBO Reports 10(5); Roman-Hernandez et al., (2009) PNAS 106(22):8888-93; Guo et al., (2002) JBC 277(48): 46753-62; Wang et al., (2008) Molecular Cell 32: 406-414. In some embodiments, the amino acid residues corresponding to the ClpS hydrophobic binding pocket identified in Schuenemann et al. are modified in order to generate a binding moiety with the desired selectivity.

In one embodiment, the binding moiety comprises a member of the UBR box recognition sequence family, or a variant of the UBR box recognition sequence family. UBR recognition boxes are described in Tasaki et al., (2009), JBC 284(3): 1884-95. For example, the binding moiety may comprise UBR1, UBR2, or a mutant, variant, or homologue thereof.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the binding agent comprises a polypeptide, such as a modified member of the ClpS family of adaptor proteins, such as a variant of an E. coli ClpS binding polypeptide, and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphereTM, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g. PTC, modified-PTC, Cbz, DNP, SNP, acetyl, guanidinyl, amino guanidine, heterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer et al., 2012, Methods Enzymol 503: 157-188.). Engineering of alternative scaffolds for new binding functions has also been reviewed by Banta et al. (2013, Annu. Rev. Biomed. Eng. 15:93-113).

The functional affinity (avidity) of a given monovalent binding agent may be increased by at least an order of magnitude by using a bivalent or higher order multimer of the monovalent binding agent (Vauquelin et al., 2013, Br J Pharmacol 168(8): 1771-1785. 2013). Avidity refers to the accumulated strength of multiple, simultaneous, non-covalent binding interactions. An individual binding interaction may be easily dissociated. However, when multiple binding interactions are present at the same time, transient dissociation of a single binding interaction does not allow the binding protein to diffuse away and the binding interaction is likely to be restored. An alternative method for increasing avidity of a binding agent is to include complementary sequences in the coding tag attached to the binding agent and the recording tag associated with the polypeptide.

In some embodiments, the binding agent is derived from a biological, naturally occurring, non-naturally occurring, or synthetic source. In some examples, the binding agent is derived from de novo protein design (Huang et al., (2016) 537(7620):320-327). In some examples, the binding agent has a structure, sequence, and/or activity designed from first principles.

Other potential scaffolds that can be engineered to generate binding agents for use in the methods described herein include: an anticalin, a lipocalin, an amino acid tRNA synthetase (aaRS), ClpS, an Affilin®, an Adnectin™, a T cell receptor, a zinc finger protein, a thioredoxin, GST A1-1, DARPin, an affimer, an affitin, an alphabody, an avimer, a monobody, an antibody, a single domain antibody, a nanobody, EETI-II, HPSTI, intrabody, PHD-finger, V(NAR) LDTI, evibody, Ig(NAR), knottin, maxibody, microbody, neocarzinostatin, pVIII, tendamistat, VLR, protein A scaffold, MTI-II, ecotin, GCN₄, Im9, kunitz domain, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2). See e.g., El-Gebali et al., (2019) Nucleic Acids Research 47:D427—D432 and Finn et al., (2013) Nucleic Acids Res. 42(Database issue):D222—D230. In some embodiments, a binding agent is derived from an enzyme which binds one or more amino acids (e.g., an aminopeptidase). In certain embodiments, a binding agent can be derived from an anticalin or an ATP-dependent Clp protease adaptor protein (ClpS).

A binding agent may preferably bind to a modified or labeled amino acid, by chemical or enzymatic means, (e.g., an amino acid that has been functionalized by a reagent (e.g., a compound)) over a non-modified or unlabeled amino acid. For example, a binding agent may preferably bind to an amino acid that has been functionalized with an acetyl moiety, Cbz moiety, guanyl moiety, dansyl moiety, PTC moiety, DNP moiety, SNP moiety, heterocyclic methanimine moiety, etc., over an amino acid that does not possess said moiety. In some embodiments, a binding agent may preferably bind to an amino acid that has been functionalized or modified as described in International Patent Publication No. WO 2019/089846. In some cases, a binding agent may bind to a post-translationally modified amino acid. Thus, in certain embodiments, an extended nucleic acid associated with the comprises coding tag information relating to amino acid sequence and post-translational modifications of the polypeptide. In some embodiments, detection of internal post-translationally modified amino acids (e.g., phosphorylation, glycosylation, succinylation, ubiquitination, S-Nitrosylation, methylation, N-acetylation, lipidation, etc.) is be accomplished prior to detection and elimination of terminal amino acids (e.g., NTAA or CTAA). In one example, a peptide is contacted with binding agents for PTM modifications, and associated coding tag information are transferred to the recording tag associated with the immobilized peptide. Once the detection and transfer of coding tag information relating to amino acid modifications is complete, the PTM modifying groups can be removed before detection and transfer of coding tag information for the primary amino acid sequence using N-terminal or C-terminal degradation methods. Thus, resulting extended nucleic acids indicate the presence of post-translational modifications in a peptide sequence, though not the sequential order, along with primary amino acid sequence information.

In some embodiments, detection of internal post-translationally modified amino acids may occur concurrently with detection of primary amino acid sequence. In one example, an NTAA (or CTAA) is contacted with a binding agent specific for a post-translationally modified amino acid, either alone or as part of a library of binding agents (e.g., library composed of binding agents for the 20 standard amino acids and selected post-translational modified amino acids). Successive cycles of terminal amino acid elimination and contact with a binding agent (or library of binding agents) follow. Thus, resulting extended nucleic acids on the recording tag associated with the immobilized peptide indicate the presence and order of post-translational modifications in the context of a primary amino acid sequence.

In certain embodiments, a macromolecule, e.g., a polypeptide, is also contacted with a non-cognate binding agent. As used herein, a non-cognate binding agent is referring to a binding agent that is selective for a different polypeptide feature or component than the particular polypeptide being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the n^{th} NTAA (i.e phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n-1 amino acid of the peptide to the n-1 NTAA (e.g., tyrosine), and the peptide was then contacted with the same three binding agents, the binding agent selective for tyrosine would be second binding agent capable of selectively binding to the n-1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

Any binding agent described comprises a coding tag containing identifying information regarding the binding agent. A coding tag is a nucleic acid molecule of about 3 bases to about 100 bases that provides unique identifying information for its associated binding agent. A coding tag may comprise about 3 to about 90 bases, about 3 to about 80 bases, about 3 to about 70 bases, about 3 to about 60 bases, about 3 bases to about 50 bases, about 3 bases to about 40 bases, about 3 bases to about 30 bases, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be composed of DNA, RNA, polynucleotide analogs, or a combination thereof. Polynucleotide analogs include PNA, gPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

A coding tag comprises an encoder sequence that provides identifying information regarding the associated binding agent. An encoder sequence is about 3 bases to about 30 bases, about 3 bases to about 20 bases, about 3 bases to about 10 bases, or about 3 bases to about 8 bases. In some embodiments, an encoder sequence is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. The length of the encoder sequence determines the number of unique encoder sequences that can be generated. Shorter encoding sequences generate a smaller number of unique encoding sequences, which may be useful when using a small number of binding agents. In a specific embodiment, a set of >50 unique encoder sequences are used for a binding agent library.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-translationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. In certain embodiments, the spacer is binding agent specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. A 5′ spacer on a coding tag may optionally contain pseudo complementary bases to a 3′ spacer on the recording tag to increase Tₘ (Lehoud et al., 2008, Nucleic Acids Res. 36:3409-3419). In other embodiments, the coding tags within a library of binding agents do not have a binding cycle specific spacer sequence.

In one example, two or more binding agents that each bind to different targets have associated coding tags share the same spacers. In some cases, coding tags associated with two or more binding agents share coding tags with the same sequence or a portion thereof.

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. In some embodiments, a spacer sequence comprises a sufficient number of bases to anneal to a complementary spacer sequence in a recording tag or extended recording tag to initiate a primer extension reaction or sticky end ligation reaction.

In some embodiments, coding tags associated with binding agents used to bind in an alternating cycles comprises different binding cycle specific spacer sequences. For example, a coding tag for binding agents used in the first binding cycle comprise a “cycle 1” specific spacer sequence, a coding tag for binding agents used in the second binding cycle comprise a “cycle 2” specific spacer sequence, a coding tag for binding agents used in the third binding cycle also comprises the “cycle 1” specific spacer sequence, a coding tag for binding agents used in the fourth binding cycle comprises the “cycle 2” specific spacer sequence. In this manner, cycle specific spacers are not needed for every cycle.

A cycle specific spacer sequence can also be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle dependent spacer sequences. More specifically, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. Coding tags of binding agents from the first binding cycle are capable of annealing to recording tags via complementary cycle 1 specific spacer sequences. Upon transfer of the coding tag information to the recording tag, the cycle 2 specific spacer sequence is positioned at the 3′ terminus of the extended recording tag at the end of binding cycle 1. Coding tags of binding agents from the second binding cycle are capable of annealing to the extended recording tags via complementary cycle 2 specific spacer sequences. Upon transfer of the coding tag information to the extended recording tag, the cycle 3 specific spacer sequence is positioned at the 3′ terminus of the extended recording tag at the end of binding cycle 2, and so on through “n” binding cycles. This embodiment provides that transfer of binding information in a particular binding cycle among multiple binding cycles will only occur on (extended) recording tags that have experienced the previous binding cycles. However, sometimes a binding agent may fail to bind to a cognate polypeptide. Oligonucleotides comprising binding cycle specific spacers after each binding cycle as a “chase” step can be used to keep the binding cycles synchronized even if the event of a binding cycle failure. For example, if a cognate binding agent fails to bind to a polypeptide during binding cycle 1, adding a chase step following binding cycle 1 using oligonucleotides comprising both a cycle 1 specific spacer, a cycle 2 specific spacer, and a “null” encoder sequence. The “null” encoder sequence can be the absence of an encoder sequence or, preferably, a specific barcode that positively identifies a “null” binding cycle. The “null” oligonucleotide is capable of annealing to the recording tag via the cycle 1 specific spacer, and the cycle 2 specific spacer is transferred to the recording tag. Thus, binding agents from binding cycle 2 are capable of annealing to the extended recording tag via the cycle 2 specific spacer despite the failed binding cycle 1 event. The “null” oligonucleotide marks binding cycle 1 as a failed binding event within the extended recording tag.

In one embodiment, binding cycle-specific encoder sequences are used in coding tags. Binding cycle-specific encoder sequences may be accomplished either via the use of completely unique analyte (e.g., NTAA)-binding cycle encoder barcodes or through a combinatoric use of an analyte (e.g., NTAA) encoder sequence joined to a cycle-specific barcode. The advantage of using a combinatoric approach is that fewer total barcodes need to be designed. For a set of 20 analyte binding agents used across 10 cycles, only 20 analyte encoder sequence barcodes and 10 binding cycle specific barcodes need to be designed. In contrast, if the binding cycle is embedded directly in the binding agent encoder sequence, then a total of 200 independent encoder barcodes may need to be designed. An advantage of embedding binding cycle information directly in the encoder sequence is that the total length of the coding tag can be minimized when employing error-correcting barcodes. The use of error-tolerant barcodes allows highly accurate barcode identification using sequencing platforms and approaches that are more error-prone, but have other advantages such as rapid speed of analysis, lower cost, and/or more portable instrumentation.

In some embodiments, a coding tag comprises a cleavable or nickable DNA strand within the second (3′) spacer sequence proximal to the binding agent. For example, the 3′ spacer may have one or more uracil bases that can be nicked by uracil-specific excision reagent (USER). USER generates a single nucleotide gap at the location of the uracil. In another example, the 3′ spacer may comprise a recognition sequence for a nicking endonuclease that hydrolyzes only one strand of a duplex. Preferably, the enzyme used for cleaving or nicking the 3′ spacer sequence acts only on one DNA strand (the 3′ spacer of the coding tag), such that the other strand within the duplex belonging to the (extended) recording tag is left intact. These embodiments is particularly useful in assays analysing proteins in their native conformation, as it allows the non-denaturing removal of the binding agent from the (extended) recording tag after primer extension has occurred and leaves a single stranded DNA spacer sequence on the extended recording tag available for subsequent binding cycles.

The coding tags may also be designed to contain palindromic sequences. Inclusion of a palindromic sequence into a coding tag allows a nascent, growing, extended recording tag to fold upon itself as coding tag information is transferred. The extended recording tag is folded into a more compact structure, effectively decreasing undesired inter-molecular binding and primer extension events.

An extended recording tag can be built up from a series of binding events using coding tags comprising analyte-specific spacers and encoder sequences. In one embodiment, a first binding event employs a binding agent with a coding tag comprised of a generic 3′ spacer primer sequence and an analyte-specific spacer sequence at the 5′ terminus for use in the next binding cycle; subsequent binding cycles then use binding agents with encoded analyte-specific 3′ spacer sequences. This design results in amplifiable library elements being created only from a correct series of cognate binding events. Off-target and cross-reactive binding interactions will lead to a non-amplifiable extended recording tag. In one example, a pair of cognate binding agents to a particular polypeptide analyte is used in two binding cycles to identify the analyte. The first cognate binding agent contains a coding tag comprised of a generic spacer 3′ sequence for priming extension on the generic spacer sequence of the recording tag, and an encoded analyte-specific spacer at the 5′ end, which will be used in the next binding cycle. For matched cognate binding agent pairs, the 3′ analyte-specific spacer of the second binding agent is matched to the 5′ analyte-specific spacer of the first binding agent. In this way, only correct binding of the cognate pair of binding agents will result in an amplifiable extended recording tag. Cross-reactive binding agents will not be able to prime extension on the recording tag, and no amplifiable extended recording tag product generated. This approach greatly enhances the specificity of the methods disclosed herein. The same principle can be applied to triplet binding agent sets, in which 3 cycles of binding are employed. In a first binding cycle, a generic 3′ Sp sequence on the recording tag interacts with a generic spacer on a binding agent coding tag. Primer extension transfers coding tag information, including an analyte specific 5′ spacer, to the recording tag. Subsequent binding cycles employ analyte specific spacers on the binding agents' coding tags.

In certain embodiments, a coding tag may further comprise a unique molecular identifier for the binding agent to which the coding tag is linked.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag comprises a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

In some embodiments, the coding tag sequence can be optimized for the particular sequencing analysis platform. In a particular embodiment, the sequencing platform is nanopore sequencing. In some embodiments, the sequencing platform has a per base error rate of >1%, >5%, >10%, >15%, >20%, >25%, or >30%. For example, if the extended nucleic acid is to be analyzed using a nanopore sequencing instrument, the barcode sequences (e.g., sequences comprising identifying information from the coding tag) can be designed to be optimally electrically distinguishable in transit through a nanopore.

In some embodiments, a coding tag may include a terminator nucleotide incorporated at the 3′ end of the 3′ spacer sequence. After a binding agent binds to a macromolecule and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag is joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin). In some cases, a coding tag may be joined to a binding agent to an unnatural amino acid, such as via a covalent interaction with an unnatural amino acid.

In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Bioconjugate Techniques, G. T. Hermanson, Academic Press (2013)).

In some embodiments, an enzyme-based strategy is used to join the binding agent to a coding tag. For example, the binding agent may be joined to a coding tag using a formylglycine (FGly)-generating enzyme (FGE). In one example, a protein, e.g., SpyLigase, is used to join the binding agent to the coding tag (Fierer et al., Proc Natl Acad Sci U S A. 2014 Apr 1; 111(13): E1176—E1181).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In some cases, a binding agent is joined to a coding tag by attaching (conjugating) using an enzyme, such as sortase-mediated labeling (See e.g., Antos et al., Curr Protoc Protein Sci. (2009) CHAPTER 15: Unit-15.3; International Patent Publication No. WO2013003555). The sortase enzyme catalyzes a transpeptidation reaction (See e.g., Falck et al, Antibodies (2018) 7(4):1-19). In some aspects, the binding agent is modified with or attached to one or more N-terminal or C-terminal glycine residues.

In some embodiments, a binding agent is joined to a coding tag using a cysteine bioconjugation method. In some embodiments, a binding agent is joined to a coding tag using 7c-clamp-mediated cysteine bioconjugation (See e.g., Zhang et al., Nat Chem. (2016) 8(2):120-128). In some cases, a binding agent is joined to a coding tag using 3-arylpropiolonitriles (APN)-mediated tagging (e.g. Koniev et al., Bioconjug Chem. 2014 Feb 19;25(2):202-206).

In some embodiments, the binding agent is linked, directly or indirectly, to a multimerization domain. Thus, monomeric, dimeric, and higher order (e.g., 3, 4, 5, or more) multimeric polypeptides comprising one or more binding agents are provided herein. In some specific embodiments, the binding agent is dimeric. In some examples, two polypeptides of the invention can be covalently or non-covalently attached to each other to form a dimer.

In some embodiments, contacting of the first binding agent and second binding agent to the polypeptide, and optionally any further binding agents (e.g., third binding agent, fourth binding agent, fifth binding agent, and so on), are performed at the same time. For example, the first binding agent and second binding agent, and optionally any further order binding agents, can be pooled together, for example to form a library of binding agents. In another example, the first binding agent and second binding agent, and optionally any further order binding agents, rather than being pooled together, are added simultaneously to the polypeptide. In one embodiment, a library of binding agents comprises at least 20 binding agents that selectively bind to the 20 standard, naturally occurring amino acids. In some embodiments, a library of binding agents may comprise binding agents that selectively bind to the modified amino acids.

In other embodiments, the first binding agent and second binding agent, and optionally any further order binding agents, are each contacted with the polypeptide in separate binding cycles, added in sequential order. In certain embodiments, multiple binding agents are used at the same time in parallel. This parallel approach saves time and reduces non-specific binding by non-cognate binding agents to a site that is bound by a cognate binding agent (because the binding agents are in competition).

In certain embodiments, the concentration of the binding agents in a solution is controlled to reduce background and/or false positive results of the assay.

In some embodiments, the concentration of a binding agent can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1,000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1,000 nM.

In some embodiments, the ratio between the soluble binding agent molecules and the immobilized macromolecule, e.g., polypeptides, can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10⁴:1, about 10⁵:1, about 10⁶:1, or higher , or any ratio in between the above listed ratios. Higher ratios between the soluble binding agent molecules and the immobilized polypeptide(s) and/or the nucleic acids can be used to drive the binding and/or the coding tag information transfer to completion. This may be particularly useful for detecting and/or analyzing low abundance polypeptides in a sample.

In some embodiments, the binding agent is compatible for use in temperatures used in the macromolecule analysis assay. The binding agent may exhibit characteristics desired such as stability, solubility, and compatibility with other components of the macromolecule analysis assay. In some examples, the binding agent is compatible with the surface which is joined (directly or indirectly) to the macromolecules (e.g., polypeptides). In some embodiments, the binding agents exhibit low non-specific binding to the surface.

E. Amino Acid Cleavage

In some embodiments, following the transfer of identifying information from a coding tag to a recording tag, at least one terminal amino acid is removed, cleaved, or eliminated from the peptide. In some embodiments, the at least one removed terminal amino acid comprises a modified amino acid. In some embodiments, the at least one removed terminal amino acid comprises an unmodified amino acid. In embodiments relating to methods of analyzing peptides or polypeptides using a degradation based approach, following contacting and binding of a first binding agent to an n terminal amino acid (e.g., NTAA) of a peptide of n amino acids and transfer of the first binding agent's coding tag information to a nucleic acid associated with the peptide, thereby generating a first order extended nucleic acid (e.g., on the recording tag), the n NTAA is eliminated as described herein. Removal of the n labeled NTAA by contacting with an enzyme or chemical reagents converts the n-1 amino acid of the peptide to an N-terminal amino acid, which is referred to herein as an n-1 NTAA. A second binding agent is contacted with the peptide and binds to the n-1 NTAA, and the second binding agent's coding tag information is transferred to the first order extended nucleic acid thereby generating a second order extended nucleic acid (e.g., for generating a concatenated n^{th} order extended nucleic acid representing the peptide). Elimination of the n-1 labeled NTAA converts the n-2 amino acid of the peptide to an N-terminal amino acid, which is referred to herein as n-2 NTAA. Additional binding, transfer, labeling, and removal, can occur as described above up to n amino acids to generate an nt^{h} order extended nucleic acid or n separate extended nucleic acids, which collectively represent the peptide. As used herein, an n “order” when used in reference to a binding agent, coding tag, or extended nucleic acid, refers to the n binding cycle, wherein the binding agent and its associated coding tag is used or the n binding cycle where the extended nucleic acid is created (e.g. on recording tag). In some embodiments, steps including the NTAA in the described exemplary approach can be performed instead with a C terminal amino acid (CTAA).

In certain embodiments relating to analyzing peptides, following binding of a terminal amino acid (N-terminal or C-terminal) by a binding agent and transfer of coding tag information to a recording tag, the terminal amino acid is removed or cleaved from the peptide to expose a new terminal amino acid. In some embodiments, the terminal amino acid is an NTAA. In other embodiments, the terminal amino acid is a CTAA. Cleavage of a terminal amino acid can be accomplished by any number of known techniques, including chemical cleavage and enzymatic cleavage. In some embodiments, an engineered enzyme that catalyzes or reagent that promotes the removal of a labeled terminal amino acid is used. For example, the terminal amino acid is labeled with a PTC, a modified-PTC, a Cbz, a DNP, a SNP, an acetyl, a guanidinyl, amino guanidine, or a heterocyclic imine (e.g., heterocyclic methanimine). In some embodiments, the terminal amino acid is removed or eliminated using any of the methods as described in International Patent Publication No. WO 2019/089846.

Enzymatic cleavage of a terminal amino acid may be accomplished by an aminopeptidase or other peptidases (e.g., a carboxypeptidase, dipeptidyl peptidase, dipeptidyl aminopeptidase, or variant, mutant, or modified protein thereof). Aminopeptidases naturally occur as monomeric and multimeric enzymes, and may be metal or ATP-dependent. Natural aminopeptidases have very limited specificity, and generically cleave N-terminal amino acids in a processive manner, cleaving one amino acid off after another. For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz, DNP, SNP, acetyl, guanidinyl, amino guanidine, heterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label. Engineered aminopeptidase mutants that bind to and cleave individual or small groups of labelled (biotinylated) NTAAs have been described (see, PCT Publication No. WO2010/065322).

In some embodiments, the removed amino acid is an amino acid modified using any of the methods or reagents provided herein. For example, the reagent may comprise an enzymatic or chemical reagent to remove one or more terminal amino acid. For example, in some cases, the reagent for eliminating the functionalized NTAA is a carboxypeptidase, or aminopeptidase, or dipeptidyl peptidase, dipeptidyl aminopeptidase, or variant, mutant, or modified protein thereof; a hydrolase or variant, mutant, or modified protein thereof; mild Edman degradation; Edmanase enzyme; TFA, a base; or any combination thereof. In some cases, the removing reagent comprises trifluoroacetic acid or hydrochloric acid. In some examples, the removing reagent comprises acylpeptide hydrolase (APH). In some embodiments, the removing reagent includes a carboxypeptidase or an aminopeptidase or a variant, mutant, or modified protein thereof; a hydrolase or a variant, mutant, or modified protein thereof; a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc).

In some embodiments, the method further includes contacting the polypeptide with a peptide coupling reagent. In some embodiments, the peptide coupling reagent is a carbodiimide compound. In some examples, the carbodiimide compound is diisopropylcarbodiimide (DIC) or 1-ethyl-3-(3-dimethylaminopropyl)carbodiimide (EDC).

F. Processing and Analysis

In some embodiments, the extended recording tag generated from performing the provided methods comprises identifying information from one or more coding tags. In some embodiments, the extended recording tags (or a portion thereof) are amplified prior to determining at least a portion of the sequence of the extended recording tag. In some embodiments, the extended recording tags (or a portion thereof) are released from the macromolecule (e.g., polypeptide) prior to analysis of the extended recording tag. In some embodiments, the method includes collecting extended recording tags.

The length of the final extended nucleic acids (e.g., on the extended recording tag) generated by the methods described herein is dependent upon multiple factors, including the length of the coding tag (e.g., encoder sequence and spacer) and the length of any other of the nucleic acids (e.g., on the recording tag, optionally including any unique molecular identifier, spacer, universal priming site, barcode(s), or combinations thereof), the number of transfer cycles performed, and whether coding tags from each binding cycle are transferred to the same extended nucleic acid or to multiple extended nucleic acids.

In some embodiments, a recording tag comprises from 5′ to 3′ direction: a universal forward (or 5′) priming sequence, a UMI, and a spacer sequence. In some embodiments, a recording tag comprises from 5′ to 3′ direction: a universal forward (or 5′) priming sequence, an optional UMI, a barcode (e.g., sample barcode, partition barcode, compartment barcode, spatial barcode, or any combination thereof), and a spacer sequence. In some other embodiments, a recording tag comprises from 5′ to 3′ direction: a universal forward (or 5′) priming sequence, a barcode (e.g., sample barcode, partition barcode, compartment barcode, spatial barcode, or any combination thereof), an optional UMI, and a spacer sequence.

After the transfer of the final tag information to the extended recording tag from a coding tag, the tag can be capped by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT—3′—SEQ ID NO: 2) or an Illumina P5 primer (5′—AATGATACGGCGACCACCGA-3′—SEQ ID NO: 1). The sense or antisense P7 may be appended, depending on strand sense of the nucleic acid to which the identifying information from the coding tag is transferred to. An extended nucleic acid library can be cleaved or amplified directly from the solid support (e.g., beads) and used in traditional next generation sequencing assays and protocols.

In some embodiments, a primer extension reaction is performed on a library of single stranded extended nucleic acids (e.g., extended on the recording tag) to copy complementary strands thereof. In some embodiments, the peptide sequencing assay (e.g., ProteoCode assay), comprises several chemical and enzymatic steps in a cyclical progression. In some cases, one advantage of a single molecule assay is the robustness to inefficiencies in the various cyclical chemical/enzymatic steps. In some embodiments, the use of cycle-specific barcodes present in the coding tag sequence may be advantageous.

Extended nucleic acids (e.g., extended recording tags) can be processed and analyzed using a variety of nucleic acid sequencing methods. In some embodiments, extended recording tags containing the information from one or more coding tags and any other nucleic acid components are processed and analyzed. In some embodiments, the collection of extended recording can be concatenated. In some embodiments, the extended recording tag can be amplified prior to determining the sequence.

A library of nucleic acids (e.g., extended nucleic acids) may be amplified in a variety of ways. A library of nucleic acids (e.g., recording tags comprising information from one or more probe tags) undergo exponential amplification, e.g., via PCR or emulsion PCR. Emulsion PCR is known to produce more uniform amplification (Hori, Fukano et al., Biochem Biophys Res Commun (2007) 352(2): 323-328). Alternatively, a library of nucleic acids (e.g., extended nucleic acids) may undergo linear amplification, e.g., via in vitro transcription of template DNA using T7 RNA polymerase. The library of nucleic acids (e.g., extended nucleic acids) can be amplified using primers compatible with the universal forward priming site and universal reverse priming site contained therein. A library of nucleic acids (e.g., the recording tag) can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜1 mg of beads (™10 ng), 200 μM dNTP, 1 μM of each forward and reverse amplification primers, 0.5 μl (1U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of nucleic acids (e.g., extended nucleic acids) can undergo target enrichment. In some embodiments, target enrichment can be used to selectively capture or amplify extended nucleic acids representing macromolecules (e.g., polypeptides) of interest from a library of extended nucleic acids before sequencing. In some aspects, target enrichment for protein sequencing is challenging because of the high cost and difficulty in producing highly-specific binding agents for target proteins. In some cases, antibodies are notoriously non-specific and difficult to scale production across thousands of proteins. In some embodiments, the methods of the present disclosure circumvent this problem by converting the protein code into a nucleic acid code which can then make use of a wide range of targeted DNA enrichment strategies available for DNA libraries. In some cases, peptides of interest can be enriched in a sample by enriching their corresponding extended nucleic acids. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., (2010) Nature Methods 7: 111-118; Bodi et al., J. Biomol. Tech. (2013) 24:73-86; Ballester et al., (2016) Expert Review of Molecular Diagnostics 357-372; Mertes et al., (2011) Brief Funct. Genomics 10:374-386; Nilsson et al., (1994) Science 265:2085-8; each of which are incorporated herein by reference in their entirety).

In one embodiment, a library of nucleic acids (e.g., extended nucleic acids) is enriched via a hybrid capture-based assay. In a hybrid-capture based assay, the library of extended nucleic acids is hybridized to target-specific oligonucleotides that are labeled with an affinity tag (e.g., biotin). Extended nucleic acids hybridized to the target-specific oligonucleotides are “pulled down” via their affinity tags using an affinity ligand (e.g., streptavidin coated beads), and background (non-specific) extended nucleic acids are washed away. The enriched extended nucleic acids (e.g., extended nucleic acids) are then obtained for positive enrichment (e.g., eluted from the beads). In some embodiments, oligonucleotides complementary to the corresponding extended nucleic acid library representations of peptides of interest can be used in a hybrid capture assay. In some embodiments, sequential rounds or enrichment can also be carried out, with the same or different bait sets.

To enrich the entire length of a polypeptide in a library of extended nucleic acids representing fragments thereof (e.g., peptides), “tiled” bait oligonucleotides can be designed across the entire nucleic acid representation of the protein.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

Targeted enrichment methods can also be used in a negative selection mode to selectively remove extended nucleic acids from a library before sequencing. Examples of undesirable extended nucleic acids that can be removed are those representing over abundant polypeptide species, e.g., for proteins, albumin, immunoglobulins, etc.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment. The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended nucleic acid library. This approach works best for defined length libraries originating from peptides generated by site-specific protease digestion such as trypsin, LysC, GluC, etc. In one example, normalization can be accomplished by denaturing a double-stranded library and allowing the library elements to re-anneal. The abundant library elements re-anneal more quickly than less abundant elements due to the second-order rate constant of bimolecular hybridization kinetics (Bochman, Paeschke et al. 2012). The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., (2002) Genome Res. 12:1935-42) which destroys the dsDNA library elements.

Any combination of fractionation, enrichment, and subtraction methods, of the polypeptides before attachment to the solid support and/or of the resulting extended nucleic acid library can economize sequencing reads and improve measurement of low abundance species. In some embodiments, a library of nucleic acids (e.g., extended nucleic acids) is concatenated by ligation or end-complementary PCR to create a long DNA molecule comprising multiple different extended recorder tags (Du et al., (2003) BioTechniques 35:66-72; Muecke et al., (2008) Structure 16:837-841; U.S. Patent No. 5,834,252, each of which is incorporated by reference in its entirety). This embodiment is preferable for nanopore sequencing in which long strands of DNA are analyzed by the nanopore sequencing device.

In some embodiments, the recording tag or extended recording tag comprises information from one or more coding tags is analyzed and/or sequenced. In some embodiments, direct single molecule analysis is performed on the nucleic acids (e.g., extended nucleic acids) (see, e.g., Harris et al., (2008) Science 320:106-109). The nucleic acids (e.g., extended nucleic acids) can be analyzed directly on the solid support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labeled of decoding oligonucleotides (Gunderson et al., (2004) Genome Res. 14:970-7) can be used to ascertain both the identity and order of the coding tags within the extended nucleic acids (e.g., on the recording tag). In some embodiments, the binding agents may be labeled with cycle-specific coding tags as described above (see also, Gunderson et al., (2004) Genome Res. 14:970-7).

In some examples, the labels can be read out using traditional arrays or sequence-based methods. The methods described herein can be used in conjunction with a variety of sequencing techniques. In some embodiments, the process to determine the nucleotide sequence of a target nucleic acid can be an automated process. Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy. In some embodiments, suitable sequencing methods for use in the invention include, but are not limited to, sequencing by hybridization, sequencing by synthesis technology (e.g., HiSeq™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeg™, Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times) —this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science (2006) 311:1544-1546).

Some embodiments of the sequencing methods described herein include sequencing by synthesis (SBS) technologies, for example, pyrosequencing techniques. Pyrosequencing detects the release of inorganic pyrophosphate (PPi) as particular nucleotides are incorporated into the nascent strand (Ronaghi et al, Analytical Biochemistry 242(1): 84-9 (1996); Ronaghi, M. Genome Res. 11(1):3-11 (2001); Ronaghi et al, Science 281(5375):363 (1998); U.S. Pat. Nos. 6,210,891; 6,258,568 and 6,274,320, each of which is incorporated by reference in its entirety).

In another exemplary type of SBS, cycle sequencing is accomplished by stepwise addition of reversible terminator nucleotides containing, for example, a cleavable or photobleachable dye label as described, for example, in U.S. Pat. Nos. 7,427,67, 7,414, 1163 and 7,057,026, each of which is incorporated by reference in its entirety. This approach, which is being commercialized by Illumina Inc., is also described in International Patent Application Publication Nos. WO 91/06678 and WO 07/123744, each of which is incorporated by reference in its entirety. The availability of fluorescently-labeled terminators, in which both the termination can be reversed and the fluorescent label cleaved, facilitates efficient cyclic reversible termination (CRT) sequencing. Polymerases can also be co-engineered to efficiently incorporate and extend from these modified nucleotides.

Additional exemplary SBS systems and methods which can be utilized with the methods and compositions described herein are described in U.S. Patent Application Publication No. 2007/0166705, U.S. Patent Application Publication No. 2006/0188901, U.S. Patent No. 7057026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199 and PCT Publication No. WO 07/010251, each of which is incorporated by reference in its entirety.

Some embodiments of the sequencing technology described herein can utilize sequencing by ligation techniques. Such techniques utilize DNA ligase to incorporate nucleotides and identify the incorporation of such nucleotides. Exemplary SBS systems and methods which can be utilized with the compositions and methods described herein are described in U.S. Pat. No 6,969,488, 6,172,218, and U.S. Pat. No. 6,306,597, each of which is incorporated by reference in its entirety.

The sequencing methods described herein can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically coupled to a surface in a spatially distinguishable manner. For example, the target nucleic acids can be bound by direct covalent attachment, attachment to a bead or other particle or associated with a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail herein.

In some embodiments, the analysis of the sequence information of any of the labels (e.g., in the extended recording tag), or any portion thereof (e.g., a universal primer, a spacer, a UMI, a barcode), can be done using a single-molecule sequencing method, such as a nanopore based sequencing technology. In one aspect, the single-molecule sequencing method is a direct single-molecule sequencing method. See International Patent Application Publication No WO 2017/125565 for certain aspects of exemplary nanopore based sequencing, the content of which is incorporated by reference in its entirety. Nanopore sequencing of DNA and RNA may be achieved by strand sequencing and/or exosequencing of DNA and RNA. Strand sequencing comprises methods whereby nucleotide bases of a sample polynucleotide strand are determined directly as the nucleotides of the polynucleotide template are threaded through a nanopore. Alternatively, strand sequencing of the polynucleotide strand determines the sequence of the template indirectly by determining nucleotides that are incorporated into a growing strand that is complementary to that of the sample template strand.

In some embodiments, DNA, e.g., single stranded DNA, may be sequenced by detecting tags of tagged nucleotides that are released from the nucleotide base as the nucleotide is incorporated by a polymerase into a strand complementary to that of a template associated with the polymerase in an enzyme-polymer complex. The single molecule nanopore-based sequencing by synthesis (Nano-SBS) technique that uses tagged nucleotides is described, for example, in International Patent Application Publication No WO2014/074727, which is incorporated by reference in its entirety. Accordingly, in some embodiments, the enzyme-polynucleotide complex that may be attached to the inserted nanopore may be a DNA polymerase-DNA complex. In some embodiments, the DNA polymerase-DNA complex may be attached to a wild-type or variant monomeric nanopore. In some embodiments, the DNA polymerase-DNA complex may be attached to a wild-type, variant, or modified variant homo-oligomeric nanopore. In some embodiments, the DNA polymerase-DNA complex may be attached to a wild-type, a variant, or a modified variant hetero-oligomeric nanopore. In some embodiments, the DNA polymerase-DNA complex may be attached to a wild-type, variant, or modified variant aHL nanopore. In other embodiments, the DNA polymerase-DNA complex may be attached to a wild-type OmpG nanopore or variants thereof.

In other embodiments, the enzyme-polynucleotide complex may be an RNA polymerase-RNA complex. The RNA polymerase-RNA complex may be attached to a wild-type or variant oligomeric or monomeric nanopore. In some embodiments, the RNA polymerase-RNA complex is attached to a wild-type or variant OmpG nanopore. In other embodiments, the RNA polymerase-RNA complex is attached to a wild-type or variant aHL nanopore. In yet other embodiments, the enzyme-polynucleotide complex may be a reverse transcriptase-RNA complex. The reverse transcriptase-RNA complex may be attached to a wild-type or variant oligomeric or monomeric nanopore. In some embodiments, the reverse transcriptase-RNA complex is attached to a wild-type or variant OmpG nanopore. In other embodiments, the reverse transcriptase-RNA complex is attached to a wild-type or variant aHL nanopore. In some embodiments, individual nucleic acids may be sequenced by the identification of nucleoside 5′-monophosphates as they are released by processive exonucleases (Astier et al., 2006, J Am Chem Soc 128:1705-1710). Accordingly, in some embodiments, the enzyme-polynucleotide complex that may be attached to the inserted nanopore may be an exonuclease-polynucleotide complex. In some embodiments, the exonuclease-polynucleotide complex may be attached to a wild-type or variant monomeric nanopore. In some embodiments, the exonuclease-polynucleotide complex may be attached to a wild-type or variant homo-oligomeric nanopore. In some embodiments, the exonuclease-polynucleotide complex may be attached to a wild-type or variant hetero-oligomeric nanopore. In some embodiments, the exonuclease-polynucleotide complex may be attached to a wild-type aHL nanopore or variants thereof. In other embodiments, the exonuclease-polynucleotide complex may be attached to a wild-type OmpG nanopore or variants thereof.

In some embodiments, a non-nucleic acid polymer may also be move through a nanopore and be sequenced. For example, proteins and polypeptides can move through nanopores, and sequencing of a protein or a polynucleotide using a nanopore can be performed by controlling the unfolding and translocation of the protein through the nanopore. The controlled unfolding and subsequent translocation can be achieved by the action of an unfoldase enzyme coupled to the protein to be sequenced (see e.g., Nivala et al., 2013, Nature Biotechnol 31:247-250). In some embodiments, the enzyme-polymer complex that is attached to the nanopore in the membrane may be an enzyme-polypeptide complex, e.g., an unfoldase-protein complex. In some embodiments, the unfoldase-protein complex may be attached to a wild-type or variant monomeric nanopore. In some embodiments, the unfoldase-protein complex may be attached to a wild-type or variant homo-oligomeirc nanopore. In some embodiments, the unfoldase-protein complex may be attached to a wild-type or variant hetero-oligomeric nanopore. In some embodiments, the unfoldase-protein complex may be attached to a wild-type aHL nanopore or variants thereof. In other embodiments, the unfoldase-protein complex may be attached to a wild-type OmpG nanopore or variants thereof.

In some embodiments, other non-nucleic acid polymers may also be sequenced, for example, by moving through a nanopore. For example, WO 1996013606 A1 describes exo-sequencing of saccharide material, such as a polysaccharide including heparan sulphate (HS) and heparin, and U.S. Pat. No. 8,846,363 B2 discloses enzymes (such as a sulfatase from Flavobacterium heparinum) that can be applied (e.g., in tandem) toward the exo-sequencing of a polysaccharide, such as heparin-derived oligosaccharides. Both patent documents are incorporated herein by reference in their entireties for all purposes.

Enzymes of enzyme-polymer complexes and enzyme-nanopore complexes may include polynucleotide and polypeptide processing enzymes, e.g. DNA and RNA polymerases, reverse transcriptases, exonucleases, unfoldases, ligases, and sulfatases. The enzyme of the enzyme-polymer complex and enzyme-nanopore complex can be a wild-type enzyme, or it can be a variant form of the wild-type enzyme. Variant enzymes can be engineered to possess characteristics that are altered relative to those of the parent enzyme. In some embodiments, the enzyme of the enzyme-polymer complex that is altered is a polymerase. The altered characteristics of the polymerase enzyme include changes in enzyme activity, fidelity, processivity, elongation rate, stability, or solubility. The polymerase can be mutated to reduce the rate at which the polymerase incorporates a nucleotide into a nucleic acid strand (e.g., a growing nucleic acid strand). The reduced velocities (and improved sensitivities) can be achieved by a combination of site-specific mutagenesis of the nanopore protein and the incorporation of DNA processing enzymes, e.g., DNA polymerase, into the nanopore.

In some cases, the rate at which a nucleotide is incorporated into a nucleic acid strand can be reduced by functionalizing the nucleotide and/or template strand to provide steric hindrance, such as, for example, through methylation of the template nucleic acid strand. In some instances, the rate is reduced by incorporating methylated nucleotides.

The enzymes of the enzyme-polymer complex and enzyme-nanopore complex may be modified to comprise one or more attachment components and/or attachment sites that serve to link the enzyme-polymer complex to the nanopore inserted into the membrane. Similarly, the nanopore of the enzyme-nanopore complex and the nanopore to which the enzyme-polymer complex is attached may also be modified to comprise one or more attachment components and/or attachment sites to link the nanopore to the enzyme-polymer complex.

The nanopores of the nanopore sequencing complex include without limitation biological nanopores, solid state nanopores, and hybrid biological-solid state nanopores. In some embodiments, the nanopore sequencing complex includes solid state nanopores such as graphene, (ITO) indium tin oxide, pore lines with carbon nanotubes, or other inorganic solid state layers, or a combination of any of the foregoing. Biological nanopores of the nanopore sequencing complexes include OmpG from E. coli sp., Salmonella sp., Shigella sp., and Pseudomonas sp., and alpha hemolysin from S. aureus sp., MspA from M. smegmatis sp. The nanopores may be wild-type nanopores, variant nanopores, or modified variant nanopores. Variant nanopores can be engineered to possess characteristics that are altered relative to those of the parental enzyme (see, for example, U.S. Pat. No. 10,301,361 B2). In some embodiments, the characteristics are altered relative to the wild-type enzyme. In some embodiments, the variant nanopore of the nanopore sequencing complex is engineered to reduce the ionic current noise of the parental nanopore from which it is derived. An example of a variant nanopore having an altered characteristic is the OmpG nanopore having one or more mutations at the constriction zone (see, for example, U.S. Pat. No. 10,752,658 B2), which decrease the ionic noise level relative to that of the parent OmpG. The reduced ionic current noise provides for the use of these OmpG nanopore variants in single molecule sensing of polynucleotides and proteins. In other embodiments, the variant OmpG polypeptide can be further mutated to bind molecular adapters, which while resident in the pore slow the movement of analytes, e.g., nucleotide bases, through the pore and consequently improve the accuracy of the identification of the analyte. Modified variant nanopores are often or typically multimeric nanopores whose subunits have been engineered to affect inter-subunit interaction (US 20170088890 A1, entitled “Alpha-Hemolysin Variants”; US 20210293748 A1). Altered subunit interactions can be exploited to specify the sequence and order with which monomers oligomerize to form the multimeric nanopore in a lipid bilayer. This technique provides control of the stoichiometry of the subunits that form the nanopore. An example of a multimeric nanopore whose subunits can be modified to determine the sequence of interaction of subunits during oligomerization is an aHL nanopore.

The enzyme-polymer complex can be attached to the nanopore in any suitable way. Attaching enzyme-polymer complexes to nanopores may be achieved using the SpyTag/SpyCatcher peptide system (Zakeri et al., 2012, PNAS109:E690-E697) native chemical ligation (Thapa et al., 2014, Molecules 19:14461-14483), sortase system (Wu and Guo, 2012, J Carbohydr Chem 31 :48-66; Heck et al., 2013, Appl Microbiol Biotechnol 97:461-475), transglutaminase systems (Dennler et al., 2014, Bioconjug Chem 25:569-578), formylglycine linkage (Rashidian et al., 2013, Bioconjug Chem 24:1277-1294), or other chemical ligation techniques known in the art.

In some embodiments, the enzyme is linked to the nanopore using Solulink™ chemistry; see e.g., US 20200216887 A1. Solulink™ can be a reaction between HyNic (6-hydrazino-nicotinic acid, an aromatic hydrazine) and 4FB (4-formylbenzoate, an aromatic aldehyde). In some instances, the polymerase is linked to the nanopore using Click chemistry (available from Life Technologies, for example; see also U.S. Pat. No. 9,862,997 B2). In some cases, zinc finger mutations are introduced into the hemolysin molecule and then a molecule is used (e.g., a DNA intermediate molecule) to link the enzyme to the zinc finger sites on the hemolysin.

In some embodiments, the information from analysis (e.g., sequencing) of at least a portion of the extended recording tag can be used to associate the sequences determined to corresponding a polypeptide and align to the proteome. In some cases, following sequencing of the nucleic acid libraries (e.g., of extended nucleic acids), the resulting sequences can be collapsed by their UMIs and then associated to their corresponding polypeptides and aligned to the totality of the proteome. In some cases, resulting sequences can also be collapsed by their compartment tags and associated to their corresponding compartmental proteome, which in a particular embodiment contains only a single or a very limited number of protein molecules. In some embodiments, both protein identification and quantification can be derived from this digital peptide information.

The methods disclosed herein can be used for analysis, including detection, quantitation and/or sequencing, of a plurality of macromolecules simultaneously (multiplexing). Multiplexing as used herein refers to analysis of a plurality of macromolecules (e.g. polypeptides) in the same assay. The plurality of macromolecules can be derived from the same sample or different samples. The plurality of macromolecules can be derived from the same subject or different subjects. The plurality of macromolecules that are analyzed can be different macromolecules, or the same macromolecule derived from different samples. A plurality of macromolecules includes 2 or more macromolecules, 5 or more macromolecules, 10 or more macromolecules, 50 or more macromolecules, 100 or more macromolecules, 500 or more macromolecules, 1000 or more macromolecules, 5,000 or more macromolecules, 10,000 or more macromolecules, 50,000 or more macromolecules, 100,000 or more macromolecules, 500,000 or more macromolecules, or 1,000,000 or more macromolecules.

### VII. KITS AND ARTICLES OF MANUFACTURE

Provided herein are kits and articles of manufacture comprising components for preparing and analyzing macromolecules (e.g., proteins, polypeptides, or peptides). The kits and articles of manufacture may include any one or more of the reagents and components used in the methods described above. In some embodiments, the kits optionally include instructions for use. In some embodiments, the kits comprise one or more of the following components: recoding tag(s), reagent(s) for attaching the recording tag, reagent(s) for transferring information from the probe tag to the recording tag, binding agent(s), reagent(s) for transferring identifying information from the coding tag to the recording tag, sequencing reagent(s), solid support(s), enzyme(s), buffer(s), and/or sample processing reagent(s) (e.g. fixation and permeabilization reagent(s).

In another aspect, disclosed herein is a kit for analyzing a polypeptide, comprising: a library of binding agents, wherein each binding agent comprises a binding moiety and a coding tag comprising identifying information regarding the binding moiety. In some embodiments, the binding moiety is capable of binding to one or more N-terminal, internal, or C-terminal amino acids of the polypeptide. In some embodiments, the binding moiety is capable of binding to one or more modified N-terminal, internal, or C-terminal amino acids of the polypeptide. In some cases, N-terminal, internal, or C-terminal amino acids are modified by a functionalizing reagent.

In some embodiments, the kits and articles of manufacture further comprise a plurality of barcodes. The barcode may include a compartment barcode, a partition barcode, a sample barcode, a fraction barcode, or any combination thereof. In some cases, the barcode comprises a unique molecule identifier (UMI). In some examples, the barcode comprises a DNA molecule, DNA with pseudo-complementary bases, an RNA molecule, a BNA molecule, an XNA molecule, a LNA molecule, a PNA molecule, a gPNA molecule, a non-nucleic acid sequenceable polymer, e.g., a polysaccharide, a polypeptide, a peptide, or a polyamide, or a combination thereof. In some embodiments, the barcodes are configured to attach the macromolecules, e.g., the proteins, in the sample or to attach to nucleic components associated with the macromolecules, e.g., the proteins.

In some embodiments, the kit further comprises reagents for treating the macromolecules, e.g., the proteins. Any combination of fractionation, enrichment, and subtraction methods, of the macromolecules, e.g., the proteins, may be performed. For example, the reagent may be used to fragment or digest the macromolecules, e.g., the proteins. In some cases, the kit comprises reagents and components to fractionate, isolate, subtract, enrich the macromolecules, e.g., the proteins. In some examples, the kits further comprises a protease such as trypsin, LysN, or LysC.

In some embodiments, the kit includes one or more reagents for nucleic acid sequence analysis. In some examples, the reagent for sequence analysis is for use in sequencing by synthesis, sequencing by ligation, single molecule sequencing, single molecule fluorescent sequencing, sequencing by hybridization, polony sequencing, ion semiconductor sequencing, pyrosequencing, single molecule real-time sequencing, nanopore-based sequencing, or direct imaging of DNA using advanced microscopy, or any combination thereof.

In some embodiments, the kit also comprises one or more buffers or reaction fluids necessary for any of the desired reaction to occur. Buffers including wash buffers, reaction buffers, and binding buffers, elution buffers and the like are known to those or ordinary skill in the arts. In some embodiments, the kits further include buffers and other components to accompany other reagents described herein.

Reagents and kit components may be provided in any suitable container. In some embodiments, the kits further include buffers and other components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

In some embodiments, the kits or articles of manufacture may further comprise instruction(s) on the methods and uses described herein. In some embodiments, the instructions are directed to methods of analyzing the macromolecules (e.g., proteins, polypeptides, or peptides). The kits described herein may also include other materials desirable from a commercial and user standpoint, including other buffers, diluents, filters, syringes, and package inserts with instructions for performing any methods described herein. Any of the components of the kits may be sterilized and/or sealed.

Any of the above-mentioned kit and components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

### VIII. EXAMPLARY EMBODIMENTS

The following enumerated embodiments represent certain aspects and examples of the invention:

Embodiment 1. A binder that specifically binds to an N-terminally modified target polypeptide, wherein:

said N-terminally modified target polypeptide is derived from a target polypeptide and said N-terminally modified target polypeptide has a formula:

M-P1-P2-polypeptide, said M being an N-terminal modification, said P1 being the N-terminal amino acid residue of said target polypeptide, and P2 being a penultimate terminal amino acid residue of said target polypeptide; and

said binder specifically binds to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

Embodiment 2. The binder of embodiment 1, wherein the length of the target polypeptide and/or the N-terminally modified target polypeptide is greater than 4 amino acids, greater than 5 amino acids, greater than 6 amino acids, greater than 7 amino acids, greater than 8 amino acids, greater than 9 amino acids, greater than 10 amino acids, greater than 11 amino acids, greater than 12 amino acids, greater than 13 amino acids, greater than 14 amino acids, greater than 15 amino acids, greater than 20 amino acids, greater than 25 amino acids, or greater than 30 amino acids.

Embodiment 3. The binder of embodiment 1 or 2, wherein the P1 comprises a naturally-occurring amino acid residue.

Embodiment 4. The binder of embodiment 3, wherein the P1 is selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

Embodiment 5. The binder of any one of embodiments 1-4, wherein the P1 comprises a modification, e.g., a naturally-occurring or a non-natural modification.

Embodiment 6. The binder of any one of embodiments 1-5, wherein the P1 comprises an amino acid with a post-translational modification.

Embodiment 7. The binder of any one of embodiments 1-6, wherein the P2 comprises a naturally-occurring amino acid residue.

Embodiment 8. The binder of embodiment 7, wherein the P2 is selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

Embodiment 9. The binder of embodiment 7 or 8, wherein the P2 comprises a modification, e.g., a naturally-occurring or a non-natural modification.

Embodiment 10. The binder of any one of embodiments 7-9, wherein the P2 comprises an amino acid with a post-translational modification.

Embodiment 11. The binder of any one of embodiments 1-10, wherein the M comprises a synthetic N-terminal modification.

Embodiment 12. The binder of any one of embodiments 1-11, wherein the M comprises an amino acid moiety and/or has a size, e.g., length axis or volume, shape, and/or configuration similar to or exceeding a natural amino acid, preferably having a volume greater than the volume of glycine and less than about 1,000 Å³.

Embodiment 13. The binder of embodiment 12, wherein the M comprises an amino acid moiety.

Embodiment 14. The binder of embodiment 13, wherein the M is a bipartite N-terminal modification (NTM) that comprises a natural or unnatural amino acid portion (NTMaa) and an N-terminal blocking group (NTM_{blk}), the amino acid portion (NTMaa) and the N-terminal blocking group (NTM_{blk}) being optionally connected with an amide bond.

Embodiment 15. The binder of embodiment 12, wherein the M does not comprise an amino acid moiety.

Embodiment 16. The binder of embodiment 15, wherein the M is a bipartite N-terminal modification (NTM) that comprises a small chemical entity having a size, e.g., length axis or volume, shape, and/or configuration similar to or exceeding a natural amino acid, and an N-terminal blocking group (NTM_{blk}),

the small chemical entity and the N-terminal blocking group optionally connected with an amide bond, and/or

optionally, the small chemical entity having a size, e.g., length axis of ˜5-10 Å and volume of 100-1000 Å³.

Embodiment 17. The binder of any one of embodiments 1-16, wherein the M comprises a group selected from:

(1) Formula (3′):

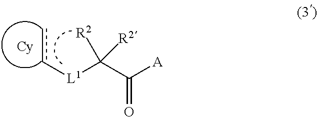

wherein A represents the point of attachment of the group to P1;

Cy is a 5 to 7 membered ring or an 8-10 membered bicyclic ring system, and Cy may be absent or present;

when present, ring Cy may be saturated, unsaturated, or aromatic, and the dashed bond may be a single bond, double bond, or aromatic bond;

when Cy is present, it may be a carbocyclic ring, or it may contain one to three heteroatoms selected from N, O, B, and S as ring members; and Cy is optionally substituted with one to six groups (or with one to four groups when Cy is aromatic) selected from halo, CN, NH₂, NH(CH₃), N(CH₃)₂, protected amine (e.g., N₃, NO₂, NHFmoc, NHBoc), C(O)NR2, NHC(O)R, B(OR)₂, aryl, —SR⁴, —S(O)ₙR⁴, —NR⁴SO₂R⁴, —SO₂N(R⁴)₂, heteroaryl, C₁-C₂ alkyl, C₁-C₂ haloalkyl, C₁-C₂ haloalkoxy, and —OR⁴;

when ring Cy is absent, the dashed bond may be a single bond or a double bond, and the dashed bond is optionally substituted by one or two groups selected from halo, CN, C₁-C₂ alkyl, C₁-C₂ haloalkyl, C₁-C₂ haloalkoxy, CO₂R⁴, and —OR⁴; each L¹ is independently a bond or C₁-C₂ alkylene, C₁-C₂ haloalkylene, NHC(O), SO₂, or NHSO₂;

R² and R^{2′} can each be H or a side chain of an amino acid, e.g. one of the side chains of the 20 common amino acid side chains, optionally protected amino acid side chains, post-translationally modified amino acid side chains, unnatural amino acid sidechains;

R² side chains are independently selected from side chains of an amino acid selected from Alanine, aspartic acid, asparagine, glutamic acid, glutamine, glycine, (2-, 3-, or 4-pyridyl-) alanine, phenylglycine, 4-fluorophenylglycine, leucine, norleucine, isoleucine, cycloleucine, valine, dimethylglycine, methionine, methionine sulfoxide, phenylalanine, halophenylalanine, haloalkylphenylalanine, cyclopropylalanine, (2-thienyl)alanine, cyclopropylglycine, serine, threonine, cysteine, carbamidomethylcysteine, trifluoromethylcysteine, tyrosine, tryptophan, histidine, acetyllysine, proline, (2- or 3-)azetidine carboxylic acid, piperidine carboxylic acid, methylated lysine, citrulline, nitroarginine, norvaline, phosphoserine, phosphothreonine, and phosphotyrosine;

or R² or R^{2′} can be an aryl, heteroaryl, bicyclic aryl, or bicyclic heteroaryl, each of which is optionally substituted with up to three groups independently selected from halo, cyano, azido, amino, C₁-C₂ alkyl, C₁-C₂ haloalkyl, C₁-C₂ alkoxy, and C₁-C₂ haloalkoxy;

[symbol] represents an optional link between R² and L¹, forming a 5-6 membered ring;

n at each occurrence is independently 1 or 2; and

each R and R⁴ is independently selected from H, C₁₋₂ alkyl, and C₁-C₂ haloalkyl; (2) Formula (4′):

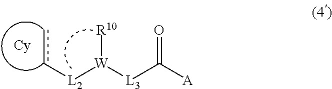

wherein A represents the point of attachment of the group to P1;

W is a bond or a group selected from alkyl, cycloalkyl, heterocyclyl, aryl, heteroaryl, and bicyclic heteroaryl, each of which is optionally substituted with up to four groups independently selected from halo, OH, cyano, azido, —SR⁴, —S(O)ₙR⁴, —NR⁴SO₂R⁴, —SO₂N(R⁴)₂, —B(OR⁴)₂, oxo (unless W is aromatic), amino, C₁-C₂ alkyl, C₁-C₂ haloalkyl, C₁-C₂ alkoxy, and C₁-C₂ haloalkoxy;

when W is a ring, ring W may be saturated, unsaturated, or aromatic; when W is a heterocyclic or heteroaromatic ring, it may contain one or two heteroatoms selected from N, O and S as ring members;

curved dotted line represents an optional linkage connecting R¹⁰ and L² into a 5-6 membered ring, optionally including an additional N, O or S as a ring member;

R¹⁰ is selected from H, halo, CN, NH₂, NH(CH₃), N(CH₃)₂, NO₂ NHFmoc, NHBoc, C(O)NR₂, NHC(O)R, NHC(O)OR, B(OR)₂, aryl, heteroaryl, C₁-C₂ alkyl, C₁-C₂ haloalkyl, C₁-C₂ haloalkoxy, and —OR⁴; and R¹⁰ is absent when W is a bond;

L² and L³ are independently selected from a bond, CH₂, SO₂R, NHSO₂R, C(═O)R, RNHC(═O), RNCH₃C(═O), C₁-C₂ alkylene, C₁-C₂ haloalkylene, or triazole;

each R is independently selected from C₁₋₆ alkyl, phenyl, and benzyl, each of which is optionally substituted with up to three groups selected from halo, CN, C₁-C₂ haloalkyl, C₁-C₂ haloalkoxy, CO₂R⁴, and —OR⁴;

Cy is a 5 to 7 membered ring or an 8-10 membered bicyclic ring system, and Cy may be absent or present;

when present, ring Cy may be saturated, unsaturated, or aromatic, and the dashed bond may be a single bond, double bond, or aromatic bond;

when Cy is present, it may be a carbocyclic ring, or it may contain one to three heteroatoms selected from N, O, B and S as ring members; and Cy is optionally substituted with one to six groups (or with one to four groups when Cy is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, —SR⁴, —S(O)ₙR⁴, —NR⁴SO₂R⁴, —SO₂N(R⁴)₂, and —OR⁴;

when ring Cy is absent, the dashed bond may be a single bond or a double bond, and the dashed bond is optionally substituted by one or two groups selected from halo, CN, C₁-C₂ alkyl, C₁-C₂ haloalkyl, C₁-C₂ haloalkoxy, CO₂R⁴, and —OR⁴;

each L¹ is independently a bond or C₁-C₂ alkylene, C₁-C₂ haloalkylene, NHC(O), SO₂, or NHSO₂;

n at each occurrence is independently 1 or 2; and

R⁴ is independently selected at each occurrence from H, C₁-C₂ alkyl, and C₁-C₂ haloalkyl;

(3) Formula (5′):

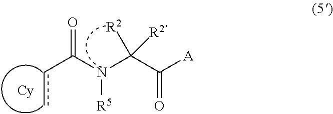

wherein A represents the point of attachment of the group to P1;

[symbol] represents an optional link between R² and nitrogen, forming a 5-6 membered ring: when the optional link is present, R⁵ is absent;

Cy is a 5 to 7 membered ring or an 8-10 membered bicyclic ring system, and Cy may be absent or present;

when present, ring Cy may be saturated, unsaturated, or aromatic, and the dashed bond may be a single bond, double bond, or aromatic bond;

when Cy is present, it may be a carbocyclic ring, or it may contain one to three heteroatoms selected from N, O, B, and S as ring members; and Cy is optionally substituted with one to six groups (or with one to four groups when Cy is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, —SR⁴, —S(O)ₙR⁴, —NR⁴SO₂R⁴, —SO₂N(R⁴)₂, and —OR⁴;

when ring Cy is absent, the dashed bond may be a single bond or a double bond, and the dashed bond is optionally substituted by one or two groups selected from halo, CN, C₁-C₂ alkyl, C₁-C₂ haloalkyl, C₁-C₂ haloalkoxy, CO₂R⁴, and -OR⁴;

R² and R²′ can each be the side chain of an amino acid, e.g. one of the side chains of the 20 common amino acid side chains, optionally protected amino acid side chains, post-translationally modified amino acid side chains, unnatural amino acid sidechains; or

R² and R^{2′} can each be H or a group selected from aryl, heteroaryl, bicyclic aryl, bicyclic heteroaryl, and heterocyclyl, each of which is optionally substituted with one to six groups (or with one to four groups when R² or R²′ is aromatic) selected from halo, CN, NH₂, NH(CH₃), N(CH₃)₂, protected amine (e.g., N₃, NO₂, NHFmoc, NHBoc), C(O)NR2, NHC(O)R, B(OR)₂, aryl, heteroaryl, C₁-C₂ alkyl, C₁-C₂ haloalkyl, C₁-C₂ haloalkoxy, and —OR⁴;

each R and R⁴ is independently selected at each occurrence from H, C₁-C₂ alkyl, and C₁-C₂ haloalkyl;

n at each occurrence is independently 1 or 2; and

R⁵ is independently selected at each occurrence from H, C₁-C₂ alkyl, C₁-C₂ haloalkyl, C₁-C₂ alkoxy, and C₁-C₂ haloalkoxy;

(4) Formula (6′):

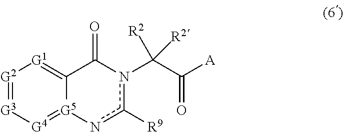

wherein A represents the point of attachment of the group to P1;

G¹-G⁵ are each independently selected from CH, CJ, BN, BO, and N, provided not more than 3 of G¹-G⁵ are N;

the dashed bonds can be single bonds or double bonds;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR^{S}, —C(—O)Ar, and tetrazole, where Ar represents a phenyl or 5-6 membered heteroaryl ring that is optionally substituted with one or two groups selected from halo, CN, R⁸ and OR⁸;

R² and R^{2′} can each be the side chain of an amino acid, e.g. one of the side chains of the 20 common amino acid side chains, optionally protected amino acid side chains, post-translationally modified amino acid side chains, unnatural amino acid sidechains; or

R² and R^{2′} can each be H or a group selected from aryl, heteroaryl, bicyclic aryl, bicyclic heteroaryl, and heterocyclyl, each of which is optionally substituted with one to six groups (or with one to four groups when R² or R²′ is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, and —OR⁴;

each R, R⁴ and R⁸ is independently selected at each occurrence from H, C₁-C₂ alkyl, and C₁-C₂ haloalkyl; and

n at each occurrence is independently 1 or 2; and

R⁹ is H, CH₃, benzyl, substituted benzyl;

(5) Formula (7′):

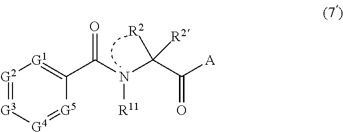

wherein A represents the point of attachment of the group to P1;

G¹-G⁵ are each independently selected from CH, CJ, BN, BO, and N, provided not more than 3 of G¹-G⁵ are N;

[symbol] represents an optional link between R² and the nitrogen atom, forming a 5-6 membered ring: when the link is present, R″ is absent;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR⁸, —C(—O)Ar, and tetrazole, where Ar represents a phenyl or 5-6 membered heteroaryl ring that is optionally substituted with one or two groups selected from halo, CN, R⁸ and OR⁸;

R² and R^{2′} can each be the side chain of an amino acid, e.g. one of the side chains of the 20 common amino acid side chains, optionally protected amino acid side chains, post-translationally modified amino acid side chains, unnatural amino acid sidechains;

or R² and R^{2′} can each be H or a group selected from aryl, heteroaryl, bicyclic aryl, bicyclic heteroaryl, and heterocyclyl, each of which is optionally substituted with one to six groups (or with one to four groups when R² or R²′ is aromatic) selected from halo, CN, NH₂, NH(CH₃), N(CH₃)₂, protected amine (e.g., N₃, NO₂, NHFmoc, NHBoc), C(O)NR₂, NHC(O)R, B(OR)₂, aryl, heteroaryl, C₁-C₂ alkyl, C₁-C₂ haloalkyl, C₁-C₂ haloalkoxy, and —OR⁴;

each R, R⁴ and R⁸ is independently selected at each occurrence from H, C₁-C₂ alkyl, and C₁-C₂ haloalkyl;

n at each occurrence is independently 1 or 2; and

R¹¹ is H, CH₃, benzyl, or substituted benzyl; and (6) Formula (8′):

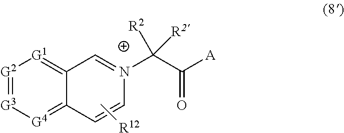

wherein A represents the point of attachment of the group to P1;

G¹-G⁵ are each independently selected from CH, CJ, BN, BO, and N, provided not more than 3 of G¹-G⁵ are N;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR^{S}, —C(—O)Ar, and tetrazole, where Ar represents a phenyl or 5-6 membered heteroaryl ring that is optionally substituted with one or two groups selected from halo, CN, R⁸ and OR⁸;

R² and R²′ can each be the side chain of an amino acid, e.g. one of the side chains of the 20 common amino acid side chains, optionally protected amino acid side chains, post-translationally modified amino acid side chains, unnatural amino acid sidechains;

or R² and R²′ can each be H or a group selected from aryl, heteroaryl, bicyclic aryl, bicyclic heteroaryl, and heterocyclyl, each of which is optionally substituted with one to six groups (or with one to four groups when R² or R²′ is aromatic) selected from halo, CN, NH₂, NH(CH₃), N(CH₃)₂, protected amine (e.g., N₃, NO₂, NHFmoc, NHBoc), C(O)NR2, NHC(O)R, B(OR)₂, aryl, heteroaryl, C₁-C₂ alkyl, C₁-C₂ haloalkyl, C₁-C₂ haloalkoxy, and -OR⁴;

each R, R⁴ and R⁸ is independently selected at each occurrence from H, C₁-C₂ alkyl, and C₁-C₂ haloalkyl;

n at each occurrence is independently 1 or 2; and

R¹² represents one or two optional substituents on the pyridinium ring, which are independently selected from C₁-C₂ alkyl, C₁-C₂ haloalkyl, C₁-C₂ haloalkoxy, and halo and

(7) Formula (10):

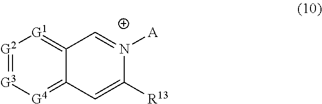

wherein A represents the point of attachment of the group to P1 of a target polypeptide;

G¹-G⁴ are each independently selected from CH, CJ, and N, provided not more than 3 of G¹-G⁴ are N;

J at each occurrence is independently selected from H, C₁-C₂ alkyl, NO₂, C₁-C₂ haloalkyl, C₁-C₂ haloalkoxy, halo, —OR⁸, —N(R⁸)₂, —S(O)ₙR⁸, —NR⁸SO₂R⁸, —SO₂N(R⁸)₂, SO₃R⁸, —B(OR⁸)₂, C(═O)R⁸, CN, CON(R⁸)₂, —COOR^{S}, —C(—O)Ar, and tetrazole, where Ar represents a phenyl or 5-6 membered heteroaryl ring that is optionally substituted with one or two groups selected from halo, CN, R⁹ and OR⁹;

each R⁸ and each R⁹ is independently selected from H, C₁-C₂ alkyl, and C₁-C₂ haloalkyl;

n at each occurrence is independently 1 or 2; and

R¹³ is selected from H, C₁-C₂ alkyl, C₁-C₂ alkoxy, C₁-C₂ haloalkyl, and C₁-C₂ haloalkoxy.

Embodiment 18. The binder of any one of embodiments 1-17, wherein the interaction between the binder and the M has minimal or no impact or influence on the binding specificity between the binder and the N-terminally modified target polypeptide.

Embodiment 19. The binder of any one of embodiments 1-18, wherein the interaction between the binder and the M at least partially determines the binding strength or binding efficiency between the binder and the N-terminally modified target polypeptide.

Embodiment 20. The binder of any one of embodiments 1-19, wherein there is minimal or no interaction between the binder and the P2.

Embodiment 21. The binder of any one of embodiments 1-20, wherein: there is minimal interaction between the binder and the P2, and the minimal interaction between the binder and the P2 has minimal or no impact or influence on the binding specificity, strength and/or efficiency between the binder and the N-terminally modified target polypeptide; and/or

P1-P2 occupies a volume or shape encompassing a cavity or pocket of the binder that effectively precludes the P2 from entering into or interacting with an affinity determining region of the binder.

Embodiment 22. The binder of any one of embodiments 1-21, which specifically binds to multiple N-terminally modified target polypeptides that comprise the same P1 residue.

Embodiment 23. The binder of any one of embodiments 1-21, which specifically binds to multiple N-terminally modified target polypeptides that comprise different P1 residues.

Embodiment 24. The binder of embodiment 23, which specifically binds to multiple N-terminally modified target polypeptides that comprise 2, 3, 4, 5, 6, 7, 8, 9 or 10 different P1 residues.

Embodiment 25. The binder of any one of embodiments 1-24, which comprises or is an engineered anticalin.

Embodiment 26. The binder of embodiment 25, wherein the engineered anticalin is derived or evolved from an eucaryotic lipocalin, such as a lipocalin from a human, a cow, a pig, an insect, e.g., a butterfly.

Embodiment 27. The binder of embodiment 25 or 26, wherein the engineered anticalin is derived or evolved from a core or kernel lipocalin, or an outlier lipocalin.

Embodiment 28. The binder of any one of embodiments 25-27, wherein the engineered anticalin comprises an anticalin b-barrel core.

Embodiment 29. The binder of embodiment 28, wherein, upon binding to an N-terminally modified target polypeptide, the M-P1 residues occupy the anticalin b-barrel core.

Embodiment 30. The binder of any one of embodiments 25-29, wherein the engineered anticalin specifically binds to an N-terminally modified target polypeptide with a P1 residue that is selected from the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

Embodiment 31. The binder of embodiment 30, wherein the binder comprises an amino acid sequence having at least about 80% sequence identity to the amino acid sequence selected from the group consisting of SEQ ID NO: 7—SEQ ID NO: 20.

Embodiment 32. The binder of any one of embodiments 25-30, wherein the engineered anticalin is derived or evolved from a lipocalin that comprises an amino acid sequence set forth in SEQ ID NO: 19 and has a mutation selected from the group consisting of V33T, L36R, Y52R, T54L, L70M, R79S, W81E, F85Q, L96E, N₉₈L, H100T, R101W, Y102H, Y108W, F125S, K127P, K136R, Y140L and a combination thereof.

Embodiment 33. The binder of any one of embodiments 25-30, wherein the engineered anticalin comprises an amino acid sequence that has at least 80%, 90%, 95% or more identity to an amino acid sequence set forth in SEQ ID NO: 19 .

Embodiment 34. The binder of any one of embodiments 1-33, which has a binding signal and/or affinity towards a modified target polypeptide comprising a specific P1 residue that is at least 2-fold or higher as compared to the binder's binding signal and/or affinity towards an otherwise identical modified target polypeptide but comprising a different P1 residue.

Embodiment 35. A nucleic acid encoding the engineered anticalin of any of embodiments 1-34.

Embodiment 36. A vector comprising the nucleic acid of embodiment 35, e.g., an expression vector.

Embodiment 37. A host cell comprising the nucleic acid of embodiment 35 or the vector of embodiment 36.

Embodiment 38. The host cell of embodiment 38, wherein the host cell is a mammalian host cell.

Embodiment 39. A modified cleavase comprising a mutation, e.g., one or more amino acid modification(s), in an unmodified cleavase, wherein:

said modified cleavase is derived from a dipeptidyl peptidase of Thermomonas hydrothermahs or Caldithrix abyssii and removes or is configured to remove a single N-terminally modified amino acid from a target polypeptide.

Embodiment 40. The modified cleavase of embodiment 39, wherein the modified cleavase is derived from a dipeptidyl peptidase of Thermomonas hydrothermalis comprising an amino acid sequence set forth in SEQ ID NO: 3 [WT sequence with the signal peptide] or SEQ ID NO: 4 [WT sequence without the signal peptide].

Embodiment 41. A method of treating a target polypeptide, which method comprises:

a) contacting a target polypeptide with an N-terminal modifier agent to form an N-terminally modified target polypeptide having a formula:

M-P1-P2-polypeptide, said M being an N-terminal modification, said P1 being the N-terminal amino acid residue of said target polypeptide, and P2 being a penultimate terminal amino acid residue of said target polypeptide; and

b) contacting a binder with said N-terminally modified target polypeptide to allow said binder to specifically bind to said N-terminally modified target polypeptide through interaction between said binder and said M and P1 of said N-terminally modified target polypeptide, wherein the binding specificity between said binder and said N-terminally modified target polypeptide is predominantly or substantially determined by said interaction between said binder and said P1 of said N-terminally modified target polypeptide.

Embodiment 42. The method of embodiment 41, wherein the M is a bipartite N-terminal modification (NTM) that comprises a natural or unnatural amino acid portion (NTMaa) and an N-terminal blocking group (NTM_{blk}), the amino acid portion (NTMaa) and the N-terminal blocking group (NTM_{blk}) being optionally connected with an amide bond.

Embodiment 43. The method of embodiment 41, wherein the M does not comprise an amino acid moiety.

Embodiment 44. The method of embodiment 41, wherein the M is a bipartite N-terminal modification (NTM) that comprises a small (or small molecule) chemical entity having a size, e.g., length axis or volume, shape, and/or configuration similar to or exceeding a natural amino acid, and an N-terminal blocking group (NTM_{blk}),

the small (or small molecule) chemical entity and the N-terminal blocking group optionally connected with an amide bond, and/or

optionally, the small (or small molecule) chemical entity having a size, e.g., length axis of ˜5-10 Å and volume of 100-1,000 Å³.

Embodiment 45. The method of any one of embodiments 41-44, wherein the binder comprises an amino acid sequence having at least about 80% sequence identity to the amino acid sequence selected from the group consisting of SEQ ID NO: 7—SEQ ID NO: 20.

Embodiment 46. The method of any one of embodiments 41-45, which further comprises a step:

c) cleaving the peptide bond between the P1 and P2 to form a polypeptide wherein the P2 becomes N-terminal amino acid residue of the polypeptide.

Embodiment 47. The method of any one of embodiments 41-46, wherein the N-terminal modifier agent comprises a compound of any one of Formulas (A)-(D), and optionally a peptide coupling reagent, wherein
(A)

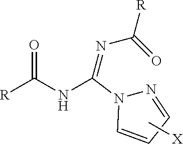

wherein R is CH₃, CF₃, OC(CH₃)₃, or OCH₂C₆H₅, and X is H, CH₃, CF₃, CF₂H, or OCH₃;
(B)

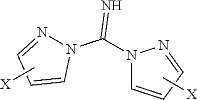

wherein X is H, CH₃, CF₃, CF₂H, OCH₃, or SO₂NH₂;
(C)

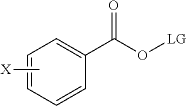

wherein X is H, F, Cl, OCH₃, OCF₃, CN, or SO₂NH₂, and LG is succinimide, pentafluorophenyl, or tetrafluorophenyl; and
(D)

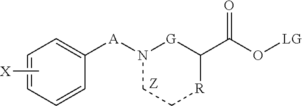

wherein X is H, F, Cl, NH₂, OCH₃, OCF₃, CN, or SO₂NH₂, A=CONH or SO₂, G=0 or 1 CH₂,

R is any amino acid or unnatural amino acid, and Z ring=0 (not there), 1, 2, or 3 CH₂.

Embodiment 48. The method of embodiment 47, wherein the peptide coupling reagent is an aminium, uronium, or carbodiimide coupling reagent.

Embodiment 49. The method of embodiment 47, wherein the peptide coupling reagent is a compound of Formula (1) or (2), wherein:

Formula (1) is

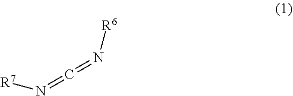

or a salt or conjugate thereof,

wherein

R⁶ and R⁷ are each independently C₁₋₆ alkyl, —CO₂C₁₋₄ alkyl, —OR^{k}, aryl, heteroaryl, cycloalkyl or heterocyclyl, wherein the C₁₋₆ alkyl, —CO₂C₁₋₄ alkyl, —OR^{k}, aryl, and cycloalkyl are each unsubstituted or substituted; and

R^{k} is H, C₁₋₆ alkyl, or heterocyclyl, wherein the C₁₋₆ alkyl and heterocyclyl are each unsubstituted or substituted; wherein heterocyclyl can be 5-8 membered ring comprising one or two heteroatoms selected from N, O and S as ring members, where the heteroaryl can be a 5-6 membered single ring or 8-10 membered bicyclic ring, each of which comprises one to three heteroatoms selected from N, O and S as ring members; and

Formula (2) is:

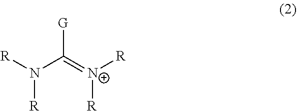

wherein:

each R is independently C₁₋₄ alkyl, optionally substituted with up to three groups selected from halo, C₁₋₂alkoxy, C₁₋₂haloalkyl, and C₁₋₂ haloalkoxy;

and two R groups on the same N can optionally cyclize to form a 5-7 membered ring optionally containing an additional heteroatom selected from N, O and S as a ring member, and optionally substituted with one or two groups selected from oxo, C₁₋₂ alkyl, C₁₋₂alkoxy, C₁₋₂haloalkyl, and C₁₋₂ haloalkoxy; and

G is selected from halo, benzotriazolyloxy, halobenzotriazolyloxy, pyridinotriazolyloxy, benzotriazolyl-N-oxide, pyridinotriazolyl-N-oxide, —O—(N-succinimide), 1-cyano-2-ethoxy oxoethylideneaminooxy, and —O—(N-phthalimide).

Embodiment 50. The method of embodiment 47, wherein the peptide coupling reagent is selected from dicyclohexyl carbodiimide (DCC), diisopropyl carbodiimide (DIPC), 1-ethyl-3-(3-dimethylaminopropyl)carbodiimide (EDC), 1-cyclohexyl-(2-morpholinoethyl)carbodiimide tosylate (CMCT), COMU, HATU, HBTU, TBTU, HCTU, and TSTU, PyBOP, PyAOP, PyOxim, and BOP, and (3-(diethoxyphosphoryloxy)-1,2,3-benzotriazin-4(3H)-one) (DEPBT).

Embodiment 51. The method of embodiment 46, wherein the peptide bond between the P1 and P2 is cleaved using a modified cleavase, e.g., a modified cleavase of any one of embodiments 39-40.

Embodiment 52. The method of any one of embodiments 46-51, wherein step c) is conducted while the binder is bound with the N-terminally modified target polypeptide.

Embodiment 53. The method of any one of embodiments 46-52, wherein steps a)- c) are repeated one or more times to form a polypeptide having newly exposed N-terminal amino acid residue.

Embodiment 54. The method of any one of embodiments 46-53, wherein step b) comprises contacting a plurality of binders with the N-terminally modified target polypeptide to allow the binders to specifically bind to the N-terminally modified target polypeptide.

Embodiment 55. The method of any one of embodiments 41-54, wherein the binder comprises a coding tag with identifying information regarding the binder.

Embodiment 56. The method of any one of embodiments 46-55, which further comprises a step:

d) transferring the identifying information of the coding tag to a recording tag attached to the N-terminally modified target polypeptide, thereby generating an extended recording tag on the -terminally modified target polypeptide.

Embodiment 57. The method of embodiment 56, wherein step d) is performed after step b), but before step c).

Embodiment 58. The method of embodiment 56 or 57, wherein the steps of:

a) contacting a target polypeptide with an N-terminal modifier agent;

b) contacting a binder with the N-terminally modified target polypeptide;

c) transferring the identifying information of the coding tag to a recording tag attached to the N-terminally modified target polypeptide; and

d) cleaving the peptide bond between the P1 and p2 to form a polypeptide wherein the P2 becomes N-terminal amino acid residue of the polypeptide, are repeated in sequential order to generate one or more additional extended recording tags.

Embodiment 59. A kit of treating a target polypeptide, which kit comprises:

a) an N-terminal modifier agent that is configured to contact a target polypeptide to form an N-terminally modified target polypeptide having a formula:

M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; and

b) an engineered binder that is configured to specifically bind to said N-terminally modified target polypeptide through interaction between the engineered binder and the M-P1 of the N-terminally modified target polypeptide, wherein the engineered binder comprises an amino acid sequence having at least about 80% sequence identity to the amino acid sequence selected from the group consisting of SEQ ID NO: 7—SEQ ID NO: 20.

Embodiment 60. The kit of embodiment 59, wherein binding specificity between said binder and said N-terminally modified target polypeptide is predominantly or substantially determined by said interaction between said binder and said P1 of said N-terminally modified target polypeptide.

Embodiment 61. The kit of embodiment 59 or 60, wherein the N-terminal modifier agent is selected from the group consisting of compounds of the following formula:
(A)

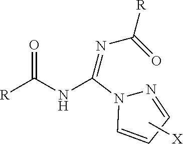

wherein R is CH₃, CF₃, OC(CH₃)₃, or OCH₂C₆H₅, and X is H, CH₃, CF₃, CF₂H, or OCH₃;
(B)

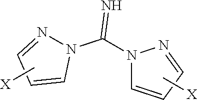

wherein X is H, CH₃, CF₃, CF₂H, OCH₃, or SO₂NH₂;
(C)

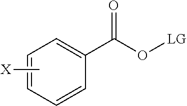

wherein X is H, F, Cl, OCH₃, OCF₃, CN, or SO₂NH₂,

and LG is succinimide, pentafluorophenyl, or tetrafluorophenyl; and
(D)

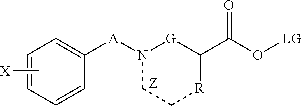

wherein X is H, F, Cl, NH₂, OCH₃, OCF₃, CN, or SO₂NH₂, A=CONH or SO₂, G=0 or 1 CH₂,

R is any amino acid or unnatural amino acid, and

Z ring=0 (not there), 1, 2, or 3 CH₂.

Embodiment 62. The kit of any one of embodiments 59-61, wherein the engineered binder comprises an amino acid sequence having at least about 89% sequence identity to the amino acid sequence selected from the group consisting of SEQ ID NO: 7—SEQ ID NO: 20.

Embodiment 63. The kit of any one of embodiments 59-62, wherein the engineered binder further comprises a detectable label or a nucleic acid tag.

Embodiment 64. A compound of the formula:

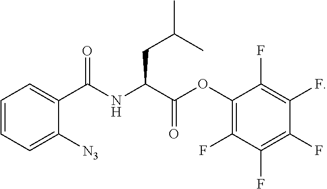 [SMILES: CC(C)C[C@H](NC(=O)c1ccccc1N=[N+]=[N-])C(=O)Oc1c(F)c(F)c(F)c(F)c1F]

Embodiment 65. An engineered binder that specifically binds to an N-terminally modified target polypeptide modified by an N-terminal modifier agent, wherein: (i) the N-terminally modified target polypeptide has a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; (ii) the engineered binder specifically binds to the N-terminally modified target polypeptide through interaction between the engineered binder and the M-P1 of the N-terminally modified target polypeptide; and (iii) the engineered binder comprises an amino acid sequence having at least about 80% sequence identity to the amino acid sequence selected from the group consisting of SEQ ID NO: 7—SEQ ID NO: 20.

Embodiment 66. The engineered binder of Embodiment 65, wherein the N-terminal modification is a chemical entity having a volume from about 100 Å³ to about 1000 Å³.

Embodiment 67. The engineered binder of Embodiment 65 or 66, wherein the N-terminal modifier agent is selected from the group consisting of compounds of the following formula:
(A)

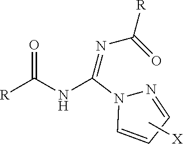

wherein R is CH₃, CF₃, OC(CH₃)3, or OCH₂C₆H₅, and X is H, CH₃, CF₃, CF₂H, or OCH₃;
(B)

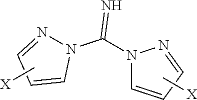

wherein X is H, CH₃, CF₃, CF₂H, OCH₃, or SO₂NH₂;
(C)

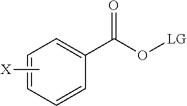

wherein X is H, F, Cl, OCH₃, OCF₃, CN, or SO₂NH₂, and LG is succinimide, pentafluorophenyl, or tetrafluorophenyl; and
(D)

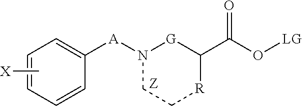

wherein X is H, F, Cl, NH₂, OCH₃, OCF₃, CN, or SO₂NH₂, A=CONH or SO₂, G=0 or 1 CH₂,

R is any amino acid or unnatural amino acid, and

Z ring=0 (not there), 1, 2, or 3 CH₂.

Embodiment 68. The engineered binder of Embodiment 65, wherein the N-terminal modification (M) comprises an N-terminal blocking group and, optionally, a natural or unnatural amino acid moiety; wherein the natural or unnatural amino acid moiety comprises a compound selected from the group consisting of: a naturally-occurring amino acid residue, 3-(3′-pyridyl)-L-alanine, L-cyclohexylglycine, α-aminoisobutyric acid, 3-(4′-pyridyl)-L-alanine, L-azetidine-2-carboxylic acid, isonipecotic acid, L-phenylglycine, β-(2-thienyl)-L-alanine, 3-(4-thiazolyl)-L-alanine, 1-aminocyclopentane-1-carboxylic acid, (2-trifluoromethyl)-L-Phenylalanine, L-cyclopropylalanine, 3-(2′-pyridyl)-L-alanine, beta-cyano-L-alanine, α-methyl-L-4-Fluorophenylalanine, α-methyl-D-4-fluorophenylalanine, 3-amino-2,2-difluoro-propionic acid, O-sulfo-L-tyrosine sodium salt, L-2-furylalanine, 1-aminocyclopropane-1-carboxylic acid, 3,5-dinitro-L-tyrosine, pentafluoro-L-phenylalanine, 3,5-difluoro-L-phenylalanine, 3-fluoro-L-phenylalanine, N-cyclopentylglycine, 1-(amino)cyclohexanecarboxylic acid, N-methylalanine, 4-amino-tetrahydropyran-4-carboxylic acid, 4-amino-1,1-dioxothiane-4-carboxylic acid, 4-amino-1-methyl-4-piperidinecarboxylic acid, 2-amino-N-(2,4-dimethoxybenzyl)acetamido)acetic acid, and a N-alkylated derivative thereof; and the N-terminal blocking group comprises a compound selected from the group consisting of: 4-methylbenzoic acid, 4-(dimethylamio)benzoic acid, nicotinic acid, 3-aminonicotinic acid, 2-pyrazinecarbooxylic acid, 5-amino-2-fluoro-isonicotinic acid, 2,3-pyrazinedicarboxylic acid, 4,7-Difluoroisobenzofuran-1,3-dicarboxylic acid, 4-chloro-2-aminobenzoic acid, 4-nitro-2-aminobenzoic acid, 7-methoxy-1h-benzo[d][1,3]oxazine-2,4-dione, 4-carboxy-2-aminobenzoic acid, 6-(Trifluoromethyl)-2,4-dihydro-1h-3,1-benzoxazine-2,4-dione, 7-(Trifluoromethyl)-1h-benzo[d][1,3]oxazine-2,4-dione, 6-fluoro-2-aminobenzoic acid, 4-fluoro-2-aminobenzoic acid, 5-methoxy-2-aminobenzoic acid, 4-fluorobenzoic acid, 4-(trifluoromethyl)benzoic acid, 2-ethynyl-6-fluorobenzaldehyde, 2-aminobenzoic acid, Succinic anhydride, 3,6-Difluoropyridine-2-carboxylic acid, 2-Fluoronicotinic acid, 5-Bromo-2-hydroxynicotinic acid, 4-(Trifluoromethyl)pyrimidine-5-carboxylic acid, 2-Oxo-1,2-dihydropyridine-3-carboxylic acid, 5-Methyl-2-aminobenzoic acid, 6-Fluoropicolinic acid, 3-Methyl-2-aminobenzoic acid, 4-Methyl-2-aminobenzoic acid, 2-Amino-6-methylbenzoic acid, 2-Amino-6-fluorobenzoic acid, 2-Amino-5-fluorobenzoic acid, 2-Amino-3-fluorobenzoic acid, 2-Amino-4-fluorobenzoic acid, 2-Aminonicotinic acid, 4-Aminonicotinic acid, 3-Aminopicolinic acid, 2-Amino-4,5-difluorobenzoic acid, 3,4-difluorobenzoic acid, 3,4,5-difluorobenzoic acid, 3-(Methoxycarbonyl)bicyclo[1.1.1]pentane-1-carboxylic acid, 3,3-Difluorocyclobutane-1-carboxylic acid, 1-Methyl-2-oxo-piperidine-4-carboxylic acid, Tetrahydropyran-4-carboxylic acid, 5-Fluoroorotic acid, 3-Fluoro-4-nitrobenzoic acid, 3-(Difluoromethyl)-1-methyl-1H-pyrazole-4-carboxylic acid, 4-(Difluoromethoxy)benzoic acid, 1-(Difluoromethyl)-1h-pyrazole-3-carboxylic acid, 4-(Methanesulfonylamino)benzoic acid, 5-Fluoro-6-methoxynicotinic acid, Tetrahydro-2H-thiopyran-4-carboxylic acid 1,1-dioxide, 4-(1H-Tetrazol-5-yl)benzoic acid, 1,2,3-Thiadiazole-4-carboxylic acid, 1,3-Benzodioxole-4-carboxylic acid, 2,1,3-Benzoxadiazole-5-carboxylic acid, 1-Benzyl-3-methyl-1h-pyrazole-5-carboxylic acid, 1-Cyclopropyl-6,7-difluoro-1,4-dihydro-4-oxoquinoline-3-carboxylic acid, 3,4-Dichlorobenzoic acid, 5-Fluoro-6-methylpyridine-2-carboxylic acid, 4,5-Dimethyl-2-(1h-pyrrol-1-yl)thiophene-3-carboxylic acid, 1,3-Dimethyl-1h-thieno[2,3-c]pyrazole-5-carboxylic acid, 1-[(4-Fluorobenzene)sulfonyl]piperidine-3-carboxylic acid, 1-(4-Fluorobenzyl)-5-oxopyrrolidine carboxylic acid, 3-Fluoro-4-methoxybenzoic acid, 4-Fluoro-3-nitrobenzoic acid, 6-Fluoro oxochromene-2-carboxylic acid, 3-Fluorophenylacetic acid, 4-Fluoro-3-(trifluoromethyl)benzoic acid, 5-Furan-2-yl-isoxazole-3-carboxylic acid, 1-Isopropyl-2-(trifluoromethyl)-1h-benzimidazole-5-carboxylic acid, Levofloxacin carboxylic acid, 3,5,7-Trifluoroadamantane carboxylic acid, 3,4,5-Trimethoxybenzoic acid, 2-Oxo-2,3-dihydro-1h-benzo[d]imidazole carboxylic acid, 1-Methyl-3-(trifluoromethyl)-1h-pyrazole-5-carboxylic acid, 2-Morpholin-4-yl-isonicotinic acid, 1,3-Oxazole-4-carboxylic acid, 4-Carboxybenzenesulfonamide, and 3,4-difluorobenzenesulfonyl chloride.

Embodiment 69. The engineered binder of Embodiment 66, wherein the N-terminal modification comprises an amino acid moiety.

Embodiment 70. The engineered binder of Embodiment 66 having a substrate binding pocket with a volume from about 200 A³ to about 2000 Å³.

Embodiment 71. The engineered binder of any of Embodiments 65-70, wherein the engineered binder comprises an amino acid sequence having at least about 89% sequence identity to the amino acid sequence selected from the group consisting of SEQ ID NO: 7—SEQ ID NO: 20.

Embodiment 72. The engineered binder of any of Embodiments 65-71, wherein binding specificity between the engineered binder and the N-terminally modified target polypeptide is predominantly or substantially determined by interaction between the engineered binder and the M-P 1 of the N-terminally modified target polypeptide.

Embodiment 73. The engineered binder of Embodiment 65, wherein the engineered binder is capable of specifically binding to each N-terminally modified target polypeptide from a plurality of N-terminally modified target polypeptides, wherein the plurality of N-terminally modified target polypeptides comprises at least 10 N-terminally modified target polypeptides that are modified with the same N-terminal modifier agent, have the same modified NTAA residue, and have different P2 residues.

Embodiment 74. The engineered binder of any of Embodiments 65-73, having an affinity towards an N-terminally modified target polypeptide comprising a specific P1 residue that is at least 2-fold higher as compared to affinity of the engineered binder towards an otherwise identical N-terminally modified target polypeptide but comprising a different P1 residue.

Embodiment 75. The engineered binder of any of Embodiments 65-74, wherein the N-terminally modified target polypeptide is immobilized on a solid support.

Embodiment 76. The engineered binder of any of Embodiments 65-75, further comprising a detectable label or a nucleic acid tag.

Embodiment 77. A set of engineered binders, comprising at least two engineered binders, wherein:

each engineered binder from the set of engineered binders is configured to specifically bind to an N-terminally modified target polypeptide modified with an N-terminal modifier agent and having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;

(ii) each engineered binder from the set of engineered binders is configured to specifically bind to the N-terminally modified target polypeptide through interaction between the engineered binder and the modified NTAA residue of the N-terminally modified target polypeptide, wherein engineered binders from the set of engineered binders are configured to specifically bind to different modified NTAA residues of target polypeptides modified with the same or different N-terminal modifier agents; and

(iii) at least one engineered binder from the set of engineered binders comprises an amino acid sequence having at least about 80% sequence identity to the amino acid sequence selected from the group consisting of SEQ ID NO: 7—SEQ ID NO: 20.

Embodiment 78. The set of engineered binders of Embodiment 77, wherein at least one engineered binder from the set of engineered binders comprises an amino acid sequence having at least about 89% sequence identity to the amino acid sequence selected from the group consisting of SEQ ID NO: 7—SEQ ID NO: 20.

Embodiment 79. The set of engineered binders of Embodiment 77, wherein at least two engineered binders from the set of engineered binders comprise an amino acid sequence having at least about 89% sequence identity to the amino acid sequence selected from the group consisting of SEQ ID NO: 7—SEQ ID NO: 20.

Embodiment 80. The set of engineered binders of Embodiment 77, which further comprises least one engineered binder from the set of engineered binders comprising an amino acid sequence having at least about 80% sequence identity to the amino acid sequence set forth in SEQ ID NO: 63.

Embodiment 81. The set of engineered binders of any of Embodiments 77-80, wherein each engineered binder from the set of engineered binders is configured to specifically bind to each N-terminally modified target polypeptide from a plurality of N-terminally modified target polypeptides, wherein the plurality of N-terminally modified target polypeptides comprises at least 10 N-terminally modified target polypeptides that are modified with the same N-terminal modifier agent, have the same modified NTAA residue, and have different P2 residues.

Embodiment 82. The set of engineered binders of any of Embodiments 77-81, wherein for each engineered binder from the set of engineered binders binding specificity between the engineered binder and the N-terminally modified target polypeptide is predominantly or substantially determined by interaction between the engineered binder and the modified NTAA residue.

Embodiment 83. The set of engineered binders of any of Embodiments 77-82, wherein each engineered binder from the set of engineered binders comprises a detectable label or a nucleic acid tag.

Embodiment 84. A method of treating a target polypeptide, the method comprises:
(i) contacting a target polypeptide with an N-terminal modifier agent to form an N-terminally modified polypeptide having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; and
(ii) contacting an engineered binder with the N-terminally modified target polypeptide to allow the engineered binder to specifically bind to the N-terminally modified target polypeptide through interaction between the engineered binder and the modified NTAA residue of the N-terminally modified target polypeptide, wherein the engineered binder comprises an amino acid sequence having at least about 80% sequence identity to the amino acid sequence selected from the group consisting of SEQ ID NO: 7—SEQ ID NO: 20.

Embodiment 85. The method of Embodiment 84, wherein the N-terminal modification is a chemical entity having a volume from about 100 A³. to about 1000 A³.

Embodiment 86. The method of Embodiment 84 or 85, wherein the N-terminal modifier agent is selected from the group consisting of compounds of the following formula:
(A)

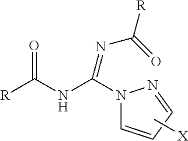

wherein R is CH₃, CF₃OC(CH₃)₃, or OCH₂H₅, and X is H, CH₃, CF₃, CF₂H, or OCH₃;
(B)

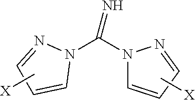

wherein X is H, CH₃, CF₃, CF₂H, OCH₃, or SO₂NH₂;
(C)

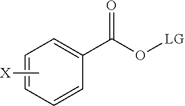

wherein X is H, F, Cl, OCH₃, OCF₃, CN, or SO₂NH₂, and LG is succinimide, pentafluorophenyl, or tetrafluorophenyl; and
(D)

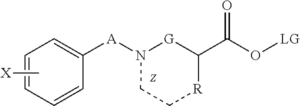

wherein X is H, F, Cl, NH₂, OCH₃, OCF₃, CN, or SO₂NH₂, A=CONH or SO₂, G=0 or 1 CH₂,

R is any amino acid or unnatural amino acid, and

Z ring =0 (not there), 1, 2, or 3 CH₂.

Embodiment 87. The method of any of Embodiments 84-86, wherein the engineered binder comprises an amino acid sequence having at least about 89% sequence identity to the amino acid sequence selected from the group consisting of SEQ ID NO: 7—SEQ ID NO: 20.

Embodiment 88. The method of any of Embodiments 84-87, further comprising removing the modified NTAA residue from the N-terminally modified target polypeptide, thereby exposing a new NTAA residue.

Embodiment 89. The method of any of Embodiments 84-88, further comprising immobilizing the target polypeptide on a solid support before step (i).

Embodiment 90. The method of Embodiment 86, wherein the N-terminal modifier agent further comprises a peptide coupling reagent, such as described in Embodiments 48-50.

Embodiment 91. The method of any of Embodiments 84-90, wherein the engineered binder comprises a detectable label or a nucleic acid tag.

Embodiment 92. The method of any of Embodiments 84-90, wherein the engineered binder comprises a nucleic acid coding tag comprising identifying information regarding the engineered binder.

Embodiment 93. The method of any of Embodiments 84-92, wherein a set of engineered binders according to Embodiment 77 is contacted with the N-terminally modified target polypeptide during step (ii) of the method.

Embodiment 94. The method of Embodiment 93, wherein each engineered binder from the set of engineered binders comprises a nucleic acid coding tag comprising identifying information regarding the corresponding engineered binder.

Embodiment 95. The method of Embodiment 94, wherein the target polypeptide is associated directly or via a linker with nucleic acid recording tag, and wherein the method further comprises:

(iv) upon binding of an engineered binder from the set of engineered binders to the N-terminally modified target polypeptide, transferring the identifying information from the nucleic acid coding tag of the engineered binder to the nucleic acid recording tag associated with the N-terminally modified target polypeptide to generate an extended nucleic acid recording tag; and

(v) analyzing the extended nucleic acid recording tag. Embodiment 96. An isolated nucleic acid molecule comprising a polynucleotide having a sequence encoding the engineered binder of Embodiment 65.

### IX. EXAMPLES

The following examples are offered to illustrate but not to limit the methods, compositions, and uses provided herein. Certain aspects of the present invention, including, but not limited to, embodiments for the Proteocode™ polypeptide sequencing assay, information transfer between coding tags and recording tags, methods of making nucleotide-polypeptide conjugates, methods for attachment of nucleotide-polypeptide conjugates to a support, methods of generating barcodes, methods of generating specific binders recognizing an N-terminal amino acid of a polypeptide, reagents and methods for modifying and/or removing an N-terminal amino acid from a polypeptide, methods for analyzing extended recording tags were disclosed in earlier published application US 20190145982 A1, US 20200348308 A1, US 20200348307 A1, US 20210208150 A1, WO 2020/223000, the contents of which are incorporated herein by reference in its entirety.

Compounds used in the invention can be made by methods known in the art in view of the following examples. A representative method for attaching an NTM to a target polypeptide is as follows, using a representative NTM of Formula (5) to attach an NTM to the NTAA of a taret polypeptide:

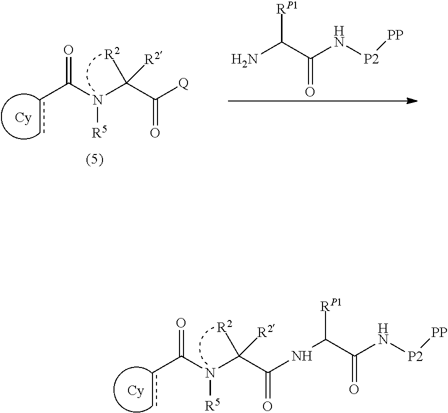

In this general reaction scheme, R^{P1} is the side chain of the N-terminal amino acid of a target polypeptide, P2 is the penultimate residue of the polypeptide, and PP represents the remainder of the target polypeptide: R^{P1} is typically selected from the 20 common amino acid side chains, optionally protected amino acid side chains, posttranslationally modified amino acid side chains, and unnatural amino acid sidechains; for example a side chain of any of these amino acids: Alanine, aspartic acid, isoaspartic acid, asparagine, N-glycosylated asparagine, glutamic acid, glutamine, glycine, (2-, 3-, or 4-pyridyl-)alanine, phenylglycine, 4-fluorophenylglycine, leucine, isoleucine, valine, dimethylglycine, methionine, methionine sulfoxide, phenylalanine, serine, phosphoserine, O-glycosylated serine, threonine, phosphothreonine, O-glycosylated threonine, cysteine, carbamidomethylcysteine, S-glycosylated cysteine, selenocysteine, sulfenic acid, sulfinic acid, sulfonic acid, tyrosine, sulfotyrosine, phosphotyrosine, nitrosotyrosine, tryptophan, histidine, N-acetyllysine, N-methyllysine, N,N-dimethyllysine, N,N,N-trimethyllysine, N-azidolysine , citrulline, nitroarginine, methylarginine, dimethylarginine, proline, hydroxyproline, or a salt thereof. The features in Formula (5) are as described herein for chemical reagents of Formula (5).

Reagents comprised of active esters (e.g., compounds of Formulas (3)-(9) wherein Q is an R^{Q} as described for the Formula) are dissolved in one of the following polar organic solvents; acetonitrile (ACN), N,N-dimethylformamide (DMF), N,N-dimethylacetamide (DMAc), N-methyl-2-pyrrolidone (NMP), sulfolane, dimethylsulfoxide (DMSO), cyrene, 1,3-dimethyl-2-imidazolidinone (DMI), and 1,3-Dimethyl-3,4,5,6-tetrahydro-2(1H)-pyrimidinone (DMPU)

Buffers used for this reaction are typically selected from: Sodium acetate, potassium acetate, ammonium acetate, sodium phosphate, potassium phosphate, ammonium phosphate, PBS, MES, MOPS, HEPES, Tris-HC₁, NEMA, PIPES, HEPPSO, triethylammonium acetate, triethanolammonium acetate, citrate, cit-phos, CAPS, CAPSO, bicarbonate, carbonate-bicarbonate, carbonate, borate, and bis-tris, where the pH of the buffer is in a range of 4-12; typically 6-11, and preferably 7-10.

A synthetic peptide (H-IHAGYAW-OH; SEQ ID NO: 67) was functionalized with [4] to show viability of the title compound's ability to install a modified Leucine for selective cleavage. A solution of [4] (150 mM in dimethylacetamide; DMAc) was prepared fresh. The peptide was also dissolved in DMAc to 10 mM concentration. Then in a 1.5 mL tube, 50 μL of acetonitrile and 25 μL MOPS buffer (pH 7.6) were added. To that, 10 μL of the 10 mM peptide solution was added in and mixed. Lastly, 154, of the 150 mM [4] was added in and the solution in the tube was placed in a thermomixer at 40° C. for 60 minutes.

After the 60 minute incubation of the IHAGYAW peptide (SEQ ID NO: 67), 100 of 0.5M TCEP (tris(2-carboxyethyl)phosphine) solution was added and incubated for 20 minutes at 40° C. to reduce the azide to amine. A 20 μL aliquot was removed and added to the 0.05M MES (2-(N-morpholino)ethanesulfonic acid) with 0.1% Tween 20 at pH 6.4 solution containing the modified cleavase. The modified cleavase and solution of labeled peptide were then incubated at 65° C. for 1-18h. The progress of the cleavage event was monitored by taking aliquots of the reaction and injecting on the LCMS.

### Example 1 Development of Anticalin M-P1 Binders with Minimal P2 Bias. Initial Anticalin Scaffold Selection

Lipocalins were used herein as starting scaffolds for directed evolution toward engineered anticalins specifically recognizing modified NTAA residues of target polypeptides. Anticalins have an intrinsic cup-like binding pocket, highly stable structure, good recombinant expression in E. coli., binding pocket evolvability using phage display, and demonstrated potential for strong and specific binding to small molecules. Many anticalins have an intrinsic ability to bind a modified-dipeptide residue.

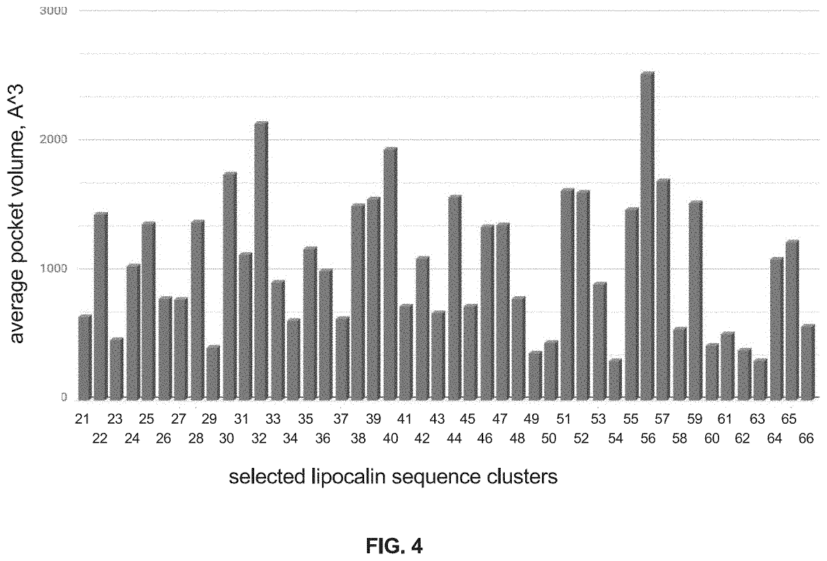

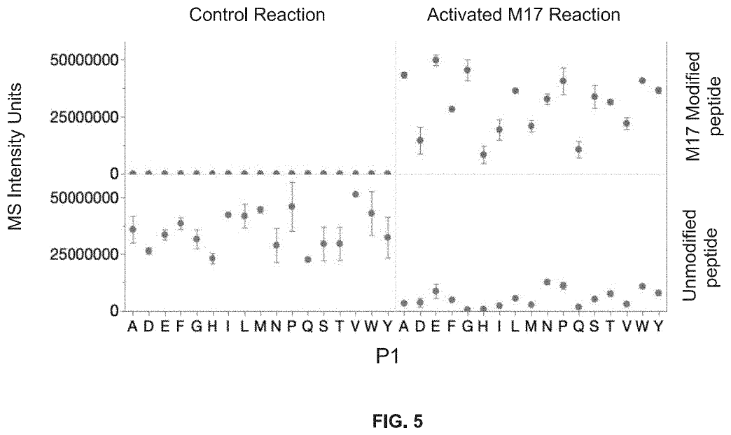

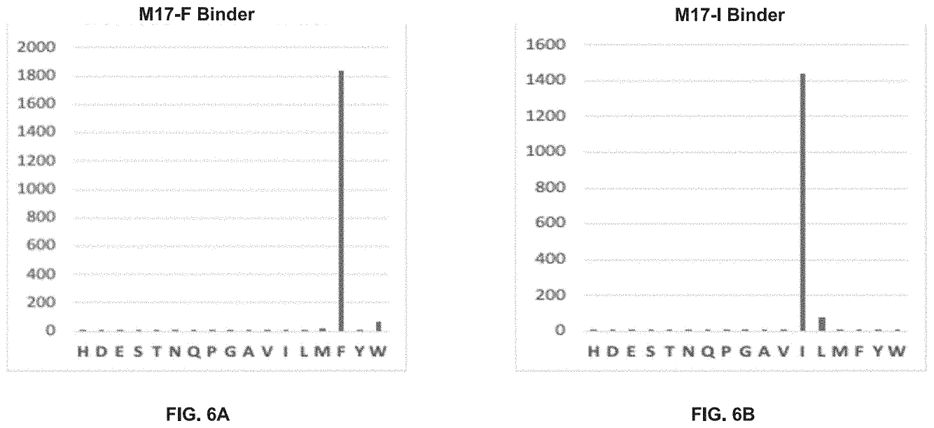

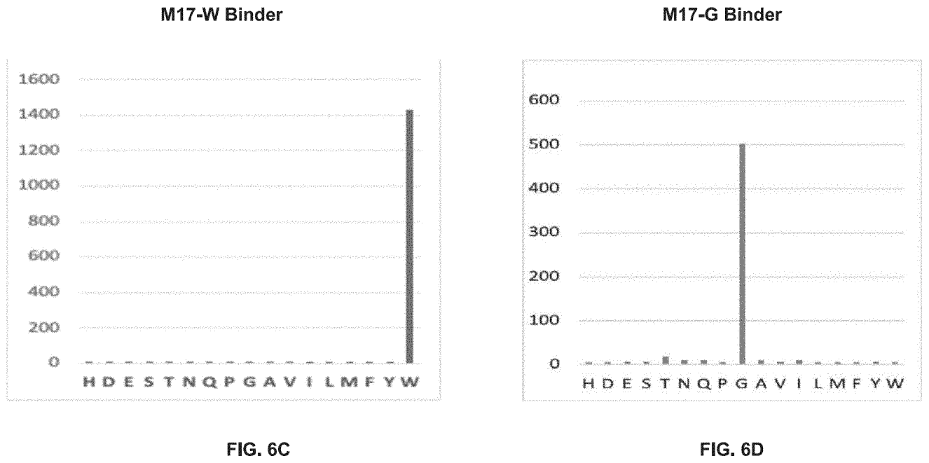

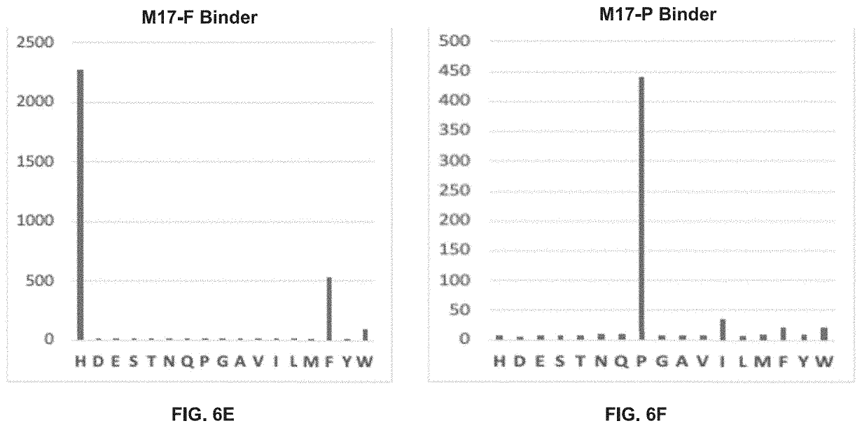

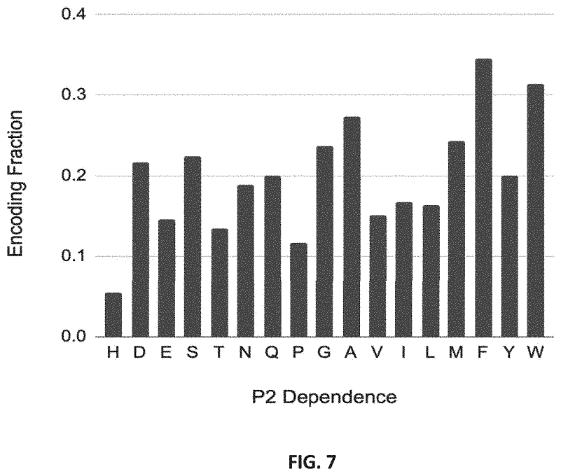

To cover lipocalin scaffolds as potential engineered binders, all lipocalin-like crystal structures present in the RCSB PDB database and containing the characteristic structure (lipocalin -barrel core) were pooled together and clustered based on 90% sequence identity within individual clusters. The clustered lipocalins were evaluated based on substrate pocket volume being within 200-2000 Å³, and the representative lipocalins (cluster centers, one from each cluster) were selected for expression in E. coli cells (FIG. 4). Among those that showed good expression levels in E. coli cells, representative lipocalins having sequences as set forth in SEQ ID NOs: 7-20 were further selected for phage display library generation and panning. The selected substrate pocket volume was chosen based on estimate that the M-P1 moiety of the target polypeptide, where M is an N-terminal modification and P1 is NTAA, would occupy the anticalin barrel core with the P1 sidechain oriented closer to the surface of the substrate pocket, and interaction with the penultimate terminal amino acid (P2) residue of the target polypeptide would be minimized. This design forces the P2 residue of the peptide to be located just outside the substrate pocket or affinity determining region of engineered anticalin, contributing less energy to the binding with the substrate.

### Example 2. Binder Engineering from the Lipocalin Scaffolds Selected in Example 1

Binder engineering involves defining potential binding sites through rational, structure-based approaches on a parental scaffold and generating libraries that contain degenerate NNK codons at multiple, defined positions using Kunkel mutagenesis and phage display selection. Kunkel mutagenesis is a known site-directed mutagenesis strategy that introduces point mutations by annealing mutation-containing oligonucleotides to single-stranded uracil-containing single strand DNA (dU-ssDNA) templates. Exemplary Kunkel mutagenesis and phage display selection methods are described in U.S. Pat. No. 9,102,711 B2; U.S. Pat. No. 10,906,968 B2; Kunkel, Proc. Natl. Acad. Sci. USA, 1985, 83(2):488-492.

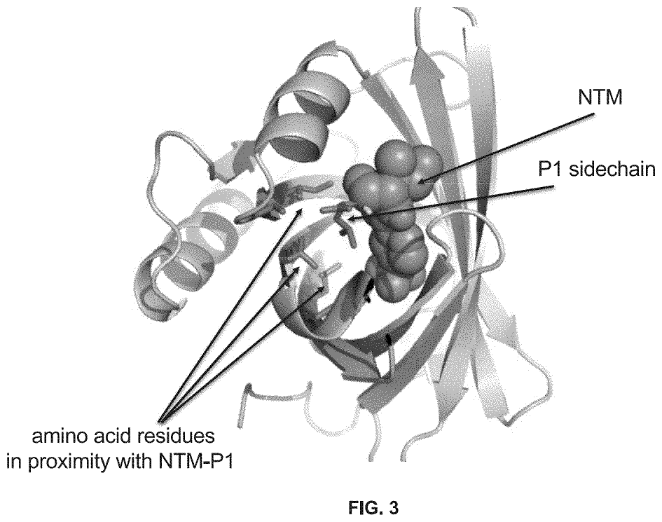

In this example, high diversity (˜10¹⁰) phage libraries using NNK variant site encoding were constructed targeting residues positions within the substrate-binding pockets of the selected lipocalins (see e.g., FIG. 3). Phosphorylated primers were obtained that possess degenerate codons at intended positions and were annealed to uracilated ssDNA containing the parental sequence of the same binder of interest with introduced SacII sites. After polymerase extension and ligation, the heteroduplex DNA was transformed into custom TG1RM cells (Lucigen TG1 Electrocompetent Cells containing a pCDF-lb plasmid expressing SacII enzyme), which removed undesired template DNA with SacII sites resulting in 10⁹-10¹⁰ libraries. Monovalent phage libraries were packaged using standard helper phage and precipitated using PEG/NaCl solution. Using standard protocols, phage libraries were panned against different N-terminally modified target peptides. NTAA modification was applied to target peptides during binder screening and maturation to increase substrate surface available for interaction with the binder, which would result in selection of binders with higher affinity and P1 specificity.

For each round of phage display selection, precipitated phage in the presence of peptide and protein competitors were first depleted against beads coated with off-target peptides for 1 hour at 24° C. and then panned against beads coated with target peptides for 1 hour at 24° C. After washing 6 times with PBST, beads-bound phages were eluted using 0.2 M pH=2.2 glycine for 10 min at 24° C. and then subsequently used to infect mid-log phase TG1 cells. Once the final round of selection was complete, the output was profiled in a phage-based, multiplexed binding assay (Luminex, DiaSorin, USA) against a panel of N-terminally modified target peptides and underwent next-generation sequencing to obtain clone enrichment sequence information. Luminex enables analysis of binding of phage libraries against multiple peptide targets immobilized on beads in a single assay well. This is accomplished by spatially separating immunoassays performed on beads that contain unique fluorophore cores that exhibit distinct excitation/emission profiles. Multiple target peptide-specific beads are combined in a single well of a multi-well microplate to detect and quantify multiple targets simultaneously. Specific binders were isolated against a variety of N-terminally modified target peptides. Based on the sequence identities after enrichment, consensus mutations or mutational hotspots were identified and binders were expressed and purified for testing in the encoding assay.

### Example 3. Binder Maturation

Binder maturation for affinity and specificity involved multiple cycles of error prone PCR prior to library construction via Kunkel mutagenesis and phage display selection, performed essentially as described in Example 2. Briefly, 60-90 cycles of error prone PCR on a parental binder generated PCR amplicons with an average of 4-6 random amino acid mutations per 100 amino acids. The dsDNA amplicon was digested by lambda exonuclease into “megaprimer” ssDNA, which was used to generate heteroduplex DNA by annealing to uracilated ssDNA of the vector containing the parental sequence of the same binder of interest with introduced SacII sites. After polymerase extension and ligation, the heteroduplex DNA was transformed into custom TG1RM cells (Lucigen TG1 Electrocompetent Cells containing a pCDF-lb plasmid expressing SacII enzyme), which removed undesired template DNA with SacII sites resulting in 109-1010 libraries. Monovalent phage libraries were packaged using standard helper phage and precipitated using PEGNaCl solution. For each round of phage display selection, precipitated phage in the presence of peptide and protein competitors were first depleted against beads coated with off-target peptides for 1 hour at 24° C. and then panned against beads coated with target peptides for 1 hour at 24° C. After washing 6 times with PBST, beads-bound phages were eluted using 0.2 M pH=2.2 glycine for 10 min at 24° C. and then subsequently used to infect mid-log phase TG1 cells. Once the final round of selection was completed, the output was profiled in a phage-based, multiplexed binding assay (Luminex, DiaSorin, USA) against a panel of N-terminally modified target peptides and underwent next-generation sequencing to obtain clone enrichment sequence information. Based on the sequence identities after enrichment, consensus mutations or mutational hotspots were identified and binders were expressed and purified for testing in the encoding assay.

### Example 4. Binder Expression and Purification

Plasmid DNA was received from a vendor generated source containing the identified engineered binder conjugated with an N-terminal hexa-histidine tag and a C-terminal SpyCatcher domain. Plasmids were transformed into chemically competent E. coli cells using standard methods. Recovery was done by adding 150 ul of warm SOC and incubation for 1 hour at 30° C. After recovery, 80 ul of transformed culture was added to 1 ml 2YT containing corresponding antibiotic. The culture was grown overnight and then used to generate stock in glycerol. The stock was then used to inoculate an overnight culture of 2YT containing corresponding antibiotic, and the culture was grown overnight for —20 hours at 37° C. This culture was subsequently used to inoculate another larger volume culture of 2YT containing corresponding antibiotic at a 100-fold dilution. The culture was then left at 37° C. for 3-4 hours until an optical density of 0.6 was reached. Temperature was then lowered to 15° C. and protein expression was induced with a final concentration of 0.5 mM IPTG. The cultures were grown for an additional 16-20 hours and the cells were harvested by centrifugation at 4,000 rpm for 20 min. The cellular pellets were stored at −80° C. until ready for use.

Stored cellular pellets were resuspend in 25 mM Tris pH 7.9, 500 mM NaCl, and 10 mM imidazole with included protease inhibitor and were lysed by sonication. The clarified lysate was loaded onto an AKTA FPLC using a tandem purification method of nickel affinity and size-exclusion chromatography. The retained protein was eluted from the nickel affinity column using 25 mM Tris pH 7.9, 500 mM NaCl, 300 mM imidazole directly onto the size-exclusion column. The size-exclusion buffer was 25 mM PO4 pH 7.4 with 150 mM NaCl, and after elution and concentration, glycerol was added to final concentration of 10%. Proteins were aliquoted, frozen, and stored at −80° C.

### Example 5. Evaluation of Binding Efficiencies of Binders via the Multiplex Encoding Assay

To evaluate binding efficiencies of selected purified anticalin binders, a previously developed ProteoCode™ assay (disclosed in detail in US 20190145982 A1, US 20200348308 A1, US 20200348307 A1) was used. This variant of the ProteoCode™ assay comprises contacting binder-coding tag conjugates with the N-terminally modified immobilized peptides associated with the recording tags. If affinity of the binder to the modified NTAA of the immobilized peptides is strong enough (typically, Kd should be less than 500 nM, and preferably, less than 200 nM), the coding tag and the recording tag form hybridization complex via hybridization of the corresponding spacer regions to allow transfer of barcode information from the coding tag to the recording tag via a primer extension reaction (the encoding reaction), generating extended recording tag. Sequencing of extended recording tags after the encoding cycle may be used to identify binder(s) that was(were) bound to the immobilized peptide. At the same time, estimating fractions of the recording tags being extended (encoded) during primer extension reaction provides estimate of efficiency of the encoding reaction, which directly correlates with binding affinity of the binder to the particular modified NTAA.

The described encoding assay was used to generate binding profiles for the selected anticalin binders across a set of 288 peptides (17×17 combination of different P1 and P2 residues) modified with a specific N-terminal modifier agent. For the encoding assay, selected binding agents engineered from lipocalin scaffolds as described in the previous Examples 1-4 were used. Each binding agent was conjugated to a corresponding nucleic acid coding tag comprising barcode with identifying information regarding the binding agent. The coding tag specific for the binding agent was attached to SpyTag via a PEG linker, and the resulting fusions were reacted with binding agent-SpyCatcher fusion protein via SpyTag-SpyCatcher interaction, essentially as described in US 2021/0208150 A1. Briefly, amine-functionalized oligonucleotide coding tags were conjugated to a heterobifunctional linker containing an NHS ester, PEG24 linker and maleimide. Excess linker was removed by acetone purification, and excess linker in solution was removed by centrifugation. Purified oligonucleotide-PEG24-maleimide was incubated overnight with SpyTag peptide forming a conjugate via a cysteine residue. The sample was spun down to remove precipitate and the supernatant was transferred to a 10k molecular weight filter to remove excess SpyTag peptide. After multiple washes, the final bioconjugate of SpyTag peptide containing a PEG24 linker and coding tag oligonucleotide was obtained and subsequently combined with the binder/SpyCatcher fusion protein spontaneously forming the final binder-fused coding tag conjugate.

An array of target peptide-recording tag conjugates having a variety of different NTAAs was generated (17X17 combination of different P1 and P2 residues). The peptides containing C-terminally attached 6-Azido-L-lysine were reacted with DBCO-C₂-modified 17 nt oligonucleotides in 100 mM HEPES, pH=7.0 at 60° C. for 1 hour. Each NTAA peptide-oligonucleotide conjugate was ligated to two different 15 nt DNA fragments containing a 7 nt barcode and an 8 nt spacer sequence using splint DNA and T4 DNA ligase to generate a peptide-recording tag conjugate with two different barcodes. A total of 576 peptide-recording tag conjugates were prepared and pooled for ligation and immobilization on short hairpin capture DNAs attached to the beads (NETS-Activated Sepharose High Performance, Cytiva, USA).

The capture DNAs were attached to the beads using trans-cyclooctene (TCO) and methyltetrazine (mTet)-based click chemistry. TCO-modified short hairpin capture DNAs (16 basepair stem, 4 base loop, 17 base 5′ overhang) were reacted with mTet-coated beads. The peptide-recording tag pools (20 nM) were annealed to the hairpin capture DNAs attached to the beads in 0.5 M NaC_{1, 50} mM sodium citrate, 0.02% SDS, pH 7.0, and incubated for 30 minutes at 37° C. The beads were washed once with lx phosphate buffer, 0.1% Tween 20 and resuspended in lx Quick ligation solution (New England Biolabs, USA) with T4 DNA ligase. After a 30 min incubation at 25° C., the beads were washed once with lx phosphate buffer, 0.1% Tween 20, three times with 0.1 M NaOH, 0.1% Tween 20, three times with 1× phosphate buffer, 0.1% Tween 20, and resuspended in 50 μL of PBST.

Before the encoding assay, the beads with immobilized target peptide-recording tag conjugates were treated with an N-terminal modifier agent by methods disclosed below in Example 11 to modify the N-terminal of the immobilized peptides. The modified beads with peptide conjugates were washed once with 70% Ethanol, washed once with water and resuspended in PBST. The coding tags attached to the binding agents form a loop with 12 bp duplex and 9 nt spacer at the 3′, which is complementary to the 3′ spacer of the recording tag on the beads.

The cycle of the encoding assay described in this example consists of contacting the immobilized peptides with a anticalin-based binding agent-coding tag conjugate. For this, each binding agent (50 nM) was incubated with the recording tag-peptide conjugates immobilized on the beads for 30 min at 25° C., followed by washing twice with 1× phosphate buffer, pH 7.3, 500 mM NaCl, 0.1% Tween 20. This was followed by transferring information of the coding tag to the recording tags associated with the target peptides by a primer extension reaction after partial hybridization between the coding tag and the recording tag through a shared spacer region using a DNA polymerase having 5′-to-3′ polymerization activity and having substantially reduced 3′-to-5′ exonuclease activity. Extension was performed by addition of 50 mM Tris-HCl, pH 7.5, 2 mM MgSO_{4, 50} mM NaCl, 1 mM DTT, 0.1 mg/mL BSA, 0.1% Tween 20, dNTP mixture (125 uM of each) and 0.125 U/uL of Klenow fragment (3′->5′ exo-) (MCLAB, USA) at 25° C. for 15 min, followed by one wash of lx phosphate buffer, 0.1% Tween 20, twice with 0.1 M NaOH +0.1% Tween 20, and twice with lx phosphate buffer, 0.1% Tween 20. After the recording tag extension, the binding agent-coding tag conjugate was washed away, and the sample was capped by introducing with primer binding site for PCR and NGS with incubation of 400 nM of an end capping oligo with 0.125 U/uL of WT Klenow fragment (3′->5′ exo-), dNTPs (each at 125 uM), 50 mM Tris-HCl (pH, 7.5), 2 mM MgSO₄, 50 mM NaCl, 1 mM DTT, 0.1% Tween 20, and 0.1 mg/mL BSA at 25° C. for 10 min. The beads were washed once with lx phosphate buffer, 0.1% Tween 20, twice with 0.1 M NaOH +0.1% Tween 20, and twice with 1× phosphate buffer, 0.1% Tween 20. Then, the extended recording tags were amplified and analyzed by nucleic acid sequencing.

Sequencing of recording tags after the encoding cycle was used to estimate fractions of the recording tags being extended (encoded) during primer extension reactions. The efficiencies of the encoding reactions were evaluated based on yield (based on fractions of recording tag reads contained barcode information of the coding tag (encoded)) and background signal (fractions of recording tag reads contained barcode information that are associated with a non-cognate peptide).

### Example 6. High Affinity, High Specificity Binders Against Modified N-Terminal Amino Acid (NTAA) Residues of Target Polypeptides Generated from the Lipocalin Scaffold

An exemplary lipocalin scaffold (sequence set forth in SEQ ID NO: 20) was used to generate a panel of binders specific for selected modified N-terminal amino acid (NTAA) residues (M-P1) of target polypeptides.

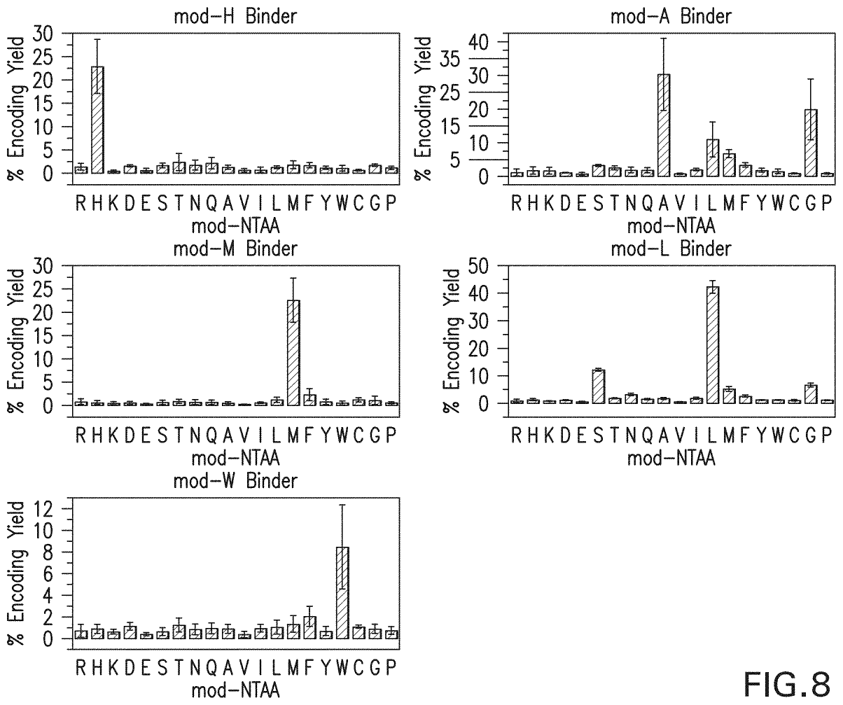

Binder engineering and maturation from the lipocalin scaffold were performed essentially as described in Examples 2 and 3. The crystal structure of the scaffold is available in the PDB database (3apv), and it was used to guide selection of key residues in the structure for modification during engineering and maturation. M90 N-terminal modification (NTM) was installed on target peptides to provide more binding surface and achieve better specificity during engineering. Specific binders were successfully selected against M90-modified H, M, W, A, L and F NTAA residues. Sequences of engineered binders specific for M90-modified H, M, W, A, L and F were as follows: SEQ ID NO: 55, SEQ ID NO: 56, SEQ ID NO: 52, SEQ ID NO: 54, SEQ ID NO: 53, SEQ ID NO: 51, and the specificity data are shown in FIG. 8. FIG. 8 demonstrates specificity profiling of M90-NTAA anticalin-based binders obtained using the Proteocode assay as described in Example 5. Multiplex beads were produced by covalently attaching equimolar amounts of the indicated P1 peptides to the surface. Fractions of encoded recording tags were calculated for a range of M90-P1 residues (shown on horizontal axis in FIG. 8), which correlate positively with binder specificity for particular M90-P1 as discussed in Example 5. Dissociation constants (Kd) were determined using BioLayer Interferometry (BLI) (ForteBio) for selected binders and were in the range of 40-200 nM. For the described binders, an affinity towards an N-terminally modified target polypeptide comprising a specific P1 residue was at least 2-fold higher as compared to affinity of the same engineered binder towards an otherwise identical N-terminally modified target polypeptide but comprising a different P1 residue.

### Example 7. High Specificity Binders Against Modified N-Terminal Amino Acid (NTAA) Residues of Target Polypeptides Generated from Different Lipocalin Scaffolds

A range of selected lipocalin scaffolds having permissible substrate binding pocket geometry were used for generating phage display libraries and engineering binders specific for modified NTAA residues of target polypeptides. The following scaffolds were used (corresponding sequence as set forth in the Sequence Listing and crystal structure number in the PDB database are indicated): SEQ ID NO: 7, 2k23; SEQ ID NO: 8, 4os3; SEQ ID NO: 9, lgm6; SEQ ID NO: 10, ljzu; SEQ ID NO: 11, 5t43; SEQ ID NO: 12, 1pee; SEQ ID NO: 13, 3apx; SEQ ID NO: 14, 3qkg; SEQ ID NO: 15, 2ova; SEQ ID NO: 16, lgt4; SEQ ID NO: 17, liiu; SEQ ID NO: 18, lbj7; SEQ ID NO: 19, 3s26. The crystal structures of the scaffolds were used to guide selection of key residues in the structure for modification during engineering and maturation. Binder engineering and maturation from the lipocalin scaffold were performed essentially as described in Examples 2 and 3. A diverse set of N-terminal modifier agents was installed on target peptides to provide more binding surface and achieve better specificity during engineering. The particular N-terminal modifier agents used in this Example were as follows: M12, M44, M48, M52, M2014, representing several different NTM chemistries. These NTMs are representative examples of compounds having general structure as disclosed in Exemplary Embodiments 17, 67 and 86 above. Example 11 below shows structures of these N-terminal modifier agents, as well as the installation methods.

The N-terminal modifications were chosen based on size (having a volume from about 100 Å³ to about 1000 Å³, and preferably, from about 100 Å³ to about 500 Å³), and also based on ability to interact with substrate binding pockets of lipocalin scaffolds, forming hydrogen bond-based, hydrophobic or other non-covalent interactions. The aim for an engineered lipocalin-based binder is to specifically bind to the N-terminally modified target polypeptide through interaction between the engineered binder and the M-P1 of the N-terminally modified target polypeptide, so that, preferably, binding specificity between the engineered binder and the N-terminally modified target polypeptide is predominantly or substantially determined by interaction between the engineered binder and the M-P1 of the N-terminally modified target polypeptide. It can be achieved with a proper geometry of substrate binding pocket of the engineered anticalin binder, when there is minimal or no interaction between the binder and the P2 residue of the target polypeptide. When P1-P2 part occupies a volume encompassing the substrate binding pocket of the engineered binder, and P1 residues is modified with an NTM having a volume similar to a volume of an amino acid residue, it would effectively preclude the P2 residue from entering into or interacting with an affinity determining region of the engineered binder interacting with the N-terminally modified target polypeptide.

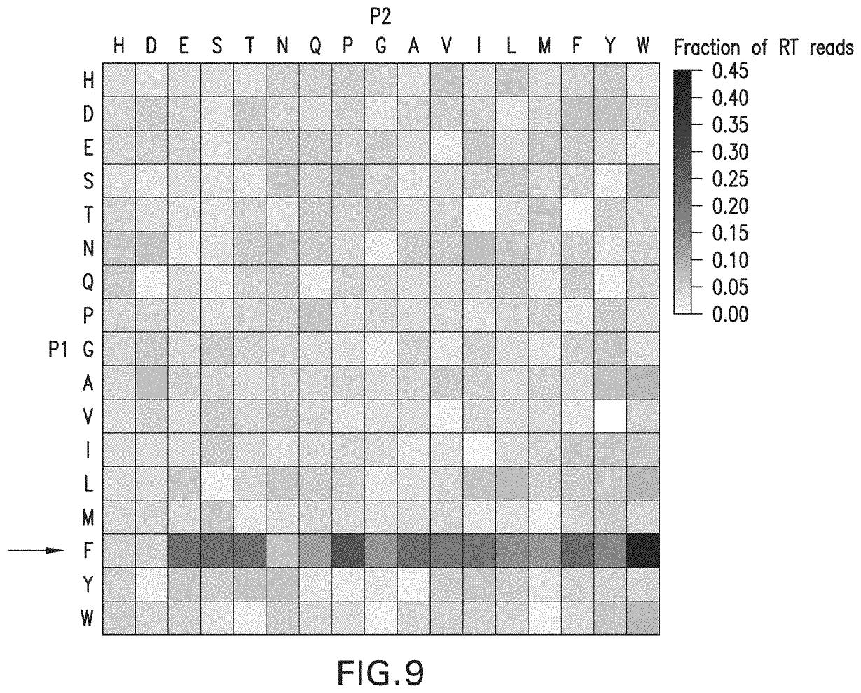

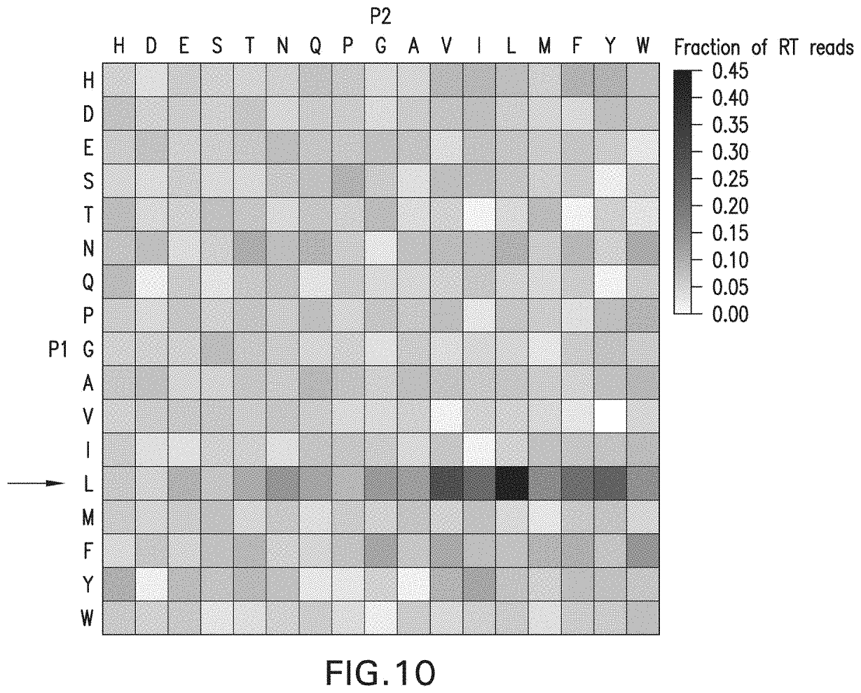

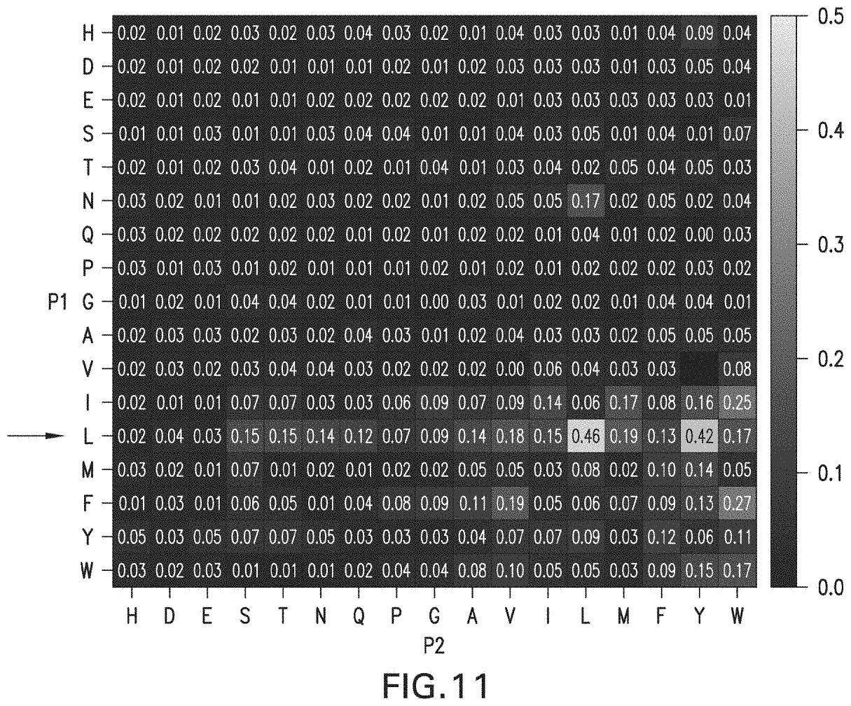

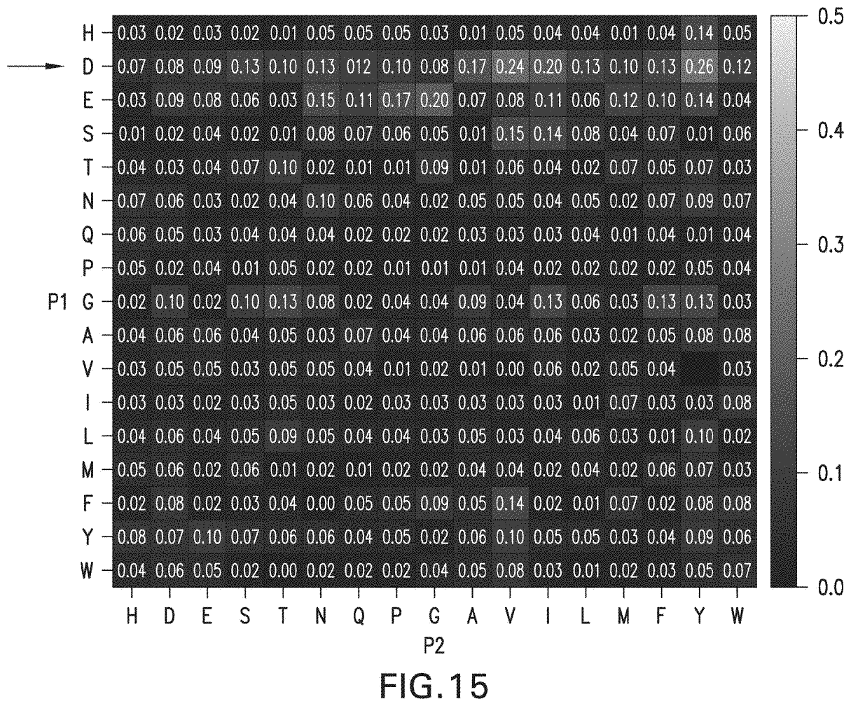

Thus, an engineered binder should have relatively high selectivity towards a modified P1 (M-P1) residue and broad tolerance for different P2 residues. To evaluate whether the engineered binders selected from different lipocalin-based scaffolds possess these features, heatmap arrays were generated, where each cell of the array represents an encoding efficiency of the given binder that binds to a specific combination of P1-P2 residues of the target polypeptide. To generate such heatmap arrays, encoding data (fractions of the recording tags being encoded) were collected in parallel as described in Example 5 for an immobilized set of 288 peptides (17×17 combination of different P1 and P2 residues) and plotted as two-dimensional matrix for diverse P1-P2 combinations (see FIG. 9- FIG. 15). Encoding efficiencies are shown as black/white gradient, wherein the more intense white color represents higher encoding efficiency (FIGS. 11-15, except for FIG. 9 and FIG. 10, where the opposite black/white gradient coding was employed).

One example of heatmap data is shown on FIG. 9 and FIG. 10. For experiments shown in these Figures, an immobilized set of 288 peptides (17×17 combination of different P1 and P2 residues) were modified with M12 NTM as described in Example 11, followed by interaction with engineered binder conjugated with a coding tag. FIG. 9 shows results of multiple parallel encoding reactions for the binder engineered from the scaffold having sequence as set forth in SEQ ID NO: 14 to specifically recognize M12-modified F NTAA residue of target peptides (different P1 amino acid residues of target peptides are shown on the y-axis and different P2 amino acid residues of target peptides are shown on the x-axis with fraction of recording tags encoded for each combination is shown as gradient intensity within the respective box based on the described encoding assay), and the binder's sequence is as set forth in SEQ ID NO: 50 of the Sequence Listing. The heatmap clearly illustrates binder's specificity towards M12-modified F NTAA residue located at the N-terminus of target peptides across multiple P2 (at least 14 different P2 residues are permissible to have an encoding efficiency higher than 0.15). FIG. 10 shows results of multiple parallel encoding reactions for the binder engineered from the scaffold having sequence as set forth in SEQ ID NO: 8 to specifically recognize M12-modified L NTAA residue of target peptides, and the binder's sequence is as set forth in SEQ ID NO: 49 of the Sequence Listing. Again, at least 11 different P2 residues are permissible for the binder to have an encoding efficiency higher than 0.15 in the encoding assay.

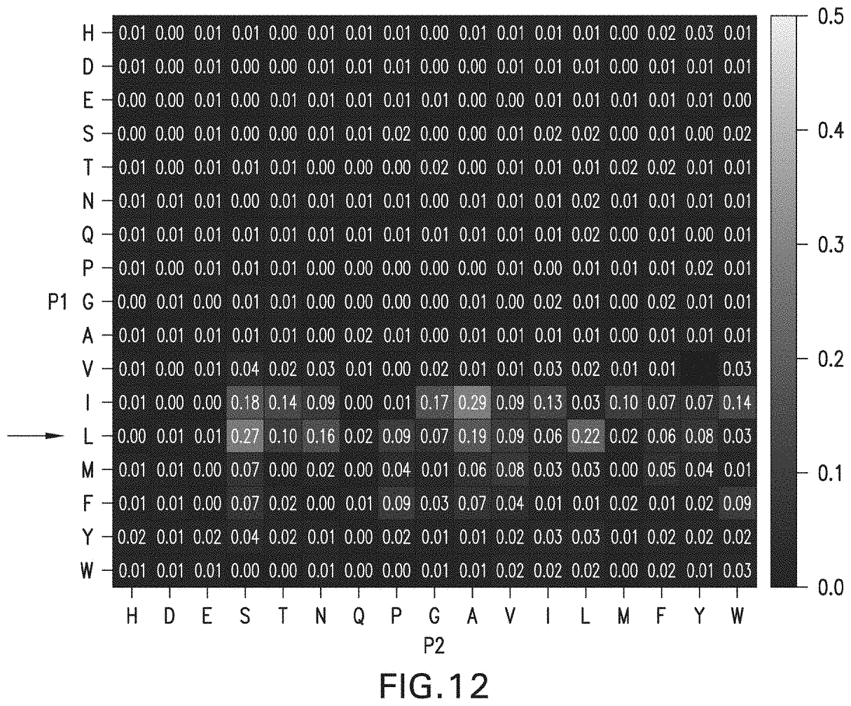

Another example of heatmap data is shown on FIG. 11 and FIG. 12, illustrating the ability of binders derived from two distinct scaffolds (starting scaffold sequences are as set forth in SEQ ID NO: 7 and SEQ ID NO: 8) to possess similar specificity toward M44-P1 amino acid residues of target peptides. Again, different P1 amino acid residues of target peptides are shown on the y-axis and different P2 amino acid residues of target peptides are shown on the x-axis with fraction of recording tags encoded for each combination is shown as gradient intensity and quantified as a number within the respective box based on the described encoding assay. FIG. 11 shows results of multiple parallel encoding reactions for the binder engineered from the scaffold having sequence as set forth in SEQ ID NO: 7 to specifically recognize M44-modified L NTAA residue of target peptides, and the binder's sequence is as set forth in SEQ ID NO: 31. FIG. 12 shows results of multiple parallel encoding reactions for the binder engineered from the scaffold having sequence as set forth in SEQ ID NO: 8 to specifically recognize M44-modified L NTAA residue of target peptides, and the binder's sequence is as set forth in SEQ ID NO: 21.

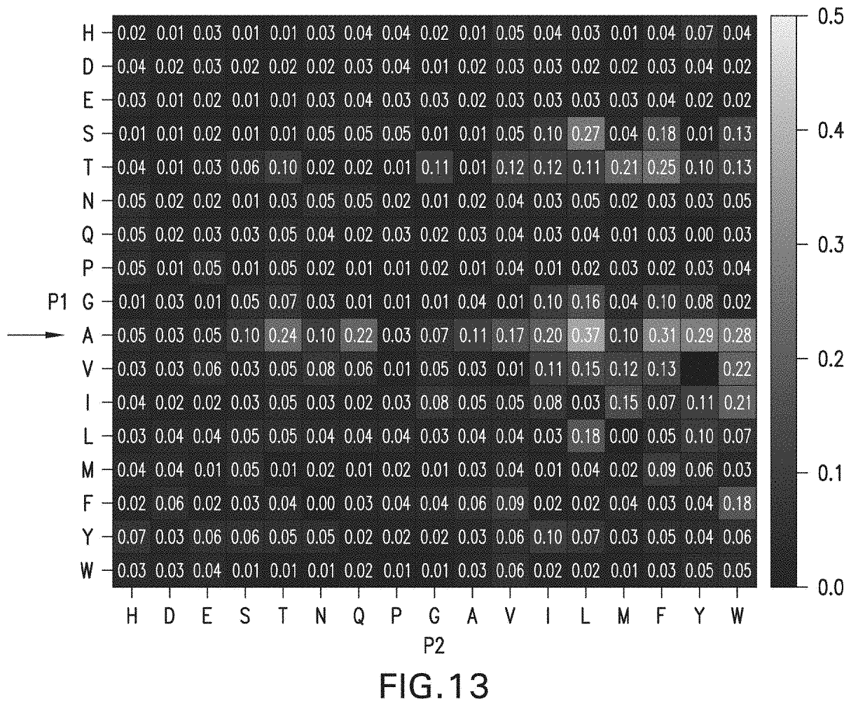

Another example of heatmap data is shown on FIG. 13 and FIG. 12, illustrating the ability of binders derived from the same scaffold (starting scaffold sequence is as set forth in SEQ ID NO: 8) to possess two separate P1 specificities toward M44-P1 amino acid residues of target peptides. FIG. 13 shows results of multiple parallel encoding reactions for the binder engineered from the scaffold having sequence as set forth in SEQ ID NO: 8 to specifically recognize M44-modified A NTAA residue of target peptides, and the binder's sequence is as set forth in SEQ ID NO: 42, whereas a different binder (SEQ ID NO: 21) engineered from the same scaffold to specifically recognize M44-modified L NTAA residue of target peptides was evaluated in FIG. 12 (see above).

Another example of heatmap data is shown on FIG. 14A-B, illustrating the ability of binder to develop better P1 specificities toward M17-P1 amino acid residues of target peptides. FIG. 14A shows results of multiple parallel encoding reactions for the binder engineered from the scaffold having sequence as set forth in SEQ ID NO: 10 to specifically recognize M17-modified Y NTAA residue of target peptides, and the binder's sequence is as set forth in SEQ ID NO: 61. The binder was further processed through additional maturation round performed as described in Example 3, and after introducing six additional mutations, the upgraded binder (SEQ ID NO: 62) exhibited increase in binding affinity, as well as a higher P2 tolerance (FIG. 14B, Table 1).

### Example 8. Quantification of Engineered Binder's P1 Selectivity and P2 Tolerability based on Calculating Corresponding P1 and P2 Gini Scores

To quantify engineered binder's P1 selectivity and P2 tolerance, relative P1 selectivity towards a modified P1 (M-P1) residue and relative P2 tolerance for different P2 residues were calculated as corresponding Gini coefficients. The Gini coefficient is a single number that demonstrates a degree of inequality in a distribution (a measure of inequality). It is used to estimate how far a given distribution deviates from a totally equal distribution. The Gini coefficient is defined as follows.

For a population uniform on the values yᵢ, i =1 to n, indexed in non-decreasing order (yᵢ≤yᵢ+i):

$$G = \frac{1}{n} ( n + 1 - 2 ( \frac{{\sum }_{i = 1}^{n} ( n + 1 - i ) {y}_{i}}{{\sum }_{i = 1}^{n} {y}_{i}} ) )$$

This may be simplified to:

$$G = \frac{2 {\sum }_{i = 1}^{n} {iy}_{i}}{n {\sum }_{i = 1}^{n} {y}_{i}} - \frac{n + 1}{n} . \qquad ( Equation 1 )$$

This formula applies to any population, since each member can be assigned its own yᵢ (Damgaard, Christian. “Gini Coefficient.” From MathWorld—A Wolfram Web Resource). To calculate Gini coefficient for engineered binder's P1 selectivity based on heatmap data, the above formula was used, where n represents number of P1 residues (n=17), and yᵢ represent fractions of recording tags encoded on the ith most encoding P1. Similarly, to calculate Gini coefficient for engineered binder's P2 tolerance based on heatmap data, the above formula was used, where n represents number of P2 residues (n=17), and yᵢ represent fractions of recording tags encoded on the ith most encoding P2. Higher P1 indicates more selectivity towards the particular M-P1 residue the binders specifically binds to, whereas lower P2 score indicates less selectivity towards particular P2 residue (and higher tolerance). These scores provide only relative estimation of selectivity, and they were arbitrary set to be: P1 score more than 0.15 for a binder to be considered as specific; and P2 score less than 0.4 for a binder to be considered P2-independent. It should be noted that the scores may be further improved through further binder selection and maturation process.

For preferred engineered binders to be used in the ProteoCode™ assay or in another assay, binding specificity between the engineered binder and the N-terminally modified target polypeptide is predominantly or substantially determined by interaction between the engineered binder and the M-P1 of the N-terminally modified target polypeptide. It implies or indicates that such engineered binder will have a high P1 score (for example, more than 0.25) and will have a low P2 score (for example, less than 0.3). Depending on particular assay, more or less specific binders can be employed. Alternative measurements of binder's P1 selectivity and P2 tolerance can be utilized, and different threshold values for P1 selectivity and P2 tolerance can be set.

To evaluate M-P1 specificity (via P1 selectivity and P2 tolerance) of selected binders engineered by the methods described in Example 2 and 3, P1 and P2 scores were calculated for the binders based on multiplex encoding data (heatmap data) and shown in Table 1. Corresponding binder sequences (based on SEQ ID NOs) are as set forth in the Sequence Listing. Starting scaffolds for the binders are shown in the second column of Table 1 (based on SEQ ID NOs) together with the NTM used to modify P1 residue.

TABLE 1
M-P1 specificity, P1 selectivity and P2 tolerance
of selected engineered binders.

| SEQ ID | SEQ | Specificity | | |
|---|---|---|---|---|
| NO of the | ID NO of the | towards | | |
| binder | scaffold and NTM | P1 | P1 score | P2 score |
| 21 | 8, M = M44 | L | 0.411533 | 0.4081645 |
| 22 | 8, M = M44 | L | 0.417622 | 0.4313397 |
| 23 | 8, M = M44 | F | 0.387456 | 0.4557343 |
| 24 | 8, M = M44 | L | 0.451653 | 0.3751609 |
| 25 | 8, M = M44 | L | 0.477998 | 0.5427553 |
| 26 | 8, M = M44 | L | 0.501239 | 0.4982774 |
| 27 | 8, M = M48 | L | 0.05714 | 0.311647 |
| 28 | 8, M = M48 | F | 0.39152 | 0.318293 |
| 29 | 8, M = M48 | F | 0.188785 | 0.3870709 |
| 30 | 7, M = M44 | F | 0.433229 | 0.3778745 |
| 31 | 7, M = M44 | L | 0.33245 | 0.3314838 |
| 32 | 7, M = M44 | F | 0.172855 | 0.3787853 |
| 33 | 11, M = M44 | G | 0.151808 | 0.2342439 |
| 34 | 11, M = M52 | L | 0.403674 | 0.2822373 |
| 35 | 11, M = M52 | L | 0.418448 | 0.9095673 |
| 36 | 7, M = M44 | I | 0.325818 | 0.1969959 |
| 37 | 7, M = M44 | A | 0.319006 | 0.1952591 |
| 38 | 8, M = M44 | L | 0.403483 | 0.2512549 |
| 39 | 8, M = M44 | L | 0.286281 | 0.4258292 |
| 40 | 8, M = M44 | I | 0.357229 | 0.1435108 |
| 41 | 8, M = M44 | I | 0.322721 | 0.2171574 |
| 42 | 8, M = M44 | A | 0.310459 | 0.3759861 |
| 43 | 10, M = M44 | F | 0.407562 | 0.3591912 |
| 44 | 10, M = M44 | F | 0.240715 | 0.4848608 |
| 45 | 10, M = M44 | Y | 0.300988 | 0.4259081 |
| 46 | 10, M = M44 | L | 0.225384 | 0.289012 |
| 47 | 10, M = M44 | A | 0.172919 | 0.4659644 |
| 48 | 19, M = M2014 | Y | 0.238347 | 0.2553231 |
| 61 | 10, M = M17 | Y | 0.1955124 | 0.2251778 |
| 62 | 10, M = M17 | Y | 0.2039607 | 0.12768142 |
| 64 | 64, M = M64 | D | 0.181079134 | 0.19409754 |
| 65 | 64, M = M64 | D | 0.199750049 | 0.19789691 |
| 66 | 64, M = M64 | D | 0.239483593 | 0.21612967 |

Engineered binders presented in the Sequence Listing and in Table 1 show diversity across M-P1 specificity, P1 selectivity and P2 tolerance. First, at least 6 different NTMs from different structural classes were successfully used to generate binder's specificity towards different M-P1s located at the N-terminus of target peptides. As a result, multiple combinations of M-P1s were targeted (see Table 1). Second, binders of different P1 selectivity exist for the same M-P1 group present in target peptides. For example, the binders (SEQ ID NO: 30 and 32) share specificity towards particular M-P1 (M44-F) located at the N-terminus of target peptides, and have nearly identical P2 tolerance, but they differ drastically in P1 selectivity, with the binder having sequence as set forth in SEQ ID NO: 30 being more selective towards F NTAA residue. The same is true for the binders having sequence as set forth in SEQ ID NO: 28 and 29. Third, binders of different P2 selectivity (tolerance) exist for the same P1 selectivity and the same M-P1 group present in target peptides. For example, the binders (SEQ ID NO: 34 and 35) share specificity towards particular M-P1 (M52-L) located at the N-terminus of target peptides, and have nearly identical P1 selectivity, but they differ drastically in P2 selectivity (tolerance), with the second binder being very P2 selective (which is not desired when a specific NTAA binder is required for an assay). Finally, multiple selected lipocalin scaffolds were used successfully to engineer binders specific for particular M-P1 residue of a target polypeptide (see SEQ ID NOs: 7-20 of the Sequence Listing).

Sequences of engineered binders differ significantly from corresponding starting lipocalin scaffolds, and each of the engineered binders with sequences as set forth in SEQ ID NOs: 21-62 contains 15-22 amino acid substitutions from the corresponding starting scaffold. Since most amino acid substitutions were designed to be on the substrate-interaction region of the binders, geometry of substrate-binding pockets of the scaffolds and atomic interactions within them were significantly changed during engineering and maturation process.

Engineered binders shown in Table 1 typically have about 89-91% sequence identity with corresponding starting scaffolds. Note that these binders may be further processed through another maturation round for improving their characteristics, such as M-P1 affinity, P1 selectivity and/or P2 tolerance, as shown for example in FIG. 14A-B. In the next maturation round new amino acid substitutions will likely be introduced, and the updated binder's sequence may be further away from the sequence of the corresponding starting scaffold, such that it will have about 80 or 85% sequence identity with the corresponding starting scaffold. Moreover, conservative amino acid substitutions can be made in the binder's sequence that would improve its characteristics unrelated to the M-P1 binding, such as improve binder's stability or increase expression level of the binder in bacterial cells. Such conservative amino acid substitutions are known to skilled in the art, and the updated binder's sequence may have less than about 80% sequence identity with the corresponding starting scaffold (for example, may have about 70% or 75% sequence identity with the corresponding starting scaffold).

### Example 9. High Specificity Binders Against Modified N-Terminal Amino Acid (NTAA) Residues of Target Polypeptides Generated from a Non-Lipocalin Scaffold

Based on the data shown in Example 8, lipocalin-based engineered binders have a specificity bias towards hydrophobic NTAA residues (see Table 1 for specificity data for the selected binders). Indeed, among about 28 binders derived from the lipocalin-based scaffolds (SEQ ID NO: 21—SEQ ID NO: 48) most of the binders show specificity towards F, L and I modified NTAA residues, while some binders are specific towards A, G and Y modified NTAA residues. The reason for this bias lies in structures of lipocalin-based scaffolds, since many lipocalins naturally bind hydrophobic substrates. While in some cases lipocalin-based scaffolds can be engineered towards specificity to polar NTAA residues, an alternative solution is to engineer binders towards specific polar or charged NTAA residues from non-lipocalin scaffolds using the methods described in Examples 2 and 3.

Examples of such engineered binders derived from a non-lipocalin-based scaffold of SEQ ID NO: 63 are the binders having sequences as set forth in SEQ ID NO: 64—SEQ ID NO: 66 and having specificity towards M64-D located at the N-terminus of target peptides. An example of heatmap data for a representative M64-D-specific binder is shown on FIG. 15. FIG. 15 shows results of multiple parallel encoding reactions for the binder engineered from the scaffold having sequence as set forth in SEQ ID NO: 63 to specifically recognize M64-modified D NTAA residue of target peptides, and the binder's sequence is as set forth in SEQ ID NO: 66. Such binders can be used in combination with anticalin binders disclosed above to identify diverse NTAA residues of target peptides.

### Example 10. A set of M-P1 Specific Binders can be Used to Decode Identity of Multiple P1 NTAA Residues of Target Peptides

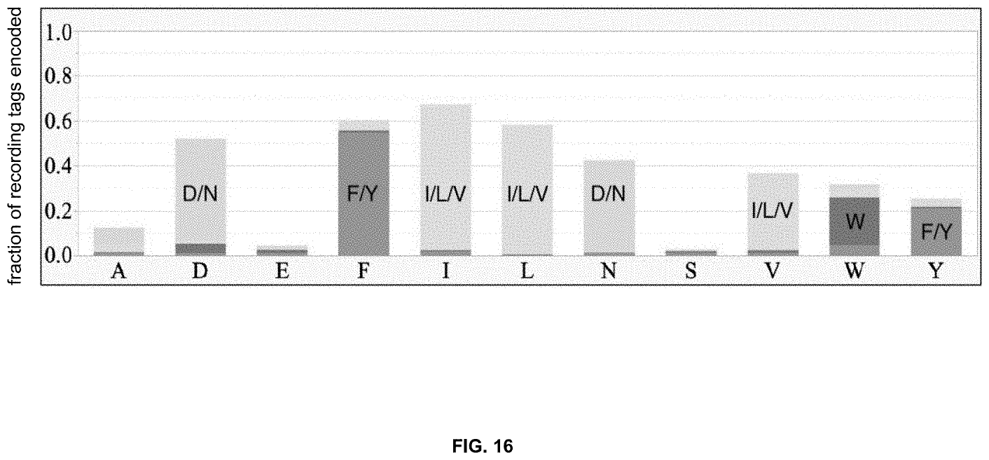

A key element toward developing robust N-terminal binding agents is a demonstration that respective NTM-P1 binding properties are retained when multiple binding agents are present simultaneously, i.e., when they are working as a set of binders. This inherently scalable feature enables systematic improvements to NTM-P1 coverage, including PTM detection, as further unique binders can be introduced into the Proteocode assay. To confirm this feature, four representative binders were selected with the following NTM-P1 coverage: F/Y, I/L/V, D/N, W. Sequences of engineered binders specific for M12-modified F/Y; I/L/V; W and D/N residues were as follows: SEQ ID NO: 57, SEQ ID NO: 58, SEQ ID NO: 59, SEQ ID NO: 60. Binders were initially prepared individually as described Example 4, then added to the encoding reaction as an equimolar mixture of 50 nM of each binder, in a final buffer formulation of lx phosphate buffer, 0.1% Tween 20. Binding properties were evaluated via encoding using the Proteocode assay, as previously described in Example 5. FIG. 16 shows results of encoding reactions for the set of four M12-NTAA anticalin-based binders having the described specificities. On the x-axis of FIG. 16, specific NTAA residues (modified with M12 for the assay) are shown that generated the encoding signal above the background, indicating specific binding of the binder with the indicated modified NTAA. FIG. 16 confirms that binding specificities are indeed retained when binders are used as a set, with a high degree of NTM-P1 discrimination and a low degree of cross-reactivity. These results highlight the scalable nature of adding multiple binding agents simultaneously within the Proteocode assay to enable large scale protein sequencing, identification, quantitation and/or further characterization. Multiple NTAA residues of target peptides can be identified using even imperfect M-P1-specific binders.

### Example 11. Structures and Installation Methods for Exemplary N-Terminal Modifier Agents Used for Modification of NTAA Residues of Polypeptides

While general structures of N-terminal modifier agents are disclosed in Exemplary Embodiments 17, 67 and 86 above, some exemplary N-terminal modifier agents used in the above Examples are shown below, together with the methods of NTM installation.

N-terminal modifier agent for M=M90

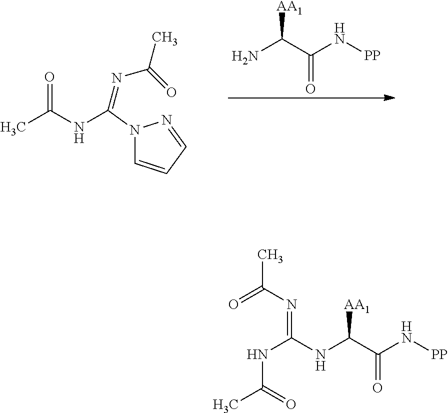

Exemplary method of installing M90 onto N-terminal amino acid of peptide: peptide(s), in solution or on solid-support, was dissolved in 90 uL of 0.2M N-ethylmorpholinium acetate (NEMA), pH=8.0 to an effective concentration of no more than 5 mM. Separately, the M90 reagent was dissolved in dimethylacetamide (DMA; 0.2M stock concentration). Then, 10 uL of M90 stock solution was added to the peptide-NEMA solution and incubated at 40° C. for 30 minutes. Upon completion, the peptide(s) was functionalized as shown in the above scheme.

N-terminal modifier agent for M=M12

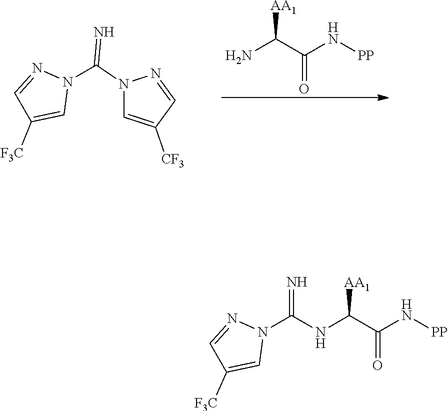

Exemplary method of installing M12 onto N-terminal amino acid of peptide(s): peptide(s), in solution or on solid-support, was dissolved in 50 uL of 0.2 M 3-morpholinopropane-1-sulfonic acid (MOPS), pH=7.6 to an effective concentration of no more than 10 mM. Separately, the M12 reagent was dissolved in dimethylacetamide (DMA; 0.06 M stock concentration). Then, 50 uL of M12 stock solution was added to the peptide-MOPS solution and incubated at 40° C. for 30 minutes. Upon completion, the peptide(s) was functionalized as shown in the above scheme.

N-terminal modifier agent for M=M15

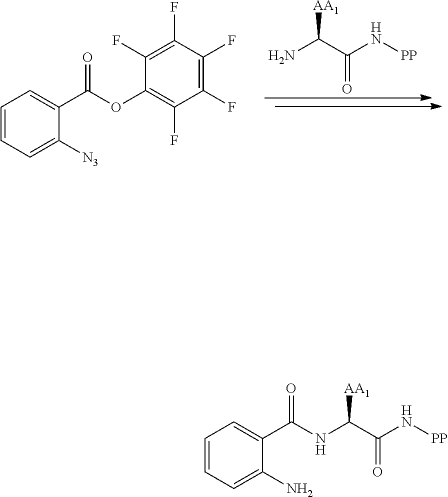

N-terminal modifier agent for M=M17

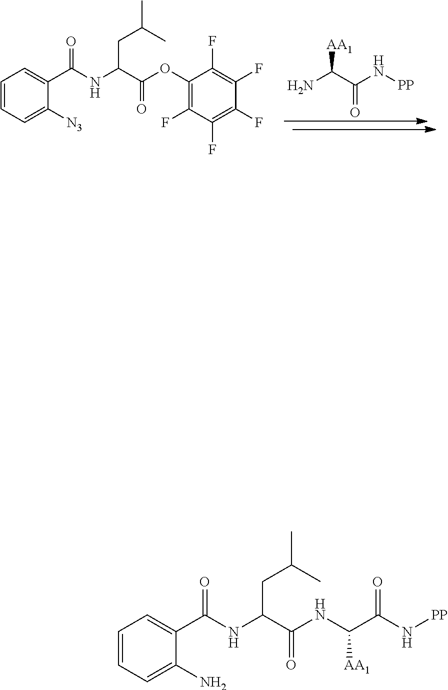

Exemplary method of installing M15 and M17 onto N-terminal amino acid of peptide(s): peptide(s), in solution or on solid-support, was dissolved in 50 uL of 0.2 M 3-morpholinopropane-1-sulfonic acid (MOPS), pH=7.6 to an effective concentration of no more than 10 mM. Separately, the M12 reagent was dissolved in dimethylacetamide (DMA; 0.06M stock concentration). Then, 50 uL of M12 stock solution was added to the peptide-MOPS solution and incubated at 40° C. for 30 minutes. Upon completion, the peptide(s) was functionalized with the modification-bearing azide. To obtain the desired modification as shown in the above schemes, a secondary incubation step with a 50 uL aliquot of 0.5M TCEP was added and incubated at 40° C. for 30 minutes.

N-terminal modifier agent for M=M44

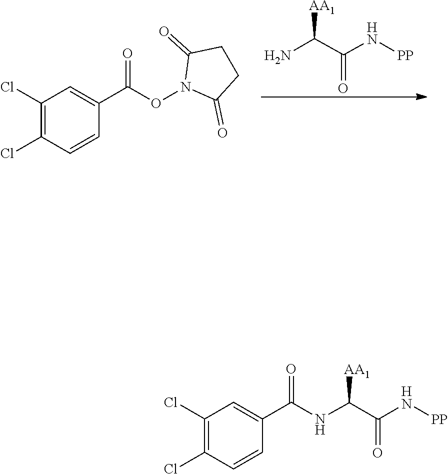

N-terminal modifier agent for M=M48

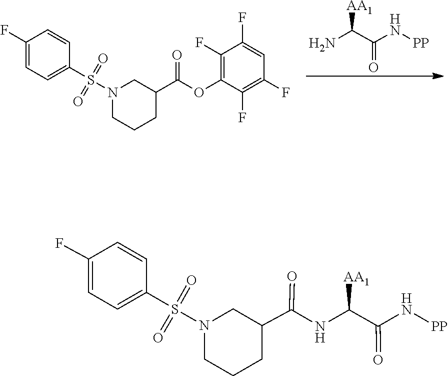

N-terminal modifier agent for M=M52

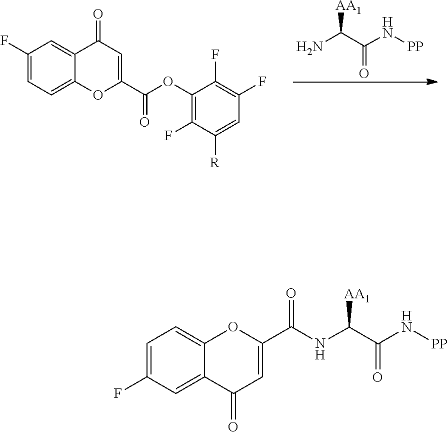

N-terminal modifier agent for M=M1914

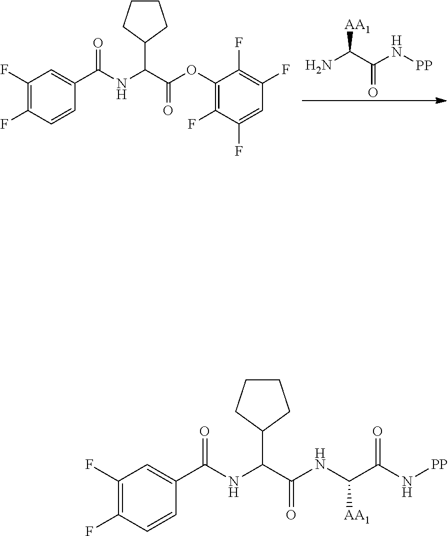

N-terminal modifier agent for M=M2014

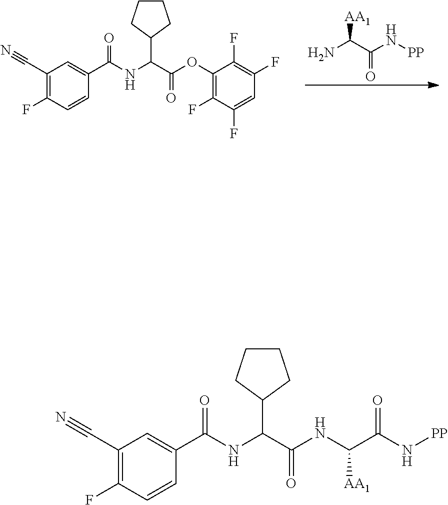

N-terminal modifier agent for M=M64

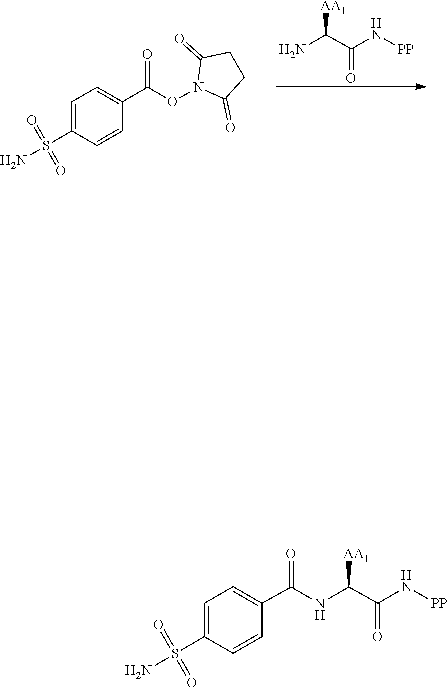

Exemplary method of installing M44, M48, M52, M64, M1914, and M2014 onto N-terminal amino acid of peptide(s): peptide(s), in solution or on solid-support, was dissolved in 25 uL of 0.4 M MOPS buffer, pH=7.6 and 25 uL of acetonitrile (ACN). Separately, the active ester reagent was dissolved in 25 uL DMA and 25uL ACN to a concentration of 0.05 M stock solution. Then, 50 uL of the active ester stock solution was added to the peptide-ACN:MOPS solution and incubated at 65° C. for 60 minutes. Upon completion, the peptide(s) was functionalized with the respective modification as shown in the above schemes.

Exemplary method of synthesis of M=M17.

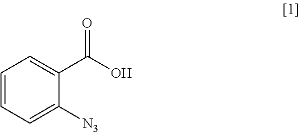 [SMILES: [N-]=[N+]=Nc1ccccc1C(=O)O]

Synthesis of 2-azidobenzoic acid [1]: To a 100 mL round-bottom flask equipped with a magnetic stirbar, lg of isatoic anhydride (6.13 mmol) was dissolved in a mixture of tetrahydrofuran (THF) and 5 equiv. (30.65 mmol) of sodium hydroxide (NaOH) in water. The mixture was stirred vigorously at room temperature for 30 minutes. LCMS of the solution showed that complete hydrolysis of the anhydride had taken place (forming the 2-aminobenzoic acid), so the solution was placed in an ice bath and acidified by addition of 20 equiv. (122.6 mmol) of conc. HCl. To this, 1.2 equiv. of sodium nitrite (NaNO_{2; 7.36} mmol) dissolved in water was added dropwise and allowed to stir at 0° C. for 20 minutes. Then, 1.5 equiv. of sodium azide (NaN₃; 9.195 mmol) was dissolved in water and added dropwise to the solution and proceeded to react for 15 minutes. The upon completion monitored by LCMS, the solution was extracted (3×50 mL) with ethyl acetate (EtOAc), washed with brine, and dried over Na2SO₄. The pooled organic solution was filtered, condensed, taken up in minimal diethyl ether (Et₂O), and precipitated with n-heptane. The solution was filtered and the remaining orange-brown powder collected was used without further purification (>99% pure by LCMS; 932mg, 93% yield).

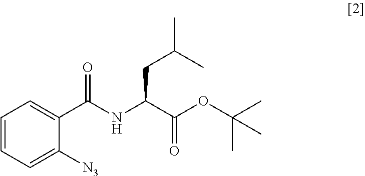 [SMILES: CC(C)C[C@H](NC(=O)c1ccccc1N=[N+]=[N-])C(=O)OC(C)(C)C]

Synthesis of N-(2-azidobenzamid)-L-leucine-O-tert-butyl ester [2]: To a 100 mL round-bottom flask containing a magnetic stirbar, 632 mg of [1] (3.874 mmol) was added and dissolved in anhydrous N,N-dimethylformamide (DMF), followed by 1.2 equiv. of diisopropylethylamine (DIPEA; 4.469 mmol). The solution was allowed to stir at room temperature for 10 minutes and then 1.1 equiv. of COMU ((1-Cyano-2-ethoxy-2-oxoethylidenaminooxy)dimethylamino-morpholino-carbenium hexafluorophosphate; 4.261 mmol) was added to the solution and continued to stir for 30 minutes. In a separate vial, 1.2 equiv. of L-leucine-O-tert-butyl ester HCl (4.469 mmol) was dissolved in dichloromethane (DCM) and 2.4 equiv. of DIPEA (8.938 mmol). After 30 minutes, the leucine solution was added dropwise to the [1]-containing solution and allowed to react for 18 hours. Upon completion, the solution was diluted in 150 mL of EtOAc and was washed with 1M HCl, then sat. NaHCO₃, and lastly brine. The organic layer was dried over Na2SO₄, filtered, and condensed. The remaining oil was dissolved in a minimal volume of DCM and dry-loaded onto silica gel for purification on ISCO CombiFlash (0-50% EtOAc in n-heptane). The fractions containing the desired product [2] were pooled, condensed in vacuo, and analyzed by LCMS. This resulted in 1.121g of [2] isolated (>98% purity; 87% yield) as a waxy solid.

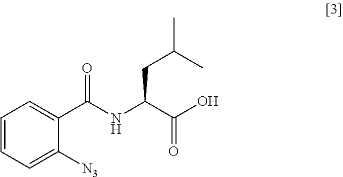 [SMILES: CC(C)C[C@H](NC(=O)c1ccccc1N=[N+]=[N-])C(=O)O]

Synthesis of N-(2-azidobenzamid)-L-leucine [3]: To a 200 mL round-bottom flask containing 1.121g of [2], a stirbar was added and the solid was dissolved in 40 mL of DCM. To this solution, 15 mL of trifluoroacetic acid (TFA) was carefully added and the solution was allowed to stir at room temperature for 5 hours. Upon completion (monitored by TLC), the stirbar was removed, washed with DCM and n-heptane, and the solution was condensed in vacuo. The remaining residue was washed with n-heptane and condensed in vacuo until most of the TFA was removed. The oil was dissolved in a minimal volume of DCM, dry-loaded onto silica gel, and purified on ISCO CombiFlash (0-70% EtOAc in n-heptane). The fractions containing the desired product [3] were pooled, condensed, and analyzed by LCMS. This produced 932 mg of [3] (>99% purity; 99% yield) as an amorphous solid.

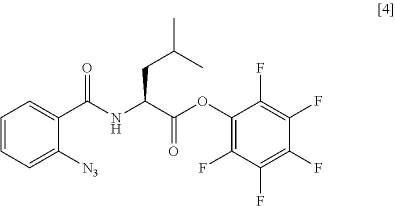 [SMILES: CC(C)C[C@H](NC(=O)c1ccccc1N=[N+]=[N-])C(=O)Oc1c(F)c(F)c(F)c(F)c1F]

Synthesis of N-(2-azidobenzamid)-L-leucine-O-(2,3,4,5,6-pentafluorophenyl) ester [4]: To a 20 mL amber vial equipped with a stirbar, 296 mg of [3] (0.890 mmol) was added and dissolved in 3 mL of anhydrous THF. To this, 1.1 equiv. 2,3,4,5,6-pentafluorophenol (0.980 mmol) was added and stirred until dissolved. In a separate vial, 1.0 equiv. of N,N′-dicyclohexylcarbodiimide (DCC; 0.890 mmol) was dissolved in THF and added dropwise to the stirred solution of [3]. The reaction was stirred at 25° C. for 3.5 hours and upon completion was diluted in EtOAc, filtered to remove DCU (dicyclohexylurea), and condensed in vacuo. The resulting oil was taken up in minimal volume of DCM and purified by ISCO CombiFlash (0-50% EtOAc in n-heptane). The resulting fractions containing the desired product were pooled, condensed, and placed under high vacuum to afford 392 mg of [4] as a waxy solid (>95% purity; 99% yield).

### Example 12. Development of Thermophilic Cleavase Enzymes for Removal of M-P1 from M-Modified Peptides

A genetic selection approach was used to evolve dipeptidyl peptidase enzyme to cleave a single labelled N-terminal amino acid from a peptide, essentially as described in patent applications WO 2020/198264 A1 and U.S. Pat. No. 17/213,169 A1. High diversity combinatorial libraries for different dipeptidyl peptidases were created and transformed into an E. coli selection strain. Structure-based design was used to define variant sites for library creation. Peptides with different N-terminally modified P1 amino acids were used to evolve Cleavase enzymes for the respective targets.

Briefly, a genetic selection-based approach to cleavase engineering enables high-throughput enzyme selection (Evnin, L. B., J. R. Vasquez and C. S. Craik (1990). “Substrate specificity of trypsin investigated by using a genetic selection.” Proc Natl Acad Sci U S A 87(17): 6659-6663). The selection makes use of short N-terminally modified peptides that contain the auxotrophic amino acid. The peptides readily enter the periplasm of a bacterium but are unable to enter the cytoplasm due to the inability of transporters to recognize the modified N-terminus (Smith, M. W., D. R. Tyreman, G. M. Payne, N. J. Marshall and J. W. Payne (1999).

“Substrate specificity of the periplasmic dipeptide-binding protein from Escherichia coli: experimental basis for the design of peptide prodrugs.” Microbiology 145 (Pt 10): 2891-2901). To relieve the amino acid auxotrophy during growth on minimal media, a cleavase scaffold is expressed on a plasmid and targeted to the periplasm, via a pelB leader sequence. The active cleavase variant removes the N-terminally modified amino acid, revealing a native peptide amino terminus. This allows the rest of the peptide, which contains the essential amino acid, to be uptaken and support growth of the bacterium. For the present studies, an arginine auxotroph was used which demonstrated an absence of background growth on peptides modified with different N-terminal modifications.

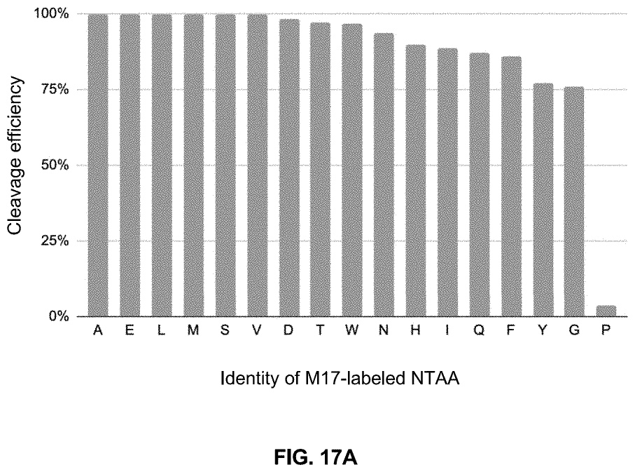

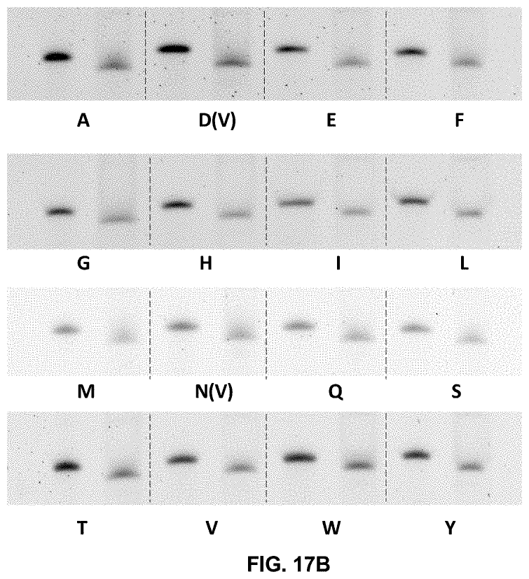

Using this genetic selection approach, active cleavase variants from an S46 DPP library {N₂₁₄X,W215X,R219X,N₃₂₉X,D673X; X =20 amino acids} and error prone library from Thermomonas hydrothermahs were identified with the following mutations: {N₂₁₄M,W215G,R219T,N₃₂₉R,D673A,G674V}. This variant was further evolved by creating an additional library with variant sites as follows {N₂₁₄M,W215G,R219T,N₃₂₉R; N₃₃₃X,I651X,A671X,D673A,G674X,N₆₈₂X,M692X; X=20 amino acids} and by genetic selection generated a set of enzymatic cleavases. Each evolved cleavase was individually assayed on all M-P1 targets. In this assay, individual cleavase clone was expressed and purified, and then incubated with each peptide substrate for 3 hours at 52° C. Six μM enzyme in 5 mM phosphate buffer at pH=8 were used. The UV absorbance of both product and starting material in the final reaction was measured on HPLC and converted to percentage of conversion (FIG. 17A). Collectively, they can provide broad activity for removal of almost all M-P1 residues (see FIG. 17A-B for exemplary cleavage of M17-modified NTAAs of a model polypeptide (M17-P1-AR, where P1 is one of the 17 natural amino acids, excluding C, K, R) with the Cleavase enzymes derived from the scaffold of SEQ ID NO: 4, where M17 is N-(2-azidobenzamid)-L-leucine). The enzymes can efficiently cleave M17-labeled polypeptides between P1 and P2 amino acid residues, thus are configured to remove a single modified terminal amino acid from the polypeptide (FIG. 17A and FIG. 17B). To accommodate the M17 label in the substrate binding site of dipeptidyl aminopeptidase, all modified cleavase enzymes contained the following mutations at the conserved residues that form an amine binding site in unmodified dipeptidyl aminopeptidases: N₂₁₄M, W215G, R219T, N₃₂₉R, D673A (the indicated residue numbers correspond to positions of SEQ ID NO: 4). The cleavage efficiency of the evolved enzymes depended on the nature of the P1 residue.

This set of M17-P1 active cleavases dovetails with the set of M17-P1 binders enabling the ProteoCode assay to perform stepwise peptide modification, encoding and M-P1 elimination, resulting in stepwise identification of terminal amino acid residues of the peptide, and eventually, the peptide sequencing and identification.

1. An engineered binder that specifically binds to an N-terminally modified target polypeptide modified by an N-terminal modifier agent, wherein:
(i) the N-terminally modified target polypeptide has a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;
(ii) the engineered binder specifically binds to the N-terminally modified target polypeptide through interaction between the engineered binder and the M-P1 of the N-terminally modified target polypeptide; and
(iii) the engineered binder comprises an amino acid sequence having at least about 80% sequence identity to the amino acid sequence selected from the group consisting of SEQ ID NO: 7—SEQ ID NO: 20.

2. The engineered binder of claim 1, wherein the N-terminal modification is a chemical entity having a volume from about 100 Å³ to about 1000 Å³.

3. The engineered binder of claim 1, wherein the N-terminal modifier agent is selected from the group consisting of compounds of the following formula: 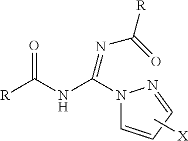
(A)
wherein R is CH₃, CF₃, OC(CH₃)₃, or OCH₂C₆H₅,
and X is H, CH₃, CF₃, CF₂H, or OCH₃; 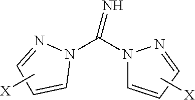
(B)
wherein X is H, CH₃, CF₃, CF₂H, OCH₃, or SO₂NH₂; 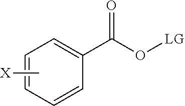
(C)
wherein X is H, F, Cl, OCH₃, OCF₃, CN, or SO₂NH₂,
and LG is succinimide, pentafluorophenyl, or tetrafluorophenyl; and 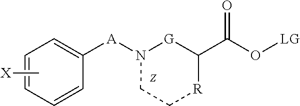
(D)
wherein X is H, F, Cl, NH₂, OCH₃, OCF₃, CN, or SO₂NH₂, A=CONH or SO₂, G=0 or 1 CH₂,
R is any amino acid or unnatural amino acid, and
Z ring=0 (not there), 1, 2, or 3 CH₂.

4. The engineered binder of claim 1, wherein the N-terminal modification (M) comprises an N-terminal blocking group and, optionally, a natural or unnatural amino acid moiety;
wherein the natural or unnatural amino acid moiety comprises a compound selected from the group consisting of: a naturally-occurring amino acid residue, 3-(3′-pyridyl)-L-alanine, L-cyclohexylglycine, α-aminoisobutyric acid, 3-(4′-pyridyl)-L-alanine, L-azetidine-2-carboxylic acid, isonipecotic acid, L-phenylglycine, β-(2-thienyl)-L-alanine, 3-(4-thiazolyl)-L-alanine, 1-aminocyclopentane-1-carboxylic acid, (2-trifluoromethyl)-L-Phenylalanine, L-cyclopropylalanine, 3-(2′-pyridyl)-L-alanine, beta-cyano-L-alanine, α-methyl-L-4-Fluorophenylalanine, α-methyl-D-4-fluorophenylalanine, 3-amino-2,2-difluoro-propionic acid, O-sulfo-L-tyrosine sodium salt, L-2-furylalanine, 1-aminocyclopropane-1-carboxylic acid, 3,5-dinitro-L-tyrosine, pentafluoro-L-phenylalanine, 3,5-difluoro-L-phenylalanine, 3-fluoro-L-phenylalanine, N-cyclopentylglycine, 1-(amino)cyclohexanecarboxylic acid, N-methylalanine, 4-amino-tetrahydropyran-4-carboxylic acid, 4-amino-1,1-dioxothiane-4-carboxylic acid, 4-amino-1-methyl-4-piperidinecarboxylic acid, 2-amino-N-(2,4-dimethoxybenzyl)acetamido)acetic acid, and a N-alkylated derivative thereof;
and the N-terminal blocking group comprises a compound selected from the group consisting of: 4-methylbenzoic acid, 4-(dimethylamio)benzoic acid, nicotinic acid, 3-aminonicotinic acid, 2-pyrazinecarbooxylic acid, 5-amino-2-fluoro-isonicotinic acid, 2,3-pyrazinedicarboxylic acid, 4,7-Difluoroisobenzofuran-1,3-dicarboxylic acid, 4-chloro-2-aminobenzoic acid, 4-nitro-2-aminobenzoic acid, 7-methoxy-1h-benzo[d][1,3]oxazine-2,4-dione, 4-carboxy-2-aminobenzoic acid, 6-(Trifluoromethyl)-2,4-dihydro-1h-3,1-benzoxazine-2,4-dione, 7-(Trifluoromethyl)-1h-benzo[d][1,3]oxazine-2,4-dione, 6-fluoro-2-aminobenzoic acid, 4-fluoro-2-aminobenzoic acid, 5-methoxy-2-aminobenzoic acid, 4-fluorobenzoic acid, 4-(trifluoromethyl)benzoic acid, 2-ethynyl fluorobenzaldehyde, 2-aminobenzoic acid, Succinic anhydride, 3,6-Difluoropyridine-2-carboxylic acid, 2-Fluoronicotinic acid, 5-Bromo-2-hydroxynicotinic acid, 4-(Trifluoromethyl)pyrimidine carboxylic acid, 2-Oxo-1,2-dihydropyridine-3-carboxylic acid, 5-Methyl-2-aminobenzoic acid, 6-Fluoropicolinic acid, 3-Methyl-2-aminobenzoic acid, 4-Methyl-2-aminobenzoic acid, 2-Amino methylbenzoic acid, 2-Amino-6-fluorobenzoic acid, 2-Amino-5-fluorobenzoic acid, 2-Amino-3-fluorobenzoic acid, 2-Amino-4-fluorobenzoic acid, 2-Aminonicotinic acid, 4-Aminonicotinic acid, 3-Aminopicolinic acid, 2-Amino-4,5-difluorobenzoic acid, 3,4-difluorobenzoic acid, 3,4,5-difluorobenzoic acid, 3-(Methoxycarbonyl)bicyclo[1.1.1]pentane-1-carboxylic acid, 3,3-Difluorocyclobutane-1-carboxylic acid, 1-Methyl-2-oxo-piperidine-4-carboxylic acid, Tetrahydropyran-4-carboxylic acid, 5-Fluoroorotic acid, 3-Fluoro-4-nitrobenzoic acid, 3-(Difluoromethyl)-1-methyl-1H-pyrazole-4-carboxylic acid, 4-(Difluoromethoxy)benzoic acid, 1-(Difluoromethyl)-1h-pyrazole-3-carboxylic acid, 4-(Methanesulfonylamino)benzoic acid, 5-Fluoro-6-methoxynicotinic acid, Tetrahydro-2H-thiopyran-4-carboxylic acid 1,1-dioxide, 4-(1H-Tetrazol-5-yl)benzoic acid, 1,2,3-Thiadiazole-4-carboxylic acid, 1,3-Benzodioxole-4-carboxylic acid, 2,1,3-Benzoxadiazole-5-carboxylic acid, 1-Benzyl-3-methyl-1h-pyrazole-5-carboxylic acid, 1-Cyclopropyl-6,7-difluoro-1,4-dihydro-4-oxoquinoline-3-carboxylic acid, 3,4-Dichlorobenzoic acid, 5-Fluoro-6-methylpyridine-2-carboxylic acid, 4,5-Dimethyl-2-(1h-pyrrol-1-yl)thiophene-3-carboxylic acid, 1,3-Dimethyl-1h-thieno[2,3-c]pyrazole-5-carboxylic acid, 1-[(4-Fluorobenzene)sulfonyl]piperidine-3-carboxylic acid, 1-(4-Fluorobenzyl)-5-oxopyrrolidine-3-carboxylic acid, 3-Fluoro-4-methoxybenzoic acid, 4-Fluoro-3-nitrobenzoic acid, 6-Fluoro-4-oxochromene-2-carboxylic acid, 3-Fluorophenylacetic acid, 4-Fluoro-3-(trifluoromethyl)benzoic acid, 5-Furan-2-yl-isoxazole-3-carboxylic acid, 1-Isopropyl-2-(trifluoromethyl)-1h-benzimidazole-5-carboxylic acid, Levofloxacin carboxylic acid, 3,5,7-Trifluoroadamantane-1-carboxylic acid, 3,4,5-Trimethoxybenzoic acid, 2-Oxo-2,3-dihydro-1h-benzo[d]imidazole-4-carboxylic acid, 1-Methyl-3-(trifluoromethyl)-1h-pyrazole-5-carboxylic acid, 2-Morpholin-4-yl-isonicotinic acid, 1,3-Oxazole-4-carboxylic acid, 4-Carboxybenzenesulfonamide, and 3,4-difluorobenzenesulfonyl chloride.

5. (canceled)

6. The engineered binder of claim 2 having a substrate binding pocket with a volume from about 200 Å³ to about 2000 Å³.

7. The engineered binder of claim 1, wherein the engineered binder comprises an amino acid sequence having at least about 89% sequence identity to the amino acid sequence selected from the group consisting of SEQ ID NO: 7—SEQ ID NO: 20.

8. The engineered binder of claim 1, wherein binding specificity between the engineered binder and the N-terminally modified target polypeptide is predominantly or substantially determined by interaction between the engineered binder and the M-P1 of the N-terminally modified target polypeptide.

9. The engineered binder of claim 8, wherein the engineered binder is capable of specifically binding to each N-terminally modified target polypeptide from a plurality of N-terminally modified target polypeptides, wherein the plurality of N-terminally modified target polypeptides comprises at least 10 N-terminally modified target polypeptides that are modified with the same N-terminal modifier agent, have the same modified NTAA residue, and have different P2 residues.

10. The engineered binder of claim 1, having an affinity towards an N-terminally modified target polypeptide comprising a specific P1 residue that is at least 2-fold higher as compared to affinity of the engineered binder towards an otherwise identical N-terminally modified target polypeptide but comprising a different P1 residue.

11. The engineered binder of claim 1, wherein the N-terminally modified target polypeptide is immobilized on a solid support.

12. The engineered binder of claim 1, further comprising a detectable label or a nucleic acid tag.

13. A set of engineered binders, comprising at least two engineered binders, wherein:
each engineered binder from the set of engineered binders is configured to specifically bind to an N-terminally modified target polypeptide modified with an N-terminal modifier agent and having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;
(ii) each engineered binder from the set of engineered binders is configured to specifically bind to the N-terminally modified target polypeptide through interaction between the engineered binder and the modified NTAA residue of the N-terminally modified target polypeptide, wherein engineered binders from the set of engineered binders are configured to specifically bind to different modified NTAA residues of target polypeptides modified with the same or different N-terminal modifier agents; and
at least one engineered binder from the set of engineered binders comprises an amino acid sequence having at least about 80% sequence identity to the amino acid sequence selected from the group consisting of SEQ ID NO: 7—SEQ ID NO: 20.

14. The set of engineered binders of claim 13, wherein at least one engineered binder from the set of engineered binders comprises an amino acid sequence having at least about 89% sequence identity to the amino acid sequence selected from the group consisting of SEQ ID NO: 7—SEQ ID NO: 20.

15. (canceled)

16. The set of engineered binders of claim 13, which further comprises least one engineered binder from the set of engineered binders comprising an amino acid sequence having at least about 80% sequence identity to the amino acid sequence set forth in SEQ ID NO: 63.

17. The set of engineered binders of claim 13, wherein each engineered binder from the set of engineered binders is configured to specifically bind to each N-terminally modified target polypeptide from a plurality of N-terminally modified target polypeptides, wherein the plurality of N-terminally modified target polypeptides comprises at least 10 N-terminally modified target polypeptides that are modified with the same N-terminal modifier agent, have the same modified NTAA residue, and have different P2 residues.

18. (canceled)

19. The set of engineered binders of claim 13, wherein each engineered binder from the set of engineered binders comprises a detectable label or a nucleic acid tag.

20. A method of treating a target polypeptide, the method comprises:
(i) contacting a target polypeptide with an N-terminal modifier agent to form an N-terminally modified polypeptide having a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide; and contacting an engineered binder with the N-terminally modified target polypeptide to allow the engineered binder to specifically bind to the N-terminally modified target polypeptide through interaction between the engineered binder and the modified NTAA residue of the N-terminally modified target polypeptide, wherein the engineered binder comprises an amino acid sequence having at least about 80% sequence identity to the amino acid sequence selected from the group consisting of SEQ ID NO: 7—SEQ ID NO: 20.

21. (canceled)

22. The method of claim 20, wherein the N-terminal modifier agent is selected from the group consisting of compounds of the following formula: 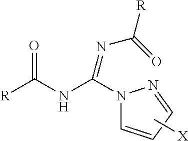
(A)
wherein R is CH₃, CF₃, OC(CH₃)₃, or OCH₂C₆H₅,
and X is H, CH₃, CF₃, CF₂H, or OCH₃; 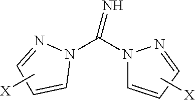
(B)
wherein X is H, CH₃, CF₃, CF₂H, OCH₃, or SO₂NH₂; 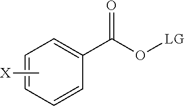
(C)
wherein X is H, F, Cl, OCH₃, OCF₃, CN, or SO₂NH₂,
and LG is succinimide, pentafluorophenyl, or tetrafluorophenyl; and 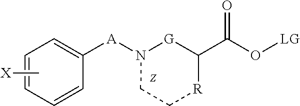
(D)
wherein X is H, F, Cl, NH₂, OCH₃, OCF₃, CN, or SO₂NH₂, A=CONH or SO₂, G=0 or 1 CH₂,
R is any amino acid or unnatural amino acid moiety, and
Z ring=0 (not there), 1, 2, or 3 CH₂.

23-24. (canceled)

25. The method of claim 20, further comprising immobilizing the target polypeptide on a solid support before step (i).

26. The method of claim 22, wherein the N-terminal modifier agent further comprises a peptide coupling reagent.

27. (canceled)

28. An isolated nucleic acid molecule comprising a polynucleotide having a sequence encoding the engineered binder of claim 1.
